SEQUENCE LISTING (292 sequences)

SEQ ID NO: 1 - protein, 126 aa, Synthetic construct
  source 1..126: note = PAD2 VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQVLVRGFFSHEDDAVDIWGQGTTVTVSS

SEQ ID NO: 2 - protein, 106 aa, Synthetic construct
  source 1..106: note = PAD2 VL
SYVLTQPPSVSVSPGQTASITCSGDKVGDKYVSWYQQKPGQSPVLVIYQDSQRPSGIPERFSGSNSGNTATLTISGTQAMDEADYYCQTWAPDVLLFGSGTKVTVL

SEQ ID NO: 3 - protein, 5 aa, Synthetic construct
  source 1..5: note = PAD2 HCDR1 (Kabat) AA
SYAMS

SEQ ID NO: 4 - protein, 17 aa, Synthetic construct
  source 1..17: note = PAD2 HCDR2 (Kabat) AA
AISGSGGSTYYADSVKG

SEQ ID NO: 5 - protein, 17 aa, Synthetic construct
  source 1..17: note = PAD2 HCDR3 (Kabat) AA
QVLVRGFFSHEDDAVDI

SEQ ID NO: 6 - protein, 30 aa, Synthetic construct
  source 1..30: note = PAD2 HFW1 (Kabat) AA
EVQLLESGGGLVQPGGSLRLSCAASGFTFS

SEQ ID NO: 7 - protein, 14 aa, Synthetic construct
  source 1..14: note = PAD2 HFW2 (Kabat) AA
WVRQAPGKGLEWVS

SEQ ID NO: 8 - protein, 32 aa, Synthetic construct
  source 1..32: note = PAD2 HFW3 (Kabat) AA
RFTISRDNSKNTLYLQMNSLRAEDTAVYYCAR

SEQ ID NO: 9 - protein, 11 aa, Synthetic construct
  source 1..11: note = PAD2 HFW4 (Kabat) AA
WGQGTTVTVSS

SEQ ID NO: 10 - protein, 11 aa, Synthetic construct
  source 1..11: note = PAD2 LCDR1 (Kabat) A
SGDKVGDKYVS

SEQ ID NO: 11 - protein, 7 aa, Synthetic construct
  source 1..7: note = PAD2 LCDR2 (Kabat) AA
QDSQRPS

SEQ ID NO: 12 - protein, 9 aa, Synthetic construct
  source 1..9: note = PAD2 LCDR3 (Kabat) AA
QTWAPDVLL

SEQ ID NO: 13 - protein, 22 aa, Synthetic construct
  source 1..22: note = PAD2 LFW1 (Kabat) AA
SYVLTQPPSVSVSPGQTASITC

SEQ ID NO: 14 - protein, 15 aa, Synthetic construct
  source 1..15: note = PAD2 LFW2 (Kabat) AA
WYQQKPGQSPVLVIY

SEQ ID NO: 15 - protein, 32 aa, Synthetic construct
  source 1..32: note = PAD2 LFW3 (Kabat) AA
GIPERFSGSNSGNTATLTISGTQAMDEADYYC

SEQ ID NO: 16 - protein, 10 aa, Synthetic construct
  source 1..10: note = PAD2 LFW4 (Kabat) AA
FGSGTKVTVL

SEQ ID NO: 17 - protein, 5 aa, Synthetic construct
  source 1..5: note = PAD4 HCDR1 (Kabat) AA
DYFVS

SEQ ID NO: 18 - protein, 17 aa, Synthetic construct
  source 1..17: note = PAD4 HCDR2 (Kabat) AA
FINAANTFTYYADSVRG

SEQ ID NO: 19 - protein, 19 aa, Synthetic construct
  source 1..19: note = PAD4 HCDR3 (Kabat) AA
ANDDVDDIVAPGRGYYMDV

SEQ ID NO: 20 - protein, 30 aa, Synthetic construct
  source 1..30: note = PAD4 HFW1 (Kabat) AA
QVQLVESGGGLVKPGGSLRLSCAASGSTLS

SEQ ID NO: 21 - protein, 14 aa, Synthetic construct
  source 1..14: note = PAD4 HFW2 (Kabat) AA
WIRQAPGKGLEWVS

SEQ ID NO: 22 - protein, 32 aa, Synthetic construct
  source 1..32: note = HFW3 (Kabat) AA
RFTISRDNAKNSVYLQMNSLRAEDTAVYYCSS

SEQ ID NO: 23 - protein, 11 aa, Synthetic construct
  source 1..11: note = PAD4 HFW4 (Kabat) AA
WGRGTLVTVSS

SEQ ID NO: 24 - protein, 14 aa, Synthetic construct
  source 1..14: note = PAD4 LCDR1 (Kabat) AA
TGTSGDVGRYSHVS

SEQ ID NO: 25 - protein, 7 aa, Synthetic construct
  source 1..7: note = PAD4 LCDR2 (Kabat) AA
NVYERPS

SEQ ID NO: 26 - protein, 11 aa, Synthetic construct
  source 1..11: note = PAD4 LCDR3 (Kabat) AA
SSHSRSSTPVL

SEQ ID NO: 27 - protein, 22 aa, Synthetic construct
  source 1..22: note = PAD4 LFW1 (Kabat) AA
QSALTQPRSVSGSPGQSVTISC

SEQ ID NO: 28 - protein, 15 aa, Synthetic construct
WYQQHPGKAPKLIIY

SEQ ID NO: 29 - protein, 32 aa, Synthetic construct
  source 1..32: note = LFW3 (Kabat) AA
GVPDRFSGSKSGNTASLTISGLQAEDEADYYC

SEQ ID NO: 30 - protein, 10 aa, Synthetic construct
  source 1..10: note = PAD4 LFW4 (Kabat) AA
FGGGTKLTVL

SEQ ID NO: 31 - protein, 128 aa, Synthetic construct
  source 1..128: note = PAD4 VH
QVQLVESGGGLVKPGGSLRLSCAASGSTLSDYFVSWIRQAPGKGLEWVSFINAANTFTYYADSVRGRFTISRDNAKNSVYLQMNSLRAEDTAVYYCSSANDDVDDIVAPGRGYYMDVWGRGTLVTVSS

SEQ ID NO: 32 - protein, 111 aa, Synthetic construct
  source 1..111: note = VL (Kabat) AA
QSALTQPRSVSGSPGQSVTISCTGTSGDVGRYSHVSWYQQHPGKAPKLIIYNVYERPSGVPDRFSGSKSGNTASLTISGLQAEDEADYYCSSHSRSSTPVLFGGGTKLTVL

SEQ ID NO: 33 - protein, 20 aa, Synthetic construct
  source 1..20: note = scFv VH-VL Linker
GGGGSGGGGSGGGGSGGGGS

SEQ ID NO: 34 - protein, 224 aa, Synthetic construct
  source 1..224: note = PAD2 Fab HC (VH-CH1) Clones 08, 10, 12
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQVLVRGFFSHEDDAVDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 35 - protein, 212 aa, Synthetic construct
  source 1..212: note = PAD2 Fab LC (VL-CL) Clones 08, 10, 12
SYVLTQPPSVSVSPGQTASITCSGDKVGDKYVSWYQQKPGQSPVLVIYQDSQRPSGIPERFSGSNSGNTATLTISGTQAMDEADYYCQTWAPDVLLFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 36 - protein, 106 aa, Synthetic construct
  source 1..106: note = Lambda CL Clones 07-12
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 37 - protein, 98 aa, Synthetic construct
  source 1..98: note = CH Clones 07-12
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 38 - protein, 252 aa, Synthetic construct
  source 1..252: note = PAD2 scFv ((VL-VH with linker (G4S)4) Clones 07, 09, 11
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQVLVRGFFSHEDDAVDIWGQGTTVTVSSGGGGSGGGGSGGGGSGGGGSSYVLTQPPSVSVSPGQTASITCSGDKVGDKYVSWYQQKPGQSPVLVIYQDSQRPSGIPERFSGSNSGNTATLTISGTQAMDEADYYCQTWAPDVLLFGSGTKVTVL

SEQ ID NO: 39 - protein, 259 aa, Synthetic construct
  source 1..259: note = PAD4 scFv (VH-VL with (G4S)4 Clones 08, 10, 12
QVQLVESGGGLVKPGGSLRLSCAASGSTLSDYFVSWIRQAPGKGLEWVSFINAANTFTYYADSVRGRFTISRDNAKNSVYLQMNSLRAEDTAVYYCSSANDDVDDIVAPGRGYYMDVWGRGTLVTVSSGGGGSGGGGSGGGGSGGGGSQSALTQPRSVSGSPGQSVTISCTGTSGDVGRYSHVSWYQQHPGKAPKLIIYNVYERPSGVPDRFSGSKSGNTASLTISGLQAEDEADYYCSSHSRSSTPVLFGGGTKLTVL

SEQ ID NO: 40 - protein, 226 aa, Synthetic construct
  source 1..226: note = PAD4 Fab HC (VH-CH1) Clones 07, 09, 11
QVQLVESGGGLVKPGGSLRLSCAASGSTLSDYFVSWIRQAPGKGLEWVSFINAANTFTYYADSVRGRFTISRDNAKNSVYLQMNSLRAEDTAVYYCSSANDDVDDIVAPGRGYYMDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 41 - protein, 217 aa, Synthetic construct
  source 1..217: note = PAD4 Fab LC (VL-CL) Clones 07, 09, 11
QSALTQPRSVSGSPGQSVTISCTGTSGDVGRYSHVSWYQQHPGKAPKLIIYNVYERPSGVPDRFSGSKSGNTASLTISGLQAEDEADYYCSSHSRSSTPVLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 42 - protein, 725 aa, Synthetic construct
  source 1..725: note = Bis3 full heavy chain sequence FQQ-YTE (VH-CH1-CH2-CH3) Clone 12
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQVLVRGFFSHEDDAVDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLVESGGGLVKPGGSLRLSCAASGSTLSDYFVSWIRQAPGKGLEWVSFINAANTFTYYADSVRGRFTISRDNAKNSVYLQMNSLRAEDTAVYYCSSANDDVDDIVAPGRGYYMDVWGRGTLVTVSSGGGGSGGGGSGGGGSGGGGSQSALTQPRSVSGSPGQSVTISCTGTSGDVGRYSHVSWYQQHPGKAPKLIIYNVYERPSGVPDRFSGSKSGNTASLTISGLQAEDEADYYCSSHSRSSTPVLFGGGTKLTVL

SEQ ID NO: 43 - protein, 330 aa, Synthetic construct
  source 1..330: note = Bis3 IgG1 FQQ-YTE Fc Clone 07
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - skipped

SEQ ID NO: 45 - protein, 15 aa, Synthetic construct
  source 1..15: note = Bis3 IgG1 Hinge Clones 07-12
EPKSCDKTHTCPPCP

SEQ ID NO: 46 - protein, 110 aa, Synthetic construct
  source 1..110: note = Bis3 IgG CH2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 47 - protein, 110 aa, Synthetic construct
  source 1..110: note = Bis3 IgG1 TM CH2 Clones 07 and 08
APEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAK

SEQ ID NO: 48 - protein, 110 aa, Synthetic construct
  source 1..110: note = Bis3 IgG1 TM-YTE CH2 Clones 09 and 10
APEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAK

SEQ ID NO: 49 - protein, 110 aa, Synthetic construct
  source 1..110: note = Bis3 IgG1 FQQ-YTE CH2 Clones 11 and 12
APEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAK

SEQ ID NO: 50 - protein, 107 aa, Synthetic construct
  source 1..107: note = Bis3 IgG1 CH3 Clone 07-12
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 10 aa, Synthetic construct
  source 1..10: note = Fc-scFv Linker Clones 07-12
GGGGSGGGGS

SEQ ID NO: 52 - skipped

SEQ ID NO: 53 - protein, 937 aa, Synthetic construct
  source 1..937: note = Bis3 TM Clone 07
QSALTQPRSVSGSPGQSVTISCTGTSGDVGRYSHVSWYQQHPGKAPKLIIYNVYERPSGVPDRFSGSKSGNTASLTISGLQAEDEADYYCSSHSRSSTPVLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSQVQLVESGGGLVKPGGSLRLSCAASGSTLSDYFVSWIRQAPGKGLEWVSFINAANTFTYYADSVRGRFTISRDNAKNSVYLQMNSLRAEDTAVYYCSSANDDVDDIVAPGRGYYMDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQVLVRGFFSHEDDAVDIWGQGTTVTVSSGGGGSGGGGSGGGGSGGGGSSYVLTQPPSVSVSPGQTASITCSGDKVGDKYVSWYQQKPGQSPVLVIYQDSQRPSGIPERFSGSNSGNTATLTISGTQAMDEADYYCQTWAPDVLLFGSGTKVTVL

SEQ ID NO: 54 - protein, 937 aa, Synthetic construct
  source 1..937: note = Bis3 TM-YTE Clone 09
QSALTQPRSVSGSPGQSVTISCTGTSGDVGRYSHVSWYQQHPGKAPKLIIYNVYERPSGVPDRFSGSKSGNTASLTISGLQAEDEADYYCSSHSRSSTPVLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSQVQLVESGGGLVKPGGSLRLSCAASGSTLSDYFVSWIRQAPGKGLEWVSFINAANTFTYYADSVRGRFTISRDNAKNSVYLQMNSLRAEDTAVYYCSSANDDVDDIVAPGRGYYMDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQVLVRGFFSHEDDAVDIWGQGTTVTVSSGGGGSGGGGSGGGGSGGGGSSYVLTQPPSVSVSPGQTASITCSGDKVGDKYVSWYQQKPGQSPVLVIYQDSQRPSGIPERFSGSNSGNTATLTISGTQAMDEADYYCQTWAPDVLLFGSGTKVTVL

SEQ ID NO: 55 - protein, 937 aa, Synthetic construct
  source 1..937: note = Bis3 FQQ YTE Clone 11
QSALTQPRSVSGSPGQSVTISCTGTSGDVGRYSHVSWYQQHPGKAPKLIIYNVYERPSGVPDRFSGSKSGNTASLTISGLQAEDEADYYCSSHSRSSTPVLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSQVQLVESGGGLVKPGGSLRLSCAASGSTLSDYFVSWIRQAPGKGLEWVSFINAANTFTYYADSVRGRFTISRDNAKNSVYLQMNSLRAEDTAVYYCSSANDDVDDIVAPGRGYYMDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQVLVRGFFSHEDDAVDIWGQGTTVTVSSGGGGSGGGGSGGGGSGGGGSSYVLTQPPSVSVSPGQTASITCSGDKVGDKYVSWYQQKPGQSPVLVIYQDSQRPSGIPERFSGSNSGNTATLTISGTQAMDEADYYCQTWAPDVLLFGSGTKVTVL

SEQ ID NO: 56 - protein, 937 aa, Synthetic construct
  source 1..937: note = Bis3 TM Clone 08
SYVLTQPPSVSVSPGQTASITCSGDKVGDKYVSWYQQKPGQSPVLVIYQDSQRPSGIPERFSGSNSGNTATLTISGTQAMDEADYYCQTWAPDVLLFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQVLVRGFFSHEDDAVDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLVESGGGLVKPGGSLRLSCAASGSTLSDYFVSWIRQAPGKGLEWVSFINAANTFTYYADSVRGRFTISRDNAKNSVYLQMNSLRAEDTAVYYCSSANDDVDDIVAPGRGYYMDVWGRGTLVTVSSGGGGSGGGGSGGGGSGGGGSQSALTQPRSVSGSPGQSVTISCTGTSGDVGRYSHVSWYQQHPGKAPKLIIYNVYERPSGVPDRFSGSKSGNTASLTISGLQAEDEADYYCSSHSRSSTPVLFGGGTKLTVL

SEQ ID NO: 57 - protein, 937 aa, Synthetic construct
  source 1..937: note = Bis3 TM YTE Clone 10
SYVLTQPPSVSVSPGQTASITCSGDKVGDKYVSWYQQKPGQSPVLVIYQDSQRPSGIPERFSGSNSGNTATLTISGTQAMDEADYYCQTWAPDVLLFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQVLVRGFFSHEDDAVDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLVESGGGLVKPGGSLRLSCAASGSTLSDYFVSWIRQAPGKGLEWVSFINAANTFTYYADSVRGRFTISRDNAKNSVYLQMNSLRAEDTAVYYCSSANDDVDDIVAPGRGYYMDVWGRGTLVTVSSGGGGSGGGGSGGGGSGGGGSQSALTQPRSVSGSPGQSVTISCTGTSGDVGRYSHVSWYQQHPGKAPKLIIYNVYERPSGVPDRFSGSKSGNTASLTISGLQAEDEADYYCSSHSRSSTPVLFGGGTKLTVL

SEQ ID NO: 58 - protein, 937 aa, Synthetic construct
  source 1..937: note = Bis3 FQQ YTE Clone 12
SYVLTQPPSVSVSPGQTASITCSGDKVGDKYVSWYQQKPGQSPVLVIYQDSQRPSGIPERFSGSNSGNTATLTISGTQAMDEADYYCQTWAPDVLLFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQVLVRGFFSHEDDAVDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLVESGGGLVKPGGSLRLSCAASGSTLSDYFVSWIRQAPGKGLEWVSFINAANTFTYYADSVRGRFTISRDNAKNSVYLQMNSLRAEDTAVYYCSSANDDVDDIVAPGRGYYMDVWGRGTLVTVSSGGGGSGGGGSGGGGSGGGGSQSALTQPRSVSGSPGQSVTISCTGTSGDVGRYSHVSWYQQHPGKAPKLIIYNVYERPSGVPDRFSGSKSGNTASLTISGLQAEDEADYYCSSHSRSSTPVLFGGGTKLTVL

SEQ ID NO: 59 - protein, 456 aa, Synthetic construct
  source 1..456: note = PAD2 full heavy chain - 'hole' arm (without RF mutation) FQQ-YTE Clone 06
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQVLVRGFFSHEDDAVDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 60 - protein, 456 aa, Synthetic construct
  source 1..456: note = Clone 06 PAD2 full heavy chain - 'hole' arm (with RF mutation) (VH-CH1-CH2-CH3) FQQ-YTE Clone 06
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQVLVRGFFSHEDDAVDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 61 - protein, 458 aa, Synthetic construct
  source 1..458: note = PAD4 full heavy chain - 'knob' arm (VH-CH1-CH2-CH3) FQQ-YTE Clone 06
QVQLVESGGGLVKPGGSLRLSCAASGSTLSDYFVSWIRQAPGKGLEWVSFINAANTFTYYADSVRGRFTISRDNAKNSVYLQMNSLRAEDTAVYYCSSANDDVDDIVAPGRGYYMDVWGRGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 213 aa, Synthetic construct
  source 1..213: note = PAD2 full kappa light chain (VL-CL) Clone 02, 04, 06
SYVLTQPPSVSVSPGQTASITCSGDKVGDKYVSWYQQKPGQSPVLVIYQDSQRPSGIPERFSGSNSGNTATLTISGTQAMDEADYYCQTWAPDVLLFGSGTKVTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 218 aa, Synthetic construct
  source 1..218: note = PAD4 full kappa light chain (VL-CL) Clone 01, 03, 05
QSALTQPRSVSGSPGQSVTISCTGTSGDVGRYSHVSWYQQHPGKAPKLIIYNVYERPSGVPDRFSGSKSGNTASLTISGLQAEDEADYYCSSHSRSSTPVLFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 212 aa, Synthetic construct
  source 1..212: note = PAD2 full lambda light chain (VL-CL) Clone 01, 03, 05
SYVLTQPPSVSVSPGQTASITCSGDKVGDKYVSWYQQKPGQSPVLVIYQDSQRPSGIPERFSGSNSGNTATLTISGTQAMDEADYYCQTWAPDVLLFGSGTKVTVLGQPKLAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 65 - protein, 217 aa, Synthetic construct
  source 1..217: note = PAD4 full lambda light (VL-CL) Clone 02, 04, 06
QSALTQPRSVSGSPGQSVTISCTGTSGDVGRYSHVSWYQQHPGKAPKLIIYNVYERPSGVPDRFSGSKSGNTASLTISGLQAEDEADYYCSSHSRSSTPVLFGGGTKLTVLGQPKLAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 66 - protein, 330 aa, Synthetic construct
  source 1..330: note = PAD2 FQQ-YTE 'hole' Fc with RF (CH1-CH2-CH3) Clone 06
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 67 - protein, 458 aa, Synthetic construct
  source 1..458: note = PAD4 HC hole TM (VH- CH1-CH2-CH3) Clone 01
QVQLVESGGGLVKPGGSLRLSCAASGSTLSDYFVSWIRQAPGKGLEWVSFINAANTFTYYADSVRGRFTISRDNAKNSVYLQMNSLRAEDTAVYYCSSANDDVDDIVAPGRGYYMDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 68 - protein, 458 aa, Synthetic construct
  source 1..458: note = PAD4 HC hole TM YTE (VH- CH1-CH2-CH3) Clone 03
QVQLVESGGGLVKPGGSLRLSCAASGSTLSDYFVSWIRQAPGKGLEWVSFINAANTFTYYADSVRGRFTISRDNAKNSVYLQMNSLRAEDTAVYYCSSANDDVDDIVAPGRGYYMDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 69 - protein, 458 aa, Synthetic construct
  source 1..458: note = PAD4 HC hole RF FQQ YTE (VH- CH1-CH2-CH3) Clone 05
QVQLVESGGGLVKPGGSLRLSCAASGSTLSDYFVSWIRQAPGKGLEWVSFINAANTFTYYADSVRGRFTISRDNAKNSVYLQMNSLRAEDTAVYYCSSANDDVDDIVAPGRGYYMDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 70 - protein, 456 aa, Synthetic construct
  source 1..456: note = PAD2 HC knob TM (VH- CH1-CH2-CH3) Clone 01
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQVLVRGFFSHEDDAVDIWGQGTTVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 456 aa, Synthetic construct
  source 1..456: note = PAD2 HC knob TM YTE (VH- CH1-CH2-CH3) Clone 03
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQVLVRGFFSHEDDAVDIWGQGTTVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 456 aa, Synthetic construct
  source 1..456: note = PAD2 HC knob FQQ YTE (VH- CH1-CH2-CH3) Clone 05
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQVLVRGFFSHEDDAVDIWGQGTTVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 456 aa, Synthetic construct
  source 1..456: note = PAD2 HC hole RF TM (VH- CH1-CH2-CH3) Clone 02
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQVLVRGFFSHEDDAVDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 74 - protein, 456 aa, Synthetic construct
  source 1..456: note = PAD2 HC hole TM YTE (VH- CH1-CH2-CH3) Clone 04
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQVLVRGFFSHEDDAVDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 75 - protein, 19 aa, Synthetic construct
  source 1..19: note = HCDR3 (Kabat) AA
ATENVDDIVAPGRGYYMDV

SEQ ID NO: 76 - protein, 458 aa, Synthetic construct
  source 1..458: note = PAD4 HC knob TM (VH- CH1-CH2-CH3) Clone 02
QVQLVESGGGLVKPGGSLRLSCAASGSTLSDYFVSWIRQAPGKGLEWVSFINAANTFTYYADSVRGRFTISRDNAKNSVYLQMNSLRAEDTAVYYCSSANDDVDDIVAPGRGYYMDVWGRGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 458 aa, Synthetic construct
  source 1..458: note = PAD4 HC knob TM YTE (VH- CH1-CH2-CH3) Clone 04
QVQLVESGGGLVKPGGSLRLSCAASGSTLSDYFVSWIRQAPGKGLEWVSFINAANTFTYYADSVRGRFTISRDNAKNSVYLQMNSLRAEDTAVYYCSSANDDVDDIVAPGRGYYMDVWGRGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - skipped

SEQ ID NO: 79 - protein, 106 aa, Synthetic construct
  source 1..106: note = VL (Kabat) AA
SYVLTQPPSVSVSPGQTASITCSGDKVGDKYVSWYQQKPGQSPVLVIYQDSQRPSGIPERFSGSNSGNTATLTISGTQAMDEADYYCQTWYDDALTFGSGTKVTVL

SEQ ID NO: 80 - protein, 340 aa, Synthetic construct
  source 1..340: note = R347 CH1-CH2-CH3 FQQ-YTE
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGS

SEQ ID NO: 81 - protein, 110 aa, Synthetic construct
  source 1..110: note = R347 VL
ELVLTQPASVSGSPGQSITISCTGTSSDVGGYNYVSWYQQHPGKAPKLMIYDVSKRPSGVSNRFSGSKSGNTASLTISGLQAEDEADYYCSSYTSSSTLVFGGGTKLTVL

SEQ ID NO: 82 - protein, 120 aa, Synthetic construct
  source 1..120: note = R347 VH
EVQLLESGGGLVQPGGSLRLSCTTSGFTFNTYAMSWVRQAPGKGLEWLSGINNNGRTAFYADSVKGRFTISRDNSKNTLYLQINSLRADDTAVYFCAKDVRFIAVPGDSWGQGTLVTVSS

SEQ ID NO: 83 - protein, 250 aa, Synthetic construct
  source 1..250: note = R347 scFv (VH-VL with (G4S)4)
EVQLLESGGGLVQPGGSLRLSCTTSGFTFNTYAMSWVRQAPGKGLEWLSGINNNGRTAFYADSVKGRFTISRDNSKNTLYLQINSLRADDTAVYFCAKDVRFIAVPGDSWGQGTLVTVSSGGGGSGGGGSGGGGSGGGGSELVLTQPASVSGSPGQSITISCTGTSSDVGGYNYVSWYQQHPGKAPKLMIYDVSKRPSGVSNRFSGSKSGNTASLTISGLQAEDEADYYCSSYTSSSTLVFGGGTKLTVL

SEQ ID NO: 84 - protein, 926 aa, Synthetic construct
  source 1..926: note = R347 Bis3 FQQ-YTE
ELVLTQPASVSGSPGQSITISCTGTSSDVGGYNYVSWYQQHPGKAPKLMIYDVSKRPSGVSNRFSGSKSGNTASLTISGLQAEDEADYYCSSYTSSSTLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSEVQLLESGGGLVQPGGSLRLSCTTSGFTFNTYAMSWVRQAPGKGLEWLSGINNNGRTAFYADSVKGRFTISRDNSKNTLYLQINSLRADDTAVYFCAKDVRFIAVPGDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSEVQLLESGGGLVQPGGSLRLSCTTSGFTFNTYAMSWVRQAPGKGLEWLSGINNNGRTAFYADSVKGRFTISRDNSKNTLYLQINSLRADDTAVYFCAKDVRFIAVPGDSWGQGTLVTVSSGGGGSGGGGSGGGGSGGGGSELVLTQPASVSGSPGQSITISCTGTSSDVGGYNYVSWYQQHPGKAPKLMIYDVSKRPSGVSNRFSGSKSGNTASLTISGLQAEDEADYYCSSYTSSSTLVFGGGTKLTVL

SEQ ID NO: 85 - protein, 450 aa, Synthetic construct
  source 1..450: note = R347 hole heavy chain
EVQLLESGGGLVQPGGSLRLSCTTSGFTFNTYAMSWVRQAPGKGLEWLSGINNNGRTAFYADSVKGRFTISRDNSKNTLYLQINSLRADDTAVYFCAKDVRFIAVPGDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 86 - protein, 450 aa, Synthetic construct
  source 1..450: note = R347 knob heavy chain
EVQLLESGGGLVQPGGSLRLSCTTSGFTFNTYAMSWVRQAPGKGLEWLSGINNNGRTAFYADSVKGRFTISRDNSKNTLYLQINSLRADDTAVYFCAKDVRFIAVPGDSWGQGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 217 aa, Synthetic construct
  source 1..217: note = R347 kappa light chain
ELVLTQPASVSGSPGQSITISCTGTSSDVGGYNYVSWYQQHPGKAPKLMIYDVSKRPSGVSNRFSGSKSGNTASLTISGLQAEDEADYYCSSYTSSSTLVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 216 aa, Synthetic construct
  source 1..216: note = R347 lambda light chai
ELVLTQPASVSGSPGQSITISCTGTSSDVGGYNYVSWYQQHPGKAPKLMIYDVSKRPSGVSNRFSGSKSGNTASLTISGLQAEDEADYYCSSYTSSSTLVFGGGTKLTVLGQPKLAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 89 - protein, 17 aa, Synthetic construct
  source 1..17: note = HCDR2 (Kabat) AA
YISSFGTFTYYADSVRG

SEQ ID NO: 90 - protein, 128 aa, Synthetic construct
  source 1..128: note = VH (Kabat) AA
QVQLVESGGGLVKPGGSLRLSCAASGSTLSDYFVSWIRQAPGKGLEWVSYISSFGTFTYYADSVRGRFTISRDNAKNTVYLQMNSLRAEDTAVYYCSSATENVDDIVAPGRGYYMDVWGRGTLVTVSS

SEQ ID NO: 91 - protein, 32 aa, Synthetic construct
  source 1..32: note = HFW3 (Kabat) AA
RFTISRDNAKNTVYLQMNSLRAEDTAVYYCSS

SEQ ID NO: 92 - protein, 111 aa, Synthetic construct
  source 1..111: note = VL (Kabat) AA
QSALTQPRSVSGSPGQSVTISCTGTRGDVGRYNHVSWYQQHPGKAPKLIIYNVYERPSGVPDRFSGSKSGNTASLTISGLQAEDEADYYCSSHSRSSTPVLFGGGTKLTVL

SEQ ID NO: 93 - protein, 14 aa, Synthetic construct
  source 1..14: note = LCDR1 (Kabat) AA
TGTRGDVGRYNHVS

SEQ ID NO: 94 - protein, 128 aa, Synthetic construct
  source 1..128: note = VH (Kabat) AA
QVQLVESGGGLVKPGGSLRLSCAASGSTLSDYFVSWIRQAPGKGLEWVSFVNSADTFTYYADSVRGRFTISRDNAKNTVYLQMNSLRAEDTAVYYCSSATEEVDDIVAPGRGYYMDVWGRGTLVTVSS

SEQ ID NO: 95 - protein, 17 aa, Synthetic construct
  source 1..17: note = HCDR2 (Kabat) AA
FVNSADTFTYYADSVRG

SEQ ID NO: 96 - protein, 19 aa, Synthetic construct
  source 1..19: note = HCDR3 (Kabat) AA
ATEEVDDIVAPGRGYYMDV

SEQ ID NO: 97 - protein, 128 aa, Synthetic construct
  source 1..128: note = VH (Kabat) AA
QVQLVESGGGLVKPGGSLRLSCAASGSTLSDYFVSWIRQAPGKGLEWVSYISSYGHYYTYADSVRGRFTISRDNAKNTVYLQMNSLRAEDTAVYYCASKPDDVDDIVAPGRGYYMDVWGRGTLVTVSS

SEQ ID NO: 98 - protein, 17 aa, Synthetic construct
  source 1..17: note = HCDR2 (Kabat) AA
YISSYGHYYTYADSVRG

SEQ ID NO: 99 - protein, 19 aa, Synthetic construct
  source 1..19: note = HCDR3 (Kabat) AA
KPDDVDDIVAPGRGYYMDV

SEQ ID NO: 100 - protein, 32 aa, Synthetic construct
  source 1..32: note = HFW3 (Kabat) AA
RFTISRDNAKNTVYLQMNSLRAEDTAVYYCAS

SEQ ID NO: 101 - protein, 128 aa, Synthetic construct
  source 1..128: note = VH (Kabat) AA
QVQLVESGGGLVKPGGSLRLSCAASGSTLSDYFVSWIRQAPGKGLEWVSVDSSYSTFTYYADSVRGRFTISRDNAKNTVYLQMNSLRAEDTAVYYCTSATVSVDDIVAPGRGYYMDVWGRGTLVTVSS

SEQ ID NO: 102 - protein, 17 aa, Synthetic construct
  source 1..17: note = HCDR2 (Kabat) AA
VDSSYSTFTYYADSVRG

SEQ ID NO: 103 - protein, 19 aa, Synthetic construct
  source 1..19: note = HCDR3 (Kabat) AA
ATVSVDDIVAPGRGYYMDV

SEQ ID NO: 104-105 - skipped (2 entries)

SEQ ID NO: 106 - protein, 128 aa, Synthetic construct
  source 1..128: note = VH (Kabat) AA
QVQLVESGGGLVKPGGSLRLSCADSGSTLSDYFVSWIRQAPGKGLEWVSFINSADTFTYYADSVRGRFTISRDNAKNTVYLQMNSLRAEDTAVYYCATDIEVVDDIVAPGRGYYMDVWGRGTLVTVSS

SEQ ID NO: 107 - protein, 17 aa, Synthetic construct
  source 1..17: note = HCDR2 (Kabat) AA
FINSADTFTYYADSVRG

SEQ ID NO: 108 - protein, 19 aa, Synthetic construct
  source 1..19: note = HCDR3 (Kabat) AA
DIEVVDDIVAPGRGYYMDV

SEQ ID NO: 109 - protein, 30 aa, Synthetic construct
  source 1..30: note = HFW1 (Kabat) AA
QVQLVESGGGLVKPGGSLRLSCADSGSTLS

SEQ ID NO: 110 - protein, 128 aa, Synthetic construct
  source 1..128: note = VH (Kabat) AA
QVQLVESGGGLVKPGGSLRLSCAASGSTLSDYFVSWIRQAPGKGLEWVSYISSFGTFTYYADSVRGRFTISRDNAKNTVYLQMNSLRAEDTAVYYCATEQDFVDDIVAPGRGYYMDVWGRGTLVTVSS

SEQ ID NO: 111 - protein, 19 aa, Synthetic construct
  source 1..19: note = HCDR3 (Kabat) AA
EQDFVDDIVAPGRGYYMDV

SEQ ID NO: 112 - protein, 32 aa, Synthetic construct
  source 1..32: note = HFW3 (Kabat) AA
RFTISRDNAKNTVYLQMNSLRAEDTAVYYCAT

SEQ ID NO: 113 - protein, 111 aa, Synthetic construct
  source 1..111: note = VL (Kabat) AA
QSALTQPRSVSGSPGQSVTISCTGTRGDVGRYNHVSWYQQHPGKAPKLIIYNVYERPSGVPDRFSGSKSGNTASLTISGLQAEDEADYYCSSHSRVNPPVLFGGGTKLTVL

SEQ ID NO: 114 - protein, 11 aa, Synthetic construct
  source 1..11: note = LCDR3 (Kabat) AA
SSHSRVNPPVL

SEQ ID NO: 115 - protein, 128 aa, Synthetic construct
  source 1..128: note = VH (Kabat) AA
QVQLVESGGGLVKPGGSLRLSCAASGSTLSDYFVSWIRQAPGKGLEWVSYISSFGTFTYYADSVRGRFTISRDNAKNTVYLQMNSLRAEDTAVYYCSTEASFVDDIVAPGRGYYMDVWGRGTLVTVSS

SEQ ID NO: 116 - protein, 19 aa, Synthetic construct
  source 1..19: note = HCDR3 (Kabat) AA
EASFVDDIVAPGRGYYMDV

SEQ ID NO: 117 - protein, 32 aa, Synthetic construct
  source 1..32: note = HFW3 (Kabat) AA
RFTISRDNAKNTVYLQMNSLRAEDTAVYYCST

SEQ ID NO: 118 - protein, 111 aa, Synthetic construct
  source 1..111: note = VL (Kabat) AA
QSALTQPRSVSGSPGQSVTISCTGTRGDVGRYSHVSWYQQHPGKAPKLIIYNVYERPSGVPDRFSGSKSGNTASLTISGLQAEDEADYYCSSHSRVNPPVLFGGGTKLTVL

SEQ ID NO: 119 - protein, 14 aa, Synthetic construct
  source 1..14: note = LCDR1 (Kabat) AA
TGTRGDVGRYSHVS

SEQ ID NO: 120 - protein, 128 aa, Synthetic construct
  source 1..128: note = VH (Kabat) AA
QVQLVESGGGLVKPGGSLRLSCAASGSTLSDYFVSWIRQAPGKGLEWVSYISSFGTFTYYADSVRGRFTISRDNAKNSVYLQMNSLRAEDTAVYYCSTEASFVDDIVAPGRGYYMDVWGRGTLVTVSS

SEQ ID NO: 121 - protein, 32 aa, Synthetic construct
  source 1..32: note = HFW3 (Kabat) AA
RFTISRDNAKNSVYLQMNSLRAEDTAVYYCST

SEQ ID NO: 122 - protein, 111 aa, Synthetic construct
  source 1..111: note = VL (Kabat) AA
QSALTQPRSVSGSPGQSVTISCTGTRSDVGRYNYVSWYQQHPGKAPKLIIYNVYERPSGVPDRFSGSKSGNTASLTISGLQAEDEADYYCSSHSRVNPPVLFGGGTKLTVL

SEQ ID NO: 123 - protein, 14 aa, Synthetic construct
  source 1..14: note = LCDR1 (Kabat) AA
TGTRSDVGRYNYVS

SEQ ID NO: 124 - protein, 111 aa, Synthetic construct
  source 1..111: note = VL (Kabat) AA
QSALTQPRSVSGSPGQSVTISCTGTRADVGRYNQVSWYQQHPGKAPKLIIYNVYERPSGVPDRFSGSKSGNTASLTISGLQAEDEADYYCSSHSRVNPPVLFGGGTKLTVL

SEQ ID NO: 125 - protein, 14 aa, Synthetic construct
  source 1..14: note = LCDR1 (Kabat) AA
TGTRADVGRYNQVS

SEQ ID NO: 126 - protein, 111 aa, Synthetic construct
  source 1..111: note = VL (Kabat) AA
QSALTQPRSVSGSPGQSVTISCTGTRGDVGRYNYVSWYQQHPGKAPKLIIYNVYERPSGVPDRFSGSKSGNTASLTISGLQAEDEADYYCSSHSRVNPPVLFGGGTKLTVL

SEQ ID NO: 127 - protein, 14 aa, Synthetic construct
  source 1..14: note = LCDR1 (Kabat) AA
TGTRGDVGRYNYVS

SEQ ID NO: 128 - protein, 128 aa, Synthetic construct
  source 1..128: note = VH (Kabat) AA
QVQLVESGGGLVKPGGSLRLSCAASGSTLSDYFVSWIRQAPGKGLEWVSYISSFGTFTYYADSVRGRFTISRDNAKNTVYLQMNSLRAEDTAVYYCSTATEPVDDIVAPGRGYYMDVWGRGTLVTVSS

SEQ ID NO: 129 - protein, 19 aa, Synthetic construct
  source 1..19: note = HCDR3 (Kabat) AA
ATEPVDDIVAPGRGYYMDV

SEQ ID NO: 130 - protein, 128 aa, Synthetic construct
  source 1..128: note = VH (Kabat) AA
QVQLVESGGGLVKPGGSLRLSCAASGSTLSDYFVSWIRQAPGKGLEWVSFVNSANTFTYYADSVRGRFTISRDNAKNTVYLQMNSLRAEDTAVYYCTSDAPEVDDIVAPGRGYYMDVWGRGTLVTVSS

SEQ ID NO: 131 - protein, 17 aa, Synthetic construct
  source 1..17: note = HCDR2 (Kabat) AA
FVNSANTFTYYADSVRG

SEQ ID NO: 132 - protein, 19 aa, Synthetic construct
  source 1..19: note = HCDR3 (Kabat) AA
DAPEVDDIVAPGRGYYMDV

SEQ ID NO: 133 - skipped

SEQ ID NO: 134 - protein, 32 aa, Synthetic construct
  source 1..32: note = HFW3 (Kabat) AA
RFTISRDNAKNTVYLQMNSLRAEDTAVYYCTS

SEQ ID NO: 135 - protein, 128 aa, Synthetic construct
  source 1..128: note = VH (Kabat) AA
QVQLVESGGGLVKPGGSLRLSCAASGSTLSDYFVSWIRQAPGKGLEWVSYISSFGTYYTYADSVRGRFTISRDNAKNTVYLQMNSLRAEDTAVYYCASATTNVDDIVAPGRGYYMDVWGRGTLVTVSS

SEQ ID NO: 136 - protein, 17 aa, Synthetic construct
  source 1..17: note = HCDR2 (Kabat) AA
YISSFGTYYTYADSVRG

SEQ ID NO: 137 - protein, 19 aa, Synthetic construct
  source 1..19: note = HCDR3 (Kabat) AA
ATTNVDDIVAPGRGYYMDV

SEQ ID NO: 138 - protein, 128 aa, Synthetic construct
  source 1..128: note = VH (Kabat) AA
QVQLVESGGGLVKPGGSLRLSCAASGSTLSDYFVSWIRQAPGKGLEWVSFINSANTFTYYADSVRGRFTISRDNAKNTVYLQMNSLRAEDTAVYYCSSANDDVDDIVAPGRGYYMDVWGRGTLVTVSS

SEQ ID NO: 139 - protein, 17 aa, Synthetic construct
  source 1..17: note = HCDR2 (Kabat) AA
FINSANTFTYYADSVRG

SEQ ID NO: 140 - protein, 128 aa, Synthetic construct
  source 1..128: note = VH (Kabat) AA
QVQLVESGGGLVKPGGSLRLSCAASGSTLSDYFVSWIRQAPGKGLEWVSFINSANTFTYYADSVRGRFTISRDNAKNSVYLQMNSLRAEDTAVYYCSSANDDVDDIVAPGRGYYMDVWGRGTLVTVSS

SEQ ID NO: 141 - protein, 14 aa, Synthetic construct
  source 1..14: note = LCDR1 (Kabat) AA
TGTSGDVGRYNHVS

SEQ ID NO: 142 - protein, 111 aa, Synthetic construct
  source 1..111: note = VL (Kabat) AA
QSALTQPRSVSGSPGQSVTISCTGTSGDVGRYNHVSWYQQHPGKAPKLIIYNVYERPSGVPDRFSGSKSGNTASLTISGLQAEDEADYYCSSHSRVNPPVLFGGGTKLTVL

SEQ ID NO: 143-145 - skipped (3 entries)

SEQ ID NO: 146 - protein, 111 aa, Synthetic construct
  source 1..111: note = VL (Kabat) AA
QSALTQPRSVSGSPGQSVTISCTGTRSDVGRYNHVSWYQQHPGKAPKLIIYNVYERPSGVPDRFSGSKSGNTASLTISGLQAEDEADYYCSSHSRVNPPVLFGGGTKLTVL

SEQ ID NO: 147 - protein, 14 aa, Synthetic construct
  source 1..14: note = LCDR1 (Kabat) AA
TGTRSDVGRYNHVS

SEQ ID NO: 148 - protein, 111 aa, Synthetic construct
  source 1..111: note = VL (Kabat) AA
QSALTQPRSVSGSPGQSVTISCTGTSGDVGRYNHVSWYQQHPGKAPKLIIYNVYERPSGVPDRFSGSKSGNTASLTISGLQAEDEADYYCSSHSRSSTPVLFGGGTKLTVL

SEQ ID NO: 149 - protein, 111 aa, Synthetic construct
  source 1..111: note = VL (Kabat) AA
QSALTQPRSVSGSPGQSVTISCTGTRPDVGRYNQVSWYQQHPGKAPKLIIYNVYERPSGVPDRFSGSKSGNTASLTISGLQAEDEADYYCSSHSRSSTPVLFGGGTKLTVL

SEQ ID NO: 150 - protein, 14 aa, Synthetic construct
  source 1..14: note = LCDR1 (Kabat) AA
TGTRPDVGRYNQVS

SEQ ID NO: 151 - protein, 128 aa, Synthetic construct
  source 1..128: note = VH (Kabat) AA
QVQLVESGGGLVKPGGSLRLSCAASGSTLSDYFVSWIRQAPGKGLEWVSFISSANTFTYYADSVRGRFTISRDNAKNSVYLQMNSLRAEDTAVYYCSSANDDVDDIVAPGRGYYMDVWGRGTLVTVSS

SEQ ID NO: 152 - protein, 17 aa, Synthetic construct
  source 1..17: note = HCDR2 (Kabat) AA
FISSANTFTYYADSVRG

SEQ ID NO: 153 - protein, 111 aa, Synthetic construct
  source 1..111: note = VL (Kabat) AA
QSALTQPRSVSGSPGQSVTISCTGTRGDVGRYNQVSWYQQHPGKAPKLIIYNVYERPSGVPDRFSGSKSGNTASLTISGLQAEDEADYYCSSHSRSSTPVLFGGGTKLTVL

SEQ ID NO: 154 - protein, 14 aa, Synthetic construct
  source 1..14: note = LCDR1 (Kabat) AA
TGTRGDVGRYNQVS

SEQ ID NO: 155 - protein, 128 aa, Synthetic construct
  source 1..128: note = VH (Kabat) AA
QVQLVESGGGLVKPGGSLRLSCAASGSTLSDYFVSWIRQAPGKGLEWVSFINDANTFTYYADSVRGRFTISRDNAKNTVYLQMNSLRAEDTAVYYCSSANDDVDDIVAPGRGYYMDVWGRGTLVTVSS

SEQ ID NO: 156 - protein, 128 aa, Synthetic construct; identical to SEQ ID NO: 155
  source 1..128: note = VH (Kabat) AA

SEQ ID NO: 157 - protein, 17 aa, Synthetic construct
  source 1..17: note = HCDR2 (Kabat) AA
FIKSANTFTYYADSVRG

SEQ ID NO: 158 - protein, 128 aa, Synthetic construct
  source 1..128: note = VH (Kabat) AA
QVQLVESGGGLVKPGGSLRLSCAASGSTLSDYFVSWIRQAPGKGLEWVSFIPSANTFTYYADSVRGRFTISRDNAKNTVYLQMNSLRAEDTAVYYCSSANDDVDDIVAPGRGYYMDVWGRGTLVTVSS

SEQ ID NO: 159 - protein, 17 aa, Synthetic construct
  source 1..17: note = HCDR2 (Kabat) AA
FIPSANTFTYYADSVRG

SEQ ID NO: 160 - protein, 128 aa, Synthetic construct
  source 1..128: note = VH (Kabat) AA
QVQLVESGGGLVKPGGSLRLSCAASGSTLSDYFVSWIRQAPGKGLEWVSFIASANTFTYYADSVRGRFTISRDNAKNTVYLQMNSLRAEDTAVYYCSSANDDVDDIVAPGRGYYMDVWGRGTLVTVSS

SEQ ID NO: 161 - protein, 17 aa, Synthetic construct
  source 1..17: note = HCDR2 (Kabat) AA
FIASANTFTYYADSVRG

SEQ ID NO: 162 - protein, 128 aa, Synthetic construct
  source 1..128: note = VH (Kabat) AA
QVQLVESGGGLVKPGGSLRLSCAASGSTLSDYFVSWIRQAPGKGLEWVSFITSANTFTYYADSVRGRFTISRDNAKNTVYLQMNSLRAEDTAVYYCSSANDDVDDIVAPGRGYYMDVWGRGTLVTVSS

SEQ ID NO: 163 - protein, 17 aa, Synthetic construct
  source 1..17: note = HCDR2 (Kabat) AA
FITSANTFTYYADSVRG

SEQ ID NO: 164 - protein, 128 aa, Synthetic construct
  source 1..128: note = VH (Kabat) AA
QVQLVESGGGLVKPGGSLRLSCAASGSTLSDYFVSWIRQAPGKGLEWVSFIESANTFTYYADSVRGRFTISRDNAKNTVYLQMNSLRAEDTAVYYCSSANDDVDDIVAPGRGYYMDVWGRGTLVTVSS

SEQ ID NO: 165 - protein, 17 aa, Synthetic construct
  source 1..17: note = HCDR2 (Kabat) AA
FIESANTFTYYADSVRG

SEQ ID NO: 166 - protein, 128 aa, Synthetic construct
  source 1..128: note = VH (Kabat) AA
QVQLVESGGGLVKPGGSLRLSCAASGSTLSDYFVSWIRQAPGKGLEWVSFIHSANTFTYYADSVRGRFTISRDNAKNTVYLQMNSLRAEDTAVYYCSSANDDVDDIVAPGRGYYMDVWGRGTLVTVSS

SEQ ID NO: 167 - protein, 17 aa, Synthetic construct
  source 1..17: note = HCDR2 (Kabat) AA
FIHSANTFTYYADSVRG

SEQ ID NO: 168 - protein, 128 aa, Synthetic construct
  source 1..128: note = VH (Kabat) AA
QVQLVESGGGLVKPGGSLRLSCAASGSTLSDYFVSWIRQAPGKGLEWVSFIGSANTFTYYADSVRGRFTISRDNAKNTVYLQMNSLRAEDTAVYYCSSANDDVDDIVAPGRGYYMDVWGRGTLVTVSS

SEQ ID NO: 169 - protein, 17 aa, Synthetic construct
  source 1..17: note = HCDR2 (Kabat) AA
FIGSANTFTYYADSVRG

SEQ ID NO: 170 - protein, 128 aa, Synthetic construct
  source 1..128: note = VH (Kabat) AA
QVQLVESGGGLVKPGGSLRLSCAASGSTLSDYFVSWIRQAPGKGLEWVSFIQSANTFTYYADSVRGRFTISRDNAKNTVYLQMNSLRAEDTAVYYCSSANDDVDDIVAPGRGYYMDVWGRGTLVTVSS

SEQ ID NO: 171 - protein, 17 aa, Synthetic construct
  source 1..17: note = HCDR2 (Kabat) AA
FIQSANTFTYYADSVRG

SEQ ID NO: 172 - protein, 128 aa, Synthetic construct
  source 1..128: note = VH (Kabat) AA
QVQLVESGGGLVKPGGSLRLSCAASGSTLSDYFVSWIRQAPGKGLEWVSFINPANTFTYYADSVRGRFTISRDNAKNTVYLQMNSLRAEDTAVYYCSSANDDVDDIVAPGRGYYMDVWGRGTLVTVSS

SEQ ID NO: 173 - protein, 17 aa, Synthetic construct
  source 1..17: note = HCDR2 (Kabat) AA
FINPANTFTYYADSVRG

SEQ ID NO: 174 - protein, 128 aa, Synthetic construct
  source 1..128: note = VH (Kabat) AA
QVQLVESGGGLVKPGGSLRLSCAASGSTLSDYFVSWIRQAPGKGLEWVSFINTANTFTYYADSVRGRFTISRDNAKNTVYLQMNSLRAEDTAVYYCSSANDDVDDIVAPGRGYYMDVWGRGTLVTVSS

SEQ ID NO: 175 - protein, 17 aa, Synthetic construct
  source 1..17: note = HCDR2 (Kabat) AA
FINTANTFTYYADSVRG

SEQ ID NO: 176 - protein, 128 aa, Synthetic construct
  source 1..128: note = VH (Kabat) AA
QVQLVESGGGLVKPGGSLRLSCAASGSTLSDYFVSWIRQAPGKGLEWVSFINKANTFTYYADSVRGRFTISRDNAKNTVYLQMNSLRAEDTAVYYCSSANDDVDDIVAPGRGYYMDVWGRGTLVTVSS

SEQ ID NO: 177 - protein, 17 aa, Synthetic construct
  source 1..17: note = HCDR2 (Kabat) AA
FINKANTFTYYADSVRG

SEQ ID NO: 178 - protein, 128 aa, Synthetic construct
  source 1..128: note = VH (Kabat) AA
QVQLVESGGGLVKPGGSLRLSCAASGSTLSDYFVSWIRQAPGKGLEWVSFINAANTFTYYADSVRGRFTISRDNAKNTVYLQMNSLRAEDTAVYYCSSANDDVDDIVAPGRGYYMDVWGRGTLVTVSS

SEQ ID NO: 179 - protein, 128 aa, Synthetic construct
  source 1..128: note = VH (Kabat) AA
QVQLVESGGGLVKPGGSLRLSCAASGSTLSDYFVSWIRQAPGKGLEWVSFINQANTFTYYADSVRGRFTISRDNAKNTVYLQMNSLRAEDTAVYYCSSANDDVDDIVAPGRGYYMDVWGRGTLVTVSS

SEQ ID NO: 180 - protein, 17 aa, Synthetic construct
  source 1..17: note = HCDR2 (Kabat) AA
FINQANTFTYYADSVRG

SEQ ID NO: 181 - protein, 128 aa, Synthetic construct
  source 1..128: note = VH (Kabat) AA
QVQLVESGGGLVKPGGSLRLSCAASGSTLSDYFVSWIRQAPGKGLEWVSFIPSANTFTYYADSVRGRFTISRDNAKNSVYLQMNSLRAEDTAVYYCSSANDDVDDIVAPGRGYYMDVWGRGTLVTVSS

SEQ ID NO: 182 - skipped

SEQ ID NO: 183 - protein, 128 aa, Synthetic construct
  source 1..128: note = VH (Kabat) AA
QVQLVESGGGLVKPGGSLRLSCAASGSTLSDYFVSWIRQAPGKGLEWVSFINQANTFTYYADSVRGRFTISRDNAKNSVYLQMNSLRAEDTAVYYCSSANDDVDDIVAPGRGYYMDVWGRGTLVTVSS

SEQ ID NO: 184 - skipped

SEQ ID NO: 185 - protein, 128 aa, Synthetic construct
  source 1..128: note = VH (Kabat) AA
QVQLVESGGGLVKPGGSLRLSCAASGSTLSDYFVSWIRQAPGKGLEWVSFINTANTFTYYADSVRGRFTISRDNAKNSVYLQMNSLRAEDTAVYYCSSANDDVDDIVAPGRGYYMDVWGRGTLVTVSS

SEQ ID NO: 186 - skipped

SEQ ID NO: 187 - protein, 6 aa, Synthetic construct
CXRHXC

SEQ ID NO: 188 - protein, 6 aa, Synthetic construct
CRRHXC

SEQ ID NO: 189 - protein, 6 aa, Synthetic construct
CXRHRC

SEQ ID NO: 190-191 - skipped (2 entries)

SEQ ID NO: 192 - protein, 126 aa, Synthetic construct
  source 1..126: note = VH (Kabat) AA
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARHSYTRGFFSHEDDAVDIWGQGTTVTVSS

SEQ ID NO: 193 - protein, 5 aa, Synthetic construct
  source 1..5: note = HCDR1 (Kabat) AA
SYAMS

SEQ ID NO: 194 - protein, 17 aa, Synthetic construct
  source 1..17: note = HCDR2 (Kabat) AA
AISGSGGSTYYADSVKG

SEQ ID NO: 195 - protein, 17 aa, Synthetic construct
  source 1..17: note = HCDR3 (Kabat) AA
HSYTRGFFSHEDDAVDI

SEQ ID NO: 196 - protein, 30 aa, Synthetic construct; identical to SEQ ID NO: 6
  source 1..30: note = HFW1 (Kabat) AA

SEQ ID NO: 197 - protein, 14 aa, Synthetic construct
  source 1..14: note = HFW2 (Kabat) AA
WVRQAPGKGLEWVS

SEQ ID NO: 198 - protein, 32 aa, Synthetic construct; identical to SEQ ID NO: 8
  source 1..32: note = HFW3 (Kabat) AA

SEQ ID NO: 199 - protein, 11 aa, Synthetic construct
  source 1..11: note = HFW4 (Kabat) AA
WGQGTTVTVSS

SEQ ID NO: 200 - protein, 106 aa, Synthetic construct
  source 1..106: note = VL (Kabat) AA
SYVLTQPPSVSVSPGQTASITCSGDKVGDKYVSWYQQKPGQSPVLVIYQDSQRPSGIPERFSGSNSGNTATLTISGTQAMDEADYYCEVGVDYEYVFGSGTKVTVL

SEQ ID NO: 201 - protein, 11 aa, Synthetic construct
  source 1..11: note = LCDR1 (Kabat) AA
SGDKVGDKYVS

SEQ ID NO: 202 - protein, 7 aa, Synthetic construct
  source 1..7: note = LCDR2 (Kabat) AA
QDSQRPS

SEQ ID NO: 203 - protein, 9 aa, Synthetic construct
  source 1..9: note = LCDR3 (Kabat) AA
EVGVDYEYV

SEQ ID NO: 204 - protein, 22 aa, Synthetic construct
  source 1..22: note = LFW1 (Kabat) AA
SYVLTQPPSVSVSPGQTASITC

SEQ ID NO: 205 - protein, 15 aa, Synthetic construct
  source 1..15: note = LFW2 (Kabat) AA
WYQQKPGQSPVLVIY

SEQ ID NO: 206 - protein, 32 aa, Synthetic construct; identical to SEQ ID NO: 15
  source 1..32: note = LFW3 (Kabat) AA

SEQ ID NO: 207 - protein, 10 aa, Synthetic construct
  source 1..10: note = LFW4 (Kabat) AA
FGSGTKVTVL

SEQ ID NO: 208 - protein, 126 aa, Synthetic construct
  source 1..126: note = VH (Kabat) AA
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARHSYTRGFFSHEDDAVDIWGRGTLVTVSS

SEQ ID NO: 209 - protein, 11 aa, Synthetic construct
  source 1..11: note = HFW4 (Kabat) AA
WGRGTTVTVSS

SEQ ID NO: 210 - protein, 106 aa, Synthetic construct
  source 1..106: note = VL (Kabat) AA
SYVLTQPPSVSVSPGQTASITCSGDKVGDKYVSWYQQKPGQAPVLVMYQDSQRPSGIPERISGSNSGNTATLTISGTQAVDEAEYYCEVGVDYEYVFGSGTKVTVL

SEQ ID NO: 211 - protein, 15 aa, Synthetic construct
  source 1..15: note = LFW2 (Kabat) AA
WYQQKPGQAPVLVMY

SEQ ID NO: 212 - protein, 32 aa, Synthetic construct
  source 1..32: note = LFW3 (Kabat) AA
GIPERISGSNSGNTATLTISGTQAVDEAEYYC

SEQ ID NO: 213 - protein, 126 aa, Synthetic construct
  source 1..126: note = VH (Kabat) AA
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQALVRGFFSHEDDAVDIWGQGTTVTVSS

SEQ ID NO: 214 - protein, 17 aa, Synthetic construct
  source 1..17: note = HCDR3 (Kabat) AA
QALVRGFFSHEDDAVDI

SEQ ID NO: 215 - protein, 11 aa, Synthetic construct
  source 1..11: note = HFW4 (Kabat) AA
WGRGTLVTVSS

SEQ ID NO: 216 - protein, 9 aa, Synthetic construct
  source 1..9: note = LCDR3 (Kabat) AA
QTWYDDALT

SEQ ID NO: 217 - protein, 126 aa, Synthetic construct
  source 1..126: note = VH (Kabat) AA
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQALVRGFFSHEDDAVDIWGRGTTVTVSS

SEQ ID NO: 218 - protein, 106 aa, Synthetic construct
  source 1..106: note = VL (Kabat) AA
SYVLTQPPSVSVSPGQTASITCSGDKVGDKYVSWYQQKPGQAPVLVMYQDSQRPSGIPERISGSNSGNTATLTISGTQAVDEAEYYCQTWYDDALTFGSGTKVTVL

SEQ ID NO: 219 - protein, 126 aa, Synthetic construct
  source 1..126: note = VH (Kabat) AA
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQVLVRGFFSHEDDAVDIWGRGTTVTVSS

SEQ ID NO: 220 - protein, 17 aa, Synthetic construct
  source 1..17: note = HCDR3 (Kabat) AA
QVLVRGFFSHEDDAVDI

SEQ ID NO: 221 - protein, 106 aa, Synthetic construct
  source 1..106: note = VL (Kabat) AA
SYVLTQPPSVSVSPGQTASITCSGDKVGDKYVSWYQQKPGQAPVLVMYQDSQRPSGIPERISGSNSGNTATLTISGTQAVDEAEYYCQTWAPDVLLFGSGTKVTVL

SEQ ID NO: 222 - protein, 9 aa, Synthetic construct
  source 1..9: note = LCDR3 (Kabat) AA
QTWAPDVLL

SEQ ID NO: 223 - protein, 126 aa, Synthetic construct
  source 1..126: note = VH (Kabat) AA
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQVLTRGFFSHEDDAVDIWGQGTTVTVSS

SEQ ID NO: 224 - protein, 17 aa, Synthetic construct
  source 1..17: note = HCDR3 (Kabat) AA
QVLTRGFFSHEDDAVDI

SEQ ID NO: 225 - protein, 106 aa, Synthetic construct
  source 1..106: note = VL (Kabat) AA
SYVLTQPPSVSVSPGQTASITCSGDKVGDKYVSWYQQKPGQSPVLVIYQDSQRPSGIPERFSGSNSGNTATLTISGTQAMDEADYYCHTYAEDEYVFGSGTKVTVL

SEQ ID NO: 226 - protein, 9 aa, Synthetic construct
  source 1..9: note = LCDR3 (Kabat) AA
HTYAEDEYV

SEQ ID NO: 227 - protein, 126 aa, Synthetic construct
  source 1..126: note = VH (Kabat) AA
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQVLTRGFFSHEDDAVDIWGRGTTVTVSS

SEQ ID NO: 228 - protein, 106 aa, Synthetic construct
  source 1..106: note = VL (Kabat) AA
SYVLTQPPSVSVSPGQTASITCSGDKVGDKYVSWYQQKPGQAPVLVMYQDSQRPSGIPERISGSNSGNTATLTISGTQAVDEAEYYCHTYAEDEYVFGSGTKVTVL

SEQ ID NO: 229 - protein, 126 aa, Synthetic construct
  source 1..126: note = VH (Kabat) AA
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARHSYTRGFFSHESPDLPTWGRGTTVTVSS

SEQ ID NO: 230 - protein, 17 aa, Synthetic construct
  source 1..17: note = HCDR3 (Kabat) AA
HSYTRGFFSHESPDLPT

SEQ ID NO: 231 - protein, 106 aa, Synthetic construct
  source 1..106: note = VL (Kabat) AA
SYVLTQPPSVSVSPGQTASITCSGDKVGDKYVSWYQQKPGQAPVLVMYQDSQRPSGIPERISGSNSGNTATLTISGTQAVDEAEYYCQSENAVEYVFGSGTKVTVL

SEQ ID NO: 232 - protein, 9 aa, Synthetic construct
  source 1..9: note = LCDR3 (Kabat) AA
QSENAVEYV

SEQ ID NO: 233 - protein, 115 aa, Synthetic construct
  source 1..115: note = VH (Kabat) AA
EVQLVESGAEVKKPGSSVKVSCKASGGTFSSYAISWVRQAPGQGLEWMGGIIPIFGTANYAQKFQGRVTITADESTSTAYMELSSLRSEDTAVYYCASGGWFDYWGRGTLVTVSS

SEQ ID NO: 234 - protein, 5 aa, Synthetic construct
  source 1..5: note = HCDR1 (Kabat) AA
SYAIS

SEQ ID NO: 235 - protein, 17 aa, Synthetic construct
  source 1..17: note = HCDR2 (Kabat) AA
GIIPIFGTANYAQKFQG

SEQ ID NO: 236 - protein, 6 aa, Synthetic construct
  source 1..6: note = HCDR3 (Kabat) AA
GGWFDY

SEQ ID NO: 237 - protein, 30 aa, Synthetic construct
  source 1..30: note = HFW1 (Kabat) AA
EVQLVESGAEVKKPGSSVKVSCKASGGTFS

SEQ ID NO: 238 - protein, 14 aa, Synthetic construct
  source 1..14: note = HFW2 (Kabat) AA
WVRQAPGQGLEWMG

SEQ ID NO: 239 - protein, 32 aa, Synthetic construct
  source 1..32: note = HFW3 (Kabat) AA
RVTITADESTSTAYMELSSLRSEDTAVYYCAS

SEQ ID NO: 240 - protein, 11 aa, Synthetic construct
  source 1..11: note = HFW4 (Kabat) AA
WGRGTLVTVSS

SEQ ID NO: 241 - protein, 110 aa, Synthetic construct
  source 1..110: note = VL (Kabat) AA
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAVVFGGGTKVTVL

SEQ ID NO: 242 - protein, 13 aa, Synthetic construct
  source 1..13: note = LCDR1 (Kabat) AA
SGSSSNIGNNYVS

SEQ ID NO: 243 - protein, 7 aa, Synthetic construct
  source 1..7: note = LCDR2 (Kabat) AA
DNNKRPS

SEQ ID NO: 244 - protein, 10 aa, Synthetic construct
  source 1..10: note = HCDR3 (Kabat) AA
EGGDYWYFDV

SEQ ID NO: 245 - protein, 11 aa, Synthetic construct
  source 1..11: note = LCDR3 (Kabat) AA
GTWDSSLSAVV

SEQ ID NO: 246 - protein, 22 aa, Synthetic construct
  source 1..22: note = LFW1 (Kabat) AA
QSVVTQPPSVSAAPGQKVTISC

SEQ ID NO: 247 - protein, 15 aa, Synthetic construct
  source 1..15: note = LFW2 (Kabat) AA
WYQQLPGTAPKLLIY

SEQ ID NO: 248 - protein, 32 aa, Synthetic construct
  source 1..32: note = LFW3 (Kabat) AA
GIPDRFSGSKSGTSATLGITGLQTGDEADYYC

SEQ ID NO: 249 - protein, 10 aa, Synthetic construct
  source 1..10: note = LFW4 (Kabat) AA
FGGGTKVTVL

SEQ ID NO: 250 - protein, 126 aa, Synthetic construct
  source 1..126: note = VH (Kabat) AA
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARHSYTRGFFSHEDDAVDIWGRGTTVTVSS

SEQ ID NO: 251 - protein, 9 aa, Synthetic construct
  source 1..9: note = LCDR3 (Kabat) AA
QTWDSNEYV

SEQ ID NO: 252 - protein, 125 aa, Synthetic construct
  source 1..125: note = VH (Kabat) AA
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDSGKSYSRGWYTAFDIWGRGTTVTVSS

SEQ ID NO: 253 - protein, 16 aa, Synthetic construct
  source 1..16: note = HCDR3 (Kabat) AA
DSGKSYSRGWYTAFDI

SEQ ID NO: 254 - protein, 108 aa, Synthetic construct
  source 1..108: note = VL (Kabat) AA
QAVLTQPSSVSVAPGKTATITCGGDNIGSKSVHWYQQKPGQAPLLVIFYDTDRPSGVPERFSGSNSGNTATLTISRVEAGDEADYYCQVWDSNGDHYVFGTGTKLTVL

SEQ ID NO: 255 - protein, 11 aa, Synthetic construct
  source 1..11: note = LCDR1 (Kabat) AA
GGDNIGSKSVH

SEQ ID NO: 256 - protein, 7 aa, Synthetic construct
  source 1..7: note = LCDR2 (Kabat) AA
YDTDRPS

SEQ ID NO: 257 - protein, 11 aa, Synthetic construct
  source 1..11: note = LCDR3 (Kabat) AA
QVWDSNGDHYV

SEQ ID NO: 258 - protein, 22 aa, Synthetic construct
  source 1..22: note = LFW1 (Kabat) AA
QAVLTQPSSVSVAPGKTATITC

SEQ ID NO: 259 - protein, 15 aa, Synthetic construct
  source 1..15: note = LFW2 (Kabat) AA
WYQQKPGQAPLLVIF

SEQ ID NO: 260 - protein, 32 aa, Synthetic construct
  source 1..32: note = LFW3 (Kabat) AA
GVPERFSGSNSGNTATLTISRVEAGDEADYYC

SEQ ID NO: 261 - protein, 10 aa, Synthetic construct
  source 1..10: note = LFW4 (Kabat) AA
FGTGTKLTVL

SEQ ID NO: 262 - protein, 119 aa, Synthetic construct
  source 1..119: note = VH (Kabat) AA
QVQLQQPGAELVKPGASVKLSCKASGYTFTSYWMHWVKQRPGRGPEWIGRIDPNSGGTKYNEKFKSKAILTVDKPSSTAYMQLSSLTSEDSAVYYCAREGGDYWYFDVWGAGTTVTVSS

SEQ ID NO: 263 - protein, 5 aa, Synthetic construct
  source 1..5: note = HCDR1 (Kabat) AA
SYWMH

SEQ ID NO: 264 - protein, 17 aa, Synthetic construct
  source 1..17: note = HCDR2 (Kabat) AA
RIDPNSGGTKYNEKFKS

SEQ ID NO: 265 - protein, 30 aa, Synthetic construct
  source 1..30: note = HFW1 (Kabat) AA
QVQLQQPGAELVKPGASVKLSCKASGYTFT

SEQ ID NO: 266 - protein, 14 aa, Synthetic construct
  source 1..14: note = HFW2 (Kabat) AA
WVKQRPGRGPEWIG

SEQ ID NO: 267 - protein, 32 aa, Synthetic construct
  source 1..32: note = HFW3 (Kabat) AA
KAILTVDKPSSTAYMQLSSLTSEDSAVYYCAR

SEQ ID NO: 268 - protein, 11 aa, Synthetic construct
  source 1..11: note = HFW4 (Kabat) AA
WGAGTTVTVSS

SEQ ID NO: 269 - protein, 106 aa, Synthetic construct
  source 1..106: note = VL (Kabat) AA
QIVLTQSPALMSASPGEKVTMTCSANSGLRYMYWYQQKPRSSPKPWIYLTSNLASGVPARFSGSGSGTSYSLTISSMEAEDAATYYCQQWSSIPPTFGAGTKLELK

SEQ ID NO: 270 - protein, 10 aa, Synthetic construct
  source 1..10: note = LCDR1 (Kabat) AA
SANSGLRYMY

SEQ ID NO: 271 - protein, 7 aa, Synthetic construct
  source 1..7: note = LCDR2 (Kabat) AA
LTSNLAS

SEQ ID NO: 272 - protein, 9 aa, Synthetic construct
  source 1..9: note = LCDR3 (Kabat) AA
QQWSSIPPT

SEQ ID NO: 273 - protein, 23 aa, Synthetic construct
  source 1..23: note = LFW1 (Kabat) AA
QIVLTQSPALMSASPGEKVTMTC

SEQ ID NO: 274 - protein, 15 aa, Synthetic construct
  source 1..15: note = LFW2 (Kabat) AA
WYQQKPRSSPKPWIY

SEQ ID NO: 275 - protein, 32 aa, Synthetic construct
  source 1..32: note = LFW3 (Kabat) AA
GVPARFSGSGSGTSYSLTISSMEAEDAATYYC

SEQ ID NO: 276 - protein, 10 aa, Synthetic construct
  source 1..10: note = LFW4 (Kabat) AA
FGAGTKLELK

SEQ ID NO: 277 - protein, 118 aa, Synthetic construct
  source 1..118: note = VH (Kabat) AA
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCTRTYYDDAMDYWGQGTSVTVSS

SEQ ID NO: 278 - protein, 5 aa, Synthetic construct
  source 1..5: note = HCDR1 (Kabat) AA
DYGMH

SEQ ID NO: 279 - protein, 17 aa, Synthetic construct
  source 1..17: note = HCDR2 (Kabat) AA
YISSGSSTIYYADTVKG

SEQ ID NO: 280 - protein, 9 aa, Synthetic construct
  source 1..9: note = HCDR3 (Kabat) AA
TYYDDAMDY

SEQ ID NO: 281 - protein, 30 aa, Synthetic construct
  source 1..30: note = HFW1 (Kabat) AA
EVQLVESGGGLVKPGGSRKLSCAASGFTFS

SEQ ID NO: 282 - protein, 14 aa, Synthetic construct
  source 1..14: note = HFW2 (Kabat) AA
WVRQAPEKGLEWVA

SEQ ID NO: 283 - protein, 32 aa, Synthetic construct
  source 1..32: note = HFW3 (Kabat) AA
RFTISRDNAKNTLFLQMTSLRSEDTAMYYCTR

SEQ ID NO: 284 - protein, 11 aa, Synthetic construct
  source 1..11: note = HFW4 (Kabat) AA
WGQGTSVTVSS

SEQ ID NO: 285 - protein, 107 aa, Synthetic construct
SIVMTQTPKFLLVSAGDRVTITCKASQSVSNDVVWYQQKPGQSPKLLISYASNRYTGVPDRFTGSGYGTDFTFTISSVQAEDLAVYFCQQDYSSPWTFGGGTKLEIK

SEQ ID NO: 286 - protein, 11 aa, Synthetic construct
  source 1..11: note = LCDR1 (Kabat) AA
KASQSVSNDVV

SEQ ID NO: 287 - protein, 7 aa, Synthetic construct
  source 1..7: note = LCDR2 (Kabat) AA
YASNRYT

SEQ ID NO: 288 - protein, 9 aa, Synthetic construct
  source 1..9: note = LCDR3 (Kabat) AA
QQDYSSPWT

SEQ ID NO: 289 - protein, 23 aa, Synthetic construct
  source 1..23: note = LFW1 (Kabat) AA
SIVMTQTPKFLLVSAGDRVTITC

SEQ ID NO: 290 - protein, 15 aa, Synthetic construct
  source 1..15: note = LFW2 (Kabat) AA
WYQQKPGQSPKLLIS

SEQ ID NO: 291 - protein, 32 aa, Synthetic construct
  source 1..32: note = LFW3 (Kabat) AA
GVPDRFTGSGYGTDFTFTISSVQAEDLAVYFC

SEQ ID NO: 292 - protein, 10 aa, Synthetic construct
  source 1..10: note = LFW4 (Kabat) AA
FGGGTKLEIK

(19) United States
(12) Patent Application Publication
(10) Pub. No.: US 2024/0199761 A1
(43) Pub. Date: Jun. 20, 2024

(54) TREATMENT OF AUTOIMMUNE DISEASE

(71) Applicant: AstraZeneca AB, Södertälje (SE)

(72) Inventors: Gary SIMS, Gaithersburg, MD (US); Catherine Eugenie HUNTINGTON, Cambridge (GB); Katherine Ann VOUSDEN, Cambridge (GB)

(21) Appl. No.: 18/542,918

(22) Filed: Dec. 18, 2023

Related U.S. Application Data
(60) Provisional application No. 63/476,066, filed on Dec. 19, 2022.

(51) Int. Cl. C07K 16/40; A61K 39/00

(52) U.S. Cl. CPC C07K 16/40; A61K 39/00; A61K 2039/505; C07K 2317/31; C07K 2317/35; C07K 2317/622; C07K 2317/76

(57) ABSTRACT

Antibodies for binding PADs and their use in therapy.

### 1 CROSS-REFERENCE TO RELATED APPLICATIONS

This application claims benefit under 35 U.S.C. § 119(e) of U.S. Provisional Application No. 63/476,066, filed Dec. 19, 2022, which is incorporated by reference herein.

### 2 REFERENCE TO SEQUENCE LISTING

This application incorporates by reference a Sequence Listing submitted electronically with the application as an XML file entitled “PAD2PAD4-SeqListing.xml” created on Dec. 15, 2023 and having a size of 322,184 bytes.

### 3 BACKGROUND

Rheumatoid arthritis (RA) is a common autoimmune disease with a chronic progressive phenotype. It is a chronic systemic inflammatory disease affecting small and large joints leading to progressive joint destruction, loss of function, chronic pain/fatigue with increasing disability.

Despite the development of newer therapies in the treatment of RA [1-4], particularly the advent of biologic therapies that block tumor necrosis factor alpha (TNF-alpha), interleukin-6 receptors, or deplete B cells, many patients still suffer from poorly controlled, active disease and response rates remain low. Current targeted treatments typically start with disease-modifying antirheumatic drugs (DMARDs) such a methotrexate, then targeted therapies such as newer biologics therapies are cycled based on the ‘Treat to Target’ principle of attempting to achieve remission, low disease activity, and/or ACR70 (70% improvement in activity). However, these have limited success with only 20-30% of patients achieving ACR70. There is a clear need for more effective therapies for treating RA, ideally targeting the causative pathways of disease.

Histologically, RA is characterized by synovial inflammation with T and B cell infiltrates, often organized into germinal centre-like structures, as well as macrophages, dendritic cells, and neutrophils [5-7], the latter particularly abundant in the synovial fluid in early stages of the disease. Although these histological features are not very dissimilar from other autoimmune or inflammatory conditions, it has become apparent that RA has unique features that cannot be simply explained by a traditional T cell-centric hypothesis of pathogenesis, i.e., that the initial event is loss of tolerance.

Peptidyl arginine deiminases (PADs) are a family of five isozymes (PAD1, 2, 3, 4 and 6) encoded by distinct genes in the human genome [8]. PADs are calcium-dependent enzymes that catalyze the post-transcriptional modification known as citrullination, which is the conversion of a basic charged amino acid residue arginine to a neutral residue citrulline. Citrullinated proteins induce generation of anti-citrullinated protein antibodies (ACPA) and cyclic citrullinated peptides (CCP). ACPA and CPP can contribute to perpetuation of an autoimmune response.

WO 2012026309 A9 [9] describes anti-PAD4 antibodies.

WO 2014/086365 A1 [10] describes antibodies for binding rabbit PAD2 (rPAD2).

WO 2016/155745 A1 [11] suggests mouse monoclonal antibodies cross-reactive for PAD2, PAD4 and PAD3.

WO 2016143753 A1 [12] describes anti-PAD4 antibodies.

Aosasa et al. 2021 describes chimeric anti-PAD2 antibodies [13].

Thus, there remains a need for effective compositions to treat autoimmune diseases such as RA.

### 4 SUMMARY

The invention relates to anti-PAD4 antibodies with high affinity and specificity for human and cynomolgus PAD4. The invention relates to anti-PAD2 antibodies with high affinity and specificity for human, cynomolgus and mouse PAD2.

The invention further relates to bispecific antibodies with high affinity and specificity for PAD2 (human, mouse and cyno) and PAD4 (human and cyno, or mouse).

The invention also relates to anti-PAD4 and anti-PAD2 antibodies and anti-PAD2/PAD4 bispecifics that are highly potent at inhibiting PAD4 and/or PAD2 activity and that are potent at inhibiting PAD activity in the synovial fluid of rheumatoid arthritis (RA) patients.

The present invention relates to the treatment of autoimmune disease through targeting of both PAD2 and PAD4. The invention relates particularly to the use of an anti-PAD2 and an anti-PAD4 antibody in combination, or an anti-PAD2/4 bispecific, in the treatment of autoimmune disease characterised by elevated PAD activity, such as RA. The invention is supported by data, provided herein for the first time, showing that the activities of PAD2 and PAD4 in RA disease are non-redundant. Targeting both PAD2 and PAD4 is shown to be surprisingly both necessary and sufficient to abolish PAD activity in the whole blood, serum and synovial fluid of RA patients.

### 5 BRIEF DESCRIPTION OF THE DRAWINGS

FIG. 1: PAD2 and PAD4 dependent generation of citrullinated RA antigens

ELISA measuring generation of citrullinated antigens (fibrinogen beta chain and alpha enolase) by PAD2 and PAD4.

FIG. 2A-B: Potency of prior art anti-PAD4 antibodies

Potency of prior art humanised anti-PAD4 antibodies, assessed by H3 histone citrullination assay (FIG. 2A) or BAEE PAD activity assay (Cayman Chemical) (FIG. 2B).

FIG. 3A-D: PAD2 and PAD4 expression

FIG. 3A: PAD4 and PAD2 protein levels in serum from Rheumatoid Arthritis (RA) patients compared to healthy donors (HD). (RA) n=90, Healthy Donor (HD) n=24. Bars indicate median.

Median for PAD2 in HD is <LLOD. Mann-Whitney test. ****p<0.0001. PADs measured with Cayman ELISA kits. FIG. 3B: PAD2 and PAD4 protein levels are high in RA synovial fluid. RA Synovial Fluid (SF) n=5. Bars indicates median values. Synovial fluid was diluted and PAD levels were measured with Cayman ELISA kits. FIG. 3C: RNA expression profile of PAD4 in immune cells from human and Cynomolgus (cyno). FIG. 3D: RNA expression profile of PAD4 in immune cells from human and Cynomolgus (cyno). Expression of PAD2 and PAD4 is shown relative to expression in human B cells.

FIG. 4A-B: Cell surface quantification of PAD2 and PAD4

Quantification of PAD2 (FIG. 4B) and PAD4 (FIG. 4A) on the surface of immune cells.

FIG. 5: PAD4 and anti-PAD4 Fabs

Interferometric scattering (ISCAT) of individual protein molecules in close proximity of the surface. Anti-PAD4=Clone 42 (48LO0063, IgG or Fab).

FIG. 6: PAD4 and anti-PAD4 IgG

Interferometric scattering (ISCAT) of individual protein molecules in close proximity of the surface. Anti-PAD4=Clone 42 (48LO0063, IgG or Fab).

FIG. 7: PAD2 and anti-PAD2 Fab/IgG

iSCAT for PAD2 and anti-PAD2 Fabs and IgGs. Anti-PAD2=Clone 22 (IgG or Fab).

FIG. 8A-B: iSCAT summary

FIG. 8A: PAD4; FIG. 8B: PAD2.

FIG. 9A-B: Potency assay optimisation

FIG. 9A: Optimisation of potency assay parameters. FIG. 9B: PAD4 Fab Histone H3 assay. 3 hrs 45 mins PAD4 incubation. [PAD4]=15 pg·ml.

FIG. 10A-B: Bispecific formats

FIG. 10A: Monovalent Duet mAbs. FIG. 10B: Bivalent Bispecific Bis3.

FIG. 11: Histone-H3 activity assay

The histone H3 substrate is coated on the plate where active PADs in the sample deaminate the argine residues to form citrulline. These citrullinated epitopes are then detected through standard immunoassay methods. This assay can be used to demonstrate target engagement in both the circulation and synovial compartments (where PAD2 activity is higher) without sample dilution.

FIG. 12A-B: Inhibition of PAD activity

FIG. 12A: Inhibition of PAD2 in the synovial fluid (1000-fold dilution). FIG. 12B inhibition in neutrophil supernatant (250-fold dilution). PAD activity in diluted synovial fluid samples was determined using a Histone-H3 PAD activity assay. EDTA sequesters calcium and inhibits PAD activity and serves as a background control.

FIG. 13: PAD2 antibody affinities

Binding affinity, K_{D} (nM), for anti-PAD2 monoclonal antibodies (as Fabs). Biotinylated PAD2 was captured onto a CM5/C1-Streptavidin surface. Affinity: K_{D} (nM). Data shown are averages, n=2-9 experiments (Table 72).

FIG. 14: PAD4 antibody affinities

Binding affinity, K_{D} (nM), for anti-PAD4 monoclonal antibodies (as Fabs). Biotinylated PAD4 was captured onto a CM5/C1-Streptavidin surface. Affinity: K_{D} (nM). Data shown are averages of n=1-6 experiments (Table 74).

FIG. 15: DuetMab PAD2/PAD4 binding behaviour

iSCAT for DuetMabs for PAD2/PAD4 bispecifics.

FIG. 16: Bis3 PAD2/PAD4 binding behaviour

iSCAT for Bis3 PAD2/PAD4 bispecifics.

FIG. 17: DuetMab versus Bis3 binding

Schematic showing the difference between Bis3 and DuetMab format binding to PAD2 and PAD4 dimers.

FIG. 18: Effect of Fc modifications on thermostability

Thermostability of the bispecific formats in the context of different Fc modifications were assessed by Nano-DSF. Tₒₙₛₑₜ values shown for Clones 01-12, (C) (Table 92).

FIG. 19A-B: Accelerated stability

The propensity of the DuetMab and Bis3 formats to aggregate at 40° C. (FIG. 19B) and 45° C. (FIG. 19A) compared to 4° C. was assessed by HP-SEC.

FIG. 20: Cynomolgus study immunohistochemistry

FIG. 21: In vitro cytokine release, plate bound antibodies

Cytokine expression following exposure to Bis3 (Clone 12) or DuetMab (Clone 06) format antibodies (HD=high dose).

FIG. 22A-E: Potency of Bis3 format

FIG. 22A: Study protocol. FIG. 22B: PAD activity measured in plasma with Histone H3 citrullination assay, day 0 to day 36. FIG. 22C: Endogenous PAD activity measured in plasma with Histone H3 citrullination assay, day 0 to day 36. FIG. 22D: PAD activity measured in plasma with Histone H3 citrullination assay, day 0 to day 106. FIG. 22E: Endogenous PAD activity measured in plasma with Histone H3 citrullination assay, day 0 to day 106. LD: Low dose. HD: High dose. Abatacept: Abatacept. Format: Bis3 (scFv PAD4; Fab: PAD2; Name: Clone 12). Numbers refer to individual experiments.

FIG. 23A-B: Potency of anti-PAD2 and anti-PAD4 antibodies against recombinant PAD

The potency of clones 12, 22 and 42 was directly compared to those of the art anti-PAD2 and anti-PAD4 antibodies using the optimised Histone-H3 citrullination ELISA and recombinant PAD2 and PAD4 (FIG. 23A and FIG. 23B, respectively).

FIG. 24A-B: PAD2/PAD4 specificity

The affinity optimised anti-PAD4 antibodies and bispecific formats were specific for PAD2 and/or PAD4 and did not bind PAD3 (FIG. 24A). The affinity optimised clone did not bind PAD3 (FIG. 24B).

FIG. 25A-B: Comparative potency assay of Bis3 and DuetMab formats

PAD activity was measured with the histone H3 citrullination assay. FIG. 25A: Data is representative of one of five experiments using different RA synovial fluid samples. FIG. 25B: Whole blood. Data is representative of one representative sample of whole blood. Bis3 clone=Clone 12. DuetMab clone=Clone 06.

FIG. 26: Potency of anti-PAD2 and anti-PAD4 antibodies in RA synovial fluid

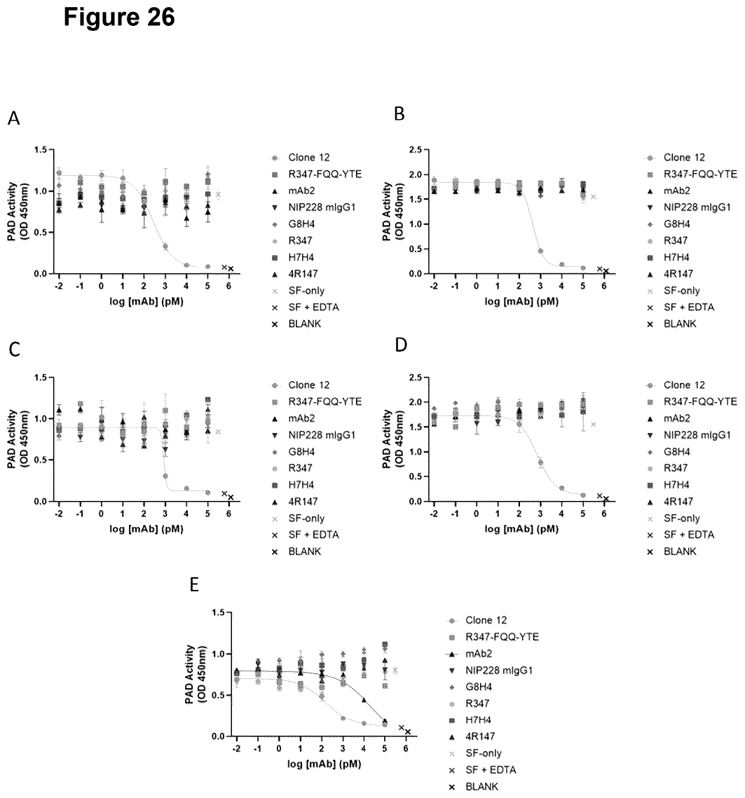

PAD activity was measured with the histone H3 citrullination assay using different RA synovial fluid samples. FIG. 26A: Sample 1. FIG. 26B: Sample 2. FIG. 26C: Sample 3. FIG. 26D: Sample 5. FIG. 26E: Sample 4.

### 6 DETAILED DESCRIPTION

Any compositions or methods provided herein can be combined with one or more of any of the other compositions and methods provided herein.

### 6.1 Sequences

The antibody or polypeptide of the invention may comprise amino acid sequences as provided in Table 1-Table 58. The antibody may have the amino acid sequence (VH, VL, HCDR1, HCDR2, HCDR3, LCDR1, LCDR2, LCDR3, full sequence, Fab, scFv, constant light chain (CL), heavy chain (HC), light chain (LC), CH1, CH2, CH3) as provided in any of Table 1-Table 58.

TABLE 1
anti-PAD2 (Clone 22)-Variable (VH/VL) (141LO0035 hIgG1 pgl-4)

| SEQ | | |
|---|---|---|
| ID | | |
| NO: | Description | Sequence |
| 1 | PAD2 VH | EVQLLESGGGLVQPGGSLRLSCAASGFTFSSYAMSWVRQAPGKGLEWV |
| | | SAISGSGGSTYYADSVKGRFTISRDNSKNTLYLQMNSLRAEDTAVYYC |
| | | ARQVLVRGFFSHEDDAVDIWGQGTTVTVSS |
| 2 | PAD2 VL | SYVLTQPPSVSVSPGQTASITCSGDKVGDKYVSWYQQKPGQSPVLVIY |
| | | QDSQRPSGIPERFSGSNSGNTATLTISGTQAMDEADYYCQTWAPDVLL |
| | | FGSGTKVTVL |
| 3 | PAD2 HCDR1 (Kabat) AA | SYAMS |
| 4 | PAD2 HCDR2 (Kabat) AA | AISGSGGSTYYADSVKG |
| 5 | PAD2 HCDR3 (Kabat) AA | QVLVRGFFSHEDDAVDI |
| 6 | PAD2 HFW1 (Kabat) AA | EVQLLESGGGLVQPGGSLRLSCAASGFTFS |
| 7 | PAD2 HFW2 (Kabat) AA | WVRQAPGKGLEWVS |
| 8 | PAD2 HFW3 (Kabat) AA | RFTISRDNSKNTLYLQMNSLRAEDTAVYYCAR |
| 9 | PAD2 HFW4 (Kabat) AA | WGQGTTVTVSS |
| 10 | PAD2 LCDR1 (Kabat) AA | SGDKVGDKYVS |
| 11 | PAD2 LCDR2 (Kabat) AA | QDSQRPS |
| 12 | PAD2 LCDR3 (Kabat) AA | QTWAPDVLL |
| 13 | PAD2 LFW1 (Kabat) AA | SYVLTQPPSVSVSPGQTASITC |
| 14 | PAD2 LFW2 (Kabat) AA | WYQQKPGQSPVLVIY |
| 15 | PAD2 LFW3 (Kabat) AA | GIPERFSGSNSGNTATLTISGTQAMDEADYYC |
| 16 | PAD2 LFW4 (Kabat) AA | FGSGTKVTVL |

TABLE 2
anti-PAD4 (Clone 42)-Variable (VH/VL) (48LO0063 hIgG1 fgl-59)

| SEQ ID NO. | Description | Sequence |
|---|---|---|
| 17 | PAD4 HCDR1 (Kabat) AA | DYFVS |
| 18 | PAD4 HCDR2 (Kabat) AA | FINAANTFTYYADSVRG |
| 19 | PAD4 HCDR3 (Kabat) AA | ANDDVDDIVAPGRGYYMDV |
| 20 | PAD4 HFW1 (Kabat) AA | QVQLVESGGGLVKPGGSLRLSCAASGSTLS |
| 21 | PAD4 HFW2 (Kabat) AA | WIRQAPGKGLEWVS |
| 22 | PAD4 HFW3 (Kabat) AA | RFTISRDNAKNSVYLQMNSLRAEDTAVYYCSS |
| 23 | PAD4 HFW4 (Kabat) AA | WGRGTLVTVSS |
| 24 | PAD4 LCDR1 (Kabat) AA | TGTSGDVGRYSHVS |
| 25 | PAD4 LCDR2 (Kabat) AA | NVYERPS |
| 26 | PAD4 LCDR3 (Kabat) AA | SSHSRSSTPVL |
| 27 | PAD4 LFW1 (Kabat) AA | QSALTQPRSVSGSPGQSVTISC |
| 28 | PAD4 LFW2 (Kabat) AA | WYQQHPGKAPKLIIY |
| 29 | PAD4 LFW3 (Kabat) AA | GVPDRESGSKSGNTASLTISGLQAEDEADYYC |
| 30 | PAD4 LFW4 (Kabat) AA | FGGGTKLTVL |
| 31 | PAD4 VH | QVQLVESGGGLVKPGGSLRLSCAASGSTLSDYFVSWIRQAPGKGLEWV |
| | | SFINAANTFTYYADSVRGRFTISRDNAKNSVYLQMNSLRAEDTAVYYC |
| | | SSANDDVDDIVAPGRGYYMDVWGRGTLVTVSS |
| 32 | PAD4 VL | QSALTQPRSVSGSPGQSVTISCTGTSGDVGRYSHVSWYQQHPGKAPKL |
| | | IIYNVYERPSGVPDRESGSKSGNTASLTISGLQAEDEADYYCSSHSRS |
| | | STPVLFGGGTKLTVL |

TABLE 3
48LO0010 hIgG1 ngl-2 (anti-PAD4)

| | SEQ | |
|---|---|---|
| | ID | |
| Description | NO: | Sequence |
| VH (Kabat) AA | 90 | QVQLVESGGGLVKPG |
| | | GSLRLSCAASGSTLS |
| | | DYFVSWIRQAPGKGL |
| | | EWVSYISSFGTETYY |
| | | ADSVRGRFTISRDNA |
| | | KNTVYLQMNSLRAED |
| | | TAVYYCSSATENVDD |
| | | IVAPGRGYYMDVWGR |
| | | GTLVTVSS |
| HCDR1 (Kabat) AA | 17 | DYFVS |
| HCDR2 (Kabat) AA | 89 | YISSFGTFTYYADS |
| | | VRG |
| HCDR3 (Kabat) AA | 75 | ATENVDDIVAPGRGY |
| | | YMDV |
| HFW1 (Kabat) AA | 20 | QVQLVESGGGLVKPG |
| | | GSLRLSCAASGSTLS |
| HFW2 (Kabat) AA | 21 | WIRQAPGKGLEWVS |
| HFW3 (Kabat) AA | 91 | RFTISRDNAKNTVYL |
| | | QMNSLRAEDTAVYYC |
| | | SS |
| HFW4 (Kabat) AA | 23 | WGRGTLVTVSS |
| VL (Kabat) AA | 92 | QSALTQPRSVSGSPG |
| | | QSVTISCTGTRGDVG |
| | | RYNHVSWYQQHPGKA |
| | | PKLIIYNVYERPSGV |
| | | PDRFSGSKSGNTASL |
| | | TISGLQAEDEADYYC |
| | | SSHSRSSTPVLFGGG |
| | | TKLTVL |
| LCDR1 (Kabat) AA | 93 | TGTRGDVGRYNHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPG |
| | | QSVTISC |
| LFW2 (Kabat) AA | 28 | WYQQHPGKAPKLIIY |
| LFW3 (Kabat) AA | 29 | GVPDRFSGSKSGNTA |
| | | SLTISGLQAEDEADY |
| | | YC |
| LFW4 (Kabat) AA | 30 | FGGGTKLTVL |

TABLE 4
48LO0032 hIgG1 ngl-2 (anti-PAD4)

| Description | SEQ ID NO: | Sequence |
|---|---|---|
| VH (Kabat) AA | 94 | QVQLVESGGGLVKPG |
| | | GSLRLSCAASGSTLS |
| | | DYFVSWIRQAPGKGL |
| | | EWVSFVNSADTFTYY |
| | | ADSVRGRFTISRDNA |
| | | KNTVYLQMNSLRAED |
| | | TAVYYCSSATEEVDD |
| | | IVAPGRGYYMDVWGR |
| | | GTLVTVSS |
| HCDR1 (Kabat) AA | 17 | DYFVS |
| HCDR2 (Kabat) AA | 95 | FVNSADTFTYYADS |
| | | VRG |
| HCDR3 (Kabat) AA | 96 | ATEEVDDIVAPGRG |
| | | YYMDV |
| HFW1 (Kabat) AA | 20 | QVQLVESGGGLVKPG |
| | | GSLRLSCAASGSTLS |
| HFW2 (Kabat) AA | 21 | WIRQAPGKGLEWVS |
| HFW3 (Kabat) AA | 91 | RFTISRDNAKNTVYLQ |
| | | MNSLRAEDTAVYYCSS |
| HFW4 (Kabat) AA | 23 | WGRGTLVTVSS |
| VL (Kabat) AA | 92 | QSALTQPRSVSGSPG |
| | | QSVTISCTGTRGDVG |
| | | RYNHVSWYQQHPGKA |
| | | PKLIIYNVYERPSGV |
| | | PDRESGSKSGNTASL |
| | | TISGLQAEDEADYYC |
| | | SSHSRSSTPVLFGGG |
| | | TKLTVL |
| LCDR1 (Kabat) AA | 93 | TGTRGDVGRYNHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGS |
| | | PGQSVTISC |
| LFW2 (Kabat) AA | 28 | WYQQHPGKAPKLIIY |
| LFW3 (Kabat) AA | 29 | GVPDRFSGSKSGNTA |
| | | SLTISGLQAEDEADY |
| | | YC |
| LFW4 (Kabat) AA | 30 | FGGGTKLTVL |

TABLE 5
48LO0033 hIgG1 ngl-3 (anti-PAD4)

| Description | SEQ ID NO: | Sequence |
|---|---|---|
| VH (Kabat) AA | 97 | QVQLVESGGGLVKPG |
| | | GSLRLSCAASGSTLS |
| | | DYFVSWIRQAPGKGL |
| | | EWVSYISSYGHYYTY |
| | | ADSVRGRFTISRDNA |
| | | KNTVYLQMNSLRAED |
| | | TAVYYCASKPDDVDD |
| | | IVAPGRGYYMDVWGR |
| | | GTLVTVSS |
| HCDR1 (Kabat) AA | 17 | DYFVS |
| HCDR2 (Kabat) AA | 98 | YISSYGHYYTYADSVRG |
| HCDR3 (Kabat) AA | 99 | KPDDVDDIVAPGRGYY |
| | | MDV |
| HFW1 (Kabat) AA | 20 | QVQLVESGGGLVKPG |
| | | GSLRLSCAASGSTLS |
| HFW2 (Kabat) AA | 21 | WIRQAPGKGLEWVS |
| HFW3 (Kabat) AA | 100 | RFTISRDNAKNTVYLQ |
| | | MNSLRAEDTAVYYCAS |
| HFW4 (Kabat) AA | 23 | WGRGTLVTVSS |
| VL (Kabat) AA | 92 | QSALTQPRSVSGSPG |
| | | QSVTISCTGTRGDVG |
| | | RYNHVSWYQQHPGKA |
| | | PKLIIYNVYERPSGV |
| | | PDRESGSKSGNTASL |
| | | TISGLQAEDEADYYC |
| | | SSHSRSSTPVLFGGG |
| | | TKLTVL |
| LCDR1 (Kabat) AA | 93 | TGTRGDVGRYNHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPG |
| | | QSVTISC |
| LFW2 (Kabat) AA | 28 | WYQQHPGKAPKLIIY |
| LFW3 (Kabat) AA | 29 | GVPDRFSGSKSGNTA |
| | | SLTISGLQAEDEADY |
| | | YC |
| LFW4 (Kabat) AA | 30 | FGGGTKLTVL |

TABLE 6
48LO0036 hIgG1 ngl-3 (anti-PAD4)

| Description | SEQ ID NO: | Sequence |
|---|---|---|
| VH (Kabat) AA | 10 | QVQLVESGGGLVKPG |
| | | GSLRLSCAASGSTLS |
| | | DYFVSWIRQAPGKGL |
| | | EWVSVDSSYSTETYY |
| | | ADSVRGRFTISRDNA |
| | | KNTVYLQMNSLRAED |
| | | TAVYYCTSATVSVDD |
| | | IVAPGRGYYMDVWGR |
| | | GTLVTVSS |
| HCDR1 (Kabat) AA | 17 | DYFVS |
| HCDR2 (Kabat) AA | 102 | VDSSYSTFTYYADSVRG |
| HCDR3 (Kabat) AA | 103 | ATVSVDDIVAPGRGYY |
| | | MDV |
| HFW1 (Kabat) AA | 20 | QVQLVESGGGLVKPGG |
| | | SLRLSCAASGSTLS |
| HFW2 (Kabat) AA | 21 | WIRQAPGKGLEWVS |
| HFW3 (Kabat) AA | 134 | RFTISRDNAKNTVYLQ |
| | | MNSLRAEDTAVYYCTS |
| HFW4 (Kabat) AA | 23 | WGRGTLVTVSS |
| VL (Kabat) AA | 92 | QSALTQPRSVSGSPG |
| | | QSVTISCTGTRGDVG |
| | | RYNHVSWYQQHPGKA |
| | | PKLIIYNVYERPSGV |
| | | PDRESGSKSGNTASL |
| | | TISGLQAEDEADYYC |
| | | SSHSRSSTPVLFGGG |
| | | TKLTVL |
| LCDR1 (Kabat) AA | 93 | TGTRGDVGRYNHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPG |
| | | QSVTISC |
| LFW2 (Kabat) AA | 28 | WYQQHPGKAPKLIIY |
| LFW3 (Kabat) AA | 29 | GVPDRFSGSKSGNTAS |
| | | LTISGLQAEDEADYYC |
| LFW4 (Kabat) AA | 30 | FGGGTKLTVL |

TABLE 7
48LO0040 hIgG1 ngl-3 (anti-PAD4)

| Description | SEQ ID NO: | Sequence |
|---|---|---|
| VH (Kabat) AA | 106 | QVQLVESGGGLVKPG |
| | | GSLRLSCADSGSTLS |
| | | DYFVSWIRQAPGKGL |
| | | EWVSFINSADTFTYY |
| | | ADSVRGRFTISRDNA |
| | | KNTVYLQMNSLRAED |
| | | TAVYYCATDIEVVDD |
| | | IVAPGRGYYMDVWGR |
| | | GTLVTVSS |
| HCDR1 (Kabat) AA | 17 | DYFVS |
| HCDR2 (Kabat) AA | 107 | FINSADTFTYYADS |
| | | VRG |
| HCDR3 (Kabat) AA | 108 | DIEVVDDIVAPGRGY |
| | | YMDV |
| HFW1 (Kabat) AA | 109 | QVQLVESGGGLVKPG |
| | | GSLRLSCADSGSTLS |
| HFW2 (Kabat) AA | 21 | WIRQAPGKGLEWVS |
| HFW3 (Kabat) AA | 112 | RFTISRDNAKNTVYLQ |
| | | MNSLRAEDTAVYYCAT |
| HFW4 (Kabat) AA | 23 | WGRGTLVTVSS |
| VL (Kabat) AA | 92 | QSALTQPRSVSGSPG |
| | | QSVTISCTGTRGDVG |
| | | RYNHVSWYQQHPGKA |
| | | PKLIIYNVYERPSGV |
| | | PDRFSGSKSGNTASL |
| | | TISGLQAEDEADYYC |
| | | SSHSRSSTPVLFGGG |
| | | TKLTVL |
| LCDR1 (Kabat) AA | 93 | TGTRGDVGRYNHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPG |
| | | QSVTISC |
| LFW2 (Kabat) AA | 28 | WYQQHPGKAPKLITY |
| LFW3 (Kabat) AA | 29 | GVPDRESGSKSGNTA |
| | | SLTISGLQAEDEADY |
| | | YC |
| LFW4 (Kabat) AA | 30 | FGGGTKLTVL |

TABLE 8
48LO0048 hIgG1 ngl-3 (anti-PAD4)

| Description | SEQ ID NO: | Sequence |
|---|---|---|
| VH (Kabat) AA | 110 | QVQLVESGGGLVKPG |
| | | GSLRLSCAASGSTLS |
| | | DYFVSWIRQAPGKGL |
| | | EWVSYISSFGTFTYY |
| | | ADSVRGRFTISRDNA |
| | | KNTVYLQMNSLRAED |
| | | TAVYYCATEQDFVDD |
| | | IVAPGRGYYMDVWGR |
| | | GTLVTVSS |
| HCDR1 (Kabat) AA | 17 | DYFVS |
| HCDR2 (Kabat) AA | 89 | YISSFGTFTYYADSV |
| | | RG |
| HCDR3 (Kabat) AA | 111 | EQDFVDDIVAPGRGY |
| | | YMDV |
| HFW1 (Kabat) AA | 20 | QVQLVESGGGLVKPGG |
| | | SLRLSCAASGSTLS |
| HFW2 (Kabat) AA | 21 | WIRQAPGKGLEWVS |
| HFW3 (Kabat) AA | 112 | RFTISRDNAKNTVYL |
| | | QMNSLRAEDTAVYYC |
| | | AT |
| HFW4 (Kabat) AA | 23 | WGRGTLVTVSS |
| VL (Kabat) AA | 113 | QSALTQPRSVSGSPG |
| | | QSVTISCTGTRGDVG |
| | | RYNHVSWYQQHPGKA |
| | | PKLIIYNVYERPSGV |
| | | PDRFSGSKSGNTASL |
| | | TISGLQAEDEADYYC |
| | | SSHSRVNPPVLFGGG |
| | | TKLTVL |
| LCDR1 (Kabat) AA | 93 | TGTRGDVGRYNHVS |
| LCDR2 (Kabat) AA | 25 | NVYERPS |
| LCDR3 (Kabat) AA | 114 | SSHSRVNPPVL |
| LFW1 (Kabat) AA | 27 | QSALTQPRSVSGS |
| | | PGQSVTISC |
| LFW2 (Kabat) AA | 28 | WYQQHPGKAPKLIIY |
| LFW3 (Kabat) AA | 29 | GVPDRFSGSKSGNTA |
| | | SLTISGLQAEDEADY |
| | | YC |
| LFW4 (Kabat) AA | 30 | FGGGTKLTVL |

TABLE 9
48LO0049 hIgG1 ngl-3 (anti-PAD4)

| Description | SEQ ID NO: | Sequence |
|---|---|---|
| VH (Kabat) AA | 115 | QVQLVESGGGLVKPG |
| | | GSLRLSCAASGSTLS |
| | | DYFVSWIRQAPGKGL |
| | | EWVSYISSFGTFTYY |
| | | ADSVRGRFTISRDNA |
| | | KNTVYLQMNSLRAED |
| | | TAVYYCSTEASFVDD |
| | | IVAPGRGYYMDVWGR |
| | | GTLVTVSS |
| HCDR1 (Kabat) AA | 17 | DYFVS |
| HCDR2 (Kabat) AA | 89 | YISSFGTFTYYADS |
| | | VRG |
| HCDR3 (Kabat) AA | 116 | EASFVDDIVAPGRGY |
| | | YMDV |
| HFW1 (Kabat) AA | 20 | QVQLVESGGGLVKPG |
| | | GSLRLSCAASGSTLS |
| HFW2 (Kabat) AA | 21 | WIRQAPGKGLEWVS |
| HFW3 (Kabat) AA | 117 | RFTISRDNAKNTVYL |
| | | QMNSLRAEDTAVYYC |
| | | ST |
| HFW4 (Kabat) AA | 23 | WGRGTLVTVSS |
| VL (Kabat) AA | 113 | QSALTQPRSVSGSPG |
| | | QSVTISCTGTRGDVG |
| | | RYNHVSWYQQHPGKA |
| | | PKLIIYNVYERPSGV |
| | | PDRESGSKSGNTASL |
| | | TISGLQAEDEADYYC |
| | | SSHSRVNPPVLEGGG |
| | | TKLTVL |
| LCDR1 (Kabat) AA | 93 | TGTRGDVGRYNHVS |
| LCDR2 (Kabat) AA | 25 | NVYERPS |
| LCDR3 (Kabat) AA | 114 | SSHSRVNPPVL |
| LFW1 (Kabat) AA | 27 | QSALTQPRSVSGS |
| | | PGQSVTISC |
| LFW2 (Kabat) AA | 28 | WYQQHPGKAPKLIIY |
| LFW3 (Kabat) AA | 29 | GVPDRFSGSKSGNTA |
| | | SLTISGLQAEDEADY |
| | | YC |
| LFW4 (Kabat) AA | 30 | FGGGTKLTVL |

TABLE 10
48LO0049 hIgG1 pgl-9 (anti-PAD4)

| Description | SEQ ID NO: | Sequence |
|---|---|---|
| VH (Kabat) AA | 115 | QVQLVESGGGLVKPG |
| | | GSLRLSCAASGSTLS |
| | | DYFVSWIRQAPGKGL |
| | | EWVSYISSFGTFTYY |
| | | ADSVRGRFTISRDNA |
| | | KNTVYLQMNSLRAED |
| | | TAVYYCSTEASFVDD |
| | | IVAPGRGYYMDVWGR |
| | | GTLVTVSS |
| HCDR1 (Kabat) AA | 17 | DYFVS |
| HCDR2 (Kabat) AA | 89 | YISSFGTFTYYADSVRG |
| HCDR3 (Kabat) AA | 116 | EASFVDDIVAPGRGYY |
| | | MDV |
| HFW1 (Kabat) AA | 20 | QVQLVESGGGLVKPGG |
| | | SLRLSCAASGSTLS |
| HFW2 (Kabat) AA | 21 | WIRQAPGKGLEWVS |
| HFW3 (Kabat) AA | 117 | RFTISRDNAKNTVYL |
| | | QMNSLRAEDTAVYYC |
| | | ST |
| HFW4 (Kabat) AA | 23 | WGRGTLVTVSS |
| VL (Kabat) AA | 142 | QSALTQPRSVSGSPG |
| | | QSVTISCTGTSGDVG |
| | | RYNHVSWYQQHPGKA |
| | | PKLIIYNVYERPSGV |
| | | PDRFSGSKSGNTASL |
| | | TISGLQAEDEADYYC |
| | | SSHSRVNPPVLFGGG |
| | | TKLTVL |
| LCDR1 (Kabat) AA | 141 | TGTSGDVGRYNHVS |
| LCDR2 (Kabat) AA | 25 | NVYERPS |
| LCDR3 (Kabat) AA | 114 | SSHSRVNPPVL |
| LFW1 (Kabat) AA | 27 | QSALTQPRSVSGS |
| | | PGQSVTISC |
| LFW2 (Kabat) AA | 28 | WYQQHPGKAPKLIIY |
| LFW3 (Kabat) AA | 29 | GVPDRESGSKSGNTA |
| | | SLTISGLQAEDEADY |
| | | YC |
| LFW4 (Kabat) AA | 30 | FGGGTKLTVL |

TABLE 11
48LO0049 hIgG1 pgl-10 (anti-PAD4)

| Description | SEQ ID NO: | Sequence |
|---|---|---|
| VH (Kabat) AA | 115 | QVQLVESGGGLVKPG |
| | | GSLRLSCAASGSTLS |
| | | DYFVSWIRQAPGKGL |
| | | EWVSYISSFGTFTYY |
| | | ADSVRGRFTISRDNA |
| | | KNTVYLQMNSLRAED |
| | | TAVYYCSTEASFVDD |
| | | IVAPGRGYYMDVWGR |
| | | GTLVTVSS |
| HCDR1 (Kabat) AA | 17 | DYFVS |
| HCDR2 (Kabat) AA | 89 | YISSFGTFTYYAD |
| | | SVRG |
| HCDR3 (Kabat) AA | 116 | EASFVDDIVAPGRG |
| | | YYMDV |
| HFW1 (Kabat) AA | 20 | QVQLVESGGGLVKPG |
| | | GSLRLSCAASGSTLS |
| HFW2 (Kabat) AA | 21 | WIRQAPGKGLEWVS |
| HFW3 (Kabat) AA | 117 | RFTISRDNAKNTVYL |
| | | QMNSLRAEDTAVYYC |
| | | ST |
| HFW4 (Kabat) AA | 23 | WGRGTLVTVSS |
| VL (Kabat) AA | 146 | QSALTQPRSVSGSPG |
| | | QSVTISCTGTRSDVG |
| | | RYNHVSWYQQHPGKA |
| | | PKLIIYNVYERPSGV |
| | | PDRESGSKSGNTASL |
| | | TISGLQAEDEADYYC |
| | | SSHSRVNPPVLFGGG |
| | | TKLTVL |
| LCDR1 (Kabat) AA | 147 | TGTRSDVGRYNHVS |
| LCDR2 (Kabat) AA | 25 | NVYERPS |
| LCDR3 (Kabat) AA | 114 | SSHSRVNPPVL |
| LFW1 (Kabat) AA | 27 | QSALTQPRSVSGS |
| | | PGQSVTISC |
| LFW2 (Kabat) AA | 28 | WYQQHPGKAPKLIIY |
| LFW3 (Kabat) AA | 29 | GVPDRFSGSKSGNTA |
| | | SLTISGLQAEDEADY |
| | | YC |
| LFW4 (Kabat) AA | 30 | FGGGTKLTVL |

TABLE 12
48LO0049 hIgG1 pgl-12 (anti-PAD4)

| Description | SEQ ID NO: | Sequence |
|---|---|---|
| VH (Kabat) AA | 115 | QVQLVESGGGLVKPG |
| | | GSLRLSCAASGSTLS |
| | | DYFVSWIRQAPGKGL |
| | | EWVSYISSFGTFTYY |
| | | ADSVRGRFTISRDNA |
| | | KNTVYLQMNSLRAED |
| | | TAVYYCSTEASFVDD |
| | | IVAPGRGYYMDVWGR |
| | | GTLVTVSS |
| HCDR1 (Kabat) AA | 17 | DYFVS |
| HCDR2 (Kabat) AA | 89 | YISSFGTFTYYADSVRG |
| HCDR3 (Kabat) AA | 116 | EASFVDDIVAPGRGYYMDV |
| HFW1 (Kabat) AA | 20 | QVQLVESGGGLVKPG |
| | | GSLRLSCAASGSTLS |
| HFW2 (Kabat) AA | 21 | WIRQAPGKGLEWVS |
| HFW3 (Kabat) AA | 117 | RFTISRDNAKNTVYL |
| | | QMNSLRAEDTAVYYC |
| | | ST |
| HFW4 (Kabat) AA | 23 | WGRGTLVTVSS |
| VL (Kabat) AA | 118 | QSALTQPRSVSGSPG |
| | | QSVTISCTGTRGDVG |
| | | RYSHVSWYQQHPGKA |
| | | PKLIIYNVYERPSGV |
| | | PDRESGSKSGNTASL |
| | | TISGLQAEDEADYYC |
| | | SSHSRVNPPVLFGGG |
| | | TKLTVL |
| LCDR1 (Kabat) AA | 119 | TGTRGDVGRYSHVS |
| LCDR2 (Kabat) AA | 25 | NVYERPS |
| LCDR3 (Kabat) AA | 114 | SSHSRVNPPVL |
| LFW1 (Kabat) AA | 27 | QSALTQPRSVSGSP |
| | | GQSVTISC |
| LFW2 (Kabat) AA | 28 | WYQQHPGKAPKLIIY |
| LFW3 (Kabat) AA | 29 | GVPDRFSGSKSGNTA |
| | | SLTISGLQAEDEADY |
| | | YC |
| LFW4 (Kabat) AA | 30 | FGGGTKLTVL |

TABLE 13
48LO0049 hIgG1 fgl-23 (anti-PAD4)

| Description | SEQ ID NO: | Sequence |
|---|---|---|
| VH (Kabat) AA | 120 | QVQLVESGGGLVKPG |
| | | GSLRLSCAASGSTLS |
| | | DYFVSWIRQAPGKGL |
| | | EWVSYISSFGTFTYY |
| | | ADSVRGRFTISRDNA |
| | | KNSVYLQMNSLRAED |
| | | TAVYYCSTEASFVDD |
| | | IVAPGRGYYMDVWGR |
| | | GTLVTVSS |
| HCDR1 (Kabat) AA | 17 | DYFVS |
| HCDR2 (Kabat) AA | 89 | YISSFGTFTYYAD |
| | | SVRG |
| HCDR3 (Kabat) AA | 116 | EASFVDDIVAPGRG |
| | | YYMDV |
| HFW1 (Kabat) AA | 20 | QVQLVESGGGLVKPG |
| | | GSLRLSCAASGSTLS |
| HFW2 (Kabat) AA | 21 | WIRQAPGKGLEWVS |
| HFW3 (Kabat) AA | 121 | RFTISRDNAKNSVYL |
| | | QMNSLRAEDTAVYYC |
| | | ST |
| HFW4 (Kabat) AA | 23 | WGRGTLVTVSS |
| VL (Kabat) AA | 122 | QSALTQPRSVSGSPG |
| | | QSVTISCTGTRSDVG |
| | | RYNYVSWYQQHPGKA |
| | | PKLIIYNVYERPSGV |
| | | PDRESGSKSGNTASL |
| | | TISGLQAEDEADYYC |
| | | SSHSRVNPPVLFGGG |
| | | TKLTVL |
| LCDR1 (Kabat) AA | 123 | TGTRSDVGRYNYVS |
| LCDR2 (Kabat) AA | 25 | NVYERPS |
| LCDR3 (Kabat) AA | 114 | SSHSRVNPPVL |
| LFW1 (Kabat) AA | 27 | QSALTQPRSVSGSPG |
| | | QSVTISC |
| LFW2 (Kabat) AA | 28 | WYQQHPGKAPKLIIY |
| LFW3 (Kabat) AA | 29 | GVPDRFSGSKSGNTAS |
| | | LTISGLQAEDEADYYC |
| LFW4 (Kabat) AA | 30 | FGGGTKLTVL |

TABLE 14
48LO0049 hIgG1 fgl-25 (anti-PAD4)

| Description | SEQ ID NO: | Sequence |
|---|---|---|
| VH (Kabat) AA | 120 | QVQLVESGGGLVKPG |
| | | GSLRLSCAASGSTLS |
| | | DYFVSWIRQAPGKGL |
| | | EWVSYISSFGTFTYY |
| | | ADSVRGRFTISRDNA |
| | | KNSVYLQMNSLRAED |
| | | TAVYYCSTEASFVDD |
| | | IVAPGRGYYMDVWGR |
| | | GTLVTVSS |
| HCDR1 (Kabat) AA | 17 | DYFVS |
| HCDR2 (Kabat) AA | 89 | YISSFGTFTYYAD |
| | | SVRG |
| HCDR3 (Kabat) AA | 116 | EASFVDDIVAPGR |
| | | GYYMDV |
| HFW1 (Kabat) AA | 20 | QVQLVESGGGLVKPG |
| | | GSLRLSCAASGSTLS |
| HFW2 (Kabat) AA | 21 | WIRQAPGKGLEWVS |
| HFW3 (Kabat) AA | 121 | RFTISRDNAKNSVYL |
| | | QMNSLRAEDTAVYYC |
| | | ST |
| HFW4 (Kabat) AA | 23 | WGRGTLVTVSS |
| VL (Kabat) AA | 124 | QSALTQPRSVSGSPG |
| | | QSVTISCTGTRADVG |
| | | RYNQVSWYQQHPGKA |
| | | PKLIIYNVYERPSGV |
| | | PDRFSGSKSGNTASL |
| | | TISGLQAEDEADYYC |
| | | SSHSRVNPPVLFGGG |
| | | TKLTVL |
| LCDR1 (Kabat) AA | 125 | TGTRADVGRYNQVS |
| LCDR2 (Kabat) AA | 25 | NVYERPS |
| LCDR3 (Kabat) AA | 114 | SSHSRVNPPVL |
| LFW1 (Kabat) AA | 27 | QSALTQPRSVSGSPG |
| | | QSVTISC |
| LFW2 (Kabat) AA | 28 | WYQQHPGKAPKLIIY |
| LFW3 (Kabat) AA | 29 | GVPDRFSGSKSGNTASL |
| | | TISGLQAEDEADYYC |
| LFW4 (Kabat) AA | 30 | FGGGTKLTVL |

TABLE 15
48LO0049 hIgG1 pgl-31 (anti-PAD4)

| Description | SEQ ID NO: | Sequence |
|---|---|---|
| VH (Kabat) AA | 115 | QVQLVESGGGLVKPG |
| | | GSLRLSCAASGSTLS |
| | | DYFVSWIRQAPGKGL |
| | | EWVSYISSFGTFTYY |
| | | ADSVRGRFTISRDNA |
| | | KNTVYLQMNSLRAED |
| | | TAVYYCSTEASFVDD |
| | | IVAPGRGYYMDVWGR |
| | | GTLVTVSS |
| HCDR1 (Kabat) AA | 17 | DYFVS |
| HCDR2 (Kabat) AA | 89 | YISSFGTFTYYAD |
| | | SVRG |
| HCDR3 (Kabat) AA | 116 | EASFVDDIVAPGRG |
| | | YYMDV |
| HFW1 (Kabat) AA | 20 | QVQLVESGGGLVKPG |
| | | GSLRLSCAASGSTLS |
| HFW2 (Kabat) AA | 21 | WIRQAPGKGLEWVS |
| HFW3 (Kabat) AA | 117 | RFTISRDNAKNTVYL |
| | | QMNSLRAEDTAVYYC |
| | | ST |
| HFW4 (Kabat) AA | 23 | WGRGTLVTVSS |
| VL (Kabat) AA | 126 | QSALTQPRSVSGSPG |
| | | QSVTISCTGTRGDVG |
| | | RYNYVSWYQQHPGKA |
| | | PKLIIYNVYERPSGV |
| | | PDRESGSKSGNTASL |
| | | TISGLQAEDEADYYC |
| | | SSHSRVNPPVLFGGG |
| | | TKLTVL |
| LCDR1 (Kabat) AA | 127 | TGTRGDVGRYNYVS |
| LCDR2 (Kabat) AA | 25 | NVYERPS |
| LCDR3 (Kabat) AA | 114 | SSHSRVNPPVL |
| LFW1 (Kabat) AA | 27 | QSALTQPRSVSGS |
| | | PGQSVTISC |
| LFW2 (Kabat) AA | 28 | WYQQHPGKAPKLIIY |
| LFW3 (Kabat) AA | 29 | GVPDRESGSKSGNTA |
| | | SLTISGLQAEDEADY |
| | | YC |
| LFW4 (Kabat) AA | 30 | FGGGTKLTVL |

TABLE 16
48LO0051 hIgG1 ngl-2 (anti-PAD4)

| Description | SEQ ID NO: | Sequence |
|---|---|---|
| VH (Kabat) AA | 128 | QVQLVESGGGLVKPG |
| | | GSLRLSCAASGSTLS |
| | | DYFVSWIRQAPGKGL |
| | | EWVSYISSFGTFTYY |
| | | ADSVRGRFTISRDNA |
| | | KNTVYLQMNSLRAED |
| | | TAVYYCSTATEPVDD |
| | | IVAPGRGYYMDVWGR |
| | | GTLVTVSS |
| HCDR1 (Kabat) AA | 17 | DYFVS |
| HCDR2 (Kabat) AA | 89 | YISSFGTFTYYADS |
| | | VRG |
| HCDR3 (Kabat) AA | 129 | ATEPVDDIVAPGRG |
| | | YYMDV |
| HFW1 (Kabat) AA | 20 | QVQLVESGGG |
| | | LVKPGGSLRL |
| | | SCAASGSTLS |
| HFW2 (Kabat) AA | 21 | WIRQAPGKGLEWVS |
| HFW3 (Kabat) AA | 117 | RFTISRDNAKNTVYL |
| | | QMNSLRAEDTAVYYC |
| | | ST |
| HFW4 (Kabat) AA | 23 | WGRGTLVTVSS |
| VL (Kabat) AA | 113 | QSALTQPRSVSGSPG |
| | | QSVTISCTGTRGDVG |
| | | RYNHVSWYQQHPGKA |
| | | PKLIIYNVYERPSGV |
| | | PDRFSGSKSGNTASL |
| | | TISGLQAEDEADYYC |
| | | SSHSRVNPPVLFGGG |
| | | TKLTVL |
| LCDR1 (Kabat) AA | 93 | TGTRGDVGRYN |
| | | HVS |
| LCDR2 (Kabat) AA | 25 | NVYERPS |
| LCDR3 (Kabat) AA | 114 | SSHSRVNPPVL |
| LFW1 (Kabat) AA | 27 | QSALTQPRSVS |
| | | GSPGQSVTISC |
| LFW2 (Kabat) AA | 28 | WYQQHPGKAPK |
| | | LIIY |
| LFW3 (Kabat) AA | 29 | GVPDRFSGSK |
| | | SGNTASLTIS |
| | | GLQAEDEADY |
| | | YC |
| LFW4 (Kabat) AA | 30 | FGGGTKLTVL |

TABLE 17
48LO0060 hIgG1 ngl-3 (anti-PAD4)

| Description | SEQ ID NO: | Sequence |
|---|---|---|
| VH (Kabat) AA | 130 | QVQLVESGGG |
| | | LVKPGGSLRL |
| | | SCAASGSTLS |
| | | DYFVSWIRQA |
| | | PGKGLEWVSF |
| | | VNSANTFTYY |
| | | ADSVRGRFTI |
| | | SRDNAKNTVY |
| | | LQMNSLRAED |
| | | TAVYYCTSDA |
| | | PEVDDIVAPG |
| | | RGYYMDVWGR |
| | | GTLVTVSS |
| HCDR1 (Kabat) AA | 17 | DYFVS |
| HCDR2 (Kabat) AA | 131 | FVNSANTFTY |
| | | YADSVRG |
| HCDR3 (Kabat) AA | 132 | DAPEVDDIVAP |
| | | GRGYYMDV |
| HFW1 (Kabat) AA | 20 | QVQLVESGGG |
| | | LVKPGGSLRL |
| | | SCAASGSTLS |
| HFW2 (Kabat) AA | 21 | WIRQAPGKGL |
| | | EWVS |
| HFW3 (Kabat) AA | 134 | RFTISRDNAK |
| | | NTVYLQMNSL |
| | | RAEDTAVYYC |
| | | TS |
| HFW4 (Kabat) AA | 23 | WGRGTLVTVSS |
| VL (Kabat) AA | 92 | QSALTQPRSV |
| | | SGSPGQSVTI |
| | | SCTGTRGDVG |
| | | RYNHVSWYQQ |
| | | HPGKAPKLII |
| | | YNVYERPSGV |
| | | PDRESGSKSG |
| | | NTASLTISGL |
| | | QAEDEADYYC |
| | | SSHSRSSTPV |
| | | LFGGGTKLTV |
| | | L |
| LCDR1 (Kabat) AA | 93 | TGTRGDVGRY |
| | | NHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVS |
| | | GSPGQSVTISC |
| LFW2 (Kabat) AA | 28 | WYQQHPGKAPK |
| | | LIIY |
| LFW3 (Kabat) AA | 29 | GVPDRFSGSK |
| | | SGNTASLTIS |
| | | GLQAEDEADY |
| | | YC |
| LFW4 (Kabat) AA | 30 | FGGGTKLTVL |

TABLE 18
48LO0062 hIgG1 ngl-3 (anti-PAD4)

| | SEQ | |
|---|---|---|
| | ID | |
| Description | NO: | Sequence |
| VH (Kabat) AA | 135 | QVQLVESGGG |
| | | LVKPGGSLRL |
| | | SCAASGSTLS |
| | | DYFVSWIRQA |
| | | PGKGLEWVSY |
| | | ISSFGTYYTY |
| | | ADSVRGRFTI |
| | | SRDNAKNTVY |
| | | LQMNSLRAED |
| | | TAVYYCASAT |
| | | TNVDDIVAPG |
| | | RGYYMDVWGR |
| | | GTLVTVSS |
| HCDR1 (Kabat) AA | 17 | DYFVS |
| HCDR2 (Kabat) AA | 136 | YISSFGTYYTY |
| | | ADSVRG |
| HCDR3 (Kabat) AA | 137 | ATTNVDDIVA |
| | | PGRGYYMDV |
| HFW1 (Kabat) AA | 20 | QVQLVESGGG |
| | | LVKPGGSLRL |
| | | SCAASGSTLS |
| HFW2 (Kabat) AA | 21 | WIRQAPGKGL |
| | | EWVS |
| HFW3 (Kabat) AA | 100 | RFTISRDNAK |
| | | NTVYLQMNSL |
| | | RAEDTAVYYC |
| | | AS |
| HFW4 (Kabat) AA | 23 | WGRGTLVTVSS |
| VL (Kabat) AA | 92 | QSALTQPRSV |
| | | SGSPGQSVTI |
| | | SCTGTRGDVG |
| | | RYNHVSWYQQ |
| | | HPGKAPKLII |
| | | YNVYERPSGV |
| | | PDRFSGSKSG |
| | | NTASLTISGL |
| | | QAEDEADYYC |
| | | SSHSRSSTPV |
| | | LFGGGTKLTV |
| | | L |
| LCDR1 (Kabat) AA | 93 | TGTRGDVGRY |
| | | NHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVS |
| | | GSPGQSVTISC |
| LFW2 (Kabat) AA | 28 | WYQQHPGKAP |
| | | KLIIY |
| LFW3 (Kabat) AA | 29 | GVPDRFSGSK |
| | | SGNTASLTIS |
| | | GLQAEDEADY |
| | | YC |
| LFW4 (Kabat) AA | 30 | FGGGTKLTVL |

TABLE 19
48LO0063 hIgG1 ngl-3 (anti-PAD4)

| Description | SEQ ID NO: | Sequence |
|---|---|---|
| VH (Kabat) AA | 138 | QVQLVESGGG |
| | | LVKPGGSLRL |
| | | SCAASGSTLS |
| | | DYFVSWIRQA |
| | | PGKGLEWVSF |
| | | INSANTFTYY |
| | | ADSVRGRFTI |
| | | SRDNAKNTVY |
| | | LQMNSLRAED |
| | | TAVYYCSSAN |
| | | DDVDDIVAPG |
| | | RGYYMDVWGR |
| | | GTLVTVSS |
| HCDR1 (Kabat) AA | 17 | DYFVS |
| HCDR2 (Kabat) AA | 139 | FINSANTFTY |
| | | YADSVRG |
| HCDR3 (Kabat) AA | 19 | ANDDVDDIVA |
| | | PGRGYYMDV |
| HFW1 (Kabat) AA | 20 | QVQLVESGGG |
| | | LVKPGGSLRL |
| | | SCAASGSTLS |
| HFW2 (Kabat) AA | 21 | WIRQAPGKGL |
| | | EWVS |
| HFW3 (Kabat) AA | 91 | RFTISRDNAK |
| | | NTVYLQMNSL |
| | | RAEDTAVYYC |
| | | SS |
| HFW4 (Kabat) AA | 23 | WGRGTLVTVSS |
| VL (Kabat) AA | 92 | QSALTQPRSV |
| | | SGSPGQSVTI |
| | | SCTGTRGDVG |
| | | RYNHVSWYQQ |
| | | HPGKAPKLII |
| | | YNVYERPSGV |
| | | PDRFSGSKSG |
| | | NTASLTISGL |
| | | QAEDEADYYC |
| | | SSHSRSSTPV |
| | | LFGGGTKLTV |
| | | L |
| LCDR1 (Kabat) AA | 93 | TGTRGDVGRY |
| | | NHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSV |
| | | SGSPGQSVTI |
| | | SC |
| LFW2 (Kabat) AA | 28 | WYQQHPGKAP |
| | | KLIIY |
| LFW3 (Kabat) AA | 29 | GVPDRFSGSK |
| | | SGNTASLTIS |
| | | GLQAEDEADY |
| | | YC |
| LFW4 (Kabat) AA | 30 | FGGGTKLTVL |

TABLE 20
48LO0063 hlgG1 fgl-4 (anti-PAD4)

| Description | SEQ ID NO: | Sequence |
|---|---|---|
| VH (Kabat) AA | 140 | QVQLVESGGG |
| | | LVKPGGSLRL |
| | | SCAASGSTLS |
| | | DYFVSWIRQA |
| | | PGKGLEWVSF |
| | | INSANTFTYY |
| | | ADSVRGRFTI |
| | | SRDNAKNSVY |
| | | LQMNSLRAED |
| | | TAVYYCSSAN |
| | | DDVDDIVAPG |
| | | RGYYMDVWGR |
| | | GTLVTVSS |
| HCDR1 (Kabat) AA | 17 | DYFVS |
| HCDR2 (Kabat) AA | 139 | FINSANTFTY |
| | | YADSVRG |
| HCDR3 (Kabat) AA | 19 | ANDDVDDIVA |
| | | PGRGYYMDV |
| HFW1 (Kabat) AA | 20 | QVQLVESGGG |
| | | LVKPGGSLRL |
| | | SCAASGSTLS |
| HFW2 (Kabat) AA | 21 | WIRQAPGKGLE |
| | | WVS |
| HFW3 (Kabat) AA | 22 | RFTISRDNAKNS |
| | | VYLQMNSLRAED |
| | | TAVYYCSS |
| HFW4 (Kabat) AA | 23 | WGRGTLVTVSS |
| VL (Kabat) AA | 148 | QSALTQPRSV |
| | | SGSPGQSVTI |
| | | SCTGTSGDVG |
| | | RYNHVSWYQQ |
| | | HPGKAPKLII |
| | | YNVYERPSGV |
| | | PDRESGSKSG |
| | | NTASLTISGL |
| | | QAEDEADYYC |
| | | SSHSRSSTPV |
| | | LFGGGTKLTV |
| | | L |
| LCDR1 (Kabat) AA | 141 | TGTSGDVGRY |
| | | NHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSV |
| | | SGSPGQSVTI |
| | | SC |
| LFW2 (Kabat) AA | 28 | WYQQHPGKAP |
| | | KLIIY |
| LFW3 (Kabat) AA | 29 | GVPDRFSGSK |
| | | SGNTASLTIS |
| | | GLQAEDEADY |
| | | YC |
| LFW4 (Kabat) AA | 30 | FGGGTKLTVL |

TABLE 21
48LO0063 hIgG1 fgl-6 (anti-PAD4)

| | SEQ | |
|---|---|---|
| | ID | |
| Description | NO: | Sequence |
| VH (Kabat) AA | 140 | QVQLVESGGGLVKPGGSLRL |
| | | SCAASGSTLSDYFVSWIRQA |
| | | PGKGLEWVSFINSANTFTYY |
| | | ADSVRGRFTISRDNAKNSVY |
| | | LQMNSLRAEDTAVYYCSSAN |
| | | DDVDDIVAPGRGYYMDVWGR |
| | | GTLVTVSS |
| HCDR1 (Kabat) AA | 17 | DYFVS |
| HCDR2 (Kabat) AA | 139 | FINSANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRL |
| | | SCAASGSTLS |
| HFW2 (Kabat) AA | 21 | WIRQAPGKGLEWVS |
| HFW3 (Kabat) AA | 22 | RFTISRDNAKNSVYLQMNSL |
| | | RAEDTAVYYCSS |
| HFW4 (Kabat) AA | 23 | WGRGTLVTVSS |
| VL (Kabat) AA | 32 | QSALTQPRSVSGSPGQSVTI |
| | | SCTGTSGDVGRYSHVSWYQQ |
| | | HPGKAPKLIIYNVYERPSGV |
| | | PDRESGSKSGNTASLTISGL |
| | | QAEDEADYYCSSHSRSSTPV |
| | | LFGGGTKLTVL |
| LCDR1 (Kabat) AA | 24 | TGTSGDVGRYSHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTI |
| | | SC |
| LFW2 (Kabat) AA | 28 | WYQQHPGKAPKLIIY |
| LFW3 (Kabat) AA | 29 | GVPDRFSGSKSGNTASLTIS |
| | | GLQAEDEADYYC |
| LFW4 (Kabat) AA | 30 | FGGGTKLTVL |

TABLE 22
48LO0063 hIgG1 fgl-7 (anti-PAD4)

| | SEQ | |
|---|---|---|
| | ID | |
| Description | NO: | Sequence |
| VH (Kabat) AA | 140 | QVQLVESGGGLVKPG |
| | | GSLRLSCAASGSTLS |
| | | DYFVSWIRQAPGKGL |
| | | EWVSFINSANTFTYY |
| | | ADSVRGRFTISRDNA |
| | | KNSVYLQMNSLRAED |
| | | TAVYYCSSANDDVDD |
| | | IVAPGRGYYMDVWGR |
| | | GTLVTVSS |
| HCDR1 (Kabat) AA | 17 | DYFVS |
| HCDR2 (Kabat) AA | 139 | FINSANTFTYYADSV |
| | | RG |
| HCDR3 (Kabat) AA | 19 | ANDDVDDIVAPGRGY |
| | | YMDV |
| HFW1 (Kabat) AA | 20 | QVQLVESGGGLVKPG |
| | | GSLRLSCAASGSTLS |
| HFW2 (Kabat) AA | 21 | WIRQAPGKGLEWVS |
| HFW3 (Kabat) AA | 22 | RFTISRDNAKNSVYL |
| | | QMNSLRAEDTAVYYC |
| | | SS |
| HFW4 (Kabat) AA | 23 | WGRGTLVTVSS |
| VL (Kabat) AA | 153 | QSALTQPRSVSGSPG |
| | | QSVTISCTGTRGDVG |
| | | RYNQVSWYQQHPGKA |
| | | PKLIIYNVYERPSGV |
| | | PDRESGSKSGNTASL |
| | | TISGLQAEDEADYYC |
| | | SSHSRSSTPVLFGGG |
| | | TKLTVL |
| LCDR1 (Kabat) AA | 154 | TGTRGDVGRYNQVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPG |
| | | QSVTISC |
| LFW2 (Kabat) AA | 28 | WYQQHPGKAPKLIIY |
| LFW3 (Kabat) AA | 29 | GVPDRESGSKSGNTA |
| | | SLTISGLQAEDEADY |
| | | YC |
| LFW4 (Kabat) AA | 30 | FGGGTKLTVL |

TABLE 23
48LO0063 hIgG1 fgl-8 (anti-PAD4)

| | SEQ | |
|---|---|---|
| | ID | |
| Description | NO: | Sequence |
| VH (Kabat) AA | 140 | QVQLVESGGGLVKPGGSLRL |
| | | SCAASGSTLSDYFVSWIRQ |
| | | APGKGL |
| | | EWVSFINSANTFTYYADSVR |
| | | GRFTISRDNAKNSVYLQMNS |
| | | LRAED |
| | | TAVYYCSSANDDVDDIVAPG |
| | | RGYYMDVWGRGTLVTVSS |
| HCDR1 (Kabat) AA | 17 | DYFVS |
| HCDR2 (Kabat) AA | 139 | FINSANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRL |
| | | SCAASGSTLS |
| HFW2 (Kabat) AA | 21 | WIRQAPGKGLEWVS |
| HFW3 (Kabat) AA | 22 | RFTISRDNAKNSVYLQMNSL |
| | | RAEDTAVYYCSS |
| HFW4 (Kabat) AA | 23 | WGRGTLVTVSS |
| VL (Kabat) AA | 149 | QSALTQPRSVSGSPGQSVTI |
| | | SCTGTRPDVGRYNQVSWYQQ |
| | | HPGKA |
| | | PKLIIYNVYERPSGVPDRES |
| | | GSKSGNTASLTISGLQAEDE |
| | | ADYYC |
| | | SSHSRSSTPVLFGGGTKLTV |
| | | L |
| LCDR1 (Kabat) AA | 150 | TGTRPDVGRYNQVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTI |
| | | SC |
| LFW2 (Kabat) AA | 28 | WYQQHPGKAPKLIIY |
| LFW3 (Kabat) AA | 29 | GVPDRESGSKSGNTASLTIS |
| | | GLQAEDEADYYC |
| LFW4 (Kabat) AA | 30 | FGGGTKLTVL |

TABLE 24
48LO0063 hIgG1 fgl-9 (anti-PAD4)

| | SEQ | |
|---|---|---|
| | ID | |
| Description | NO: | Sequence |
| VH (Kabat) AA | 151 | QVQLVESGGGLVKPGGSLRL |
| | | SCAASGSTLSDYFVSWIRQA |
| | | PGKGL |
| | | EWVSFISSANTFTYYADSVR |
| | | GRFTISRDNAKNSVYLQMNS |
| | | LRAED |
| | | TAVYYCSSANDDVDDIVAPG |
| | | RGYYMDVWGRGTLVTVSS |
| HCDR1 (Kabat) AA | 17 | DYFVS |
| HCDR2 (Kabat) AA | 152 | FISSANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRL |
| | | SCAASGSTLS |
| HFW2 (Kabat) AA | 21 | WIRQAPGKGLEWVS |
| HFW3 (Kabat) AA | 22 | RFTISRDNAKNSVYLQMNSL |
| | | RAEDTAVYYCSS |
| HFW4 (Kabat) AA | 23 | WGRGTLVTVSS |
| VL (Kabat) AA | 148 | QSALTQPRSVSGSPGQSVTI |
| | | SCTGTSGDVGRYNHVSWYQQ |
| | | HPGKA |
| | | PKLIIYNVYERPSGVPDRFS |
| | | GSKSGNTASLTISGLQAEDE |
| | | ADYYC |
| | | SSHSRSSTPVLFGGGTKLTV |
| | | L |
| LCDR1 (Kabat) AA | 141 | TGTSGDVGRYNHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTI |
| | | SC |
| LFW2 (Kabat) AA | 28 | WYQQHPGKAPKLIIY |
| LFW3 (Kabat) AA | 29 | GVPDRESGSKSGNTASLTIS |
| | | GLQAEDEADYYC |
| LFW4 (Kabat) AA | 30 | FGGGTKLTVL |

TABLE 25
48LO0063 hIgG1 fgl-11 (anti-PAD4)

| | SEQ | |
|---|---|---|
| | ID | |
| Description | NO: | Sequence |
| VH (Kabat) AA | 151 | QVQLVESGGGLVKPGGSLRL |
| | | SCAASGSTLSDYFVSWIRQA |
| | | PGKGL |
| | | EWVSFISSANTFTYYADSVR |
| | | GRFTISRDNAKNSVYLQMNS |
| | | LRAED |
| | | TAVYYCSSANDDVDDIVAPG |
| | | RGYYMDVWGRGTLVTVSS |
| HCDR1 (Kabat) AA | 17 | DYFVS |
| HCDR2 (Kabat) AA | 152 | FISSANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRL |
| | | SCAASGSTLS |
| HFW2 (Kabat) AA | 21 | WIRQAPGKGLEWVS |
| HFW3 (Kabat) AA | 22 | RFTISRDNAKNSVYLQMNSL |
| | | RAEDTAVYYCSS |
| HFW4 (Kabat) AA | 23 | WGRGTLVTVSS |
| VL (Kabat) AA | 32 | QSALTQPRSVSGSPGQSVTI |
| | | SCTGTSGDVGRYSHVSWYQQ |
| | | HPGKA |
| | | PKLIIYNVYERPSGVPDRES |
| | | GSKSGNTASLTISGLQAEDE |
| | | ADYYC |
| | | SSHSRSSTPVLFGGGTKLTV |
| | | L |
| LCDR1 (Kabat) AA | 24 | TGTSGDVGRYSHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTI |
| | | SC |
| LFW2 (Kabat) AA | 28 | WYQQHPGKAPKLIIY |
| LFW3 (Kabat) AA | 29 | GVPDRFSGSKSGNTASLTIS |
| | | GLQAEDEADYYC |
| LFW4 (Kabat) AA | 30 | FGGGTKLTVL |

TABLE 26
48LO0063 hIgG1 pgl-38 (anti-PAD4)

| | SEQ | |
|---|---|---|
| | ID | |
| Description | NO: | Sequence |
| VH (Kabat) AA | 155 | QVQLVESGGGLVKPGGSLRL |
| | | SCAASGSTLSDYFVSWIRQA |
| | | PGKGLE |
| | | WVSFINDANTFTYYADSVRG |
| | | RFTISRDNAKNTVYLQMNSL |
| | | RAEDTA |
| | | VYYCSSANDDVDDIVAPGRG |
| | | YYMDVWGRGTLVTVSS |
| HCDR1 (Kabat) AA | 17 | DYFVS |
| HCDR2 (Kabat) AA | | FINDANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRL |
| | | SCAASGSTLS |
| HFW2 (Kabat) AA | 21 | WIRQAPGKGLEWVS |
| HFW3 (Kabat) AA | 91 | RFTISRDNAKNTVYLQMNSL |
| | | RAEDTAVYYCSS |
| HFW4 (Kabat) AA | 23 | WGRGTLVTVSS |
| VL (Kabat) AA | 92 | QSALTQPRSVSGSPGQSVTI |
| | | SCTGTRGDVGRYNHVSWYQQ |
| | | HPGKAP |
| | | KLIIYNVYERPSGVPDRESG |
| | | SKSGNTASLTISGLQAEDEA |
| | | DYYCSS |
| | | HSRSSTPVLFGGGTKLTVL |
| LCDR1 (Kabat) AA | 93 | TGTRGDVGRYNHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTI |
| | | SC |
| LFW2 (Kabat) AA | 28 | WYQQHPGKAPKLIIY |
| LFW3 (Kabat) AA | 29 | GVPDRESGSKSGNTASLTIS |
| | | GLQAEDEADYYC |
| LFW4 (Kabat) AA | 30 | FGGGTKLTVL |

TABLE 27
48LO0063 hIgG1 pgl-39 (anti-PAD4)

| | SEQ | |
|---|---|---|
| | ID | |
| Description | NO: | Sequence |
| VH (Kabat) AA | 156 | QVQLVESGGGLVKPGGSLRL |
| | | SCAASGSTLSDYFVSWIRQA |
| | | PGKGL |
| | | EWVSFIKSANTFTYYADSVR |
| | | GRFTISRDNAKNTVYLQMNS |
| | | LRAED |
| | | TAVYYCSSANDDVDDIVAPG |
| | | RGYYMDVWGRGTLVTVSS |
| HCDR1 (Kabat) AA | 17 | DYFVS |
| HCDR2 (Kabat) AA | 157 | FIKSANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRL |
| | | SCAASGSTLS |
| HFW2 (Kabat) AA | 21 | WIRQAPGKGLEWVS |
| HFW3 (Kabat) AA | 91 | RFTISRDNAKNTVYLQMNSL |
| | | RAEDTAVYYCSS |
| HFW4 (Kabat) AA | 23 | WGRGTLVTVSS |
| VL (Kabat) AA | 92 | QSALTQPRSVSGSPGQSVTI |
| | | SCTGTRGDVGRYNHVSWYQQ |
| | | HPGKA |
| | | PKLIIYNVYERPSGVPDRFS |
| | | GSKSGNTASLTISGLQAEDE |
| | | ADYYC |
| | | SSHSRSSTPVLFGGGTKLTV |
| | | L |
| LCDR1 (Kabat) AA | 93 | TGTRGDVGRYNHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTI |
| | | SC |
| LFW2 (Kabat) AA | 28 | WYQQHPGKAPKLIIY |
| LFW3 (Kabat) AA | 29 | GVPDRFSGSKSGNTASLTIS |
| | | GLQAEDEADYYC |
| LFW4 (Kabat) AA | 30 | FGGGTKLTVL |

TABLE 28
48LO0063 hIgG1 pgl-40 (anti-PAD4)

| | SEQ | |
|---|---|---|
| | ID | |
| Description | NO: | Sequence |
| VH (Kabat) AA | 158 | QVQLVESGGGLVKPGGSLRL |
| | | SCAASGSTLSDYFVSWIRQA |
| | | PGKGLEWVSFIPSANTFTYY |
| | | ADSVRGRFTISRDNAKNTVY |
| | | LQMNSLRAED |
| | | TAVYYCSSANDDVDDIVAPG |
| | | RGYYMDVWGRGTLVTVSS |
| HCDR1 (Kabat) AA | 17 | DYFVS |
| HCDR2 (Kabat) AA | 159 | FIPSANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRL |
| | | SCAASGSTLS |
| HFW2 (Kabat) AA | 21 | WIRQAPGKGLEWVS |
| HFW3 (Kabat) AA | 91 | RFTISRDNAKNTVYLQMNSL |
| | | RAEDTAVYYCSS |
| HFW4 (Kabat) AA | 23 | WGRGTLVTVSS |
| VL (Kabat) AA | 92 | QSALTQPRSVSGSPGQSVTI |
| | | SCTGTRGDVGRYNHVSWYQQ |
| | | HPGKA |
| | | PKLIIYNVYERPSGVPDRES |
| | | GSKSGNTASLTISGLQAEDE |
| | | ADYYC |
| | | SSHSRSSTPVLFGGGTKLTV |
| | | L |
| LCDR1 (Kabat) AA | 93 | TGTRGDVGRYNHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTI |
| | | SC |
| LFW2 (Kabat) AA | 28 | WYQQHPGKAPKLIIY |
| LFW3 (Kabat) AA | 29 | GVPDRFSGSKSGNTASLTIS |
| | | GLQAEDEADYYC |
| LFW4 (Kabat) AA | 30 | FGGGTKLTVL |

TABLE 29
48LO0063 hIgG1 pgl-41 (anti-PAD4)

| | SEQ | |
|---|---|---|
| | ID | |
| Description | NO: | Sequence |
| VH (Kabat) AA | 160 | QVQLVESGGGLVKPGGSLRL |
| | | SCAASGSTLSDYFVSWIRQA |
| | | PGKGLEW |
| | | VSFIASANTFTYYADSVRGR |
| | | FTISRDNAKNTVYLQMNSLR |
| | | AEDTAVY |
| | | YCSSANDDVDDIVAPGRGYY |
| | | MDVWGRGTLVTVSS |
| HCDR1 (Kabat) AA | 17 | DYFVS |
| HCDR2 (Kabat) AA | 161 | FIASANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRL |
| | | SCAASGSTLS |
| HFW2 (Kabat) AA | 21 | WIRQAPGKGLEWVS |
| HFW3 (Kabat) AA | 91 | RFTISRDNAKNTVYLQMNSL |
| | | RAEDTAVYYCSS |
| HFW4 (Kabat) AA | 23 | WGRGTLVTVSS |
| VL (Kabat) AA | 92 | QSALTQPRSVSGSPGQSVTI |
| | | SCTGTRGDVGRYNHVSWYQQ |
| | | HPGKAPK |
| | | LIIYNVYERPSGVPDRESGS |
| | | KSGNTASLTISGLQAEDEAD |
| | | YYCSSHS |
| | | RSSTPVLFGGGTKLTVL |
| LCDR1 (Kabat) AA | 93 | TGTRGDVGRYNHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTI |
| | | SC |
| LFW2 (Kabat) AA | 28 | WYQQHPGKAPKLIIY |
| LFW3 (Kabat) AA | 29 | GVPDRESGSKSGNTASLTIS |
| | | GLQAEDEADYYC |
| LFW4 (Kabat) AA | 30 | FGGGTKLTVL |

TABLE 30
48LO0063 hIgG1 pgl-42 (anti-PAD4)

| | SEQ | |
|---|---|---|
| | ID | |
| Description | NO: | Sequence |
| VH (Kabat) AA | 162 | QVQLVESGGGLVKPGGSLRL |
| | | SCAASGSTLSDYFVSWIRQA |
| | | PGKGLE |
| | | WVSFITSANTFTYYADSVRG |
| | | RFTISRDNAKNTVYLQMNSL |
| | | RAEDTA |
| | | VYYCSSANDDVDDIVAPGRG |
| | | YYMDVWGRGTLVTVSS |
| HCDR1 (Kabat) AA | 17 | DYFVS |
| HCDR2 (Kabat) AA | 163 | FITSANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRL |
| | | SCAASGSTLS |
| HFW2 (Kabat) AA | 21 | WIRQAPGKGLEWVS |
| HFW3 (Kabat) AA | 91 | RFTISRDNAKNTVYLQMNSL |
| | | RAEDTAVYYCSS |
| HFW4 (Kabat) AA | 23 | WGRGTLVTVSS |
| VL (Kabat) AA | 92 | QSALTQPRSVSGSPGQSVTI |
| | | SCTGTRGDVGRYNHVSWYQQ |
| | | HPGKAP |
| | | KLIIYNVYERPSGVPDRESG |
| | | SKSGNTASLTISGLQAEDEA |
| | | DYYCSS |
| | | HSRSSTPVLFGGGTKLTVL |
| LCDR1 (Kabat) AA | 93 | TGTRGDVGRYNHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTI |
| | | SC |
| LFW2 (Kabat) AA | 28 | WYQQHPGKAPKLIIY |
| LFW3 (Kabat) AA | 29 | GVPDRFSGSKSGNTASLTIS |
| | | GLQAEDEADYYC |
| LFW4 (Kabat) AA | 30 | FGGGTKLTVL |

TABLE 31
48LO0063 hIgG1 pgl-43 (anti-PAD4)

| | SEQ | |
|---|---|---|
| | ID | |
| Description | NO: | Sequence |
| VH (Kabat) AA | 164 | QVQLVESGGGLVKPGGSLRL |
| | | SCAASGSTLSDYFVSWIRQA |
| | | PGKGLEW |
| | | VSFIESANTFTYYADSVRGR |
| | | FTISRDNAKNTVYLQMNSLR |
| | | AEDTAVY |
| | | YCSSANDDVDDIVAPGRGYY |
| | | MDVWGRGTLVTVSS |
| HCDR1 (Kabat) AA | 17 | DYFVS |
| HCDR2 (Kabat) AA | 165 | FIESANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRL |
| | | SCAASGSTLS |
| HFW2 (Kabat) AA | 21 | WIRQAPGKGLEWVS |
| HFW3 (Kabat) AA | 91 | RFTISRDNAKNTVYLQMNSL |
| | | RAEDTAVYYCSS |
| HFW4 (Kabat) AA | 23 | WGRGTLVTVSS |
| VL (Kabat) AA | 92 | QSALTQPRSVSGSPGQSVTI |
| | | SCTGTRGDVGRYNHVSWYQQ |
| | | HPGKAPK |
| | | LIIYNVYERPSGVPDRESGS |
| | | KSGNTASLTISGLQAEDEAD |
| | | YYCSSHS |
| | | RSSTPVLFGGGTKLTVL |
| LCDR1 (Kabat) AA | 93 | TGTRGDVGRYNHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTI |
| | | SC |
| LFW2 (Kabat) AA | 28 | WYQQHPGKAPKLIIY |
| LFW3 (Kabat) AA | 29 | GVPDRFSGSKSGNTASLTIS |
| | | GLQAEDEADYYC |
| LFW4 (Kabat) AA | 30 | FGGGTKLTVL |

TABLE 32
48LO0063 hIgG1 pgl-44 (anti-PAD4)

| | SEQ | |
|---|---|---|
| | ID | |
| Description | NO: | Sequence |
| VH (Kabat) AA | 166 | QVQLVESGGGLVKPGGSLRL |
| | | SCAASGSTLSDYFVSWIRQA |
| | | PGKGLEWVS |
| | | FIHSANTFTYYADSVRGRFT |
| | | ISRDNAKNTVYLQMNSLRAE |
| | | DTAVYYCSS |
| | | ANDDVDDIVAPGRGYYMDVW |
| | | GRGTLVTVSS |
| HCDR1 (Kabat) AA | 17 | DYFVS |
| HCDR2 (Kabat) AA | 167 | FIHSANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRL |
| | | SCAASGSTLS |
| HFW2 (Kabat) AA | 21 | WIRQAPGKGLEWVS |
| HFW3 (Kabat) AA | 91 | RFTISRDNAKNTVYLQMNSL |
| | | RAEDTAVYYCSS |
| HFW4 (Kabat) AA | 23 | WGRGTLVTVSS |
| VL (Kabat) AA | 92 | QSALTQPRSVSGSPGQSVTI |
| | | SCTGTRGDVGRYNHVSWYQQ |
| | | HPGKAPKLI |
| | | IYNVYERPSGVPDRESGSKS |
| | | GNTASLTISGLQAEDEADYY |
| | | CSSHSRSST |
| | | PVLFGGGTKLTVL |
| LCDR1 (Kabat) AA | 93 | TGTRGDVGRYNHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTI |
| | | SC |
| LFW2 (Kabat) AA | 28 | WYQQHPGKAPKLIIY |
| LFW3 (Kabat) AA | 29 | GVPDRFSGSKSGNTASLTIS |
| | | GLQAEDEADYYC |
| LFW4 (Kabat) AA | 30 | FGGGTKLTVL |

TABLE 33
48LO0063 hIgG1 pgl-45 (anti-PAD4)

| | SEQ | |
|---|---|---|
| Description | ID NO: | Sequence |
| VH (Kabat) AA | 168 | QVQLVESGGGLVKPGGSLRLSCA |
| | | ASGSTLSDYFVSWIRQAPGKGLE |
| | | WVSFIGSANTFTYYADSVRGRFT |
| | | ISRDNAKNTVYLQMNSLRAEDTA |
| | | VYYCSSANDDVDDIVAPGRGYYM |
| | | DVWGRGTLVTVSS |
| HCDR1 (Kabat) AA | 17 | DYFVS |
| HCDR2 (Kabat) AA | 169 | FIGSANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRLSCA |
| | | ASGSTLS |
| HFW2 (Kabat) AA | 21 | WIRQAPGKGLEWVS |
| HFW3 (Kabat) AA | 91 | RFTISRDNAKNTVYLQMNSLRAE |
| | | DTAVYYCSS |
| HFW4 (Kabat) AA | 23 | WGRGTLVTVSS |
| VL (Kabat) AA | 92 | QSALTQPRSVSGSPGQSVTISCT |
| | | GTRGDVGRYNHVSWYQQHPGKAP |
| | | KLIIYNVYERPSGVPDRFSGSKS |
| | | GNTASLTISGLQAEDEADYYCSS |
| | | HSRSSTPVLFGGGTKLTVL |
| LCDR1 (Kabat) AA | 93 | TGTRGDVGRYNHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTISC |
| LFW2 (Kabat) AA | 28 | WYQQHPGKAPKLIIY |
| LFW3 (Kabat) AA | 29 | GVPDRFSGSKSGNTASLTISGLQ |
| | | AEDEADYYC |
| LFW4 (Kabat) AA | 30 | FGGGTKLTVL |

TABLE 34
48LO0063 hIgG1 pgl-46 (anti-PAD4)

| | SEQ | |
|---|---|---|
| Description | ID NO: | Sequence |
| VH (Kabat) AA | 170 | QVQLVESGGGLVKPGGSLRLSCA |
| | | ASGSTLSDYFVSWIRQAPGKGLE |
| | | WVSFIQSANTFTYYADSVRGRFT |
| | | ISRDNAKNTVYLQMNSLRAEDTA |
| | | VYYCSSANDDVDDIVAPGRGYYM |
| | | DVWGRGTLVTVSS |
| HCDR1 (Kabat) AA | 17 | DYFVS |
| HCDR2 (Kabat) AA | 171 | FIQSANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRLSCA |
| | | ASGSTLS |
| HFW2 (Kabat) AA | 21 | WIRQAPGKGLEWVS |
| HFW3 (Kabat) AA | 91 | RFTISRDNAKNTVYLQMNSLRAE |
| | | DTAVYYCSS |
| HFW4 (Kabat) AA | 23 | WGRGTLVTVSS |
| VL (Kabat) AA | 92 | QSALTQPRSVSGSPGQSVTISCT |
| | | GTRGDVGRYNHVSWYQQHPGKAP |
| | | KLIIYNVYERPSGVPDRFSGSKS |
| | | GNTASLTISGLQAEDEADYYCSS |
| | | HSRSSTPVLFGGGTKLTVL |
| LCDR1 (Kabat) AA | 93 | TGTRGDVGRYNHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTISC |
| LFW2 (Kabat) AA | 28 | WYQQHPGKAPKLIIY |
| LFW3 (Kabat) AA | 29 | GVPDRFSGSKSGNTASLTISGLQ |
| | | AEDEADYYC |
| LFW4 (Kabat) AA | 30 | FGGGTKLTVL |

TABLE 35
48LO0063 hIgG1 pgl-47 (anti-PAD4)

| | SEQ | |
|---|---|---|
| Description | ID NO: | Sequence |
| VH (Kabat) AA | 172 | QVQLVESGGGLVKPGGSLRLSCA |
| | | ASGSTLSDYFVSWIRQAPGKGLE |
| | | WVSFINPANTFTYYADSVRGRFT |
| | | ISRDNAKNTVYLQMNSLRAEDTA |
| | | VYYCSSANDDVDDIVAPGRGYYM |
| | | DVWGRGTLVTVSS |
| HCDR1 (Kabat) AA | 17 | DYFVS |
| HCDR2 (Kabat) AA | 173 | FINPANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRLSCA |
| | | ASGSTLS |
| HFW2 (Kabat) AA | 21 | WIRQAPGKGLEWVS |
| HFW3 (Kabat) AA | 91 | RFTISRDNAKNTVYLQMNSLRAE |
| | | DTAVYYCSS |
| HFW4 (Kabat) AA | 23 | WGRGTLVTVSS |
| VL (Kabat) AA | 92 | QSALTQPRSVSGSPGQSVTISCT |
| | | GTRGDVGRYNHVSWYQQHPGKAP |
| | | KLIIYNVYERPSGVPDRFSGSKS |
| | | GNTASLTISGLQAEDEADYYCSS |
| | | HSRSSTPVLFGGGTKLTVL |
| LCDR1 (Kabat) AA | 93 | TGTRGDVGRYNHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTISC |
| LFW2 (Kabat) AA | 28 | WYQQHPGKAPKLIIY |
| LFW3 (Kabat) AA | 29 | GVPDRFSGSKSGNTASLTISGLQ |
| | | AEDEADYYC |
| LFW4 (Kabat) AA | 30 | FGGGTKLTVL |

TABLE 36
48LO0063 hIgG1 pgl-49 (anti-PAD4)

| | SEQ | |
|---|---|---|
| Description | ID NO: | Sequence |
| VH (Kabat) AA | 174 | QVQLVESGGGLVKPGGSLRLSCA |
| | | ASGSTLSDYFVSWIRQAPGKGLE |
| | | WVSFINTANTFTYYADSVRGRFT |
| | | ISRDNAKNTVYLQMNSLRAEDTA |
| | | VYYCSSANDDVDDIVAPGRGYYM |
| | | DVWGRGTLVTVSS |
| HCDR1 (Kabat) AA | 17 | DYFVS |
| HCDR2 (Kabat) AA | 175 | FINTANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRLSCA |
| | | ASGSTLS |
| HFW2 (Kabat) AA | 21 | WIRQAPGKGLEWVS |
| HFW3 (Kabat) AA | 91 | RFTISRDNAKNTVYLQMNSLRAE |
| | | DTAVYYCSS |
| HFW4 (Kabat) AA | 23 | WGRGTLVTVSS |
| VL (Kabat) AA | 92 | QSALTQPRSVSGSPGQSVTISCT |
| | | GTRGDVGRYNHVSWYQQHPGKAP |
| | | KLIIYNVYERPSGVPDRFSGSKS |
| | | GNTASLTISGLQAEDEADYYCSS |
| | | HSRSSTPVLFGGGTKLTVL |
| LCDR1 (Kabat) AA | 93 | TGTRGDVGRYNHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTISC |
| LFW2 (Kabat) AA | 28 | WYQQHPGKAPKLIIY |
| LFW3 (Kabat) AA | 29 | GVPDRFSGSKSGNTASLTISGLQ |
| | | AEDEADYYC |
| LFW4 (Kabat) AA | 30 | FGGGTKLTVL |

TABLE 37
48LO0063 hIgG1 pgl-51 (anti-PAD4)

| | SEQ | |
|---|---|---|
| Description | ID NO: | Sequence |
| VH (Kabat) AA | 176 | QVQLVESGGGLVKPGGSLRLSCA |
| | | ASGSTLSDYFVSWIRQAPGKGLE |
| | | WVSFINKANTFTYYADSVRGRFT |
| | | ISRDNAKNTVYLQMNSLRAEDTA |
| | | VYYCSSANDDVDDIVAPGRGYYM |
| | | DVWGRGTLVTVSS |
| HCDR1 (Kabat) AA | 17 | DYFVS |
| HCDR2 (Kabat) AA | 177 | FINKANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRLSCA |
| | | ASGSTLS |
| HFW2 (Kabat) AA | 21 | WIRQAPGKGLEWVS |
| HFW3 (Kabat) AA | 91 | RFTISRDNAKNTVYLQMNSLRAE |
| | | DTAVYYCSS |
| HFW4 (Kabat) AA | 23 | WGRGTLVTVSS |
| VL (Kabat) AA | 92 | QSALTQPRSVSGSPGQSVTISCT |
| | | GTRGDVGRYNHVSWYQQHPGKAP |
| | | KLIIYNVYERPSGVPDRFSGSKS |
| | | GNTASLTISGLQAEDEADYYCSS |
| | | HSRSSTPVLFGGGTKLTVL |
| LCDR1 (Kabat) AA | 93 | TGTRGDVGRYNHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTISC |
| LFW2 (Kabat) AA | 28 | WYQQHPGKAPKLIIY |
| LFW3 (Kabat) AA | 29 | GVPDRFSGSKSGNTASLTISGLQ |
| | | AEDEADYYC |
| LFW4 (Kabat) AA | 30 | FGGGTKLTVL |

TABLE 38
48LO0063 hIgG1 pgl-52 (anti-PAD4)

| | SEQ | |
|---|---|---|
| Description | ID NO: | Sequence |
| VH (Kabat) AA | 178 | QVQLVESGGGLVKPGGSLRLSCA |
| | | ASGSTLSDYFVSWIRQAPGKGLE |
| | | WVSFINAANTFTYYADSVRGRFT |
| | | ISRDNAKNTVYLQMNSLRAEDTA |
| | | VYYCSSANDDVDDIVAPGRGYYM |
| | | DVWGRGTLVTVSS |
| HCDR1 (Kabat) AA | 17 | DYFVS |
| HCDR2 (Kabat) AA | 18 | FINAANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRLSCA |
| | | ASGSTLS |
| HFW2 (Kabat) AA | 21 | WIRQAPGKGLEWVS |
| HFW3 (Kabat) AA | 91 | RFTISRDNAKNTVYLQMNSLRAE |
| | | DTAVYYCSS |
| HFW4 (Kabat) AA | 23 | WGRGTLVTVSS |
| VL (Kabat) AA | 92 | QSALTQPRSVSGSPGQSVTISCT |
| | | GTRGDVGRYNHVSWYQQHPGKAP |
| | | KLIIYNVYERPSGVPDRFSGSKS |
| | | GNTASLTISGLQAEDEADYYCSS |
| | | HSRSSTPVLFGGGTKLTVL |
| LCDR1 (Kabat) AA | 93 | TGTRGDVGRYNHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTISC |
| LFW2 (Kabat) AA | 28 | WYQQHPGKAPKLIIY |
| LFW3 (Kabat) AA | 29 | GVPDRFSGSKSGNTASLTISGLQ |
| | | AEDEADYYC |
| LFW4 (Kabat) AA | 30 | FGGGTKLTVL |

TABLE 39
48LO0063 hIgG1 pgl-53 (anti-PAD4)

| | SEQ | |
|---|---|---|
| Description | ID NO: | Sequence |
| VH (Kabat) AA | 179 | QVQLVESGGGLVKPGGSLRLSCA |
| | | ASGSTLSDYFVSWIRQAPGKGLE |
| | | WVSFINQANTFTYYADSVRGRFT |
| | | ISRDNAKNTVYLQMNSLRAEDTA |
| | | VYYCSSANDDVDDIVAPGRGYYM |
| | | DVWGRGTLVTVSS |
| HCDR1 (Kabat) AA | 17 | DYFVS |
| HCDR2 (Kabat) AA | 180 | FINQANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRLSCA |
| | | ASGSTLS |
| HFW2 (Kabat) AA | 21 | WIRQAPGKGLEWVS |
| HFW3 (Kabat) AA | 91 | RFTISRDNAKNTVYLQMNSLRAE |
| | | DTAVYYCSS |
| HFW4 (Kabat) AA | 23 | WGRGTLVTVSS |
| VL (Kabat) AA | 92 | QSALTQPRSVSGSPGQSVTISCT |
| | | GTRGDVGRYNHVSWYQQHPGKAP |
| | | KLIIYNVYERPSGVPDRFSGSKS |
| | | GNTASLTISGLQAEDEADYYCSS |
| | | HSRSSTPVLFGGGTKLTVL |
| LCDR1 (Kabat) AA | 93 | TGTRGDVGRYNHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTISC |
| LFW2 (Kabat) AA | 28 | WYQQHPGKAPKLIIY |
| LFW3 (Kabat) AA | 29 | GVPDRFSGSKSGNTASLTISGLQ |
| | | AEDEADYYC |
| LFW4 (Kabat) AA | 30 | FGGGTKLTVL |

TABLE 40
48LO0063 hIgG1 fgl-58 (anti-PAD4)

| | SEQ | |
|---|---|---|
| Description | ID NO: | Sequence |
| VH (Kabat) AA | 181 | QVQLVESGGGLVKPGGSLRLSCA |
| | | ASGSTLSDYFVSWIRQAPGKGLE |
| | | WVSFIPSANTFTYYADSVRGRFT |
| | | ISRDNAKNSVYLQMNSLRAEDTA |
| | | VYYCSSANDDVDDIVAPGRGYYM |
| | | DVWGRGTLVTVSS |
| HCDR1 (Kabat) AA | 17 | DYFVS |
| HCDR2 (Kabat) AA | 159 | FIPSANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRLSCA |
| | | ASGSTLS |
| HFW2 (Kabat) AA | 21 | WIRQAPGKGLEWVS |
| HFW3 (Kabat) AA | 22 | RFTISRDNAKNSVYLQMNSLRAE |
| | | DTAVYYCSS |
| HFW4 (Kabat) AA | 23 | WGRGTLVTVSS |
| VL (Kabat) AA | 32 | QSALTQPRSVSGSPGQSVTISCT |
| | | GTSGDVGRYSHVSWYQQHPGKAP |
| | | KLIIYNVYERPSGVPDRFSGSKS |
| | | GNTASLTISGLQAEDEADYYCSS |
| | | HSRSSTPVLFGGGTKLTVL |
| LCDR1 (Kabat) AA | 24 | TGTSGDVGRYSHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTISC |
| LFW2 (Kabat) AA | 28 | WYQQHPGKAPKLIIY |
| LFW3 (Kabat) AA | 29 | GVPDRFSGSKSGNTASLTISGLQ |
| | | AEDEADYYC |
| LFW4 (Kabat) AA | 30 | FGGGTKLTVL |

TABLE 41
48LO0063 hIgG1 fgl-60 (anti-PAD4)

| | SEQ | |
|---|---|---|
| Description | ID NO: | Sequence |
| VH (Kabat) AA | 183 | QVQLVESGGGLVKPGGSLRLSCA |
| | | ASGSTLSDYFVSWIRQAPGKGLE |
| | | WVSFINQANTFTYYADSVRGRFT |
| | | ISRDNAKNSVYLQMNSLRAEDTA |
| | | VYYCSSANDDVDDIVAPGRGYYM |
| | | DVWGRGTLVTVSS |
| HCDR1 (Kabat) AA | 17 | DYFVS |
| HCDR2 (Kabat) AA | 180 | FINQANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRLSCA |
| | | ASGSTLS |
| HFW2 (Kabat) AA | 21 | WIRQAPGKGLEWVS |
| HFW3 (Kabat) AA | 22 | RFTISRDNAKNSVYLQMNSLRAE |
| | | DTAVYYCSS |
| HFW4 (Kabat) AA | 23 | WGRGTLVTVSS |
| VL (Kabat) AA | 32 | QSALTQPRSVSGSPGQSVTISCT |
| | | GTSGDVGRYSHVSWYQQHPGKAP |
| | | KLIIYNVYERPSGVPDRESGSKS |
| | | GNTASLTISGLQAEDEADYYCSS |
| | | HSRSSTPVLFGGGTKLTVL |
| LCDR1 (Kabat) AA | 24 | TGTSGDVGRYSHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTISC |
| LFW2 (Kabat) AA | 28 | WYQQHPGKAPKLIIY |
| LFW3 (Kabat) AA | 29 | GVPDRFSGSKSGNTASLTISGLQ |
| | | AEDEADYYC |
| LFW4 (Kabat) AA | 30 | FGGGTKLTVL |

TABLE 42
48LO0063 hIgG1 fgl-61 (anti-PAD4)

| | SEQ | |
|---|---|---|
| Description | ID NO: | Sequence |
| VH (Kabat) AA | 185 | QVQLVESGGGLVKPGGSLRLSCA |
| | | ASGSTLSDYFVSWIRQAPGKGLE |
| | | WVSFINTANTFTYYADSVRGRFT |
| | | ISRDNAKNSVYLQMNSLRAEDTA |
| | | VYYCSSANDDVDDIVAPGRGYYM |
| | | DVWGRGTLVTVSS |
| HCDR1 (Kabat) AA | 17 | DYFVS |
| HCDR2 (Kabat) AA | 175 | FINTANTFTYYADSVRG |
| HCDR3 (Kabat) AA | 19 | ANDDVDDIVAPGRGYYMDV |
| HFW1 (Kabat) AA | 20 | QVQLVESGGGLVKPGGSLRLSCA |
| | | ASGSTLS |
| HFW2 (Kabat) AA | 21 | WIRQAPGKGLEWVS |
| HFW3 (Kabat) AA | 22 | RFTISRDNAKNSVYLQMNSLRAE |
| | | DTAVYYCSS |
| HFW4 (Kabat) AA | 23 | WGRGTLVTVSS |
| VL (Kabat) AA | 32 | QSALTQPRSVSGSPGQSVTISCT |
| | | GTSGDVGRYSHVSWYQQHPGKAP |
| | | KLIIYNVYERPSGVPDRFSGSKS |
| | | GNTASLTISGLQAEDEADYYCSS |
| | | HSRSSTPVLFGGGTKLTVL |
| LCDR1 (Kabat) AA | 24 | TGTSGDVGRYSHVS |
| LCDR2 (Kabat) AA | 25 | NVYERPS |
| LCDR3 (Kabat) AA | 26 | SSHSRSSTPVL |
| LFW1 (Kabat) AA | 27 | QSALTQPRSVSGSPGQSVTISC |
| LFW2 (Kabat) AA | 28 | WYQQHPGKAPKLIIY |
| LFW3 (Kabat) AA | 29 | GVPDRFSGSKSGNTASLTISGLQ |
| | | AEDEADYYC |
| LFW4 (Kabat) AA | 30 | FGGGTKLTVL |

TABLE 43
141LO0002 hIgG1 pgl-3 (anti-PAD2)

| | SEQ | |
|---|---|---|
| Description | ID NO: | Sequence |
| VH (Kabat) AA | 192 | EVQLLESGGGLVQPGGSLRLSCA |
| | | ASGFTFSSYAMSWVRQAPGKGLE |
| | | WVSAISGSGGSTYYADSVKGRFT |
| | | ISRDNSKNTLYLQMNSLRAEDTA |
| | | VYYCARHSYTRGFFSHEDDAVDI |
| | | WGQGTTVTVSS |
| HCDR1 (Kabat) AA | 193 | SYAMS |
| HCDR2 (Kabat) AA | 194 | AISGSGGSTYYADSVKG |
| HCDR3 (Kabat) AA | 195 | HSYTRGFFSHEDDAVDI |
| HFW1 (Kabat) AA | 196 | EVQLLESGGGLVQPGGSLRLSCA |
| | | ASGFTFS |
| HFW2 (Kabat) AA | 197 | WVRQAPGKGLEWVS |
| HFW3 (Kabat) AA | 198 | RFTISRDNSKNTLYLQMNSLRAE |
| | | DTAVYYCAR |
| HFW4 (Kabat) AA | 199 | WGQGTTVTVSS |
| VL (Kabat) AA | 200 | SYVLTQPPSVSVSPGQTASITCS |
| | | GDKVGDKYVSWYQQKPGQSPVLV |
| | | IYQDSQRPSGIPERFSGSNSGNT |
| | | ATLTISGTQAMDEADYYCEVGVD |
| | | YEYVFGSGTKVTVL |
| LCDR1 (Kabat) AA | 201 | SGDKVGDKYVS |
| LCDR2 (Kabat) AA | 202 | QDSQRPS |
| LCDR3 (Kabat) AA | 203 | EVGVDYEYV |
| LFW1 (Kabat) AA | 204 | SYVLTQPPSVSVSPGQTASITC |
| LFW2 (Kabat) AA | 205 | WYQQKPGQSPVLVIY |
| LFW3 (Kabat) AA | 206 | GIPERFSGSNSGNTATLTISGTQ |
| | | AMDEADYYC |
| LFW4 (Kabat) AA | 207 | FGSGTKVTVL |

TABLE 44
141LO0002 hIgG1 pgl-4 (anti-PAD2)

| | SEQ | |
|---|---|---|
| Description | ID NO: | Sequence |
| VH (Kabat) AA | 208 | EVQLLESGGGLVQPGGSLRLSCA |
| | | ASGFTFSSYAMSWVRQAPGKGLE |
| | | WVSAISGSGGSTYYADSVKGRFT |
| | | ISRDNSKNTLYLQMNSLRAEDTA |
| | | VYYCARHSYTRGFFSHEDDAVDI |
| | | WGRGTLVTVSS |
| HCDR1 (Kabat) AA | 193 | SYAMS |
| HCDR2 (Kabat) AA | 194 | AISGSGGSTYYADSVKG |
| HCDR3 (Kabat) AA | 195 | HSYTRGFFSHEDDAVDI |
| HFW1 (Kabat) AA | 196 | EVQLLESGGGLVQPGGSLRLSCA |
| | | ASGFTFS |
| HFW2 (Kabat) AA | 197 | WVRQAPGKGLEWVS |
| HFW3 (Kabat) AA | 198 | RFTISRDNSKNTLYLQMNSLRAE |
| | | DTAVYYCAR |
| HFW4 (Kabat) AA | 215 | WGRGTLVTVSS |
| VL (Kabat) AA | 200 | SYVLTQPPSVSVSPGQTASITCS |
| | | GDKVGDKYVSWYQQKPGQSPVLV |
| | | IYQDSQRPSGIPERFSGSNSGNT |
| | | ATLTISGTQAMDEADYYCEVGVD |
| | | YEYVFGSGTKVTVL |
| LCDR1 (Kabat) AA | 201 | SGDKVGDKYVS |
| LCDR2 (Kabat) AA | 202 | QDSQRPS |
| LCDR3 (Kabat) AA | 203 | EVGVDYEYV |
| LFW1 (Kabat) AA | 204 | SYVLTQPPSVSVSPGQTASITC |
| LFW2 (Kabat) AA | 205 | WYQQKPGQSPVLVIY |
| LFW3 (Kabat) AA | 206 | GIPERFSGSNSGNTATLTISGTQ |
| | | AMDEADYYC |
| LFW4 (Kabat) AA | 207 | FGSGTKVTVL |

TABLE 45
141LO0002 hIgG1 ngl-2 (anti-PAD2)

| | SEQ | |
|---|---|---|
| Description | ID NO: | Sequence |
| VH (Kabat) AA | 208 | EVQLLESGGGLVQPGGSLRLSCA |
| | | ASGFTFSSYAMSWVRQAPGKGLE |
| | | WVSAISGSGGSTYYADSVKGRFT |
| | | ISRDNSKNTLYLQMNSLRAEDTA |
| | | VYYCARHSYTRGFFSHEDDAVDI |
| | | WGRGTTVTVSS |
| HCDR1 (Kabat) AA | 193 | SYAMS |
| HCDR2 (Kabat) AA | 194 | AISGSGGSTYYADSVKG |
| HCDR3 (Kabat) AA | 195 | HSYTRGFFSHEDDAVDI |
| HFW1 (Kabat) AA | 196 | EVQLLESGGGLVQPGGSLRLSCA |
| | | ASGFTFS |
| HFW2 (Kabat) AA | 197 | WVRQAPGKGLEWVS |
| HFW3 (Kabat) AA | 198 | RFTISRDNSKNTLYLQMNSLRAE |
| | | DTAVYYCAR |
| HFW4 (Kabat) AA | 209 | WGRGTTVTVSS |
| VL (Kabat) AA | 210 | SYVLTQPPSVSVSPGQTASITCS |
| | | GDKVGDKYVSWYQQKPGQAPVLV |
| | | MYQDSQRPSGIPERISGSNSGNT |
| | | ATLTISGTQAVDEAEYYCEVGVD |
| | | YEYVFGSGTKVTVL |
| LCDR1 (Kabat) AA | 201 | SGDKVGDKYVS |
| LCDR2 (Kabat) AA | 202 | QDSQRPS |
| LCDR3 (Kabat) AA | 203 | EVGVDYEYV |
| LFW1 (Kabat) AA | 204 | SYVLTQPPSVSVSPGQTASITC |
| LFW2 (Kabat) AA | 211 | WYQQKPGQAPVLVMY |
| LFW3 (Kabat) AA | 212 | GIPERISGSNSGNTATLTISGTQ |
| | | AVDEAEYYC |
| LFW4 (Kabat) AA | 207 | FGSGTKVTVL |

TABLE 46
141LO0030 hIgG1 pgl-4 (anti-PAD2)

| | SEQ | |
|---|---|---|
| Description | ID NO: | Sequence |
| VH (Kabat) AA | 213 | EVQLLESGGGLVQPGGSLRLSCA |
| | | ASGFTFSSYAMSWVRQAPGKGLE |
| | | WVSAISGSGGSTYYADSVKGRFT |
| | | ISRDNSKNTLYLQMNSLRAEDTA |
| | | VYYCARQALVRGFFSHEDDAVDI |
| | | WGQGTTVTVSS |
| HCDR1 (Kabat) AA | 193 | SYAMS |
| HCDR2 (Kabat) AA | 194 | AISGSGGSTYYADSVKG |
| HCDR3 (Kabat) AA | 214 | QALVRGFFSHEDDAVDI |
| HFW1 (Kabat) AA | 196 | EVQLLESGGGLVQPGGSLRLSCA |
| | | ASGFTFS |
| HFW2 (Kabat) AA | 197 | WVRQAPGKGLEWVS |
| HFW3 (Kabat) AA | 198 | RFTISRDNSKNTLYLQMNSLRAE |
| | | DTAVYYCAR |
| HFW4 (Kabat) AA | 199 | WGQGTTVTVSS |
| VL (Kabat) AA | 79 | SYVLTQPPSVSVSPGQTASITCS |
| | | GDKVGDKYVSWYQQKPGQSPVLV |
| | | IYQDSQRPSGIPERFSGSNSGNT |
| | | ATLTISGTQAMDEADYYCQTWYD |
| | | DALTFGSGTKVTVL |
| LCDR1 (Kabat) AA | 201 | SGDKVGDKYVS |
| LCDR2 (Kabat) AA | 202 | QDSQRPS |
| LCDR3 (Kabat) AA | 216 | QTWYDDALT |
| LFW1 (Kabat) AA | 204 | SYVLTQPPSVSVSPGQTASITC |
| LFW2 (Kabat) AA | 205 | WYQQKPGQSPVLVIY |
| LFW3 (Kabat) AA | 206 | GIPERFSGSNSGNTATLTISGTQ |
| | | AMDEADYYC |
| LFW4 (Kabat) AA | 207 | FGSGTKVTVL |

TABLE 47
141LO0030 hIgG1 ngl-2 (anti-PAD2)

| | SEQ | |
|---|---|---|
| Description | ID NO: | Sequence |
| VH (Kabat) AA | 217 | EVQLLESGGGLVQPGGSLRLSCA |
| | | ASGFTFSSYAMSWVRQAPGKGLE |
| | | WVSAISGSGGSTYYADSVKGRFT |
| | | ISRDNSKNTLYLQMNSLRAEDTA |
| | | VYYCARQALVRGFFSHEDDAVDI |
| | | WGRGTTVTVSS |
| HCDR1 (Kabat) AA | 193 | SYAMS |
| HCDR2 (Kabat) AA | 194 | AISGSGGSTYYADSVKG |
| HCDR3 (Kabat) AA | 214 | QALVRGFFSHEDDAVDI |
| HFW1 (Kabat) AA | 196 | EVQLLESGGGLVQPGGSLRLSCA |
| | | ASGFTFS |
| HFW2 (Kabat) AA | 197 | WVRQAPGKGLEWVS |
| HFW3 (Kabat) AA | 198 | RFTISRDNSKNTLYLQMNSLRAE |
| | | DTAVYYCAR |
| HFW4 (Kabat) AA | 209 | WGRGTTVTVSS |
| VL (Kabat) AA | 218 | SYVLTQPPSVSVSPGQTASITCS |
| | | GDKVGDKYVSWYQQKPGQAPVLV |
| | | MYQDSQRPSGIPERISGSNSGNT |
| | | ATLTISGTQAVDEAEYYCQTWYD |
| | | DALTFGSGTKVTVL |
| LCDR1 (Kabat) AA | 201 | SGDKVGDKYVS |
| LCDR2 (Kabat) AA | 202 | QDSQRPS |
| LCDR3 (Kabat) AA | 216 | QTWYDDALT |
| LFW1 (Kabat) AA | 204 | SYVLTQPPSVSVSPGQTASITC |
| LFW2 (Kabat) AA | 211 | WYQQKPGQAPVLVMY |
| LFW3 (Kabat) AA | 212 | GIPERISGSNSGNTATLTISGTQ |
| | | AVDEAEYYC |
| LFW4 (Kabat) AA | 207 | FGSGTKVTVL |

TABLE 48
141LO0035 hlgG1 ngl-2 (anti-PAD2)

| | SEQ | |
|---|---|---|
| | ID | |
| Description | NO: | Sequence |
| VH (Kabat) AA | 219 | EVQLLESGGGLVQPGGSLRLSCAASGFTFS |
| | | SYAMSWVRQAPGKGLEWVSAISGSGGSTYY |
| | | ADSVKGRFTISRDNSKNTLYLQMNSLRAED |
| | | TAVYYCARQVLVRGFFSHEDDAVDIWGRGT |
| | | TVTVSS |
| HCDR1 (Kabat) AA | 193 | SYAMS |
| HCDR2 (Kabat) AA | 194 | AISGSGGSTYYADSVKG |
| HCDR3 (Kabat) AA | 220 | QVLVRGFFSHEDDAVDI |
| HFW1 (Kabat) AA | 196 | EVQLLESGGGLVQPGGSLRLSCAASGFTFS |
| HFW2 (Kabat) AA | 197 | WVRQAPGKGLEWVS |
| HFW3 (Kabat) AA | 198 | RFTISRDNSKNTLYLQMNSLRAEDTAVYYC |
| | | AR |
| HFW4 (Kabat) AA | 209 | WGRGTTVTVSS |
| VL (Kabat) AA | 221 | SYVLTQPPSVSVSPGQTASITCSGDKVGDK |
| | | YVSWYQQKPGQAPVLVMYQDSQRPSGIPER |
| | | ISGSNSGNTATLTISGTQAVDEAEYYCQTW |
| | | APDVLLFGSGTKVTVL |
| LCDR1 (Kabat) AA | 201 | SGDKVGDKYVS |
| LCDR2 (Kabat) AA | 202 | QDSQRPS |
| LCDR3 (Kabat) AA | 222 | QTWAPDVLL |
| LFW1 (Kabat) AA | 204 | SYVLTQPPSVSVSPGQTASITC |
| LFW2 (Kabat) AA | 211 | WYQQKPGQAPVLVMY |
| LFW3 (Kabat) AA | 212 | GIPERISGSNSGNTATLTISGTQAVDEAEY |
| | | YC |
| LFW4 (Kabat) AA | 207 | FGSGTKVTVL |

TABLE 49
141LO0039 hlgG1 pgl-4 (anti-PAD2)

| Description | SEQ ID NO: | Sequence |
|---|---|---|
| VH (Kabat) AA | 223 | EVQLLESGGGLVQPGGSLRLSCA |
| | | ASGFTFSSYAMSWVRQAPGKGLE |
| | | WVSAISGSGGSTYYADSVKGRFT |
| | | ISRDNSKNTLYLQMNSLRAEDTA |
| | | VYYCARQVLTRGFFSHEDDAVDI |
| | | WGQGTTVTVSS |
| HCDR1 (Kabat) AA | 193 | SYAMS |
| HCDR2 (Kabat) AA | 194 | AISGSGGSTYYADSVKG |
| HCDR3 (Kabat) AA | 224 | QVLTRGFFSHEDDAVDI |
| HFW1 (Kabat) AA | 196 | EVQLLESGGGLVQPGGSLRLSCA |
| | | ASGFTFS |
| HFW2 (Kabat) AA | 197 | WVRQAPGKGLEWVS |
| HFW3 (Kabat) AA | 198 | RFTISRDNSKNTLYLQMNSLRAE |
| | | DTAVYYCAR |
| HFW4 (Kabat) AA | 199 | WGQGTTVTVSS |
| VL (Kabat) AA | 225 | SYVLTQPPSVSVSPGQTASITCS |
| | | GDKVGDKYVSWYQQKPGQSPVLV |
| | | IYQDSQRPSGIPERFSGSNSGNT |
| | | ATLTISGTQAMDEADYYCHTYAE |
| | | DEYVFGSGTKVTVL |
| LCDR1 (Kabat) AA | 201 | SGDKVGDKYVS |
| LCDR2 (Kabat) AA | 202 | QDSQRPS |
| LCDR3 (Kabat) AA | 226 | HTYAEDEYV |
| LFW1 (Kabat) AA | 204 | SYVLTQPPSVSVSPGQTASITC |
| LFW2 (Kabat) AA | 205 | WYQQKPGQSPVLVIY |
| LFW3 (Kabat) AA | 206 | GIPERFSGSNSGNTATLTISGTQ |
| | | AMDEADYYC |
| LFW4 (Kabat) AA | 207 | FGSGTKVTVL |

TABLE 50
141LO0039 higG1 ngl-2 (anti-PAD2)

| | SEQ | |
|---|---|---|
| Description | ID NO: | Sequence |
| VH (Kabat) AA | 227 | EVQLLESGGGLVQPGGSLRLSCAA |
| | | SGFTFSSYAMSWVRQAPGKGLEWV |
| | | SAISGSGGSTYYADSVKGRFTISR |
| | | DNSKNTLYLQMNSLRAEDTAVYYC |
| | | ARQVLTRGFFSHEDDAVDIWGRGT |
| | | TVTVSS |
| HCDR1 (Kabat) AA | 193 | SYAMS |
| HCDR2 (Kabat) AA | 194 | AISGSGGSTYYADSVKG |
| HCDR3 (Kabat) AA | 224 | QVLTRGFFSHEDDAVDI |
| HFW1 (Kabat) AA | 196 | EVQLLESGGGLVQPGGSLRLSCAA |
| | | SGFTFS |
| HFW2 (Kabat) AA | 197 | WVRQAPGKGLEWVS |
| HFW3 (Kabat) AA | 198 | RFTISRDNSKNTLYLQMNSLRAED |
| | | TAVYYCAR |
| HFW4 (Kabat) AA | 209 | WGRGTTVTVSS |
| VL (Kabat) AA | 228 | SYVLTQPPSVSVSPGQTASITCSG |
| | | DKVGDKYVSWYQQKPGQAPVLVMY |
| | | QDSQRPSGIPERISGSNSGNTATL |
| | | TISGTQAVDEAEYYCHTYAEDEYV |
| | | EGSGTKVTVL |
| LCDR1 (Kabat) AA | 201 | SGDKVGDKYVS |
| LCDR2 (Kabat) AA | 202 | QDSQRPS |
| LCDR3 (Kabat) AA | 226 | HTYAEDEYV |
| LFW1 (Kabat) AA | 204 | SYVLTQPPSVSVSPGQTASITC |
| LFW2 (Kabat) AA | 211 | WYQQKPGQAPVLVMY |
| LFW3 (Kabat) AA | 212 | GIPERISGSNSGNTATLTISGTQA |
| | | VDEAEYYC |
| LFW4 (Kabat) AA | 207 | FGSGTKVTVL |

TABLE 51
141LO0055 hlgG1 ngl-2 (anti-PAD2)

| | SEQ | |
|---|---|---|
| Description | ID NO: | Sequence |
| VH (Kabat) AA | 229 | EVQLLESGGGLVQPGGSLRLSCAASG |
| | | FTFSSYAMSWVRQAPGKGLEWVSAIS |
| | | GSGGSTYYADSVKGRFTISRDNSKNT |
| | | LYLQMNSLRAEDTAVYYCARHSYTRG |
| | | FFSHESPDLPTWGRGTTVTVSS |
| HCDR1 (Kabat) AA | 193 | SYAMS |
| HCDR2 (Kabat) AA | 194 | AISGSGGSTYYADSVKG |
| HCDR3 (Kabat) AA | 230 | HSYTRGFFSHESPDLPT |
| HFW1 (Kabat) AA | 196 | EVQLLESGGGLVQPGGSLRLSCAASG |
| | | FTFS |
| HFW2 (Kabat) AA | 197 | WVRQAPGKGLEWVS |
| HFW3 (Kabat) AA | 198 | RFTISRDNSKNTLYLQMNSLRAEDTA |
| | | VYYCAR |
| HFW4 (Kabat) AA | 209 | WGRGTTVTVSS |
| VL (Kabat) AA | 231 | SYVLTQPPSVSVSPGQTASITCSGDK |
| | | VGDKYVSWYQQKPGQAPVLVMYQDSQ |
| | | RPSGIPERISGSNSGNTATLTISGTQ |
| | | AVDEAEYYCQSENAVEYVFGSGTKVT |
| | | VL |
| LCDR1 (Kabat) AA | 201 | SGDKVGDKYVS |
| LCDR2 (Kabat) AA | 202 | QDSQRPS |
| LCDR3 (Kabat) AA | 232 | QSENAVEYV |
| LFW1 (Kabat) AA | 204 | SYVLTQPPSVSVSPGQTASITC |
| LFW2 (Kabat) AA | 211 | WYQQKPGQAPVLVMY |
| LFW3 (Kabat) AA | 212 | GIPERISGSNSGNTATLTISGTQAVD |
| | | EAEYYC |
| LFW4 (Kabat) AA | 207 | FGSGTKVTVL |

TABLE 52
PAD40119 hlgG1 ngl-2 (anti-PAD2)

| | SEQ | |
|---|---|---|
| | ID | |
| Description | NO: | Sequence |
| VH (Kabat) AA | 233 | EVQLVESGAEVKKPGSSVKVSCKASGG |
| | | TFSSYAISWVRQAPGQGLEWMGGIIPI |
| | | FGTANYAQKFQGRVTITADESTSTAYM |
| | | ELSSLRSEDTAVYYCASGGWEDYWGRG |
| | | TLVTVSS |
| HCDR1 (Kabat) AA | 234 | SYAIS |
| HCDR2 (Kabat) AA | 235 | GIIPIFGTANYAQKFQG |
| HCDR3 (Kabat) AA | 236 | GGWFDY |
| HFW1 (Kabat) AA | 237 | EVQLVESGAEVKKPGSSVKVSCKASGG |
| | | TFS |
| HFW2 (Kabat) AA | 238 | WVRQAPGQGLEWMG |
| HFW3 (Kabat) AA | 239 | RVTITADESTSTAYMELSSLRSEDTAV |
| | | YYCAS |
| HFW4 (Kabat) AA | 240 | WGRGTLVTVSS |
| VL (Kabat) AA | 241 | QSVVTQPPSVSAAPGQKVTISCSGSSS |
| | | NIGNNYVSWYQQLPGTAPKLLIYDNNK |
| | | RPSGIPDRFSGSKSGTSATLGITGLQT |
| | | GDEADYYCGTWDSSLSAVVFGGGTKVT |
| | | VL |
| LCDR1 (Kabat) AA | 242 | SGSSSNIGNNYVS |
| LCDR2 (Kabat) AA | 243 | DNNKRPS |
| LCDR3 (Kabat) AA | 245 | GTWDSSLSAVV |
| LFW1 (Kabat) AA | 246 | QSVVTQPPSVSAAPGQKVTISC |
| LFW2 (Kabat) AA | 247 | WYQQLPGTAPKLLIY |
| LFW3 (Kabat) AA | 248 | GIPDRESGSKSGTSATLGITGLQTGDE |
| | | ADYYC |
| LFW4 (Kabat) AA | 249 | FGGGTKVTVL |

TABLE 53
PAD40141 hlgG1 ngl-2 (anti-PAD2)

| | SEQ | |
|---|---|---|
| | ID | |
| Description | NO: | Sequence |
| VH (Kabat) AA | 250 | EVQLLESGGGLVQPGGSLRLSCAASGF |
| | | TFSSYAMSWVRQAPGKGLEWVSAISGS |
| | | GGSTYYADSVKGRETISRDNSKNTLYL |
| | | QMNSLRAEDTAVYYCARHSYTRGFFSH |
| | | EDDAVDIWGRGTTVTVSS |
| HCDR1 (Kabat) AA | 193 | SYAMS |
| HCDR2 (Kabat) AA | 194 | AISGSGGSTYYADSVKG |
| HCDR3 (Kabat) AA | 195 | HSYTRGFFSHEDDAVDI |
| HFW1 (Kabat) AA | 196 | EVQLLESGGGLVQPGGSLRLSCAASGF |
| | | TFS |
| HFW2 (Kabat) AA | 197 | WVRQAPGKGLEWVS |
| HFW3 (Kabat) AA | 198 | RFTISRDNSKNTLYLQMNSLRAEDTAV |
| | | YYCAR |
| HFW4 (Kabat) AA | 209 | WGRGTTVTVSS |
| VL (Kabat) AA | 204 | SYVLTQPPSVSVSPGQTASITCSGDKV |
| | | GDKYVSWYQQKPGQAPVLVMYQDSQRP |
| | | SGIPERISGSNSGNTATLTISGTQAVD |
| | | EAEYYCQTWDSNEYVFGSGTKVTVL |
| LCDR1 (Kabat) AA | 201 | SGDKVGDKYVS |
| LCDR2 (Kabat) AA | 202 | QDSQRPS |
| LCDR3 (Kabat) AA | 251 | QTWDSNEYV |
| LFW1 (Kabat) AA | 204 | SYVLTQPPSVSVSPGQTASITC |
| LFW2 (Kabat) AA | 211 | WYQQKPGQAPVLVMY |
| LFW3 (Kabat) AA | 212 | GIPERISGSNSGNTATLTISGTQAVDE |
| | | AEYYC |
| LFW4 (Kabat) AA | 207 | FGSGTKVTVL |

TABLE 54
PAD40175 higG1 ngl-2 (anti-PAD2)

| | SEQ | |
|---|---|---|
| Description | ID NO: | Sequence |
| VH (Kabat) AA | 252 | EVQLLESGGGLVQPGGSLRLSCAASGFT |
| | | FSSYAMSWVRQAPGKGLEWVSAISGSGG |
| | | STYYADSVKGRFTISRDNSKNTLYLQMN |
| | | SLRAEDTAVYYCARDSGKSYSRGWYTAF |
| | | DIWGRGTTVTVSS |
| HCDR1 (Kabat) AA | 193 | SYAMS |
| HCDR2 (Kabat) AA | 194 | AISGSGGSTYYADSVKG |
| HCDR3 (Kabat) AA | 253 | DSGKSYSRGWYTAFDI |
| HFW1 (Kabat) AA | 196 | EVQLLESGGGLVQPGGSLRLSCAASGFT |
| | | FS |
| HFW2 (Kabat) AA | 197 | WVRQAPGKGLEWVS |
| HFW3 (Kabat) AA | 198 | RFTISRDNSKNTLYLQMNSLRAEDTAVY |
| | | YCAR |
| HFW4 (Kabat) AA | 209 | WGRGTTVTVSS |
| VL (Kabat) AA | 254 | QAVLTQPSSVSVAPGKTATITCGGDNIG |
| | | SKSVHWYQQKPGQAPLLVIFYDTDRPSG |
| | | VPERFSGSNSGNTATLTISRVEAGDEAD |
| | | YYCQVWDSNGDHYVFGTGTKLTVL |
| LCDR1 (Kabat) AA | 255 | GGDNIGSKSVH |
| LCDR2 (Kabat) AA | 256 | YDTDRPS |
| LCDR3 (Kabat) AA | 257 | QVWDSNGDHYV |
| LFW1 (Kabat) AA | 258 | QAVLTQPSSVSVAPGKTATITC |
| LFW2 (Kabat) AA | 259 | WYQQKPGQAPLLVIF |
| LFW3 (Kabat) AA | 260 | GVPERFSGSNSGNTATLTISRVEAGDEA |
| | | DYYC |
| LFW4 (Kabat) AA | 261 | FGTGTKLTVL |

TABLE 55
AB1630204 (mouse anti-PAD4)

| | SEQ | |
|---|---|---|
| | ID | |
| Description | NO: | Sequence |
| VH (Kabat) AA | 262 | QVQLQQPGAELVKPGASVKLSCKASGY |
| | | TFTSYWMHWVKQRPGRGPEWIGRIDPN |
| | | SGGTKYNEKFKSKAILTVDKPSSTAYM |
| | | QLSSLTSEDSAVYYCAREGGDYWYFDV |
| | | WGAGTTVTVSS |
| HCDR1 (Kabat) AA | 263 | SYWMH |
| HCDR2 (Kabat) AA | 264 | RIDPNSGGTKYNEKFKS |
| HCDR3 (Kabat) AA | 244 | EGGDYWYFDV |
| HFW1 (Kabat) AA | 265 | QVQLQQPGAELVKPGASVKLSCKASGY |
| | | TFT |
| HFW2 (Kabat) AA | 266 | WVKQRPGRGPEWIG |
| HFW3 (Kabat) AA | 267 | KAILTVDKPSSTAYMQLSSLTSEDSAV |
| | | YYCAR |
| HFW4 (Kabat) AA | 268 | WGAGTTVTVSS |
| VL (Kabat) AA | 269 | QIVLTQSPALMSASPGEKVTMTCSANS |
| | | GLRYMYWYQQKPRSSPKPWIYLTSNLA |
| | | SGVPARFSGSGSGTSYSLTISSMEAED |
| | | AATYYCQQWSSIPPTFGAGTKLELK |
| LCDR1 (Kabat) AA | 270 | SANSGLRYMY |
| LCDR2 (Kabat) AA | 271 | LTSNLAS |
| LCDR3 (Kabat) AA | 272 | QQWSSIPPT |
| LFW1 (Kabat) AA | 273 | QIVLTQSPALMSASPGEKVTMTC |
| LFW2 (Kabat) AA | 274 | WYQQKPRSSPKPWIY |
| LFW3 (Kabat) AA | 275 | GVPARFSGSGSGTSYSLTISSMEAEDA |
| | | ATYYC |
| LFW4 (Kabat) AA | 276 | FGAGTKLELK |

TABLE 56
AB1630205 (mouse anti-PAD4)

| | SEQ | |
|---|---|---|
| | ID | |
| Description | NO: | Sequence |
| VH (Kabat) AA | 277 | EVQLVESGGGLVKPGGSRKLSCAASGF |
| | | TFSDYGMHWVRQAPEKGLEWVAYISSG |
| | | SSTIYYADTVKGRFTISRDNAKNTLFL |
| | | QMTSLRSEDTAMYYCTRTYYDDAMDYW |
| | | GQGTSVTVSS |
| HCDR1 (Kabat) AA | 278 | DYGMH |
| HCDR2 (Kabat) AA | 279 | YISSGSSTIYYADTVKG |
| HCDR3 (Kabat) AA | 280 | TYYDDAMDY |
| HFW1 (Kabat) AA | 281 | EVQLVESGGGLVKPGGSRKLSCAASGF |
| | | TFS |
| HFW2 (Kabat) AA | 282 | WVRQAPEKGLEWVA |
| HFW3 (Kabat) AA | 283 | RFTISRDNAKNTLFLQMTSLRSEDTAM |
| | | YYCTR |
| HFW4 (Kabat) AA | 284 | WGQGTSVTVSS |
| VL (Kabat) AA | 285 | SIVMTQTPKFLLVSAGDRVTITCKASQ |
| | | SVSNDVVWYQQKPGQSPKLLISYASNR |
| | | YTGVPDRFTGSGYGTDFTFTISSVQAE |
| | | DLAVYFCQQDYSSPWTFGGGTKLEIK |
| LCDR1 (Kabat) AA | 286 | KASQSVSNDVV |
| LCDR2 (Kabat) AA | 287 | YASNRYT |
| LCDR3 (Kabat) AA | 288 | QQDYSSPWT |
| LFW1 (Kabat) AA | 289 | SIVMTQTPKFLLVSAGDRVTITC |
| LFW2 (Kabat) AA | 290 | WYQQKPGQSPKLLIS |
| LFW3 (Kabat) AA | 291 | GVPDRFTGSGYGTDFTFTISSVQAEDL |
| | | AVYFC |
| LFW4 (Kabat) AA | 292 | FGGGTKLEIK |

TABLE 57
Bivalent bispecific Bis3 (Clones 07-12)

| SEQ | | |
|---|---|---|
| ID NO. | Description | Sequence |
| 33 | scFv VH-VLLinker | GGGGSGGGGSGGGGSGGGGS |
| 34 | PAD2 Fab HC (VH-C_{H}1) | EVQLLESGGGLVQPGGSLRLSCAASGFTFSSYAMSWVRQAPGKGLEWV |
| | Clones 08, 10, 12 | SAISGSGGSTYYADSVKGRFTISRDNSKNTLYLQMNSLRAEDTAVYYC |
| | | ARQVLVRGFFSHEDDAVDIWGQGTTVTVSSASTKGPSVFPLAPSSKST |
| | | SGGTAALGCLVKDYFPEPVTVSWNSGALTSGVHTFPAVLQSSGLYSLS |
| | | SVVTVPSSSLGTQTYICNVNHKPSNTKVDKRV |
| 35 | PAD2 Fab LC (VL-C_{L}) | SYVLTQPPSVSVSPGQTASITCSGDKVGDKYVSWYQQKPGQSPVLVIY |
| | Clones 08, 10, 12 | QDSQRPSGIPERFSGSNSGNTATLTISGTQAMDEADYYCQTWAPDVLL |
| | | FGSGTKVTVLGQPKAAPSVTLFPPSSEELQANKATLVCLISDFYPGAV |
| | | TVAWKADSSPVKAGVETTTPSKQSNNKYAASSYLSLTPEQWKSHRSYS |
| | | CQVTHEGSTVEKTVAPTECS |
| 36 | Lambda C_{L} | GQPKAAPSVTLFPPSSEELQANKATLVCLISDFYPGAVTVAWKADSSP |
| | Clones 07-12 | VKAGVETTTPSKQSNNKYAASSYLSLTPEQWKSHRSYSCQVTHEGSTV |
| | | EKTVAPTECS |
| 37 | C_{H} | ASTKGPSVFPLAPSSKSTSGGTAALGCLVKDYFPEPVTVSWNSGALTS |
| | Clones 07-12 | GVHTFPAVLQSSGLYSLSSVVTVPSSSLGTQTYICNVNHKPSNTKVDK |
| | | RV |
| 38 | PAD2 scFv ((VL-VH with linker | EVQLLESGGGLVQPGGSLRLSCAASGFTFSSYAMSWVRQAPGKGLEWV |
| | (G4S)4) | SAISGSGGSTYYADSVKGRFTISRDNSKNTLYLQMNSLRAEDTAVYYC |
| | Clones 07, 09, 11 | ARQVLVRGFFSHEDDAVDIWGQGTTVTVSSGGGGSGGGGSGGGGSGGG |
| | | GSSYVLTQPPSVSVSPGQTASITCSGDKVGDKYVSWYQQKPGQSPVLV |
| | | IYQDSQRPSGIPERFSGSNSGNTATLTISGTQAMDEADYYCQTWAPDV |
| | | LLFGSGTKVTVL |
| 39 | PAD4 scFv (VH-VLwith (G4S)4 | QVQLVESGGGLVKPGGSLRLSCAASGSTLSDYFVSWIRQAPGKGLEWVSFINAA |
| | Clones 08, 10, 12 | NTFTYYADSVRGRFTISRDNAKNSVYLQMNSLRAEDTAVYYCSSANDDVDDIVA |
| | | PGRGYYMDVWGRGTLVTVSSGGGGSGGGGSGGGGSGGGGSQSALTQPRSV |
| | | SGSPGQSVTISCTGTSGDVGRYSHVSWYQQHPGKAPKLIIYNVYERPSGVPDRF |
| | | SGSKSGNTASLTISGLQAEDEADYYCSSHSRSSTPVLFGGGTKLTVL |
| 40 | PAD4 Fab HC (VH-C_{H}1) | QVQLVESGGGLVKPGGSLRLSCAASGSTLSDYFVSWIRQAPGKGLEWV |
| | Clones 07, 09, 11 | SFINAANTFTYYADSVRGRFTISRDNAKNSVYLQMNSLRAEDTAVYYC |
| | | SSANDDVDDIVAPGRGYYMDVWGRGTLVTVSSASTKGPSVFPLAPSSK |
| | | STSGGTAALGCLVKDYFPEPVTVSWNSGALTSGVHTFPAVLQSSGLYS |
| | | LSSVVTVPSSSLGTQTYICNVNHKPSNTKVDKRV |
| 41 | PAD4 Fab LC (VL-C_{L}) | QSALTQPRSVSGSPGQSVTISCTGTSGDVGRYSHVSWYQQHPGKAPKL |
| | Clones 07, 09, 11 | IIYNVYERPSGVPDRESGSKSGNTASLTISGLQAEDEADYYCSSHSRS |
| | | STPVLFGGGTKLTVLGQPKAAPSVTLFPPSSEELQANKATLVCLISDE |
| | | YPGAVTVAWKADSSPVKAGVETTTPSKQSNNKYAASSYLSLTPEQWKS |
| | | HRSYSCQVTHEGSTVEKTVAPTECS |
| 42 | Bis3 full heavy chain sequence | EVQLLESGGGLVQPGGSLRLSCAASGFTFSSYAMSWVRQAPGKGLEWV |
| | FQQ-YTE | SAISGSGGSTYYADSVKGRFTISRDNSKNTLYLQMNSLRAEDTAVYYC |
| | (VH-C_{H}1 C_{H}2 C_{H}3) | ARQVLVRGFFSHEDDAVDIWGQGTTVTVSSASTKGPSVFPLAPSSKST |
| | Clone 12 | SGGTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPCPA |
| | | PEFQGGPSVFLFPPKPKDTLYITREPEVTCVVVDVSHEDPEVKENWYV |
| | | DGVEVHNAKTKPREEQYNSTYRVVSVLTVLHQDWLNGKEYKCQVSNKA |
| | | LPAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGNVE |
| | | SCSVMHEALHNHYTQKSLSLSPGKGGGGSGGGGSQVQLVESGGGLVKP |
| | | GGSLRLSCAASGSTLSDYFVSWIRQAPGKGLEWVSFINAANTFTYYAD |
| | | SVRGRFTISRDNAKNSVYLQMNSLRAEDTAVYYCSSANDDVDDIVAPG |
| | | RGYYMDVWGRGTLVTVSSGGGGSGGGGSGGGGSGGGGSQSALTQPRSV |
| | | SGSPGQSVTISCTGTSGDVGRYSHVSWYQQHPGKAPKLIIYNVYERPS |
| | | GVPDRFSGSKSGNTASLTISGLQAEDEADYYCSSHSRSSTPVLEGGGT |
| | | KLTVL |
| 43 | Bis3 IgG1 FQQ-YTEFc | ASTKGPSVFPLAPSSKSTSGGTAALGCLVKDYFPEPVTVSWNSGALTS |
| | Clone 12 | GVHTFPAVLQSSGLYSLSSVVTVPSSSLGTQTYICNVNHKPSNTKVDK |
| | | RVEPKSCDKTHTCPPCPAPEFQGGPSVFLFPPKPKDTLYITREPEVTC |
| | | VVVDVSHEDPEVKFNWYVDGVEVHNAKTKPREEQYNSTYRVVSVLTVL |
| | | HQDWLNGKEYKCQVSNKALPAPIEKTISKAKGQPREPQVYTLPPSREE |
| | | MTKNQVSLTCLVKGFYPSDIAVEWESNGQPENNYKTTPPVLDSDGSFF |
| | | LYSKLTVDKSRWQQGNVFSCSVMHEALHNHYTQKSLSLSPGK |
| 37 | Bis3 lgG1 C_{H}1 | ASTKGPSVFPLAPSSKSTSGGTAALGCLVKDYFPEPVTVSWNSGALTS |
| | Clone 07-12 | GVHTFPAVLQSSGLYSLSSVVTVPSSSLGTQTYICNVNHKPSNTKVDK |
| | | RV |
| 45 | Bis3 IgG1 Hinge | EPKSCDKTHTCPPCP |
| | Clones 07-12 | |
| 46 | Bis3 lgG C_{H}2 | APELLGGPSVFLFPPKPKDTLMISRTPEVTCVVVDVSHEDPEVKENWY |
| | | VDGVEVHNAKTKPREEQYNSTYRVVSVLTVLHQDWLNGKEYKCKVSNK |
| | | ALPAPIEKTISKAK |
| 47 | Bis3 IgG1 TM C_{H}2 | APEFEGGPSVFLFPPKPKDTLMISRTPEVTCVVVDVSHEDPEVKENWY |
| | Clones 07 and 08 | VDGVEVHNAKTKPREEQYNSTYRVVSVLTVLHQDWLNGKEYKCKVSNK |
| | | ALPASIEKTISKAK |
| 48 | Bis3 IgG1 TM-YTE C_{H}2 | APEFEGGPSVFLFPPKPKDTLYITREPEVTCVVVDVSHEDPEVKENWY |
| | Clones 09 and 10 | VDGVEVHNAKTKPREEQYNSTYRVVSVLTVLHQDWLNGKEYKCKVSNK |
| | | ALPASIEKTISKAK |
| 49 | Bis3 IgG1 FQQ-YTE C_{H}2 | APEFQGGPSVFLFPPKPKDTLYITREPEVTCVVVDVSHEDPEVKENWY |
| | Clones 11 and 12 | VDGVEVHNAKTKPREEQYNSTYRVVSVLTVLHQDWLNGKEYKCQVSNK |
| | | ALPAPIEKTISKAK |
| 50 | Bis3 IgG1 C_{H}3 | GQPREPQVYTLPPSREEMTKNQVSLTCLVKGFYPSDIAVEWESNGQPE |
| | Clone 07-12 | NNYKTTPPVLDSDGSFFLYSKLTVDKSRWQQGNVFSCSVMHEALHNHY |
| | | TQKSLSLSPGK |
| 51 | Fc-scFv Linker | GGGGSGGGGS |
| | Clones 07-12 | |
| 53 | Bis3 TM | QSALTQPRSVSGSPGQSVTISCTGTSGDVGRYSHVSWYQQHPGKAPKL |
| | Clone 07 | IIYNVYERPSGVPDRESGSKSGNTASLTISGLQAEDEADYYCSSHSRS |
| | | STPVLFGGGTKLTVLGQPKAAPSVTLFPPSSEELQANKATLVCLISDE |
| | | YPGAVTVAWKADSSPVKAGVETTTPSKQSNNKYAASSYLSLTPEQWKS |
| | | HRSYSCQVTHEGSTVEKTVAPTECSQVQLVESGGGLVKPGGSLRLSCA |
| | | ASGSTLSDYFVSWIRQAPGKGLEWVSFINAANTFTYYADSVRGRATIS |
| | | RDNAKNSVYLQMNSLRAEDTAVYYCSSANDDVDDIVAPGRGYYMDVWG |
| | | RGTLVTVSSASTKGPSVFPLAPSSKSTSGGTAALGCLVKDYFPEPVTV |
| | | SWNSGALTSGVHTFPAVLQSSGLYSLSSVVTVPSSSLGTQTYICNVNH |
| | | KPSNTKVDKRVEPKSCDKTHTCPPCPAPEFEGGPSVFLFPPKPKDTLM |
| | | ISRTPEVTCVVVDVSHEDPEVKENWYVDGVEVHNAKTKPREEQYNSTY |
| | | RVVSVLTVLHQDWLNGKEYKCKVSNKALPASIEKTISKAKGQPREPQV |
| | | YTLPPSREEMTKNQVSLTCLVKGFYPSDIAVEWESNGQPENNYKTTPP |
| | | VLDSDGSFFLYSKLTVDKSRWQQGNVFSCSVMHEALHNHYTQKSLSLS |
| | | PGKGGGGSGGGGSEVQLLESGGGLVQPGGSLRLSCAASGFTFSSYAMS |
| | | WVRQAPGKGLEWVSAISGSGGSTYYADSVKGRFTISRDNSKNTLYLQM |
| | | NSLRAEDTAVYYCARQVLVRGFFSHEDDAVDIWGQGTTVTVSSGGGGS |
| | | GGGGSGGGGSGGGGSSYVLTQPPSVSVSPGQTASITCSGDKVGDKYVS |
| | | WYQQKPGQSPVLVIYQDSQRPSGIPERFSGSNSGNTATLTISGTQAMD |
| | | EADYYCQTWAPDVLLFGSGTKVTVL |
| 54 | Bis3 TM-YTE | QSALTQPRSVSGSPGQSVTISCTGTSGDVGRYSHVSWYQQHPGKAPKL |
| | Clone 09 | IIYNVYERPSGVPDRFSGSKSGNTASLTISGLQAEDEADYYCSSHSRS |
| | | STPVLFGGGTKLTVLGQPKAAPSVTLFPPSSEELQANKATLVCLISDE |
| | | YPGAVTVAWKADSSPVKAGVETTTPSKQSNNKYAASSYLSLTPEQWKS |
| | | HRSYSCQVTHEGSTVEKTVAPTECSQVQLVESGGGLVKPGGSLRLSCA |
| | | ASGSTLSDYFVSWIRQAPGKGLEWVSFINAANTFTYYADSVRGRFTIS |
| | | RDNAKNSVYLQMNSLRAEDTAVYYCSSANDDVDDIVAPGRGYYMDVWG |
| | | RGTLVTVSSASTKGPSVFPLAPSSKSTSGGTAALGCLVKDYFPEPVTV |
| | | SWNSGALTSGVHTFPAVLQSSGLYSLSSVVTVPSSSLGTQTYICNVNH |
| | | KPSNTKVDKRVEPKSCDKTHTCPPCPAPEFEGGPSVFLFPPKPKDTLY |
| | | ITREPEVTCVVVDVSHEDPEVKENWYVDGVEVHNAKTKPREEQYNSTY |
| | | RVVSVLTVLHQDWLNGKEYKCKVSNKALPASIEKTISKAKGQPREPQV |
| | | YTLPPSREEMTKNQVSLTCLVKGFYPSDIAVEWESNGQPENNYKTTPP |
| | | VLDSDGSFFLYSKLTVDKSRWQQGNVFSCSVMHEALHNHYTQKSLSLS |
| | | PGKggggsggggsEVQLLESGGGLVQPGGSLRLSCAASGFTFSSYAMS |
| | | WVRQAPGKGLEWVSAISGSGGSTYYADSVKGRFTISRDNSKNTLYLQM |
| | | NSLRAEDTAVYYCARQVLVRGFFSHEDDAVDIWGQGTTVTVSSGGGGS |
| | | GGGGSGGGGSGGGGSSYVLTQPPSVSVSPGQTASITCSGDKVGDKYVS |
| | | WYQQKPGQSPVLVIYQDSQRPSGIPERFSGSNSGNTATLTISGTQAMD |
| | | EADYYCQTWAPDVLLFGSGTKVTVL |
| 55 | Bis3 FQQ YTE | QSALTQPRSVSGSPGQSVTISCTGTSGDVGRYSHVSWYQQHPGKAPKL |
| | Clone 11 | IIYNVYERPSGVPDRFSGSKSGNTASLTISGLQAEDEADYYCSSHSRS |
| | | STPVLFGGGTKLTVLGQPKAAPSVTLFPPSSEELQANKATLVCLISDE |
| | | YPGAVTVAWKADSSPVKAGVETTTPSKQSNNKYAASSYLSLTPEQWKS |
| | | HRSYSCQVTHEGSTVEKTVAPTECSQVQLVESGGGLVKPGGSLRLSCA |
| | | ASGSTLSDYFVSWIRQAPGKGLEWVSFINAANTFTYYADSVRGRFTIS |
| | | RDNAKNSVYLQMNSLRAEDTAVYYCSSANDDVDDIVAPGRGYYMDVWG |
| | | RGTLVTVSSASTKGPSVFPLAPSSKSTSGGTAALGCLVKDYFPEPVTV |
| | | SWNSGALTSGVHTFPAVLQSSGLYSLSSVVTVPSSSLGTQTYICNVNH |
| | | KPSNTKVDKRVEPKSCDKTHTCPPCPAPEFQGGPSVFLFPPKPKDTLY |
| | | ITREPEVTCVVVDVSHEDPEVKENWYVDGVEVHNAKTKPREEQYNSTY |
| | | RVVSVLTVLHQDWLNGKEYKCQVSNKALPAPIEKTISKAKGQPREPQV |
| | | YTLPPSREEMTKNQVSLTCLVKGFYPSDIAVEWESNGQPENNYKTTPP |
| | | VLDSDGSFFLYSKLTVDKSRWQQGNVFSCSVMHEALHNHYTQKSLSLS |
| | | PGKggggsggggsEVQLLESGGGLVQPGGSLRLSCAASGFTFSSYAMS |
| | | WVRQAPGKGLEWVSAISGSGGSTYYADSVKGRFTISRDNSKNTLYLQM |
| | | NSLRAEDTAVYYCARQVLVRGFFSHEDDAVDIWGQGTTVTVSSGGGGS |
| | | GGGGSGGGGSGGGGSSYVLTQPPSVSVSPGQTASITCSGDKVGDKYVS |
| | | WYQQKPGQSPVLVIYQDSQRPSGIPERFSGSNSGNTATLTISGTQAMD |
| | | EADYYCQTWAPDVLLFGSGTKVTVL |
| 56 | Bis3 TM | SYVLTQPPSVSVSPGQTASITCSGDKVGDKYVSWYQQKPGQSPVLVIY |
| | Clone 08 | QDSQRPSGIPERFSGSNSGNTATLTISGTQAMDEADYYCQTWAPDVLL |
| | | FGSGTKVTVLGQPKAAPSVTLEPPSSEELQANKATLVCLISDFYPGAV |
| | | TVAWKADSSPVKAGVETTTPSKQSNNKYAASSYLSLTPEQWKSHRSYS |
| | | CQVTHEGSTVEKTVAPTECSEVQLLESGGGLVQPGGSLRLSCAASGET |
| | | FSSYAMSWVRQAPGKGLEWVSAISGSGGSTYYADSVKGRFTISRDNSK |
| | | NTLYLQMNSLRAEDTAVYYCARQVLVRGFFSHEDDAVDIWGQGTTVTV |
| | | SSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKV |
| | | DKRVEPKSCDKTHTCPPCPAPEFEGGPSVFLFPPKPKDTLMISRTPEV |
| | | TCVVVDVSHEDPEVKENWYVDGVEVHNAKTKPREEQYNSTYRVVSVLT |
| | | VLHQDWLNGKEYKCKVSNKALPASIEKTISKAKGQPREPQVYTLPPSR |
| | | EEMTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGKGGGG |
| | | SGGGGSQVQLVESGGGLVKPGGSLRLSCAASGSTLSDYFVSWIRQAPG |
| | | KGLEWVSFINAANTFTYYADSVRGRFTISRDNAKNSVYLQMNSLRAED |
| | | TAVYYCSSANDDVDDIVAPGRGYYMDVWGRGTLVTVSSGGGGSGGGGS |
| | | GGGGSGGGGSQSALTQPRSVSGSPGQSVTISCTGTSGDVGRYSHVSWY |
| | | QQHPGKAPKLIIYNVYERPSGVPDRFSGSKSGNTASLTISGLQAEDEA |
| | | DYYCSSHSRSSTPVLFGGGTKLTVL |
| 57 | Bis3 TM YTE | SYVLTQPPSVSVSPGQTASITCSGDKVGDKYVSWYQQKPGQSPVLVIY |
| | Clone 10 | QDSQRPSGIPERFSGSNSGNTATLTISGTQAMDEADYYCQTWAPDVLL |
| | | FGSGTKVTVLGQPKAAPSVTLFPPSSEELQANKATLVCLISDFYPGAV |
| | | TVAWKADSSPVKAGVETTTPSKQSNNKYAASSYLSLTPEQWKSHRSYS |
| | | CQVTHEGSTVEKTVAPTECSEVQLLESGGGLVQPGGSLRLSCAASGFT |
| | | FSSYAMSWVRQAPGKGLEWVSAISGSGGSTYYADSVKGRFTISRDNSK |
| | | NTLYLQMNSLRAEDTAVYYCARQVLVRGFFSHEDDAVDIWGQGTTVTV |
| | | SSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKV |
| | | DKRVEPKSCDKTHTCPPCPAPEFEGGPSVFLFPPKPKDTLYITREPEV |
| | | TCVVVDVSHEDPEVKFNWYVDGVEVHNAKTKPREEQYNSTYRVVSVLT |
| | | VLHQDWLNGKEYKCKVSNKALPASIEKTISKAKGQPREPQVYTLPPSR |
| | | EEMTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGKgggg |
| | | sggggsQVQLVESGGGLVKPGGSLRLSCAASGSTLSDYFVSWIRQAPG |
| | | KGLEWVSFINAANTFTYYADSVRGRFTISRDNAKNSVYLQMNSLRAED |
| | | TAVYYCSSANDDVDDIVAPGRGYYMDVWGRGTLVTVSSGGGGGGGGS |
| | | GGGGSGGGGSQSALTQPRSVSGSPGQSVTISCTGTSGDVGRYSHVSWY |
| | | QQHPGKAPKLIIYNVYERPSGVPDRESGSKSGNTASLTISGLQAEDEA |
| | | DYYCSSHSRSSTPVLFGGGTKLTVL |
| 58 | Bis3 FQQ YTE | SYVLTQPPSVSVSPGQTASITCSGDKVGDKYVSWYQQKPGQSPVLVIY |
| | Clone 12 | QDSQRPSGIPERFSGSNSGNTATLTISGTQAMDEADYYCQTWAPDVLL |
| | | FGSGTKVTVLGQPKAAPSVTLFPPSSEELQANKATLVCLISDFYPGAV |
| | | TVAWKADSSPVKAGVETTTPSKQSNNKYAASSYLSLTPEQWKSHRSYS |
| | | CQVTHEGSTVEKTVAPTECSEVQLLESGGGLVQPGGSLRLSCAASGET |
| | | FSSYAMSWVRQAPGKGLEWVSAISGSGGSTYYADSVKGRFTISRDNSK |
| | | NTLYLQMNSLRAEDTAVYYCARQVLVRGFFSHEDDAVDIWGQGTTVTV |
| | | SSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKV |
| | | DKRVEPKSCDKTHTCPPCPAPEFQGGPSVFLFPPKPKDTLYITREPEV |
| | | TCVVVDVSHEDPEVKENWYVDGVEVHNAKTKPREEQYNSTYRVVSVLT |
| | | VLHQDWLNGKEYKCQVSNKALPAPIEKTISKAKGQPREPQVYTLPPSR |
| | | EEMTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGKgggg |
| | | sggggsQVQLVESGGGLVKPGGSLRLSCAASGSTLSDYFVSWIRQAPG |
| | | KGLEWVSFINAANTFTYYADSVRGRFTISRDNAKNSVYLQMNSLRAED |
| | | TAVYYCSSANDDVDDIVAPGRGYYMDVWGRGTLVTVSSGGGGSGGGGS |
| | | GGGGSGGGGSQSALTQPRSVSGSPGQSVTISCTGTSGDVGRYSHVSWY |
| | | QQHPGKAPKLIIYNVYERPSGVPDRESGSKSGNTASLTISGLQAEDEA |
| | | DYYCSSHSRSSTPVLFGGGTKLTVL |

TABLE 58
Monovalent bispecific DuetMab (Clones 01-06)

| SEQ ID NO. | Description | Sequence |
|---|---|---|
| 59 | PAD2 full heavy chain-‘hole’ arm | EVQLLESGGGLVQPGGSLRLSCAASGFTFSSYAMSWVRQAPGKGLE |
| | (without RFmutation) | WVSAISGSGGSTYYADSVKGRFTISRDNSKNTLYLQMNSLRAEDTA |
| | FQQ-YTE | VYYCARQVLVRGFFSHEDDAVDIWGQGTTVTVSSASTKGPSVFPLA |
| | Clone 06 | PSSKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSCD |
| | | KTHTCPPCPAPEFQGGPSVFLFPPKPKDTLYITREPEVTCVVVDVS |
| | | HEDPEVKFNWYVDGVEVHNAKTKPREEQYNSTYRVVSVLTVLHQDW |
| | | LNGKEYKCQVSNKALPAPIEKTISKAKGQPREPQVCTLPPSREEMT |
| | | KNQVSLSCAVKGFYPSDIAVEWESNGQPENNYKTTPPVLDSDGSFF |
| | | LVSKLTVDKSRWQQGNVFSCSVMHEALHNHYTQKSLSLSPGK |
| 60 | Clone 06 | EVQLLESGGGLVQPGGSLRLSCAASGFTFSSYAMSWVRQAPGKGLE |
| | PAD2 full heavy chain-‘hole’ arm | WVSAISGSGGSTYYADSVKGRFTISRDNSKNTLYLQMNSLRAEDTA |
| | (with RFmutation) | VYYCARQVLVRGFFSHEDDAVDIWGQGTTVTVSSASTKGPSVFPLA |
| | (VH-C_{H}1—C_{H}2—C_{H}3) | PSSKSTSGGTAALGCLVKDYFPEPVTVSWNSGALTSGVHTFPAVLQ |
| | FQQ-YTE | SSGLYSLSSVVTVPSSSLGTQTYICNVNHKPSNTKVDKRVEPKSCD |
| | Clone 06 | KTHTCPPCPAPEFQGGPSVFLFPPKPKDTLYITREPEVTCVVVDVS |
| | | HEDPEVKFNWYVDGVEVHNAKTKPREEQYNSTYRVVSVLTVLHQDW |
| | | LNGKEYKCQVSNKALPAPIEKTISKAKGQPREPQVCTLPPSREEMT |
| | | KNQVSLSCAVKGFYPSDIAVEWESNGQPENNYKTTPPVLDSDGSFF |
| | | LVSKLTVDKSRWQQGNVFSCSVMHEALHNRFTQKSLSLSPGK |
| 61 | PAD4 full heavy chain-‘knob’ arm | QVQLVESGGGLVKPGGSLRLSCAASGSTLSDYFVSWIRQAPGKGLE |
| | (VH-C_{H}1 C_{H}2 C_{H}3) | WVSFINAANTFTYYADSVRGRFTISRDNAKNSVYLQMNSLRAEDTA |
| | FQQ-YTE | VYYCSSANDDVDDIVAPGRGYYMDVWGRGTLVTVSSASTKGPSVCP |
| | Clone 06 | LAPSSKSTSGGTAALGCLVKDYFPEPVTVSWNSGALTSGVHTFPAV |
| | | LQSSGLYSLSSVVTVPSSSLGTQTYICNVNHKPSNTKVDKRVEPKS |
| | | VDKTHTCPPCPAPEFQGGPSVFLFPPKPKDTLYITREPEVTCVVVD |
| | | VSHEDPEVKFNWYVDGVEVHNAKTKPREEQYNSTYRVVSVLTVLHQ |
| | | DWLNGKEYKCQVSNKALPAPIEKTISKAKGQPREPQVYTLPPCREE |
| | | MTKNQVSLWCLVKGFYPSDIAVEWESNGQPENNYKTTPPVLDSDGS |
| | | FFLYSKLTVDKSRWQQGNVFSCSVMHEALHNHYTQKSLSLSPGK |
| 62 | PAD2 full kappa light chain | SYVLTQPPSVSVSPGQTASITCSGDKVGDKYVSWYQQKPGQSPVLV |
| | (V_{L}-C_{L}(K)) | IYQDSQRPSGIPERFSGSNSGNTATLTISGTQAMDEADYYCQTWAP |
| | Clone 02, 04, 06 | DVLLFGSGTKVTVLRTVAAPSVFIFPPSDEQLKSGTASVVCLLNNE |
| | | YPREAKVQWKVDNALQSGNSQESVTEQDSKDSTYSLSSTLTLSKAD |
| | | YEKHKVYACEVTHQGLSSPVTKSENRGEC |
| 63 | PAD4 full kappa light chain | QSALTQPRSVSGSPGQSVTISCTGTSGDVGRYSHVSWYQQHPGKAP |
| | (V_{L}-C_{L}(K)) | KLIIYNVYERPSGVPDRESGSKSGNTASLTISGLQAEDEADYYCSS |
| | Clone 01, 03, 05 | HSRSSTPVLFGGGTKLTVLRTVAAPSVFIFPPSDEQLKSGTASVVC |
| | | LLNNFYPREAKVQWKVDNALQSGNSQESVTEQDSKDSTYSLSSTLT |
| | | LSKADYEKHKVYACEVTHQGLSSPVTKSENRGEC |
| 64 | PAD2 full lambda light chain | SYVLTQPPSVSVSPGQTASITCSGDKVGDKYVSWYQQKPGQSPVLV |
| | (V_{L}-C_{L}(λ)) | IYQDSQRPSGIPERFSGSNSGNTATLTISGTQAMDEADYYCQTWAP |
| | Clone 01, 03, 05 | DVLLFGSGTKVTVLGQPKLAPSVTLFPPCSEELQANKATLVCLISD |
| | | FYPGAVTVAWKADSSPVKAGVETTTPSKQSNNKYAASSYLSLTPEQ |
| | | WKSHRSYSCQVTHEGSTVEKTVAPTEVS |
| 65 | PAD4 full lambda light | QSALTQPRSVSGSPGQSVTISCTGTSGDVGRYSHVSWYQQHPGKAP |
| | (V_{L}-C_{L}(λ)) | KLIIYNVYERPSGVPDRESGSKSGNTASLTISGLQAEDEADYYCSS |
| | Clone 02, 04, 06 | HSRSSTPVLFGGGTKLTVLGQPKLAPSVTLFPPCSEELQANKATLV |
| | | CLISDFYPGAVTVAWKADSSPVKAGVETTTPSKQSNNKYAASSYLS |
| | | LTPEQWKSHRSYSCQVTHEGSTVEKTVAPTEVS |
| 66 | PAD2 FQQ-YTE ‘hole’ Fc | ASTKGPSVFPLAPSSKSTSGGTAALGCLVKDYFPEPVTVSWNSGAL |
| | with RF | TSGVHTFPAVLQSSGLYSLSSVVTVPSSSLGTQTYICNVNHKPSNT |
| | (C_{H}1 C_{H}2 C_{H}3) | KVDKRVEPKSCDKTHTCPPCPAPEFQGGPSVFLFPPKPKDTLYITR |
| | Clone 06 | EPEVTCVVVDVSHEDPEVKENWYVDGVEVHNAKTKPREEQYNSTYR |
| | | VVSVLTVLHQDWLNGKEYKCQVSNKALPAPIEKTISKAKGQPREPQ |
| | | VCTLPPSREEMTKNQVSLSCAVKGFYPSDIAVEWESNGQPENNYKT |
| | | TPPVLDSDGSFFLVSKLTVDKSRWQQGNVFSCSVMHEALHNHYTQK |
| | | SLSLSPGK |
| 67 | PAD4 HC ‘hole’ TM | QVQLVESGGGLVKPGGSLRLSCAASGSTLSDYFVSWIRQAPGKGLE |
| | (VH-C_{H}1 C_{H}2 C_{H}3) | WVSFINAANTFTYYADSVRGRFTISRDNAKNSVYLQMNSLRAEDTA |
| | Clone 01 | VYYCSSANDDVDDIVAPGRGYYMDVWGRGTLVTVSSASTKGPSVFP |
| | | LAPSSKSTSGGTAALGCLVKDYFPEPVTVSWNSGALTSGVHTFPAV |
| | | LQSSGLYSLSSVVTVPSSSLGTQTYICNVNHKPSNTKVDKRVEPKS |
| | | CDKTHTCPPCPAPEFEGGPSVFLFPPKPKDTLMISRTPEVTCVVVD |
| | | VSHEDPEVKFNWYVDGVEVHNAKTKPREEQYNSTYRVVSVLTVLHQ |
| | | DWLNGKEYKCKVSNKALPASIEKTISKAKGQPREPQVCTLPPSREE |
| | | MTKNQVSLSCAVKGFYPSDIAVEWESNGQPENNYKTTPPVLDSDGS |
| | | FFLVSKLTVDKSRWQQGNVFSCSVMHEALHNRFTQKSLSLSPGK |
| 68 | PAD4 HC ‘hole’ TM YTE | QVQLVESGGGLVKPGGSLRLSCAASGSTLSDYFVSWIRQAPGKGLE |
| | (VH-C_{H}1 C_{H}2 C_{H}3) | WVSFINAANTFTYYADSVRGRFTISRDNAKNSVYLQMNSLRAEDTA |
| | Clone 03 | VYYCSSANDDVDDIVAPGRGYYMDVWGRGTLVTVSSASTKGPSVFP |
| | | LAPSSKSTSGGTAALGCLVKDYFPEPVTVSWNSGALTSGVHTFPAV |
| | | LQSSGLYSLSSVVTVPSSSLGTQTYICNVNHKPSNTKVDKRVEPKS |
| | | CDKTHTCPPCPAPEFEGGPSVFLFPPKPKDTLYITREPEVTCVVVD |
| | | VSHEDPEVKENWYVDGVEVHNAKTKPREEQYNSTYRVVSVLTVLHQ |
| | | DWLNGKEYKCKVSNKALPASIEKTISKAKGQPREPQVCTLPPSREE |
| | | MTKNQVSLSCAVKGFYPSDIAVEWESNGQPENNYKTTPPVLDSDGS |
| | | FFLVSKLTVDKSRWQQGNVFSCSVMHEALHNRFTQKSLSLSPGK |
| 69 | PAD4 HC ‘hole’ RFFQQ YTE | QVQLVESGGGLVKPGGSLRLSCAASGSTLSDYFVSWIRQAPGKGLE |
| | (VH-C_{H}1 C_{H}2 C_{H}3) | WVSFINAANTFTYYADSVRGRFTISRDNAKNSVYLQMNSLRAEDTA |
| | Clone 05 | VYYCSSANDDVDDIVAPGRGYYMDVWGRGTLVTVSSASTKGPSVFP |
| | | LAPSSKSTSGGTAALGCLVKDYFPEPVTVSWNSGALTSGVHTFPAV |
| | | LQSSGLYSLSSVVTVPSSSLGTQTYICNVNHKPSNTKVDKRVEPKS |
| | | CDKTHTCPPCPAPEFQGGPSVFLFPPKPKDTLYITREPEVTCVVVD |
| | | VSHEDPEVKFNWYVDGVEVHNAKTKPREEQYNSTYRVVSVLTVLHQ |
| | | DWLNGKEYKCQVSNKALPAPIEKTISKAKGQPREPQVCTLPPSREE |
| | | MTKNQVSLSCAVKGFYPSDIAVEWESNGQPENNYKTTPPVLDSDGS |
| | | FFLVSKLTVDKSRWQQGNVFSCSVMHEALHNRFTQKSLSLSPGK |
| 70 | PAD2 HC ‘knob’ TM | EVQLLESGGGLVQPGGSLRLSCAASGFTFSSYAMSWVRQAPGKGLE |
| | (VH-C_{H}1 C_{H}2 C_{H}3) | WVSAISGSGGSTYYADSVKGRFTISRDNSKNTLYLQMNSLRAEDTA |
| | Clone 01 | VYYCARQVLVRGFFSHEDDAVDIWGQGTTVTVSSASTKGPSVCPLA |
| | | PSSKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSVD |
| | | KTHTCPPCPAPEFEGGPSVFLFPPKPKDTLMISRTPEVTCVVVDVS |
| | | HEDPEVKFNWYVDGVEVHNAKTKPREEQYNSTYRVVSVLTVLHQDW |
| | | LNGKEYKCKVSNKALPASIEKTISKAKGQPREPQVYTLPPCREEMT |
| | | KNQVSLWCLVKGFYPSDIAVEWESNGQPENNYKTTPPVLDSDGSFF |
| | | LYSKLTVDKSRWQQGNVFSCSVMHEALHNHYTQKSLSLSPGK |
| 71 | PAD2 HC ‘knob’ TM YTE | EVQLLESGGGLVQPGGSLRLSCAASGFTFSSYAMSWVRQAPGKGLE |
| | (VH-C_{H}1 C_{H}2 C_{H}3) | WVSAISGSGGSTYYADSVKGRFTISRDNSKNTLYLQMNSLRAEDTA |
| | Clone 03 | VYYCARQVLVRGFFSHEDDAVDIWGQGTTVTVSSASTKGPSVCPLA |
| | | PSSKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSVD |
| | | KTHTCPPCPAPEFEGGPSVFLFPPKPKDTLYITREPEVTCVVVDVS |
| | | HEDPEVKFNWYVDGVEVHNAKTKPREEQYNSTYRVVSVLTVLHQDW |
| | | LNGKEYKCKVSNKALPASIEKTISKAKGQPREPQVYTLPPCREEMT |
| | | KNQVSLWCLVKGFYPSDIAVEWESNGQPENNYKTTPPVLDSDGSFF |
| | | LYSKLTVDKSRWQQGNVFSCSVMHEALHNHYTQKSLSLSPGK |
| 72 | PAD2 HC ‘knob’ FQQ YTE | EVQLLESGGGLVQPGGSLRLSCAASGFTFSSYAMSWVRQAPGKGLE |
| | (VH-C_{H}1 C_{H}2 C_{H}3) | WVSAISGSGGSTYYADSVKGRFTISRDNSKNTLYLQMNSLRAEDTA |
| | Clone 05 | VYYCARQVLVRGFFSHEDDAVDIWGQGTTVTVSSASTKGPSVCPLA |
| | | PSSKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSVD |
| | | KTHTCPPCPAPEFQGGPSVFLFPPKPKDTLYITREPEVTCVVVDVS |
| | | HEDPEVKFNWYVDGVEVHNAKTKPREEQYNSTYRVVSVLTVLHQDW |
| | | LNGKEYKCQVSNKALPAPIEKTISKAKGQPREPQVYTLPPCREEMT |
| | | KNQVSLWCLVKGFYPSDIAVEWESNGQPENNYKTTPPVLDSDGSFF |
| | | LYSKLTVDKSRWQQGNVFSCSVMHEALHNHYTQKSLSLSPGK |
| 73 | PAD2 HC ‘hole’ RF TM | EVQLLESGGGLVQPGGSLRLSCAASGFTFSSYAMSWVRQAPGKGLE |
| | (VH-C_{H}1 C_{H}2 C_{H}3) | WVSAISGSGGSTYYADSVKGRFTISRDNSKNTLYLQMNSLRAEDTA |
| | Clone 02 | VYYCARQVLVRGFFSHEDDAVDIWGQGTTVTVSSASTKGPSVFPLA |
| | | PSSKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSCD |
| | | KTHTCPPCPAPEFEGGPSVFLFPPKPKDTLMISRTPEVTCVVVDVS |
| | | HEDPEVKFNWYVDGVEVHNAKTKPREEQYNSTYRVVSVLTVLHQDW |
| | | LNGKEYKCKVSNKALPASIEKTISKAKGQPREPQVCTLPPSREEMT |
| | | KNQVSLSCAVKGFYPSDIAVEWESNGQPENNYKTTPPVLDSDGSFF |
| | | LVSKLTVDKSRWQQGNVFSCSVMHEALHNRFTQKSLSLSPGK |
| 74 | PAD2 HC ‘hole’ TM YTE | EVQLLESGGGLVQPGGSLRLSCAASGFTFSSYAMSWVRQAPGKGLE |
| | (VH-C_{H}1 C_{H}2 C_{H}3) | WVSAISGSGGSTYYADSVKGRFTISRDNSKNTLYLQMNSLRAEDTA |
| | Clone 04 | VYYCARQVLVRGFFSHEDDAVDIWGQGTTVTVSSASTKGPSVFPLA |
| | | PSSKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSCD |
| | | KTHTCPPCPAPEFEGGPSVFLFPPKPKDTLYITREPEVTCVVVDVS |
| | | HEDPEVKFNWYVDGVEVHNAKTKPREEQYNSTYRVVSVLTVLHQDW |
| | | LNGKEYKCKVSNKALPASIEKTISKAKGQPREPQVCTLPPSREEMT |
| | | KNQVSLSCAVKGFYPSDIAVEWESNGQPENNYKTTPPVLDSDGSFF |
| | | LVSKLTVDKSRWQQGNVFSCSVMHEALHNHRFTQKSLSLSPGK |
| 60 | PAD2 HC ‘hole’ FQQ YTE | EVQLLESGGGLVQPGGSLRLSCAASGFTFSSYAMSWVRQAPGKGLE |
| | (VH-C_{H}1 C_{H}2 C_{H}3) | WVSAISGSGGSTYYADSVKGRFTISRDNSKNTLYLQMNSLRAEDTA |
| | Clone 06 | VYYCARQVLVRGFFSHEDDAVDIWGQGTTVTVSSASTKGPSVFPLA |
| | | PSSKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSCD |
| | | KTHTCPPCPAPEFQGGPSVFLFPPKPKDTLYITREPEVTCVVVDVS |
| | | HEDPEVKFNWYVDGVEVHNAKTKPREEQYNSTYRVVSVLTVLHQDW |
| | | LNGKEYKCQVSNKALPAPIEKTISKAKGQPREPQVCTLPPSREEMT |
| | | KNQVSLSCAVKGFYPSDIAVEWESNGQPENNYKTTPPVLDSDGSFF |
| | | LVSKLTVDKSRWQQGNVFSCSVMHEALHNRFTQKSLSLSPGK |
| 76 | PAD4 HC ‘knob’ TM | QVQLVESGGGLVKPGGSLRLSCAASGSTLSDYFVSWIRQAPGKGLE |
| | (VH-C_{H}1 C_{H}2 C_{H}3) | WVSFINAANTFTYYADSVRGRFTISRDNAKNSVYLQMNSLRAEDTA |
| | Clone 02 | VYYCSSANDDVDDIVAPGRGYYMDVWGRGTLVTVSSASTKGPSVCP |
| | | LAPSSKSTSGGTAALGCLVKDYFPEPVTVSWNSGALTSGVHTFPAV |
| | | LQSSGLYSLSSVVTVPSSSLGTQTYICNVNHKPSNTKVDKRVEPKS |
| | | VDKTHTCPPCPAPEFEGGPSVFLFPPKPKDTLMISRTPEVTCVVVD |
| | | VSHEDPEVKFNWYVDGVEVHNAKTKPREEQYNSTYRVVSVLTVLHQ |
| | | DWLNGKEYKCKVSNKALPASIEKTISKAKGQPREPQVYTLPPCREE |
| | | MTKNQVSLWCLVKGFYPSDIAVEWESNGQPENNYKTTPPVLDSDGS |
| | | FFLYSKLTVDKSRWQQGNVFSCSVMHEALHNHYTQKSLSLSPGK |
| 77 | PAD4 HC ‘knob’ TM YTE | QVQLVESGGGLVKPGGSLRLSCAASGSTLSDYFVSWIRQAPGKGLE |
| | (VH-C_{H}1 C_{H}2 C_{H}3) | WVSFINAANTFTYYADSVRGRFTISRDNAKNSVYLQMNSLRAEDTA |
| | Clone 04 | VYYCSSANDDVDDIVAPGRGYYMDVWGRGTLVTVSSASTKGPSVCP |
| | | LAPSSKSTSGGTAALGCLVKDYFPEPVTVSWNSGALTSGVHTFPAV |
| | | LQSSGLYSLSSVVTVPSSSLGTQTYICNVNHKPSNTKVDKRVEPKS |
| | | VDKTHTCPPCPAPEFEGGPSVFLFPPKPKDTLYITREPEVTCVVVD |
| | | VSHEDPEVKFNWYVDGVEVHNAKTKPREEQYNSTYRVVSVLTVLHQ |
| | | DWLNGKEYKCKVSNKALPASIEKTISKAKGQPREPQVYTLPPCREE |
| | | MTKNQVSLWCLVKGFYPSDIAVEWESNGQPENNYKTTPPVLDSDGS |
| | | FFLYSKLTVDKSRWQQGNVFSCSVMHEALHNHYTQKSLSLSPGK |
| 61 | PAD4 HC ‘knob’ FQQ YTE | QVQLVESGGGLVKPGGSLRLSCAASGSTLSDYFVSWIRQAPGKGLE |
| | (VH-C_{H}1 C_{H}2 C_{H}3) | WVSFINAANTFTYYADSVRGRFTISRDNAKNSVYLQMNSLRAEDTA |
| | Clone 06 | VYYCSSANDDVDDIVAPGRGYYMDVWGRGTLVTVSSASTKGPSVCP |
| | | LAPSSKSTSGGTAALGCLVKDYFPEPVTVSWNSGALTSGVHTFPAV |
| | | LQSSGLYSLSSVVTVPSSSLGTQTYICNVNHKPSNTKVDKRVEPKS |
| | | VDKTHTCPPCPAPEFQGGPSVFLFPPKPKDTLYITREPEVTCVVVD |
| | | VSHEDPEVKFNWYVDGVEVHNAKTKPREEQYNSTYRVVSVLTVLHQ |
| | | DWLNGKEYKCQVSNKALPAPIEKTISKAKGQPREPQVYTLPPCREE |
| | | MTKNQVSLWCLVKGFYPSDIAVEWESNGQPENNYKTTPPVLDSDGS |
| | | FFLYSKLTVDKSRWQQGNVFSCSVMHEALHNHYTQKSLSLSPGK |

TABLE 59
Bis3 control (R347)

| SEQ ID NO. | Description | Sequence |
|---|---|---|
| 36 | R347 Lambda Light Chain | GQPKAAPSVTLFPPSSEELQANKATLVCLISDFYPGAVTVAWKADS |
| | (C_{L}(λ)) | SPVKAGVETTTPSKQSNNKYAASSYLSLTPEQWKSHRSYSCQVTHE |
| | | GSTVEKTVAPTECS |
| 80 | R347 C_{H}1 C_{H}2 C_{H}3 | ASTKGPSVFPLAPSSKSTSGGTAALGCLVKDYFPEPVTVSWNSGAL |
| | FQQ-YTE | TSGVHTFPAVLQSSGLYSLSSVVTVPSSSLGTQTYICNVNHKPSNT |
| | | KVDKRVEPKSCDKTHTCPPCPAPEFQGGPSVFLFPPKPKDTLYITR |
| | | EPEVTCVVVDVSHEDPEVKENWYVDGVEVHNAKTKPREEQYNSTYR |
| | | VVSVLTVLHQDWLNGKEYKCQVSNKALPAPIEKTISKAKGQPREPQ |
| | | VYTLPPSREEMTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGKggggsggggs |
| 81 | R347 VL | ELVLTQPASVSGSPGQSITISCTGTSSDVGGYNYVSWYQQHPGKAP |
| | | KLMIYDVSKRPSGVSNRFSGSKSGNTASLTISGLQAEDEADYYCSS |
| | | YTSSSTLVFGGGTKLTVL |
| 82 | R347 VH | EVQLLESGGGLVQPGGSLRLSCTTSGFTENTYAMSWVRQAPGKGLE |
| | | WLSGINNNGRTAFYADSVKGRFTISRDNSKNTLYLQINSLRADDTA |
| | | VYFCAKDVRFIAVPGDSWGQGTLVTVSS |
| 83 | R347 scFv (VH-VL with | EVQLLESGGGLVQPGGSLRLSCTTSGFTENTYAMSWVRQAPGKGLE |
| | (G4S)4) | WLSGINNNGRTAFYADSVKGRFTISRDNSKNTLYLQINSLRADDTA |
| | | VYFCAKDVRFIAVPGDSWGQGTLVTVSSGGGGSGGGGSGGGGSGGG |
| | | GSELVLTQPASVSGSPGQSITISCTGTSSDVGGYNYVSWYQQHPGK |
| | | APKLMIYDVSKRPSGVSNRFSGSKSGNTASLTISGLQAEDEADYYC |
| | | SSYTSSSTLVFGGGTKLTVL |
| 84 | R347 Bis3 | ELVLTQPASVSGSPGQSITISCTGTSSDVGGYNYVSWYQQHPGKAP |
| | FQQ-YTE | KLMIYDVSKRPSGVSNRFSGSKSGNTASLTISGLQAEDEADYYCSS |
| | | YTSSSTLVFGGGTKLTVLGQPKAAPSVTLFPPSSEELQANKATLVC |
| | | LISDFYPGAVTVAWKADSSPVKAGVETTTPSKQSNNKYAASSYLSL |
| | | TPEQWKSHRSYSCQVTHEGSTVEKTVAPTECSEVQLLESGGGLVQP |
| | | GGSLRLSCTTSGFTENTYAMSWVRQAPGKGLEWLSGINNNGRTAFY |
| | | ADSVKGRFTISRDNSKNTLYLQINSLRADDTAVYFCAKDVRFIAVP |
| | | GDSWGQGTLVTVSSASTKGPSVFPLAPSSKSTSGGTAALGCLVKDY |
| | | FPEPVTVSWNSGALTSGVHTFPAVLQSSGLYSLSSVVTVPSSSLGT |
| | | QTYICNVNHKPSNTKVDKRVEPKSCDKTHTCPPCPAPEFQGGPSVE |
| | | LFPPKPKDTLYITREPEVTCVVVDVSHEDPEVKENWYVDGVEVHNA |
| | | KTKPREEQYNSTYRVVSVLTVLHQDWLNGKEYKCQVSNKALPAPIE |
| | | KTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSC |
| | | SVMHEALHNHYTQKSLSLSPGKggggsggggsEVQLLESGGGLVQP |
| | | GGSLRLSCTTSGFTENTYAMSWVRQAPGKGLEWLSGINNNGRTAFY |
| | | ADSVKGRFTISRDNSKNTLYLQINSLRADDTAVYFCAKDVRFIAVP |
| | | GDSWGQGTLVTVSSGGGGSGGGGSGGGGSGGGGSELVLTQPASVSG |
| | | SPGQSITISCTGTSSDVGGYNYVSWYQQHPGKAPKLMIYDVSKRPS |
| | | GVSNRFSGSKSGNTASLTISGLQAEDEADYYCSSYTSSSTLVFGGG |
| | | TKLTVL |

TABLE 60
DuetMab control (R347)

| SEQ ID NO. | Description | Sequence |
|---|---|---|
| 85 | R347 ‘hole; heavy chain | EVQLLESGGGLVQPGGSLRLSCTTSGFTENTYAMSWVRQAPGKGLE |
| | | WLSGINNNGRTAFYADSVKGRFTISRDNSKNTLYLQINSLRADDTA |
| | | VYFCAKDVRFIAVPGDSWGQGTLVTVSSASTKGPSVFPLAPSSKST |
| | | SGGTAALGCLVKDYFPEPVTVSWNSGALTSGVHTFPAVLQSSGLYS |
| | | LSSVVTVPSSSLGTQTYICNVNHKPSNTKVDKRVEPKSCDKTHTCP |
| | | PCPAPEFQGGPSVFLFPPKPKDTLYITREPEVTCVVVDVSHEDPEV |
| | | KFNWYVDGVEVHNAKTKPREEQYNSTYRVVSVLTVLHQDWLNGKEY |
| | | KCQVSNKALPAPIEKTISKAKGQPREPQVCTLPPSREEMTKNQVSL |
| | | SCAVKGFYPSDIAVEWESNGQPENNYKTTPPVLDSDGSFFLVSKLT |
| | | VDKSRWQQGNVFSCSVMHEALHNRFTQKSLSLSPGK |
| 86 | R347 ‘knob’ heavy chain | EVQLLESGGGLVQPGGSLRLSCTTSGFTENTYAMSWVRQAPGKGLE |
| | | WLSGINNNGRTAFYADSVKGRFTISRDNSKNTLYLQINSLRADDTA |
| | | VYFCAKDVRFIAVPGDSWGQGTLVTVSSASTKGPSVCPLAPSSKST |
| | | SGGTAALGCLVKDYFPEPVTVSWNSGALTSGVHTFPAVLQSSGLYS |
| | | LSSVVTVPSSSLGTQTYICNVNHKPSNTKVDKRVEPKSVDKTHTCP |
| | | PCPAPEFQGGPSVFLFPPKPKDTLYITREPEVTCVVVDVSHEDPEV |
| | | KFNWYVDGVEVHNAKTKPREEQYNSTYRVVSVLTVLHQDWLNGKEY |
| | | KCQVSNKALPAPIEKTISKAKGQPREPQVYTLPPCREEMTKNQVSL |
| | | WCLVKGFYPSDIAVEWESNGQPENNYKTTPPVLDSDGSFFLYSKLT |
| | | VDKSRWQQGNVFSCSVMHEALHNHYTQKSLSLSPGK |
| 87 | R347 kappa light chain | ELVLTQPASVSGSPGQSITISCTGTSSDVGGYNYVSWYQQHPGKAP |
| | | KLMIYDVSKRPSGVSNRFSGSKSGNTASLTISGLQAEDEADYYCSS |
| | | YTSSSTLVFGGGTKLTVLRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSENRGEC |
| 88 | R347 lambda light chain | ELVLTQPASVSGSPGQSITISCTGTSSDVGGYNYVSWYQQHPGKAP |
| | | KLMIYDVSKRPSGVSNRFSGSKSGNTASLTISGLQAEDEADYYCSS |
| | | YTSSSTLVFGGGTKLTVLGQPKLAPSVTLFPPCSEELQANKATLVC |
| | | LISDFYPGAVTVAWKADSSPVKAGVETTTPSKQSNNKYAASSYLSL |
| | | TPEQWKSHRSYSCQVTHEGSTVEKTVAPTEVS |

TABLE 61
Fc modification sequences

| | Fc variant domain sequence |
|---|---|
| SEQ ID NO: | (Position 432 to 437) |
| 187 | CXRHXC |
| 188 | CRRHXC |
| 189 | CXRHRC |

TABLE 62
PAD40048

| Description | Sequence |
|---|---|
| VH (Kabat) AA | QVQLQESGGGLVKPGGSLRLSCATSGSTLSDY |
| | FVSWIRQAPGKGLEWVSYISSFGTFTYYADSV |
| | RGRFTISRDNAKNTVYLQMNSLRADDTAVYFC |
| | ARDRLSVDDIVAPGRGYYMDVWGRGTLVTVSS |
| HCDR1 (Kabat) AA | DYFVS |
| HCDR2 (Kabat) AA | YISSFGTFTYYADSVRG |
| HCDR3 (Kabat) AA | DRLSVDDIVAPGRGYYMDV |
| HFW1 (Kabat) AA | QVQLQESGGGLVKPGGSLRLSCATSGSTLS |
| HFW2 (Kabat) AA | WIRQAPGKGLEWVS |
| HFW3 (Kabat) AA | RFTISRDNAKNTVYLQMNSLRADDTAVYFCA |
| | R |
| HFW4 (Kabat) AA | WGRGTLVTVSS |
| VL (Kabat) AA | QSALTQPRSVSGSPGQSVAISCTGTRGDVGR |
| | YNHVSWYQQHPGKAPKLIIYNVYERPSGVPD |
| | RESGSKSGNTASLTISGLQAEDEADYYCSSH |
| | SRSSTPVLFGGGTKLTVL |
| LCDR1 (Kabat) AA | TGTRGDVGRYNHVS |
| LCDR2 (Kabat) AA | NVYERPS |
| LCDR3 (Kabat) AA | SSHSRSSTPVL |
| LFW1 (Kabat) AA | QSALTQPRSVSGSPGQSVAISC |
| LFW2 (Kabat) AA | WYQQHPGKAPKLIIY |
| LFW3 (Kabat) AA | GVPDRFSGSKSGNTASLTISGLQAEDEADYY |
| | C |
| LFW4 (Kabat) AA | FGGGTKLTVL |

### 6.2 Sequence Identities

TABLE 63
PAD4 Sequence % identities

| | Identity to Clone 42 (%) | | | | | | | |
|---|---|---|---|---|---|---|---|---|
| Clone/Name | HCDR1 | HCDR2 | HCDR3 | LCDR1 | LCDR2 | LCDR3 | VH | VL |
| Clone 42 | 100 | 100 | 100 | 100 | 100 | 100 | 100 | 100 |
| 48LO0010 hIgG1 ngl-2 | 100 | 70.6 | 84.2 | 85.7 | 100 | 100 | 93 | 98.2 |
| 48LO0032 hIgG1 ngl-2 | 100 | 82.4 | 84.2 | 85.7 | 100 | 100 | 94.5 | 98.2 |
| 48LO0033 hIgG1 ngl-3 | 100 | 47.1 | 89.5 | 85.7 | 100 | 100 | 89.8 | 98.2 |
| 48LO0036 hIgG1 ngl-3 | 100 | 64.7 | 84.2 | 85.7 | 100 | 100 | 89.8 | 98.2 |
| 48LO0040 hIgG1 ngl-3 | 100 | 88.2 | 78.9 | 85.7 | 100 | 100 | 92.2 | 98.2 |
| 48LO0048 hIgG1 ngl-3 | 100 | 70.6 | 84.2 | 85.7 | 100 | 72.7 | 91.4 | 95.5 |
| 48LO0049 hIgG1 ngl-3 | 100 | 70.6 | 78.9 | 85.7 | 100 | 72.7 | 91.4 | 95.5 |
| 48LO0049 hIgG1 pgl-9 | 100 | 70.6 | 78.9 | 92.9 | 100 | 72.7 | 91.4 | 96.4 |
| 48LO0049 hIgG1 pgl-10 | 100 | 70.6 | 78.9 | 78.6 | 100 | 72.7 | 91.4 | 94.6 |
| 48LO0049 hIgG1 pgl-12 | 100 | 70.6 | 78.9 | 92.9 | 100 | 72.7 | 91.4 | 96.4 |
| 48LO0049 hIgG1 fgl-23 | 100 | 70.6 | 78.9 | 71.4 | 100 | 72.7 | 92.2 | 93.7 |
| 48LO0049 hIgG1 fgl-25 | 100 | 70.6 | 78.9 | 71.4 | 100 | 72.7 | 92.2 | 93.7 |
| 48LO0049 hIgG1 pgl-31 | 100 | 70.6 | 78.9 | 78.6 | 100 | 72.7 | 91.4 | 94.6 |
| 48LO0051 hIgG1 ngl-2 | 100 | 70.6 | 84.2 | 85.7 | 100 | 72.7 | 92.2 | 95.5 |
| 48LO0060 hIgG1 ngl-3 | 100 | 88.2 | 78.9 | 85.7 | 100 | 100 | 93.8 | 98.2 |
| 48LO0062 hIgG1 ngl-3 | 100 | 52.9 | 84.2 | 85.7 | 100 | 100 | 89.8 | 98.2 |
| 48LO0063 hIgG1 ngl-3 | 100 | 94.1 | 100 | 85.7 | 100 | 100 | 98.4 | 98.2 |
| 48LO0063 hIgG1 fgl-4 | 100 | 94.1 | 100 | 92.9 | 100 | 100 | 99.2 | 99.1 |
| 48LO0063 hIgG1 fgl-6 | 100 | 94.1 | 100 | 100 | 100 | 100 | 99.2 | 100 |
| 48LO0063 hIgG1 fgl-7 | 100 | 94.1 | 100 | 78.6 | 100 | 100 | 99.2 | 97.3 |
| 48LO0063 hIgG1 fgl-8 | 100 | 94.1 | 100 | 71.4 | 100 | 100 | 99.2 | 96.4 |
| 48LO0063 hIgG1 fgl-9 | 100 | 88.2 | 100 | 92.9 | 100 | 100 | 98.4 | 99.1 |
| 48LO0063 hIgG1 fgl-11 | 100 | 88.2 | 100 | 100 | 100 | 100 | 98.4 | 100 |
| 48LO0063 hIgG1 pgl-38 | 100 | 94.1 | 100 | 85.7 | 100 | 100 | 98.4 | 98.2 |
| 48LO0063 hIgG1 pgl-39 | 100 | 88.2 | 100 | 85.7 | 100 | 100 | 97.7 | 98.2 |
| 48LO0063 hIgG1 pgl-40 | 100 | 88.2 | 100 | 85.7 | 100 | 100 | 97.7 | 98.2 |
| 48LO0063 hIgG1 pgl-41 | 100 | 88.2 | 100 | 85.7 | 100 | 100 | 97.7 | 98.2 |
| 48LO0063 hIgG1 pgl-42 | 100 | 88.2 | 100 | 85.7 | 100 | 100 | 97.7 | 98.2 |
| 48LO0063 hIgG1 pgl-43 | 100 | 88.2 | 100 | 85.7 | 100 | 100 | 97.7 | 98.2 |
| 48LO0063 hIgG1 pgl-44 | 100 | 88.2 | 100 | 85.7 | 100 | 100 | 97.7 | 98.2 |
| 48LO0063 hIgG1 pgl-45 | 100 | 88.2 | 100 | 85.7 | 100 | 100 | 97.7 | 98.2 |
| 48LO0063 hIgG1 pgl-46 | 100 | 88.2 | 100 | 85.7 | 100 | 100 | 97.7 | 98.2 |
| 48LO0063 hIgG1 pgl-47 | 100 | 94.1 | 100 | 85.7 | 100 | 100 | 98.4 | 98.2 |
| 48LO0063 hIgG1 pgl-49 | 100 | 94.1 | 100 | 85.7 | 100 | 100 | 98.4 | 98.2 |
| 48LO0063 hIgG1 pgl-51 | 100 | 94.1 | 100 | 85.7 | 100 | 100 | 98.4 | 98.2 |
| 48LO0063 hIgG1 pgl-52 | 100 | 100 | 100 | 85.7 | 100 | 100 | 99.2 | 98.2 |
| ZZ240R-H02_2 | 100 | 94.1 | 100 | 85.7 | 100 | 100 | 98.4 | 98.2 |
| 48LO0063 hIgG1 fgl-58 | 100 | 88.2 | 100 | 100 | 100 | 100 | 98.4 | 100 |
| 48LO0063 hIgG1 fgl-60 | 100 | 94.1 | 100 | 100 | 100 | 100 | 99.2 | 100 |
| 48LO0063 hIgG1 fgl-61 | 100 | 94.1 | 100 | 100 | 100 | 100 | 99.2 | 100 |

Regions defined by Kabat;

### 6.3 General

The antibody may comprise a PAD2 binding domain that specifically binds to PAD2 and/or a PAD4 binding domain that specifically binds PAD4. The antibody may comprise a domain that specifically binds PAD2. The antibody may comprise a domain that specifically binds PAD4. The antibody may comprise a domain that specifically binds PAD2 and a domain that specifically binds PAD4. The antibody may inhibit PAD activity. The antibody may inhibit PAD-mediated citrullination of proteins. The antibody may inhibit PAD activity in the synovial fluid. The antibody may have an IC₅₀ of ≤200 pM as measured by H3 citrullination assay. The antibody may be a human antibody.

Specific PAD2 binding may be measured by PAD2 ELISA. Specific PAD4 binding may be measured by PAD4 ELISA.

The antibody may have an IC₅₀ of about 700, 650, 600, 550, 540, 530, 520, 500, 480, 460, 440, 450, 430, 420, 400, 380, 360, 340, 320, 300, 280, 260, 240, 220, 200, 180, 160, 140, 120, 100, 90, 80, 70, 60, 50, 40, 30, 20, 19, 18, 17, 16, 15, 14, 13, 12, 11, 10, 9, 8, 7, 6, 4, 2 or 1 pM, as measured by H3 citrullination assay. The IC₅₀ may relate to inhibition of PAD4 activity. The IC₅₀ may relate to inhibition of PAD2 activity. The IC₅₀ may relate to inhibition of combined PAD4 and PAD2 activity.

The antibody may inhibit PAD2 activity. The antibody may inhibit PAD2-mediated protein citrullination. The antibody may inhibit PAD activity with an IC₅₀ of ≤700 PM as measured by H3 citrullination assay. The antibody may have an IC₅₀ of about 700, 650, 600, 550, 540, 530, 520, 500, 480, 460, 440, 450, 430, 420, 400, 380, 360, 340, 320, 300, 280, 260, 240, 220, 200, 180, 160, 140, 120, 100, 90, 80, 70, 60, 50, 40, 30, 20, 19, 18, 17, 16, 15, 14, 13, 12, 11, 10, 9, 8, 7, 6, 4, 2 or 1 pM, as measured by H3 citrullination assay. The IC₅₀ may relate to inhibition of PAD4 activity. The IC₅₀ may relate to inhibition of PAD2 activity. The IC₅₀ may relate to inhibition of combined PAD4 and PAD2 activity.

The IC₅₀ may be measured by trypsin cleavage assay. The IC₅₀ may be measured by BAEE (Nα-Benzonyl-L-arginine ethyl ester hydrochloride) citrullination assay. The IC₅₀ may be measured by H3 citrullination assay.

The antibody may inhibit PAD4 activity, optionally wherein the antibody inhibits PAD4-mediated protein citrullination, optionally with an IC₅₀ of ≤100 pM as measured by H3 citrullination assay, and optionally wherein the PAD4 is recombinant PAD4.

The antibody may inhibit PAD2 in immune cells. The antibody may inhibit PAD4 in immune cells. The antibody may inhibit PAD2 and PAD4 in immune cells. The antibody may inhibit PAD activity in immune cells. The immune cells may be neutrophils. The immune cells may be monocytes. The immune cells may be neutrophils and monocytes.

The PAD2 that the antibody inhibits may be human, cynomolgus or mouse PAD2. The PAD4 that the antibody inhibits may be human, cynomolgus of mouse PAD4.

The antibody may comprise the sequence of Clone 01, Clone 02, Clone 03, Clone 04, Clone 05, Clone 06, Clone 07, Clone 08, Clone 09, Clone 10, Clone 11, Clone 12, Clone 22, or Clone 42, e.g. as provided in Table 1, Table 2, Table 57 and Table 58.

### 6.4 Specificity

The PAD2 binding domain may not specifically bind PAD3 or PAD1. The PAD4 binding domain may not specifically bind PAD3 or PAD1. PAD3 binding may be measured by PAD3 ELISA. PAD1 binding may be measured by PAD1 ELISA. The antibody may not specifically bind PAD3 or PAD1. The PAD3 may be human, cynomolgus or mouse PAD3. The PAD1 may be human, cynomolgus or mouse PAD1. The antibody may specifically bind PAD4, but not PAD1, PAD2 or PAD3. The antibody may specifically bind PAD2, but not PAD1, PAD4 or PAD3. The antibody may specifically bind PAD2 and PAD4, but not PAD1 or PAD3.

The antibody may specifically bind mouse PAD2. The antibody may specifically bind mouse PAD4. The antibody may not specifically bind PAD2. The antibody may not specifically bind PAD4.

### 6.5 Bispecific

The antibody may be a bispecific comprising a PAD2 binding domain and a PAD4 binding domain. The PAD2 binding domain may specifically bind PAD2 specifically but not PAD1, PAD4 or PAD3. The PAD4 binding domain may specifically bind PAD4 but not PAD1, PAD3 or PAD2.

### 6.6 PAD2 Affinity

The bispecific may bind human PAD2 with an affinity (K_{D}) that is equivalent to the affinity (K_{D}) of a bivalent Fab fragment or IgG comprising the same PAD2 binding domain for human PAD2. The bispecific antibody may bind human PAD2 with an affinity (K_{D}) that is within ±5 pM of the affinity (K_{D}) for human PAD2 of a bivalent Fab fragment of IgG comprising the same PAD2 binding domain. The bispecific antibody may bind PAD2 with an affinity (K_{D}) that is within ±10 pM of the affinity (K_{D}) for human PAD2 of a bivalent Fab fragment of IgG comprising the same PAD2 binding domain.

The K_{D} of the antibody or bispecific for human PAD may be less than the K_{D} of a bivalent Fab fragment of IgG comprising the same PAD2 binding domain for human PAD2. The antibody may have an affinity for human PAD2 of K_{D} 3-30 pM or 10-20 pM. The antibody may have an affinity (K_{D}) for human PAD2 of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 pM. The antibody may have an affinity (K_{D}) for cynomolgus PAD2 of about 10, 20, 30, 40, 50, 60, 70, 80, 90, 100, 110, 120, 130, 140, 150 and 160 pM. The antibody may have an affinity (K_{D}) for mouse PAD2 of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 pM. The antibody may bind human PAD2 with an affinity (K_{D}) of ≤20 pM or ≤18 pM. The affinity may be measured by surface plasmon resonance (SPR).

The bispecific may bind cynomolgus PAD2 with an affinity (K_{D}) that is equivalent to the affinity (K_{D}) of a bivalent Fab fragment of IgG comprising the same PAD2 binding domain. The bispecific antibody may bind cynomolgus PAD2 with an affinity (K_{D}) that is within ±5 pM of the affinity (K_{D}) for cynomolgus PAD2 of a bivalent Fab fragment of IgG comprising the same PAD2 binding domain. The bispecific antibody may bind PAD2 with an affinity (K_{D}) that is within ±10 pM of the affinity (K_{D}) for cynomolgus PAD2 of a bivalent Fab fragment of IgG comprising the same PAD2 binding domain. The K_{D} of the bispecific for cynomolgus PAD may be less than the K_{D} of a bivalent Fab fragment of IgG comprising the same PAD2 binding domain for cynomolgus PAD2.

The bispecific may bind mouse PAD2 with an affinity (K_{D}) that is equivalent to the affinity (K_{D}) of a bivalent Fab fragment of IgG comprising the same PAD2 binding domain for mouse PAD2. The bispecific antibody may bind mouse PAD2 with an affinity (K_{D}) that is within ±5 pM of the affinity (K_{D}) for mouse PAD2 of a bivalent Fab fragment of IgG comprising the same PAD2 binding domain. The bispecific antibody may bind PAD2 with an affinity (K_{D}) that is within ±10 pM of the affinity (K_{D}) for mouse PAD2 of a bivalent Fab fragment of IgG comprising the same PAD2 binding domain. The K_{D} of the bispecific for mouse PAD2 may be less than the K_{D} of a bivalent Fab fragment of IgG comprising the same PAD2 binding domain for mouse PAD2.

The affinity (K_{D}) of the antibody for PAD2 may be any of the affinities provided in the examples, particularly as provided in Table 68 and Table 69. The affinity K_{D} of the antibody for PAD4 may be in the range of the affinities provided in the examples, particularly as provided in Table 70 or Table 71.

The affinity (e.g. K_{D}) may be measured by surface plasmon resonance (SPR).

### 6.7 PAD4 Affinity

The bispecific may bind human PAD4 with an affinity (K_{D}) that is equivalent to the affinity (K_{D}) of a bivalent Fab fragment of IgG comprising the same PAD4 binding domain for human PAD4. The bispecific antibody may bind human PAD4 with an affinity (K_{D}) that is within ±5 pM of the affinity (K_{D}) for human PAD4 of a bivalent Fab fragment of IgG comprising the same PAD4 binding domain. The bispecific antibody may bind PAD4 with an affinity (K_{D}) that is within ±10 pM of the affinity (K_{D}) for human PAD4 of a bivalent Fab fragment of IgG comprising the same PAD4 binding domain. The bispecific antibody may bind PAD4 with an affinity (K_{D}) that is within ±20 pM of the affinity (K_{D}) for human PAD4 of a bivalent Fab fragment of IgG comprising the same PAD4 binding domain. The bispecific antibody may bind PAD4 with an affinity (K_{D}) that is within ±30 pM of the affinity (K_{D}) for human PAD4 of a bivalent Fab fragment of IgG comprising the same PAD4 binding domain.

The K_{D} of the antibody or bispecific for human PAD4 may be less than the K_{D} of a bivalent Fab fragment of IgG comprising the same PAD4 binding domain for human PAD4. The antibody may have an affinity for human PAD4 of K_{D} 5-60 pM, 7-10 pM or 6-30 pM. The antibody may have an affinity for human PAD4 of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 pM.

The antibody may have an affinity K_{D} for mouse PAD4 of about 5 to 50 pM or 5 to 45 pM. The antibody may have an affinity K_{D} for mouse PAD4 of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50.

The bispecific may bind cynomolgus PAD4 with an affinity (K_{D}) that is equivalent to the affinity (K_{D}) of a bivalent Fab fragment of IgG comprising the same PAD4 binding domain. The bispecific antibody may bind cynomolgus PAD4 with an affinity (K_{D}) that is within ±1 pM of the affinity (K_{D}) for cynomolgus PAD4 of a bivalent Fab fragment of IgG comprising the same PAD4 binding domain. The bispecific antibody may bind PAD4 with an affinity (K_{D}) that is within ±2 pM of the affinity (K_{D}) for cynomolgus PAD4 of a bivalent Fab fragment of IgG comprising the same PAD4 binding domain. The K_{D} of the bispecific for cynomolgus PAD4 may be less than the K_{D} of a bivalent Fab fragment of IgG comprising the same PAD4 binding domain for cynomolgus PAD4.

The bispecific may bind mouse PAD4 with an affinity (K_{D}) that is equivalent to the affinity (K_{D}) of a bivalent Fab fragment of IgG comprising the same PAD2 binding domain for mouse PAD4. The bispecific antibody may bind mouse PAD4 with an affinity (K_{D}) that is within ±5 pM of the affinity (K_{D}) for mouse PAD4 of a bivalent Fab fragment of IgG comprising the same PAD4 binding domain. The bispecific antibody may bind PAD4 with an affinity (K_{D}) that is within ±10 pM of the affinity (K_{D}) for mouse PAD2 of a bivalent Fab fragment of IgG comprising the same PAD4 binding domain. The K_{D} of the bispecific for mouse PAD may be less than the K_{D} of a bivalent Fab fragment of IgG comprising the same PAD4 binding domain for mouse PAD4.

The antibody may bind human PAD4 with an affinity (K_{D}) of ≤40 pM or ≤35 pM.

The affinity K_{D} of the antibody for PAD4 may be any of the affinities provided examples, particularly as provided in Table 70 or Table 71. The affinity K_{D} of the antibody for PAD4 may be in the range of the affinities provided in the examples, particularly as provided in Table 70 or Table 71.

### 6.8 Thermostability

The antibody of bispecific may have beneficial thermostability. The antibody or bispecific may have a Tₒₙₛₑₜ of ≥40° C. The antibody or bispecific may have a Tₒₙₛₑₜ of about 44, 45, 46, 47, 48, 49, 50, 51, 52, 53 or 54° C. The antibody or bispecific may have a Tₒₙₛₑₜ of 40 to 54° C. Tₒₙₛₑₜ may be measured by Nano Differential Scanning Fluorimetry (DSF). The thermostability (Tₒₙₛₑₜ) of the bispecific may be equivalent or greater than the thermostability (Tₒₙₛₑₜ) of a bivalent Fab fragment of IgG comprising the same PAD4 binding domain. The thermostability (Tₒₙₛₑₜ) of the bispecific may be equivalent or greater than the thermostability (Tₒₙₛₑₜ) of a bivalent Fab fragment of IgG comprising the same PAD2 binding domain. The thermostability (Tₒₙₛₑₜ) of the bispecific antibody may be within ±10° C. of the thermostability (Tₒₙₛₑₜ) of a bivalent Fab fragment of IgG comprising the same PAD4 binding domain. The thermostability (Tₒₙₛₑₜ) of the bispecific antibody may be within ±20° C. of the thermostability (Tₒₙₛₑₜ) of a bivalent Fab fragment of IgG comprising the same PAD4 binding domain. The thermostability (Tₒₙₛₑₜ) of the bispecific antibody may be within ±10° C. of the thermostability (Tₒₙₛₑₜ) of a bivalent Fab fragment of IgG comprising the same PAD2 binding domain. The thermostability (Tₒₙₛₑₜ) of the bispecific antibody may be within ±20° C. of the thermostability (Tₒₙₛₑₜ) of a bivalent Fab fragment of IgG comprising the same PAD2 binding domain.

The thermostability (Tₒₙₛₑₜ) of the antibody may be any of the values provided Table 91. The thermostability (Tₒₙₛₑₜ) of the antibody may be in the range of the values provided in Table 91.

### 6.9 Aggregation

The antibody may have beneficially low risk of aggregation. The propensity of the bispecific to aggregate may be no more than 2-fold greater than the propensity of a bivalent Fab of IgG to aggregate comprising the same PAD2 binding domain. The propensity of the bispecific to aggregate may be no more than 2-fold greater than the propensity of a bivalent Fab of IgG to aggregate comprising the same PAD4 binding domain. The aggregation of the antibody at 40° C. may be no more than 2-fold greater than the aggregation of a bivalent IgG1 antibody comprising the same PAD2 binding domain or PAD4 binding domain. The aggregation of the antibody at 40° C. may be no more than 2-fold greater than the aggregation of a bivalent IgG1 antibody comprising the same PAD2 binding domain or PAD4 binding domain.

### 6.10 Bis3

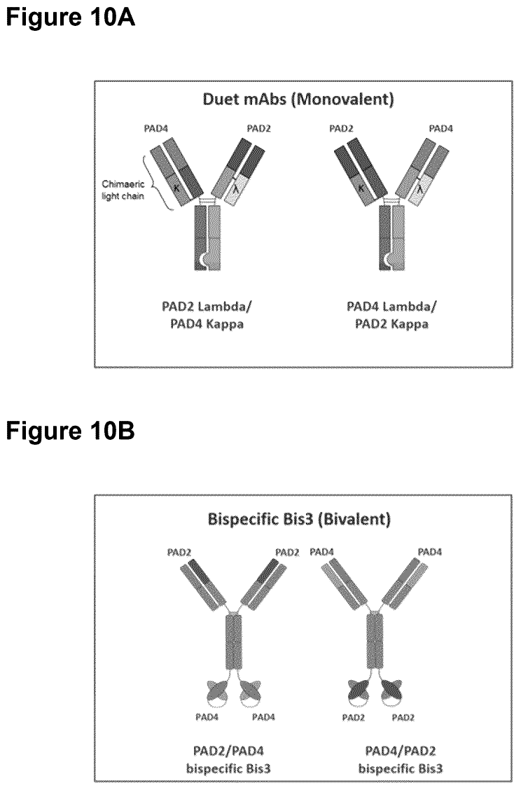

The bispecific may be a bivalent bispecific. The bispecific may be in Bis3 format, i.e. having scFv and IgG binding domains (FIG. 10B). The antibody may comprise two PAD2 binding domains such that the antibody is bivalent for PAD2 and two PAD4 binding domains such that the antibody is also bivalent for PAD4. The PAD2 and PAD4 may be human, cynomolgus and/or mouse PAD2 or PAD4. The antibody may comprise two scFv domains, wherein each scFv domain comprises a PAD2 binding domain according to the invention. The antibody may comprise two scFv domains, wherein each scFv domain comprises a PAD4 binding domain according to the invention.

The antibody may comprise two Fab domains, wherein each Fab comprises a PAD2 binding domain according to the invention. The antibody may comprise two Fab domains, wherein each Fab comprises a PAD4 binding domain according to the invention. The antibody may be a Bis3 bispecific having either of the structures shown in FIG. 10B.

The Bis3 bispecific may bind human PAD4 with an affinity (K_{D}) of 25-50 pM, wherein each scFv domain comprises a PAD4 binding domain according to the invention and each Fab domain comprises a PAD2 binding domain according to the invention. The Bis3 bispecific may bind human PAD4 with an affinity (K_{D}) of about 9 pM, wherein each scFv domain comprises a PAD2 binding domain according to the invention and each Fab domain comprises a PAD4 binding domain according to the invention.

The Bis3 bispecific may bind human PAD2 with an affinity (K_{D}) of 6 to 7 pM, wherein each scFv domain comprises a PAD4 binding domain according to the invention and each Fab domain comprises a PAD2 binding domain according to the invention. The Bis3 bispecific may bind human PAD2 with an affinity (K_{D}) of about 16 pM, wherein each scFv domain comprises a PAD2 binding domain according to the invention and each Fab domain comprises a PAD4 binding domain according to the invention. The antibody may bind human PAD2 with an affinity (K_{D}) of ≤17 pM. The antibody may bind human PAD4 with an affinity (K_{D}) of ≤35 pM.

The Bis3 bispecific may have an affinity (K_{D}) for human or cynomolgus PAD2 as provided in Table 72 or Table 73. The Bis3 may have an affinity (K_{D}) for PAD4 as provided in Table 75 or Table 74.

The antibody may be a bispecific comprising a) an IgG comprising first and second Fab domains and an Fc domain, wherein the first and second Fab domains each comprise a PAD2 binding domain which specifically binds PAD2, and b) first and second scFvs, wherein the first and second scFvs are each respectively linked to the carboxy terminal of one of the heavy chains of the Fc domain of the IgG, and wherein the first and second scFvs each comprise a PAD4 binding domain which specifically binds PAD4. The Fc domain may be an IgG or IgG1 Fc domain. The first and second scFv PAD4 binding domains may comprise SEQ ID NO: 39. The first and second Fab PAD2 binding domains may comprise a heavy chain domain comprising SEQ ID NO: 34. The first and second Fab PAD2 binding domains may comprise a light chain constant domain comprising SEQ ID NO: 36. The first and second Fab PAD2 binding domains comprise a light chain domain comprising SEQ ID NO: 35.

The antibody may be a bispecific comprising a) an IgG comprising first and second Fab domains and an Fc domain, wherein the first and second Fab domains each comprise a PAD4 binding domain that specifically binds PAD4, and b) first and second scFvs, wherein the first and second scFvs are each respectively linked to the carboxy terminal of one of the heavy chains of the Fc domain of the IgG, and wherein the first and second scFvs each comprise a PAD2 binding domain that specifically binds PAD2. The first and second scFv PAD2 binding domains may comprise SEQ ID NO: 38. The first and second Fab PAD4 binding domains comprise a heavy chain domain comprising SEQ ID NO: 40. The first and second Fab PAD4 binding domains comprise a light chain domain comprise SEQ ID NO: 41. The first and second Fab domains comprise a heavy chain constant domain may comprise SEQ ID NO: 37. The first and second Fab domains comprise a light chain constant domain comprising SEQ ID NO: 36.

The scFv may be linked to the carboxy terminal of the heavy chain by a peptide linker. The peptide linker may comprise SEQ ID NO: 51. The first and/or second scFvs of the Bis3 bispecific may comprise a VH-VL linker domain comprising SEQ ID NO: 33.

The Bis3 bispecific may comprise the sequence SEQ ID NO: 53, SEQ ID NO: 54, SEQ ID NO: 55, SEQ ID NO: 56, SEQ ID NO: 57, or SEQ ID NO: 58. The Bis3 bispecific may comprise a sequence having 90% sequence identity to SEQ ID NO: 53, SEQ ID NO: 54, SEQ ID NO: 55, SEQ ID NO: 56, SEQ ID NO: 57, or SEQ ID NO: 58.

The Bis3 bispecific may have the sequence of any of clones 07, 08, 09, 10, 11 or 12. The Bis3 bispecific may have a sequence that is at least 90% identical to the sequence of clones 07, 08, 09, 10, 11 or 12. The Bis3 bispecific may have a sequence as provided in Table 57.

### 6.11 DuetMab

The antibody may be a monovalent bispecific. The antibody may be a DuetMab. The antibody may be a DuetMab comprising either of the structures shown in FIG. 10A.

The antibody may comprise one PAD2 binding domain such that the antibody is monovalent for PAD2 and the antibody may comprise one PAD4 binding domain such that the antibody is monovalent for PAD4. The antibody may comprise an IgG comprising: a first binding region comprising a first Fab wherein the second Fab domain comprises the PAD2 binding domain, and a second binding region comprising a second Fab, wherein the second Fab comprises the PAD4 binding domain. The antibody may comprise and IgG domain having knob/hole mutations. The IgG may comprise a kappa light chain comprising SEQ ID NO: 63. The antibody may comprise a lambda light chain comprising SEQ ID NO: 64. The antibody may comprise a kappa light chain comprising SEQ ID NO: 62. The antibody may comprise a lambda light chain comprising SEQ ID NO: 65.

The DuetMab bispecific may comprise a PAD2 binding domain comprising a heavy chain comprising SEQ ID NO: 73 and a light chain comprising SEQ ID NO: 62, and a PAD4 binding region may comprise a heavy chain comprising SEQ ID NO: 76 and a light chain comprising SEQ ID NO: 65. The DuetMab bispecific may comprise a PAD2 binding domain comprising a heavy chain comprises SEQ ID NO: 74 and a light chain comprising SEQ ID NO: 62, and a PAD4 binding region comprising a heavy chain comprising SEQ ID NO: 76 and a light chain comprising SEQ ID NO: 65. The DuetMab bispecific may comprise a heavy chain comprising SEQ ID NO: 59 or SEQ ID NO: 60 and a light chain comprising SEQ ID NO: 62, and a PAD4 binding region comprising a heavy chain comprising SEQ ID NO: 61 and a light chain comprising SEQ ID NO: 65. The DuetMab may comprise a PAD2 binding domain comprises a heavy chain comprises SEQ ID NO: 70 and a light chain comprising SEQ ID NO: 64, and a PAD4 binding region comprises a heavy chain comprising SEQ ID NO: 67 and a light chain comprising SEQ ID NO: 63. The antibody may comprise a PAD2 binding domain comprising a heavy chain comprise SEQ ID NO: 71 and a light chain comprising SEQ ID NO: 64, and a PAD4 binding region comprising a heavy chain comprising SEQ ID NO: 68 and a light chain comprising SEQ ID NO: 63. The DuetMab may comprise a PAD2 binding domain comprises a heavy chain comprising SEQ ID NO: 72 and a light chain comprising SEQ ID NO: 64, and a PAD4 binding region comprises a heavy chain comprising SEQ ID NO: 69 and a light chain comprising SEQ ID NO: 63.

The DuetMab may have the sequence of any of clones 01, 02, 03, 04, 05 or 06. The DuetMab bispecific may have a sequence that is at least 90% identical to the sequence of 01, 02, 03, 04, 05, or 06. The Bis3 bispecific may have a sequence as provided in Table 58.

### 6.12 IgG

The antibody may comprise an IgG or F(ab′)₂ fragment. The antibody may comprise an IgG or F(ab′)₂ fragment that is bivalent for PAD2 or bivalent for PAD4. The antibody may be an IgG1 that is bivalent for PAD2 or PAD4. The PAD2 or PAD4 may be human, cynomolgus and/or mouse PAD2 or PAD4. The IgG or F(ab′)₂ fragment may comprise the PAD2 binding domain according to the invention. The IgG or F(ab′)₂ fragment may comprise the PAD4 binding domain according to the invention. The antibody may comprise two of the PAD2 binding domains of the invention such that the antibody is bivalent for PAD2. The antibody may comprise two of the PAD4 binding domains such that the antibody is bivalent for PAD4. The antibody may comprise two of the PAD4 binding domain of the invention, and not comprise a PAD2 binding domain according to the invention. The antibody may comprise two of the PAD2 binding domain of the invention, and not comprise a PAD4 binding domain according to the invention.

The IgG may a heavy chain with a terminal lysine. The IgG may comprise two heavy chains with a terminal lysine. The IgG may comprise a heavy chain with a terminal lysine and a heavy chain without a terminal lysine. The IgG may comprise two heavy chains without terminal lysines.

### 6.13 Fab

The antibody may comprise a Fab fragment, wherein the Fab fragment comprises the PAD2 binding domain or the PAD4 binding domain. The Fab fragment may comprise a PAD2 binding domain, wherein the Fab binds human PAD2 with an affinity (K_{D}) of ≤20 nM, ≤10 nM, ≤6 nM, or ≤1 nM. The antibody may comprise a Fab fragment, wherein the Fab fragment comprises the PAD4 binding domain, and wherein the Fab binds human PAD4 with an affinity (K_{D}) of ≤1 nM, ≤0.1 pM, ≤0.07 pM, or ≤0.05 pM. The K_{D} may be measured by surface plasmon resonance (SPR).

### 6.14 Variable Region

The variable region of the antibody may be a human variable region. The variable region may comprise rodent or murine complementarity determining regions (CDRs) and human framework regions (FRs). The variable region may be a primate (e.g., non-human primate) variable region. The variable region may comprise rodent or murine CDRs and primate (e.g., non-human primate) framework regions (FRs). The variable region may comprise the CDRs, VH, VL or framework regions of any of the antibodies described in Table 1 to Table 54 and Table 62.

### 6.14.1 PAD2 Lead

The antibody may comprise a PAD2 binding domain, wherein the PAD2 binding domain comprises a variable heavy (VH) domain sequence comprising CDRs HCDR1, HCDR2 and HCDR3, and a variable light (VL) domain sequence comprising CDRs LCDR1, LCDR2 and LCDR3, wherein the HCDR1 amino acid sequence is SEQ ID NO: 3, the HCDR2 amino acid sequence is SEQ ID NO: 4, the HCDR3 amino acid sequence is SEQ ID NO: 5, the LCDR1 amino acid sequence is SEQ ID NO: 10, the LCDR2 amino acid sequence is SEQ ID NO: 11, and/or the LCDR3 amino acid sequence is SEQ ID NO: 12. The antibody may comprise a the PAD2 binding domain comprises a VH domain comprising a sequence having at least 70, 80, 90, 91, 92, 93, 94, 95, 96, 97, 98 or 99% identity to SEQ ID NO: 1. The PAD2 binding domain may comprise a VH domain comprising SEQ ID NO: 1. The PAD2 binding domain may comprise a VL domain comprising a sequence having at least 70, 80, 90, 91, 92, 93, 94, 95, 96, 97, 98 or 99% identity to SEQ ID NO: 2. The PAD2 binding domain may comprise a VL domain sequence comprising SEQ ID NO: 2. The PAD2 binding domain may comprise a VH domain sequence comprising SEQ ID NO: 1, optionally with 1, 2, 3, 4 or 5 amino acid alterations outside the CDRs. The PAD2 binding domain may comprise a VL domain sequence comprising SEQ ID NO: 2, optionally with 1, 2, 3, 4 or 5 amino acid alterations outside the CDRs. The antibody may comprise the VH, VL, CDRs and framework region sequences of the antibody described in Table 1. The antibody may be an affinity optimised antibody of the antibodies described in Table 53 or Table 54.

### 6.14.2 PAD4 Lead

The antibody of bispecific may comprise a PAD4 binding domain comprising a variable heavy (VH) domain sequence comprising complementarity determining regions (CDRs) HCDR1, HCDR2 and HCDR3, and a variable light (VL) domain sequence comprising CDRs LCDR1, LCDR2 and LCDR3, and wherein: the HCDR1 amino acid sequence is SEQ ID NO: 17, the HCDR2 amino acid sequence is SEQ ID NO: 18, the HCDR3 amino acid sequence is SEQ ID NO: 19, the LCDR1 amino acid sequence is SEQ ID NO: 24, the LCDR2 amino acid sequence is SEQ ID NO: 25, and/or the LCDR3 amino acid sequence is SEQ ID NO: 26. The PAD4 binding domain may comprise a VH domain comprising a sequence having at least 70, 80, 90, 91, 92, 93, 94, 95, 96, 97, 98 or 99% identity to SEQ ID NO: 31. The PAD4 binding domain may comprise a VH domain sequence comprising SEQ ID NO: 31. The PAD4 binding domain may comprise a VL domain comprising a sequence having at least 70, 80, 90, 91, 92, 93, 94, 95, 96, 97, 98 or 99% identity to SEQ ID NO: 32. The PAD4 binding domain may comprise a VL sequence domain comprising SEQ ID NO: 32. The PAD4 binding domain may comprise a VH domain sequence comprising SEQ ID NO: 31, optionally with 1, 2, 3, 4 or 5 amino acid alterations outside the CDRs. The PAD4 binding domain may comprise a VL domain sequence comprising SEQ ID NO: 32, optionally with 1, 2, 3, 4 or 5 amino acid alterations outside the CDRs. The antibody may comprise the VH, VL, CDRs and framework region sequences of the antibody described in Table 2. The antibody may be an affinity optimised antibody of the antibodies described in Table 62.

### 6.14.3 PAD4 Back-Up Clones

The antibody or bispecific may comprise the sequences of any of the antibody sequences provided in Table 3 to Table 42. The antibody may comprise the CDRs, framework regions, VH or VL sequences of Clone 42, 141LO0035 hIgG1 ngl-2, 141LO0035 hIgG1 pgl-4, 141LO0055 hIgG1 ngl-2, 141LO0030 hIgG1 ngl-2, 141LO0039 hIgG1 ngl-2, 141LO0030 hIgG1 pgl-4, 141LO0002 hIgG1 pgl-4, 141LO0002 hIgG1 pgl-3, 141LO0002 hIgG1 ngl-2. 141LO0039 hIgG1 pgl-4, PAD40175 hIgG1 ngl-2, PAD40119 hIgG1 ngl-2, PAD40141 hIgG1 ngl-2. The antibody may have a sequence having 90% sequence identify to a VH sequence provided in any of Table 3 to Table 42. a sequence having 90% sequence identify to a VL sequence provided in any of Table 3 to Table 42. The antibody may have a VH, VL, HCDR1, HCDR2, HCDR3, LCDR1, LCDR2, LCDR3 with a sequence identical to the corresponding region of Clone 42 as provided in Table 63.

### 6.14.4 PAD2 Back-Up Clones

The antibody or bispecific may comprise the sequences of any of the antibody sequences provided in Table 43 to Table 54. The antibody may comprise the CDRs, framework regions, VH or VL sequences of Clone 22, 141LO0002 hIgG1 pgl-3, 141LO0002 hIgG1 pgl-4, 141LO0002 hIgG1 ngl-2, 141LO0030 hIgG1 pgl-4, 141LO0002 hIgG1 ngl-2, 141LO0035 hIgG1 ngl-2, 141LO0039 hIgG1 pgl-4, 141LO0039 hIgG1 ngl-2, 141LO0055 hIgG1 ngl-2, 141LO0055 hIgG1 ngl-2. The antibody may have a sequence having 90% sequence identify to a VH sequence provided in any of Table 43 to Table 54. a sequence having 90% sequence identify to a VL sequence provided in any of Table 43 to Table 54. The antibody may have a VH, VL, HCDR1, HCDR2, HCDR3, LCDR1, LCDR2, LCDR3 with a sequence identical to the corresponding region of Clone 42 as provided in Table 63.

### 6.15 Fc Modifications

The antibody or bispecific may comprise an Fc domain, optionally an IgG1 Fc domain. The Fc domain may have a null effector function. The Fc domain may comprise the mutations L234F, L235Q and/or K322Q, as numbered by the EU index as set forth in Kabat et al. [14]. The Fc mutations may comprise at least one half life extension conferring mutation. The Fc domain may comprise the mutations M252Y, S254T and T256E as numbered by the EU index as set forth in Kabat et al. [14]. The Fc domain may comprise a least one null effector function mutation and at least one half life extension conferring mutation. The Fc domain may comprise the mutations L234F, L235Q, K322Q, M252Y, S254T and T256E as numbered by the EU index as set forth in Kabat et al. [14]. The Fc domain may comprise the mutations L234F, L235E, P331S, M252Y, S254T and T256E as numbered by the EU index as set forth in Kabat et al. [14]. The Fc domain may comprise a CH2 domain comprising SEQ ID NO: 47, SEQ ID NO: 48 or SEQ ID NO: 49. The Fc domain may have any of the mutations described in Table 65.

The polypeptide according to the invention may comprise a Fc variant domain.

IgG Fc domains with extended half-lives are described in WO 2015/175874 (A2) [15] and WO 2002/060919 (A2) [16]. YTE increases binding to FcRn leading to extended serum half-life. YTE is a triple mutation at CH2: M252Y, S254T and T256E.

The modified Fc region may comprise amino acid substitutions at two or more of positions 432 to 437, numbered according to the EU numbering index of Kabat, relative to a human wild-type Fc region; wherein (i) positions 432 and 437 are each substituted with cysteine; (ii) position 433 is histidine or is substituted with arginine, proline, threonine, lysine, serine, alanine, methionine, or asparagine; (iii) position 434 is asparagine or is substituted with arginine, tryptophan, histidine, phenylalanine, tyrosine, serine, methionine or threonine; (iv) position 435 is histidine or is substituted with histidine; and (v) position 436 is tyrosine or phenylalanine or is substituted with leucine, arginine, isoleucine, lysine, methionine, valine, histidine, serine, or threonine; and wherein the modified human IgG1 has an increased half-life compared to the half-life of an IgG1 having the human wild-type Fc region.

The modified Fc region may comprise amino acid substitutions at two or more of positions 432 to 437, numbered according to the EU numbering index of Kabat, relative to a wild-type Fc region; wherein
a) at least one of positions 432 and 437 is substituted with cysteine; or
b) at least one of positions 432 and 437 is substituted with an amino acid selected from the group consisting of glutamine, glutamic acid, aspartic acid, lysine, arginine, and histidine;
wherein the polypeptide has an altered half-life compared to the half-life of an IgG having the wild-type Fc region. Optionally, (i) both of positions 432 and 437 are substituted with cysteine; or (ii) both of positions 432 and 437 are substituted with an amino acid independently selected from the group consisting of glutamine, glutamic acid, aspartic acid, lysine, arginine, and histidine.

The polypeptide may comprise an amino acid insertion after position 437, optionally wherein the amino acid insertion is glutamic acid.

The binding affinity of the polypeptide for FcRn at pH 6.0 may be higher than the binding affinity of the IgG having the wild-type Fc region for FcRn at pH 6. The binding affinity of the polypeptide for FcRn at pH 7.4 may be higher than the binding affinity of the IgG having the wild-type Fc region for FcRn at pH 7.4. The KD of the polypeptide for FcRn at pH 6.0 may be less than 500 nM, and the KD at pH 7.4 is at least 1000 nM.

The polypeptide may comprise a Fc variant domain that exhibits increased pH dependence of binding affinity for FcRn compared to the IgG having the wild-type Fc region. The polypeptide may comprise a Fc variant domain, wherein the modified IgG Fc domain exhibits decreased pH dependence of binding affinity for FcRn compared to the IgG having the wild-type Fc region.

The polypeptide may comprise a Fc variant domain, wherein the modified IgG Fc domain retains wild-type levels of at least one attribute selected from the group consisting of (i) binding to at least one Fc gamma receptor, (ii) binding to Clq, or (iii) effector function, optionally wherein the Fc gamma receptor is selected from the group consisting of an FcyRI receptor, an FcyRII receptor and an FcyRIII receptor. The polypeptide may have decreased effector function selected from antibody dependent cellular cytotoxicity (ADCC), complement dependent cytotoxicity (CDC), and/or antibody dependent cellular phagocytosis (ADCP). The polypeptide may comprise a Fc variant domain, wherein the Fc variant domain has amino acid substitutions of three or more of positions 432, 433, 434, 435, 436 or 437.

The polypeptide may comprise a Fc variant domain, wherein the Fc variant domain has amino acid substitutions of four or more of positions 432, 433, 434, 435, 436 or 437. Position 432 and 437 may each substituted with cysteine; position 433 may be histidine or substituted with arginine, proline, threonine, lysine, serine, alanine, methionine, or asparagine; position 434 may be asparagine or is substituted with arginine, tryptophan, histidine, phenylalanine, tyrosine, serine, methionine or threonine; position 435 may be histidine or substituted with histidine; and position 436 may be tyrosine or phenylalanine or substituted with leucine, arginine, isoleucine, lysine, methionine, valine, histidine, serine, or threonine. Position 433 may be histidine. Position 433 may be substituted with arginine, asparagine, proline, threonine, or lysine. Position 434 may be substituted with arginine, tryptophan, histidine, phenylalanine, or tyrosine. Position 434 may be substituted with arginine. Position 436 may be substituted with leucine, arginine, isoleucine, lysine, methionine, valine or histidine. Position 433 may be histidine or substituted with arginine, asparagine, proline, threonine, or lysine; position 434 may be substituted with arginine, tryptophan, histidine, phenylalanine, or tyrosine; and position 436 may be substituted with leucine, arginine, isoleucine, lysine, methionine, valine or histidine.

The polypeptide may comprise a Fc variant domain, wherein the Fc variant domain may comprise the amino acid sequence at positions 432 to 437 of CXRHXC (SEQ ID NO: 187), where position 433 is histidine or is substituted with arginine, asparagine, proline, or serine, and position 436 is substituted with arginine, leucine, isoleucine, methionine, or serine. The polypeptide may comprise the amino acid sequence at positions 432 to 437 of CRRHXC (SEQ ID NO: 188) wherein position 436 is substituted with leucine, arginine, isoleucine, lysine, methionine, valine, histidine, serine, or threonine. Position 436 may be substituted with leucine, isoleucine, serine or threonine.

The polypeptide may comprise a Fc variant domain, wherein the Fc variant domain may comprise the amino acid sequence at positions 432 to 437 of CXRHRC (SEQ ID NO: 189) wherein position 433 is arginine, proline, threonine, lysine, serine, alanine, methionine, or asparagine. The modified IgG may comprise the amino acid sequence at positions 432 to 437 of ZXXHXZ (SEQ ID NO: 92), wherein position 432 is substituted with glutamic acid, glutamine, histidine, or aspartic acid; position 433 is histidine or is substituted with arginine, alanine, lysine, threonine, leucine, proline, serine, or glutamine; position 434 is substituted with tyrosine, phenylalanine, histidine, serine or tryptophan; position 436 is tyrosine or substituted with arginine, histidine, asparagine, lysine, leucine, methionine, threonine, or valine; and position 437 is substituted with glutamine, histidine, glutamic acid, or aspartic acid.

The Fc variant domain may comprise of N3, YC37-YTE, YC56-YTE, YC59-YTE, Y3-YTE, Y31-YTE, Y12-YTE, Y83-YTE, Y37-YTE, and Y9-YTE, N3-YTE, N3E-YTE, SerN3-YTE, Y54-YTE, Y74-YTE, Y8-YTE. The Fc variant domain may have a histidine at amino acid position 435. The modified IgG Fc domain may comprise the amino acid sequence of E(R/A)(W/S/F)HRQ (SEQ ID NO: 190) at positions 432 to 437.

The polypeptide may comprise at least an FcRn-binding portion of an Fc region of an IgG molecule, wherein said FcRn-binding portion comprises amino acid substitutions at two or more of positions 432 to 437, numbered according to the EU numbering index of Kabat, relative to a wild-type FcRn-binding portion; wherein (i) at least one of positions 432 and 437 is substituted with cysteine; or (ii) at least one of positions 432 and 437 is substituted with an amino acid selected from the group consisting of glutamine, glutamic acid, aspartic acid, and histidine. The polypeptide of claim 38, wherein (i) both of positions 432 and 437 are substituted with cysteine; or (ii) both of positions 432 and 437 are substituted with an amino acid independently selected from the group consisting of glutamine, glutamic acid, aspartic acid, and histidine. Position 432 and 437 may each be substituted with cysteine; position 433 may be histidine or substituted with arginine, proline, threonine, lysine, serine, alanine, methionine, or asparagine; position 434 may be asparagine or substituted with arginine, tryptophan, histidine, phenylalanine, tyrosine, serine, methionine or threonine; position 435 may be histidine or substituted with histidine; and position 436 may be tyrosine or phenylalanine or substituted with leucine, arginine, isoleucine, lysine, methionine, valine, histidine, serine, or threonine.

The Fc variant domain may comprise the amino acid sequence at positions 432 to 437 of ZXXHXZ, wherein position 432 is substituted with glutamic acid, glutamine, histidine, or aspartic acid; position 433 is histidine or is substituted with arginine, alanine, lysine, threonine, leucine, proline, serine, or glutamine; position 434 is substituted with tyrosine, phenylalanine, histidine, serine or tryptophan; position 436 is tyrosine or substituted with arginine, histidine, asparagine, lysine, leucine, methionine, threonine, or valine; and position 437 is substituted with glutamine, histidine, glutamic acid, or aspartic acid. The Fc variant domain may comprise the amino acid sequence of E(R/A)(W/S/F)HRQ at positions 432 to 437. There may further be an amino acid insertion after position 437, wherein the amino acid insertion is glutamic acid. The FcRn binding portion of the Fc region may comprise from about amino acid residues 231-446 of an IgG molecule according to the EU numbering index of Kabat. The FcRn binding portion of the Fc region may comprises from about amino acid residues 216-446 of an IgG molecule according to the EU numbering index of Kabat.

Variant IgG Fc domains with reduced effector function and extended half-lives are described in WO2013/165690 (A1) [17]. The variant IgG Fc domain may comprise:
a) a Phenylalanine (F) amino acid at position 234;
b) an Alanine (A), Asparagine (N), Phenylalanine (F), Glutamine (Q), or Valine (V) amino acid at position 235; and,
c) an Alanine (A), Aspartic acid (D), Glutamic acid (E), Histidine (H), Asparagine (N), or Glutamine (Q) amino acid at position 322; or, an Alanine (A) or Glycine (G) amino acid at position 331,
   wherein the amino acid numbering is according to the EU index as in Kabat.

The Fc variant domain may comprise a Phenylalanine (F) amino acid at position 234; a Glutamine (Q) amino acid at position 235; and a Glutamine (Q) amino acid at position 322, wherein the amino acid numbering is according to the EU index as in Kabat.

The Fc variant domain may comprise a Phenylalanine (F) amino acid at position 234; a Glutamine (Q) amino acid at position 235; and a Glycine (G) amino acid at position 331, wherein the amino acid numbering is according to the EU index as in Kabat. The Fc variant domain may comprise a Phenylalanine (F) amino acid at position 234; an Alanine (A) amino acid at position 235; and a Glutamine (Q) amino acid at position 322, wherein the amino acid numbering is according to the EU index as in Kabat. The Fc variant domain may comprise
a) a Tyrosine (Y) amino acid at position 252, or a Serine (S) amino acid at position 252, or a Tryptophan (W) amino acid at position 252 or a Threonine (T) amino acid at position 252; and/or
b) a Threonine (T) amino acid at position 254; and/or
c) a Glutamic acid (E) amino acid at position 256, or a Serine (S) amino acid at position 256, or a Arginine (R) amino acid at position 256, or a Glutamine (Q) amino acid at position 256, or an Aspartate (D) amino acid at position 256,
wherein the amino acid numbering is according to the EU index as in Kabat.

The Fc variant domain may comprise
a) a Tyrosine (Y) amino acid at position 252; and/or
b) a Threonine (T) amino acid at position 254; and/or
c) a Glutamic acid (E) amino acid at position 256, wherein the amino acid numbering is according to the EU index as in Kabat.

The Fc variant domain may comprise:
a) a Tyrosine (Y) amino acid at position 252, or a Serine (S) amino acid at position 252, or a Tryptophan (W) amino acid at position 252 or a Threonine (T) amino acid at position 252; and
b) a Threonine (T) amino acid at position 254,
wherein the amino acid numbering is according to the EU index as in Kabat.

The Fc variant domain may comprise:
a) a Threonine (T) amino acid at position 254; and
b) a Glutamic acid (E) amino acid at position 256, or a Serine (S) amino acid at position 256, or a Arginine (R) amino acid at position 256, or a Glutamine (Q) amino acid at position 256, or an Aspartate (D) amino acid at position 256,
wherein the amino acid numbering is according to the EU index as in Kabat.

The Fc variant domain may comprise:
a) a Tyrosine (Y) amino acid at position 252, or a Serine (S) amino acid at position 252, or a Tryptophan (W) amino acid at position 252 or a Threonine (T) amino acid at position 252; and
b) a Glutamic acid (E) amino acid at position 256, or a Serine (S) amino acid at position 256, or a Arginine (R) amino acid at position 256, or a Glutamine (Q) amino acid at position 256, or an Aspartate (D) amino acid at position 256,
wherein the amino acid numbering is according to the EU index as in Kabat.

The Fc variant domain may comprise:
a) a Tyrosine (Y) amino acid at position 252, and a Threonine (T) amino acid at position 254; or,
b) a Threonine (T) amino acid at position 254 and a Glutamic acid (E) amino acid at position 256; or,
c) a Tyrosine (Y) amino acid at position 252 and a Glutamic acid (E) amino acid at position 256
wherein the amino acid numbering is according to the EU index as in Kabat.

The Fc variant domain may comprise a Tyrosine (Y) amino acid at position 252, a Threonine (T) amino acid at position 254, and, a Glutamic acid (E) amino acid at position 256, wherein the amino acid numbering is according to the EU index as in Kabat.

The Fc variant domain may comprise:
a) a Phenylalanine (F) amino acid at position 234;
b) a Glutamine (Q) amino acid at position 235;
c) a Glutamine (Q) amino acid at position 322;
d) a Tyrosine (Y) amino acid at position 252;
e) a Threonine (T) amino acid at position 254; and,
f) a Glutamic acid (E) amino acid at position 256,
wherein the amino acid numbering is according to the EU index as in Kabat.

The Fc variant domain may comprise:
a) a Phenylalanine (F) amino acid at position 234;
b) a Glutamine (Q) amino acid at position 235;
c) a Glycine (G) amino acid at position 331;
d) a Tyrosine (Y) amino acid at position 252;
e) a Threonine (T) amino acid at position 254; and,
f) a Glutamic acid (E) amino acid at position 256,
wherein the amino acid numbering is according to the EU index as in Kabat.

The polypeptide my comprise a modified Fc variant domain, wherein the polypeptide has an improved pharmacokinetic (PK) property when compared to the same polypeptide comprising a wild-type Fc domain, optionally wherein the PK property is half-life. The polypeptide may have improved FcRn binding when compared to the same polypeptide comprising a wild-type Fc domain.

The polypeptide may comprise an IgG Fc domain selected from the group consisting of human immunoglobulin G class 1 (IgG1) Fc domain, human immunoglobulin G class 2 (IgG2) Fc domain, human immunoglobulin G class 3 (IgGs) Fc domain, and human immunoglobulin G class 4 (IgG4) Fc domain.

The polypeptide may comprise a modified Fc variant domain, wherein the polypeptide has reduced Fc-mediated effector function when compared to the same polypeptide comprising a wild-type Fc domain. The effector function may be antibody-dependent cell-mediated cytotoxicity (ADCC), and/or complement-dependent cytotoxicity (CDC). The polypeptide may have a lower affinity for an Fc gamma receptor (FcyR) when compared to the same polypeptide comprising a wild-type Fc domain, optionally wherein the FcyR is a human FcyR. The FcyR may be FcyRI, FcyRII, FcyRIII., FcyRI I, FcyRIa, FcyRIIa, FcyRIIb, FcyRIII (158V), FcyRIII (158F).

The polypeptide may comprise a modified Fc variant domain, wherein the polypeptide binds with improved affinity to FcRn when compared to the same polypeptide comprising a wild-type Fc domain, optionally wherein the polypeptide has a higher affinity for FcRn at pH 6.0 than at pH 7.4.

The polypeptide may comprise a modified Fc variant domain, wherein the polypeptide binds with reduced affinity to Clq when compared to the same polypeptide comprising a wild-type Fc domain.

The polypeptide may display an increase in thermal stability when compared to the same polypeptide comprising a FES-YTE IgG Fc domain, optionally wherein thermal stability is measured by Differential Scanning calorimetry (DSC), optionally wherein the increase in thermal stability is at least 4° C.

The polypeptide may display an increase in thermal stability when compared to the same polypeptide comprising a FES-YTE IgG Fc domain, wherein thermal stability is measured by Differential Scanning Fluorimetry (DSF), optionally wherein the DSF fluorescent probe is Sypro Orange, optionally wherein the increase in thermal stability increases is at least 5ºC.

The polypeptide may display an increase in apparent solubility as measured using a polyethylene glycol (PEG) precipitation assay when compared to the same polypeptide comprising a FES-YTE IgG Fc domain.

The polypeptide may display an increase in stability as measured using an accelerated stability assay when compared to the same polypeptide comprising a FES-YTE IgG Fc domain. The accelerated stability assay comprises: (i) incubation of the polypeptide for an extended time period, and (ii) incubation at high temperature. The accelerated stability assay may be performed by incubation at a high concentration, optionally wherein the extended time period is at least one month, optionally wherein the high concentration is at least 25 mg/ml, optionally wherein the high temperature is at least 40° C. The accelerated stability assay may be performed using High Performance Size Exclusion Chromatography (HPSEC) or Dynamic Light Scattering (DLS).

The Fc may comprise an RF double mutation.

The Fc may comprise a knob-in-hole mutation.

### 6.16 Polypeptides

The invention also relates to a polypeptide comprising the antibody or bispecific of the invention. The invention also provides polypeptides comprising one or more binding domains of the antibodies defined anywhere herein. The polypeptide may comprise part or all of a PAD2 binding domain. The polypeptide may comprise part or all of a PAD4 binding domain. The polypeptide may comprise binding domains such as one or more CDRs as defined herein, or variable light or variable heavy domains as defined herein. The polypeptides may comprise binding domains that comprise all three CDRs (CDR1, CDR2 and CDR3) of a variable heavy domain sequence as defined herein. The polypeptides may comprise binding domains that comprise all three CDRs (CDR1, CDR2 and CDR3) of a variable light domain sequence as defined herein. The polypeptide may comprise a variable heavy domain of an antibody as defined herein. The polypeptide may comprise a variable light domain of an antibody as defined herein. The polypeptide may comprise a full heavy chain of an antibody as defined herein. The polypeptide may comprise a full light chain of an antibody as defined herein. The polypeptide may be an isolated polypeptides.

### 6.17 Nucleic Acid

The invention also relates to a nucleic acid encoding one or more chains of the antibody or bispecific of the invention. The invention also relates to a nucleic acid encoding a polypeptide according to the invention. The invention also relates to a vector comprising the nucleic acid, and a host cell comprising the vector.

### 6.18 Pharmaceutical Composition

The invention also relates to a pharmaceutical composition comprising the antibody or bispecific of the invention and a pharmaceutically acceptable carrier.

### 6.19 Kit

The invention also relates to a kit comprising an antibody or bispecific or pharmaceutical composition of the invention. The kit may comprise instructions for use.

### 6.20 Methods of Treatment

The invention also relates to a method of treating a disease in a subject comprising administering the antibody or pharmaceutical composition according to the invention. The subject may have an autoimmune disease. The subject may have rheumatoid arthritis (RA). The subject may have elevated levels of PAD in the synovial fluid, whole blood or serum compared to a healthy subject. The subject may have elevated levels of PAD2 in the synovial fluid, whole blood or serum compared to a healthy subject. The subject may have elevated levels of PAD4 in the synovial fluid, whole blood or serum compared to a healthy subject. The concentration of PAD4 in the subject's synovial fluid may be at least 200 ng/ml. The concentration of PAD2 in the subject's synovial fluid may be at least 20 ng/ml. The concentration of PAD2 and/or PAD4 in the subject's synovial fluid may be in the range of the values provided in Table 82. The concentration of the PAD2 and/or PAD4 in the subject's whole blood may be at least 1 ng/ml. The concentration of PAD2 and/or PAD4 in the subject's whole blood may be in the range of the values provided in Table 83. The concentration of PAD2 or PAD4 may be determined by ELISA.

The invention also relates to a method of treating a disease in a subject comprising administering an anti-PAD4 antibody in combination with an anti-PAD2 antibody to the subject. The anti-PAD4 antibody and the anti-PAD2 antibodies may be bivalent IgGs of Fab(₂) fragments comprising at least two binding domains for either PAD4 or PAD2 respectively. The anti-PAD2 antibody and anti-PAD4 antibody may be administered to the subject simultaneously, separately or sequentially.

### 6.21 EPC 2000

The invention also relates to an antibody or a pharmaceutical composition of the invention for use in a method of treating of preventing a disease in a subject. The disease may be an autoimmune disorder. The disease may be a disease characterised by increased PAD activity in the tissue relative to a healthy subject. The disease may be a disease characterised by increased PAD2 and/or PAD4 activity in the tissue relative to a healthy subject. The tissue may be synovial fluid, whole blood or serum.

### 6.22 Swiss-Style

The invention also relates to an antibody of the invention or a pharmaceutical composition of the invention for the manufacture of a medicament for the treatment of an autoimmune disorder. The treatment may comprise a method of treatment according to the invention.

### 7 TERMINOLOGY

### 7.1 Antibody

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule.

### 7.1.1 Antibody Fragment

The term “antibody fragment” refers to a portion of an intact antibody. An “antigen binding fragment,” “antigen-binding domain,” or “antigen-binding region,” refers to a portion of an intact antibody that binds to an antigen. An antigen-binding fragment can contain the antigenic determining regions of an intact antibody (e.g., the complementarity determining regions (CDR)). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

### 7.1.2 Anti-PAD2 Antibody

The terms “anti-PAD2 antibody”, “PAD2 antibody” and “antibody that binds to PAD2” are used interchangeably herein to refer to an antibody that is capable of binding to PAD2. The extent of binding of a PAD2 antibody to a non-PAD2 PAD can be less than about 10% of the binding of the antibody to PAD2 as measured, e.g., using ForteBio or Biacore. In some aspects provided herein, a PAD2 antibody is also capable of binding to PAD3. In some aspects provided herein, a PAD2 antibody does not bind to PAD3. In some aspects provided herein, a PAD2 antibody is also capable of binding to PAD1. In some aspects provided herein, a PAD2 antibody does not bind to PAD1.

### 7.1.3 Anti-PAD4 Antibody

Similarly, the terms “anti-PAD4 antibody”, “PAD4 antibody” and “antibody that binds to PAD4” are used interchangeably herein to refer to an antibody that is capable of binding to PAD4. The extent of binding of a PAD4 antibody to a non-PAD4 PAD can be less than about 10% of the binding of the antibody to PAD4 as measured, e.g., using ForteBio or Biacore. In some aspects provided herein, a PAD4 antibody is also capable of binding to PAD3. In some aspects provided herein, a PAD4 antibody does not bind to PAD3. In some aspects provided herein, a PAD4 antibody is also capable of binding to PAD1. In some aspects provided herein, a PAD4 antibody does not bind to PAD1.

### 7.1.4 Humanized Antibody

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) [18-20]. In some instances, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the replaced non-human residues to refine and optimize antibody or antigen-binding fragment thereof specificity, affinity, and/or capability. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin.

### 7.1.5 Human Antibodies

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

### 7.2 Binding Affinity

“Binding affinity” or “affinity” generally refers to the strength of the sum total of non-covalent interactions between a single binding site of a molecule (e.g., an antibody or antigen-binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen-binding fragment thereof and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g, an antibody or antigen-binding fragment thereof to an antigen, and k_{off} refers to the dissociation of, e.g, an antibody or antigen-binding fragment thereof from an antigen. The kₒₙ and k_{off} can be determined by techniques known to one of ordinary skill in the art, such as Biacore® or KinExA.

### 7.3 Bispecific

The term “bispecific antibody” means an antibody which comprises specificity for two target molecules, and includes, but is not limited to, formats such as DVD-Ig, mAb2 [21], FIT-Ig ([22]), mAb-dAb, dock and lock, Fab-arm exchange, SEEDbody, Triomab, LUZ-Y, Fcab, κλ-body, orthogonal Fab, scDiabody-Fc, diabody-Fc, tandem scFv-Fc, Fab-scFv-Fc, Fab-scFv, intrabody, BiTE, diabody, DART, TandAb, scDiabody, scDiabody-CH3, Diabody-CH3, Triple body, Miniantibody, minibody, TriBi minibody, scFv-CH3 KIH, scFv-CH-CL-scFv, F(ab′)2-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, Bis3, DuetMab, DT-IgG, DutaMab, IgG(H)-scFv), scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig and zybody. A bispecific molecule may comprise an antibody which is fused to another non-Ig format, for example a T-cell receptor binding domain; an immunoglobulin superfamily domain; an agnathan variable lymphocyte receptor; a fibronectin domain (e.g. an Adnectin™); an antibody constant domain (e.g. a CH3 domain, e.g., a CH2 and/or CH3 of an Fcab™) wherein the constant domain is not a functional CH1 domain; an scFv; an (scFv)2; an sc-diabody; an scFab; a centyrin and an epitope binding domain derived from a scaffold selected from CTLA-4 (Evibody™); a lipocalin domain; Protein A such as Z-domain of Protein A (e.g. an Affibody™ or SpA); an A-domain (e.g. an Avimer™ or Maxibody™); a heat shock protein (such as and epitope binding domain derived from GroEI and GroES); a transferrin domain (e.g. a trans-body); ankyrin repeat protein (e.g. a DARPin™); peptide aptamer; C-type lectin domain (e.g. Tetranectin™); human γ-crystallin or human ubiquitin (an affilin); a PDZ domain; scorpion toxin; and a kunitz type domain of a human protease inhibitor.

### 7.3.1 Bis3

Bis3 format bispecific antibodies comprise an IgG molecule having 2 Fab domains and 2 scFvs, wherein each scFv is appended to the C-terminal of each heavy chain (i.e. IgG-HC-scFv, [23]). The two Fab domains bind the same target protein as each other, and thus the molecules are symmetrical with respect to the Fab domains. The two scFvs bind a different target protein to the Fab domains, and each scFv binds the same target protein as each other. Therefore in one embodiment, the Fab domains may bind the first target protein (e.g. PAD2) and the scFv domains may bind the second target protein (e.g. PAD4). Alternatively, the target bound by the Fab domains and the target bound by the scFvs may be in the opposite orientation and therefore the scFv domains may bind the first target protein (e.g. PAD2) and the Fab domains may bind the second target protein (e.g. PAD4).

### 7.3.2 DuetMab

DuetMab antibodies comprise an IgG antibody having two heavy chains and two light chains. The two arms are asymmetrical, with each arm binding a different target protein. Thus the antibodies have a single binding domain for each of the two target proteins such that the antibody as a whole is bivalent, but monovalent for each target protein. DuetMab antibodies uses knobs-into-holes technology for heterodimerization of two distinct heavy chains and increases the efficiency of cognate heavy and light chain pairing by replacing the native disulphide bond in one of the CH1-CL interfaces with an engineered disulphide bond. Such antibodies maintain the structure and developability properties of natural IgGs ([23,24]).

### 7.4 C-Terminal Variants

Large-scale production of proteins involves the use of cell cultures that are known to produce proteins exhibiting varying levels of heterogeneity. One potential source of heterogeneity involves C-terminal lysine residues, such as those typically found on the heavy chains of antibody molecules. C-terminal lysines can be lost, so that individual antibodies in a production batch can vary at their C terminus as to whether a lysine residue is present (“lysine clipping”) [25]. C-terminal lysine can be potentially present on both the heavy chains of an antibody (K2), on either one of the heavy chains (K1), or neither of them (K0).

### 7.5 Complementary Determining Region

The term “complementarity determining region” or “CDR” as used herein refers to each of the regions of an antibody variable domain which are hypervariable in sequence and/or form structurally defined loops (hypervariable loops) and/or contain the antigen-contacting residues. Antibodies can comprise six CDRs, e.g., three in the VH and three in the VL.

Kabat numbering is a system of numbering amino acid residues in the heavy and light chain variable regions of an antibody or an antigen-binding fragment thereof. In some aspects, CDRs can be determined according to the Kabat numbering system [26]. Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

The EU index or EU numbering system is based on the sequential numbering of the first human IgG sequenced (the EU antibody). The numbering scheme used for substitutions and insertions in Fc regions in this specification is the EU index as in Kabat [14]. In contrast, the numbering scheme used for the variable regions (VH and VL) in this specification is the regular Kabat numbering.

Chothia refers instead to the location of the structural loops [27]. The end of the Chothia CDR-H1 loop when numbered using the Kabat numbering convention varies between H32 and H34 depending on the length of the loop (this is because the Kabat numbering scheme places the insertions at H35A and H35B; if neither 35 A nor 35B is present, the loop ends at 32; if only 35 A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

TABLE 64
Kabat and Chothia

| Loop | Kabat | AbM | Chotlia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

### 7.6 Epitope

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some aspects, the epitope to which an antibody or antigen-binding fragment thereof binds can be determined by, e.g, NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g, site-directed mutagenesis mapping).

An antibody that “binds to the same epitope” as a reference antibody refers to an antibody that binds to the same amino acid residues as the reference antibody. The ability of an antibody to bind to the same epitope as a reference antibody can be determined by a hydrogen/deuterium exchange assay [28].

### 7.7 Fc Modifications

Multiple mutation combinations in the IgG Fc have been characterized to tailor immune effector function or IgG serum persistence to fit desired biological outcomes for monoclonal antibody therapeutics. Example IgG Fc modifications are summarised in Table 65.

TABLE 65
IgG Fc modifications

| Name | Description* | Functional effect |
|---|---|---|
| Triple mutation | L234F/L235E/P331S | Effector function attenuation: |
| (TM) [29] | | Decreased binding to Fcγ |
| | | and C1q, decreased antibody |
| | | dependent antibody |
| | | mediated cytotoxicity (ADCC) |
| YTE | M252Y/S254T/T256E | Increased half-life |
| FQQ | L234F/L235Q/K322Q | Effector function attenuation - |
| FQG | L234F L235Q P331G/ | Increased thermostability |
| FAQ | L234F L235Q P331G/ | compared to TM |
| FQQ-YTE | L234F/L235Q/K322Q/ | Increased |
| | M252Y/S254T/T256E | thermostability in |
| FQG-YTE | L234F L235Q P331G/ | the context of |
| | M252Y/S254T/T256E | YTE compared to TM - |
| FAQ-YTE | L234F L235Q P331G/ | Increased half-life |
| | M252Y/S254T/T256E | |

*Amino acid numbering according to the EU index as in Kabat [14]

The TM modification abolishes Fc effector function and is a triple mutation at CH2 position: L234F; L235E and P331S [20]. The FQG, FQQ and FAQ modifications are described in detail in WO2013/65690 A1, Tsui et al. [17]) and Borrok et al. [30]. The FQQ modification is a more thermostable alternative to the TM effector function attenuation modification. YTE and N3Y are modifications that increase half-life. The N3Y modification is described in detail in WO 2015/175874 (A2). The YTE modification is described in WO 2002/060919 (A2) [16]. The Fc mutations do not affect variable region binding affinity.

### 7.8 Isolated

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells or compositions include those which have been purified to a degree that they are no longer in a form in which they are found in nature. An antibody, polynucleotide, vector, cell, or composition which is isolated may be substantially pure. As used herein, “substantially pure” refers to material which is at least 50% pure (i.e., free from contaminants), at least 90% pure, at least 95% pure, at least 98% pure, or at least 99% pure.

### 7.9 Knob-into-Hole Mutation

The “Knob-in-Hole” or also called “Knob-into-Hole” technology refers to mutations Y349C, T366S, L368A and Y407V (Hole) and S354C and T366W (Knob) both in the CH3-CH3 interface to promote heteromultimer formation has been described in U.S. Pat. Nos. 5,731,168 and 8,216,805 [31,32].

The Knob mutation refers to the substitutions S139C and T151W in the Fc. The hole mutation refers to the substitutions Y134C, T151 S, L153A, Y192V in the Fc.

### 7.10 Percent Identity

“Percent identity” refers to the extent of identity between two sequences (e.g. amino acid sequences or nucleic acid sequences). Percent identity can be determined by aligning two sequences, introducing gaps to maximize identity between the sequences. Alignments can be generated using programs known in the art. For purposes herein, alignment of nucleotide sequences can be performed with the blastn program set at default parameters, and alignment of amino acid sequences can be performed with the blastp program set at default parameters (see National Center for Biotechnology Information (NCBI): ncbi.nlm.nih.gov).

### 7.11 RF Mutation

The “RF mutation” generally refers to the mutation of the amino acids HY into RF in the CH3 domain of Fc domains, such as the mutation H435R and Y436F in CH3 domain. The RF mutation abolishes binding to protein A.

### 7.12 Potency

“Potency” is normally expressed as an IC50 value, in nM unless otherwise stated. IC50 is the median inhibitory concentration of an antigen-binding molecule. In functional assays, IC50 is the concentration that reduces a biological response by 50% of its maximum. In ligand-binding studies, IC50 is the concentration that reduces receptor binding by 50% of maximal specific binding level. IC50 can be calculated by any number of means known in the art. Improvement in potency can be determined by measuring, e.g., against a parent antibody (for example, the parent antibody prior to germlining or the parent antibody prior to affinity optimization).

### 7.13 Variable Region

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen.

### 8 EXAMPLE 1: METHODS

### 8.1 Tₒₙₛₑₜ Stability Assay

Differential scanning fluorimetry (DSF) is a fluorescence-based protein stability assay that measures protein folding state through monitoring changes in fluorescence as a function of temperature. This technique provides biophysical properties such as midpoint temperature (Tₘ) and onset temperature (Tₒₙₛₑₜ) of thermal unfolding. Nano-DSF is a dye-free DSF method that monitors the change of intrinsic fluorescence from inherent tryptophan in protein as a function of temperature, time, or denaturant concentration [33]. Protein unfolding changes the microenvironment polarity around tryptophan residues, causing a red shift of fluorescence [18]; using this principle, Nano-DSF determines Tₘ and Tₒₙₛₑₜ by measuring the ratio of the fluorescence intensity at 330 nm and 350 nm as a function of temperature.

### 8.2 Specificity Assay

Antibody samples were tested for binding to PAD1, PAD2, PAD3 and PAD4 using an ELISA method. Black high binding plates (Greiner, 781077) were prepared by adding 20 μL of a solution of streptavidin (Invitrogen, S888) at 20 μg/mL in HBSS buffer (Sigma, H8264) and incubated for 16 hours at 4ºC. Plates were equilibrated to room temperature and washed with 100 μL PBS (Oxoid, BR0014G) containing 0.1% Tween20 (Sigma, P2287) three times and 80 μL 1% BSA (Sigma, A7979) in HBSS added. After 1-hour plates were washed as described previously and 20 μL of biotinylated PAD1, PAD2, PAD3 or PAD4 was added at a concentration of 1 μg/mL prepared in a buffer containing 50 mM HEPES pH 7.3 (VWR, J848), 10 mM CaCl2 (Sigma, 21115), 120 mM NaCl (Sigma, S5150), 5 mM DTT (Sigma, 43816) and 0.1 mM CHAPS (Sigma, 19899). After 1-hour plates were washed as described previously and 20 μL antibody prepared in 50 mM HEPES PH 7.3, 10 mM CaCl2, 120 mM NaCl, 5 mM DTT and 0.1 mM CHAPS was added. After 1.5 hours plates were washed as described previously and 20 μL anti-human IgG Fc HRP (Southern Biotech, 9040-05) was added at a concentration of 50 ng/ml prepared in HBSS containing 0.5% BSA. After 1-hour plates were washed as described previously and 20 μL of QuantaBlu working solution at room temperature was added. After 30 minutes 20 μL QuantaBlu stop solution was added (QuantaBlu kit ThermoFisher, 15169). Fluorescence was measured using a 330 nm excitation filter and a 420 nm emission filter on a PHERAStar FSX plate reader (BMG Labtech).

### 8.3 Interferometric Scattering Microscopy (ISCAT)

Interferometric scattering microscopy (ISCAT) or Mass Photometry measure the light scatter of individual protein molecules in close proximity of a glass surface. Contrast is directly proportional to the molecular weight of individual protein molecules/complexes. Measurement chips were prepared by cleaning polished microscopy cover slides in deionized water and iso-propanol followed by drying in compressed air before mounting silicon cassette wells. Measurement commenced by addition of buffer (10 μL) to selected well followed by autofocus adjustment.

Protein sample was added (10 μL at approximately 50-100 nM), contrast from individual protein molecules was recorded for 60 sec within field of view (Refeyn Acquire). Resulting raw data movie was processed by image analysis (Refeyn Discover) to build mass histogram of sample.

### 8.4 Trypsin Cleavage Potency Assay

PAD activity was determined using a short peptide (Cambridge Research Biochemicals) containing arginine flanked by AlexaFluor 488, which acts as a FRET donor, and QSY7, which acts as a FRET acceptor. If arginine is deiminated to citrulline by the activity of PAD, trypsin will not cleave the peptide and fluorescence from the donor is quenched by the acceptor. Inhibition of PAD by anti-PAD scFv prevents arginine deimination and renders the peptide susceptible to trypsin cleavage. The resulting separation of donor and acceptor fluorophores allows detectable emission from the donor. 2.5 μl of sample scFv were prepared in assay buffer containing 50 mM HEPES, 5 mM DTT, 10 mM CaCl₂), and 0.01% CHAPS and preincubated with 2.5 ul PAD4 at 10 nM (final concentration). Peptide substrate was prepared as a stock solution and 5 μl was added to give a final concentration of 100 nM. Following a suitable incubation time, 10 μl trypsin at 100 nM (final concentration) was added and the reaction was allowed to proceed for at least two minutes prior to reading on an EnVision plate reader (PerkinElmer, Waltham, MA).

### 8.5 Histone-H3 PAD Potency Assay

PAD2 and PAD4 activity was measured using a Histone-H3 citrullination assay using different substrates, as described below:

### 8.5.1 Recombinant PAD

Human PAD4 (RD223) used at 0.15 ng/ml (1.8 pM); Human PAD2 (RD220) used at 0.02 ng/ml (0.24 pM); 30 minutes pre-incubation of PAD and Bispecific; 3 hour incubation.

### 8.5.2 Synovial Fluid

96-well high bind half area plates were coated overnight at 4ºC with 1 μg/ml of HIS-H3. RA Synovial fluid (DX01156) diluted in citrullination buffer was preincubated with EDTA or a serial dilution of antibodies for 30 mins, then transferred to histone H3 coated plate and incubated for 1.5 hrs at 37ºC. A rabbit anti-human citrullinated histone H3 Ab was incubated for 1 hr to detect citrullinated histone H3, followed by 1 hr a goat anti-rabbit-HRP conjugated Ab. UltraSensitive TMB substrate was used to develop the color reaction which was measured at 450 nm.

### 8.5.3 Whole Blood

Fresh whole blood was dosed overnight with the bi-specific Abs. Plasma was collected and PAD activity assessed using histone H3 PAD activity assay at multiple plasma dilutions. Whole blood from normal healthy donors was incubated overnight with Abs at 37 C. Plasma was harvested and stored frozen at −80 C. 1/5 plasma dilutions were used in histone-H3 PAD activity assays. PAD surface expression was assessed by FACS. Soluble PAD was assessed by ELISA.

### 8.6 Histone H3cit Western Blot Analysis

PAD enzymes citrullinate histone H3 into H3cit which can be measured using Western blot protein detection system. LPS exposure results in an elevation of H3cit levels in vivo due to PAD enzyme activity in the lungs of mice. H3cit expression was analyzed using Western Blot in bronchoalveolar lavage (BAL) fluid from LPS and saline exposed WT and PAD4KO mice. Mice that were dosed with anti-PAD2/anti-PAD4 had lower levels of H3Cit in BAL fluid as compared to WT mice. BAL fluid from PAD4KO mice had no H3Cit.

8.7 huFcRn Affinity Chromatography

A huFcRN coupled sepharose column was used to characterize the affinity of the samples to huFcRN. Around 40 μg/40 μL of sample was loaded onto a 1 mL column, followed by a 3 column volume (CV) linear gradient from buffer A (20 mM MES, 150 mM NaCl, pH5.5) to 40% buffer B (20 mM Tris+150 mM NaCl, pH8.8) and a 18CV linear gradient from 40% to 100% buffer B. The experiment was performed at a flow rate of 0.5 mL/min, at room temperature, using the Agilent-DAD to measure the A280 of the elution profile and the retention time.

### 8.8 Cyno Safety Study

The target engagement assay centred around the ability of both PAD2 and PAD4 to citrullinate histone H3. The histone H3 substrate is coated on the plate where active PADs in the sample deaminate the argine residues to form citrulline. These citrullinated epitopes are then detected through standard immunoassay methods.

### 8.9 Cytokine Safety Assay

The potential for the induction of cytokine release by the Bis3 and DuetMab formats (Clone 06 and Clone 12) was evaluated in 8 donors (4 healthy and 4 RA patients) using the following methodologies: a soluble stimuli whole blood assay and wet-coated immobilized stimuli isolated PBMC assay. The concentrations of cytokines IFN-γ, IL-2, 1L-6, TNF-α in collected plasma and cell culture supernatants were measured using Luminex. For each whole blood and isolated PBMC sample, negative control wells were also set-up using the same lot and volume of PBS as used to prepare the test items and controls.

### 8.10 Padi4 Knock Out Mouse Line Generation

A DNA Targeting vector was designed and cloned to modify the mouse endogenous Padi4, Peptidyl arginine deaminase, type IV, gene. The strategy was based on cloning LoxP sites into the introns flanking exons 7 and 10 of the Padi4 gene. Upon Cre induced recombination this would generate a Knock out, KO, leaving a single LoxP site 276 bp upstream of exon 7 and 736 bp downstream of exon 10. The targeting vector was used to modify the Padi4 locus, in the Primogenix, PrX, mouse Embryonic stem cells (C57Bl6/N origin), via homologous recombination. Correctly targeted cells were injected into 3.5 days old Balb C blastycyst for chimera generation. Male chimeric mice were bred to C57Bl6/N females for line expansion. The established mouse line was bred to a R26 Cre deletor line to generate the final Padi4 KO allele.

### 8.11 Biacore Affinity Analysis

### 8.11.1.1 Fabs and Monovalent Bispecifics (DuetMabs)

The affinities of the anti-PAD2 antigen binding fragments (Fabs), anti-PAD4 Fabs or DuetMabs to PAD species were measured using Biacore 8K (Cytiva) at 25° C. The experiments were carried out using recombinant human PAD2, cynomolgus PAD2, mouse PAD2, human PAD4, cynomolgus PAD4 and mouse PAD4. All species were enzymatically biotinylated on an Avi-tag. anti-PAD Fabs were expressed [34] or obtained by papain digestion of the anti-PAD IgGs. After 20 min papain incubation, the sample was injected at 0.5 ml/min onto a Superdex 200 Increase 10/300 GL column equilibrated in D-PBS and the Fab isolated. Streptavidin was covalently immobilised to a CM5 or C1 chip surface using standard amine coupling techniques. Recombinant biotinylated PAD2 and PAD4 species were titrated onto the streptavidin chip surface in 10 mM HEPES pH 7.4, 150 mM NaCl, 0.05% Surfactant P20, 1 mM CaCl₂, 1 mM DTT buffer to enable Fab or DuetMab binding. The Fabs or DuetMabs were serially diluted in 10 mM HEPES pH 7.4, 150 mM NaCl, 0.05% Surfactant P20, 1 mM CaCl₂, 1 mM DTT and flowed over the chip at 50 μl/min, with 3 minutes association and 10 minutes dissociation. Multiple buffer-only injections were made under the same conditions to allow for double reference subtraction of the final sensorgram sets. Alternatively, the Fabs were serially diluted in 10 mM HEPES pH 7.4, 150 mM NaCl, 0.05% Surfactant P20, 1 mM CaCl₂, 1 mM DTT and injected in sequentially increasing concentrations (single cycle kinetics) over the chip at 50 μl/min, with 2 minutes association and 10 minutes dissociation at the end of the complete binding cycle. A buffer-only injection was made under the same conditions to allow for double reference subtraction of the final sensorgram sets. All sensorgram sets were analysed using Biacore 8K Evaluation Software. The chip surface was fully regenerated with pulses of 3.0 M MgCl₂.

### 8.11.1.2 Bispecifics

The affinities of recombinant PAD species to anti-PAD IgGs, bivalent Bis3 anti-PAD molecules or monovalent bispecifics (DuetMabs) were measured using Biacore 8K (Cytiva) at 25ºC. The experiments were carried out using recombinant human PAD2, cynomolgus PAD2, mouse PAD2, human PAD4, cynomolgus PAD4 and mouse PAD4. Protein G′ was covalently immobilised to a C1 chip surface using standard amine coupling techniques at a concentration of 20 μg/ml in 10 mM Sodium acetate pH 3.65. The IgGs, Bis3 molecules or DuetMabs were captured onto the Protein G′ surface in 10 mM HEPES pH 7.4, 150 mM NaCl, 0.05% Surfactant P20, 1 mM CaCl₂, 1 mM DTT buffer at 5 μl/min to enable PAD binding. Alternatively, the anti-PAD molecules were chemically biotinylated using EZ link Sulfo-NHS-LC-Biotin (Thermo) and captured on a C1-Streptavidin surface (prepared as before). The PAD species were serially diluted in 10 mM HEPES pH 7.4, 150 mM NaCl, 0.05% Surfactant P20, 1 mM CaCl₂, 1 mM DTT and injected in sequentially increasing concentrations (single cycle kinetics) over the chip at 50 μl/min, with 2 minutes association and 10 minutes dissociation at the end of the complete binding cycle. A buffer-only injection was made under the same conditions to allow for double reference subtraction of the final sensorgram sets, which were analysed using Biacore 8K Evaluation Software. The Protein G′ chip surface was fully regenerated with pulses of 6 M Guanidine HCl in D-PBS to remove captured molecules together with any bound PAD. Whereas the streptavidin surface was fully regenerated with pulses of 3.0 M MgCl₂.

### 9 EXAMPLE 2: TARGETING PAD2 AND PAD4 IN THE TREATMENT OF RA

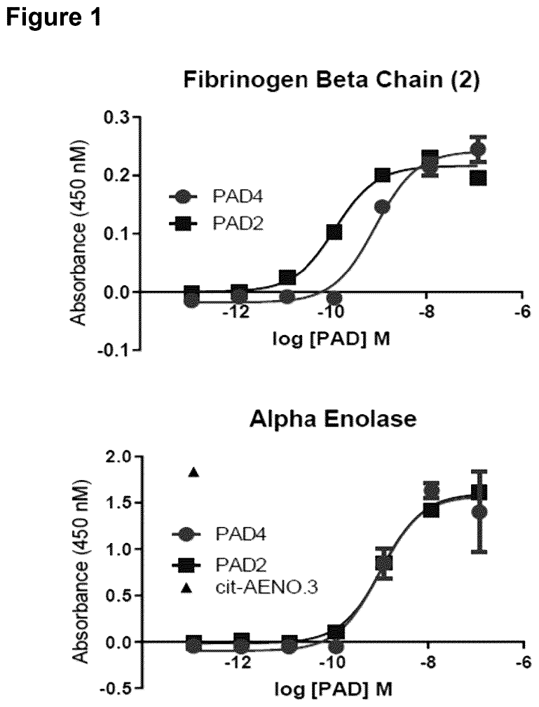

PAD2 and PAD4 drive citrullination in patients with RA. The inventors confirmed that both PAD2 and PAD4 can generate the citrullinated RA antigens fibroinogen β-chain [35] and α-enolase [36] (FIG. 1).

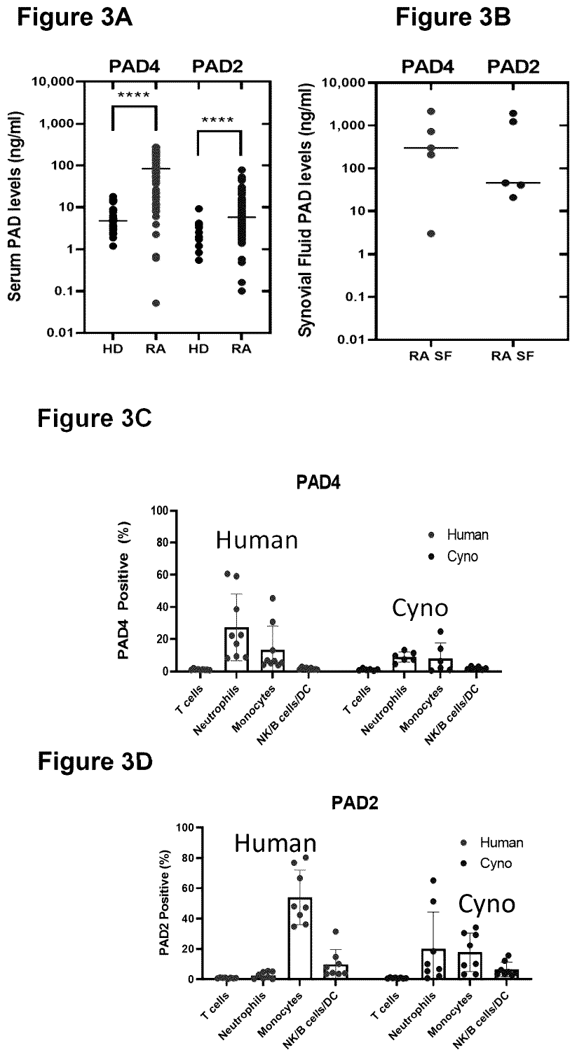

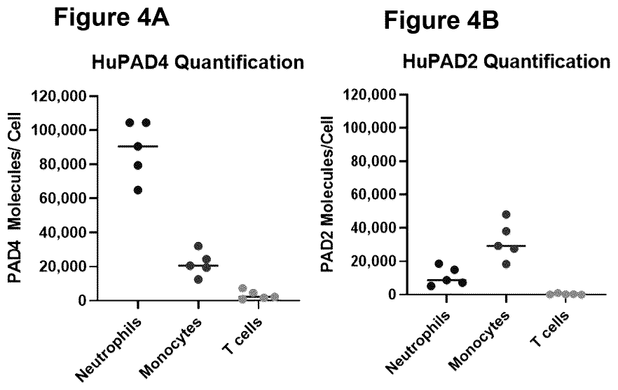

PAD4 and PAD2 levels were also found to be increased in RA serum (FIG. 3A) and synovial fluid (FIG. 3B). RNA expression profiling revealed that PAD4 is highly expressed in neutrophils and monocytes (human and cyno) (FIG. 3C and FIG. 3D). PAD2 and PAD4 were also found to be highly expressed on the cell surface of immune cells (FIG. 4A and FIG. 4B).

### 10 EXAMPLE 3: GENERATION OF ANTI-PAD2 AND ANTI-PAD2 ANTIBODIES

10.1 Generation of PAD2 antibodies

Several human antibody libraries derived from adult naïve donors were used for antibody selections. Parental antibody clones were isolated from a large single chain Fv (scFv) human antibody library derived from spleen cells from adult naïve donors and cloned into a phagemid vector based on filamentous phage M13.

PAD2 specific scFv antibodies were isolated from the phage display library in a series of repeated selection cycles on recombinant bacterial expressed human PAD2 with hybrid strands including selection cycles on recombinant bacterial expressed cynomolgus or mouse PAD2 [37]. scFv's were expressed in the bacterial periplasm and screened for their inhibitory activity in a trypsin cleavage assay. Screening hits, i.e. scFv clones which showed an inhibitory effect on PAD2 citrullination activity were subjected to DNA sequencing. Unique scFv's were expressed again in bacteria and purified by affinity. Additionally, a subset of selection outputs were subcloned for high throughput expression and screening in IgG format. Functional clones from either approach were reformatted into antibody heavy and light chain vectors for mammalian expression as IgG or Fab molecules. The anti-PAD2 antibodies were then affinity optimised.

The anti-PAD2 antibodies (e.g. PAD40141, PAD40119 and PAD40175, Table 52-Table 54) had an affinity (K_{D}) for human PAD2 of approximately 6-260 nM (Table 69).

The anti-PAD2 antibodies were then affinity optimised. The sequences of the resultant anti-PAD2 antibodies are shown in Table 1 and Table 43 to Table 54. The affinities of the anti-PAD2 antibodies are shown in Table 68 and Table 69.

### 10.2 Generation of PAD4 Antibodies

Several human antibody libraries derived from adult naïve donors were used for antibody selections. Parental antibody clones were isolated from a large single chain Fv (scFv) human antibody library derived from spleen cells from adult naïve donors and cloned into a phagemid vector based on filamentous phage M13.

PAD4-specific scFv antibodies were isolated from the phage display library in a series of repeated selection cycles on recombinant mammalian expressed human PAD4 with hybrid strands including selection cycles on recombinant mammalian expressed cynomolgus or mouse PAD4 [37]. scFv's were expressed in the bacterial periplasm and screened for their inhibitory activity in a trypsin cleavage assay. Screening hits, i.e. scFv clones which showed an inhibitory effect on PAD2 citrullination activity were subjected to DNA sequencing. Unique scFv's were expressed again in bacteria and purified by affinity. Additionally, a subset of selection outputs were subcloned for high throughput expression and screening in IgG format. Functional clones from either approach were reformatted into antibody heavy and light chain vectors for mammalian expression as IgG or Fab molecules.

A representative anti-PAD4 antibody (PAD40048) had an affinity (K_{D}) for human PAD4 of approximately 87 nM ( ). The sequence of PAD40048 is provided in Table 62.

The anti-PAD4 antibodies were then affinity optimised. The sequences of the resultant anti-PAD4 antibodies are shown in Table 43 to Table 54. The affinities of the PAD4 antibodies are shown in Table 74 and Table 75.

PAD4 mouse surrogate antibodies were also identified, the affinities of which are shown in Table 80. The sequences of the surrogate affinity optimised antibodies are shown in Table 55 and Table 56.

### 11 EXAMPLE 4: ISCAT ANALYSIS FOR ANTI-PAD4 AND ANTI-PAD2 BINDING BEHAVIOUR

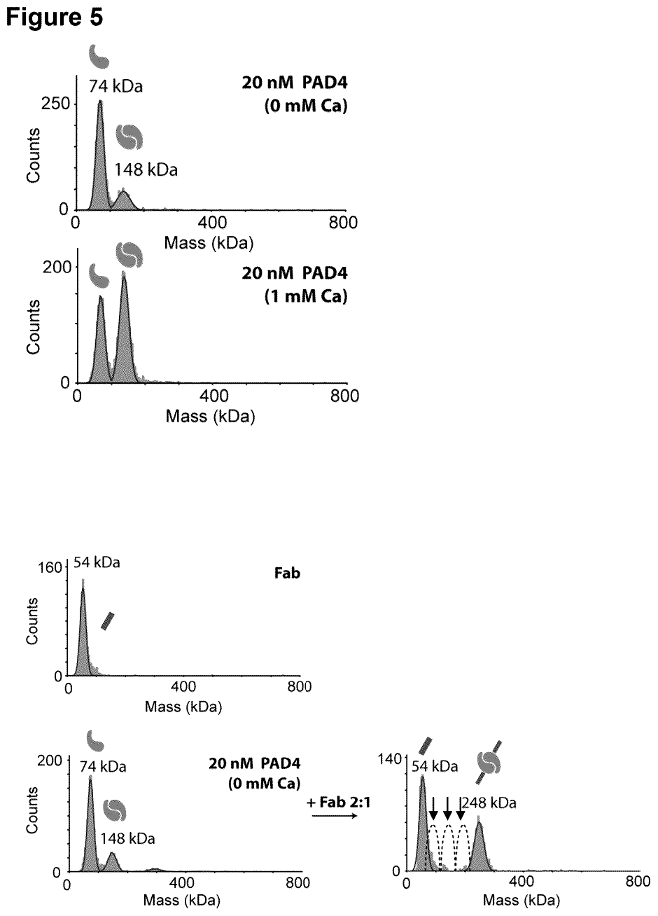

iSCAT analysis revealed that the Fab format of the anti-PAD4 affinity optimised antibody clearly favoured the heterotetramer form, suggesting that the Fab stabilises the dimeric form of PAD4. Strikingly there was also a complete absence of PAD4 monomers with Fab and PAD4 dimers with only one Fab, reinforcing the same conclusion (FIG. 5, arrows).

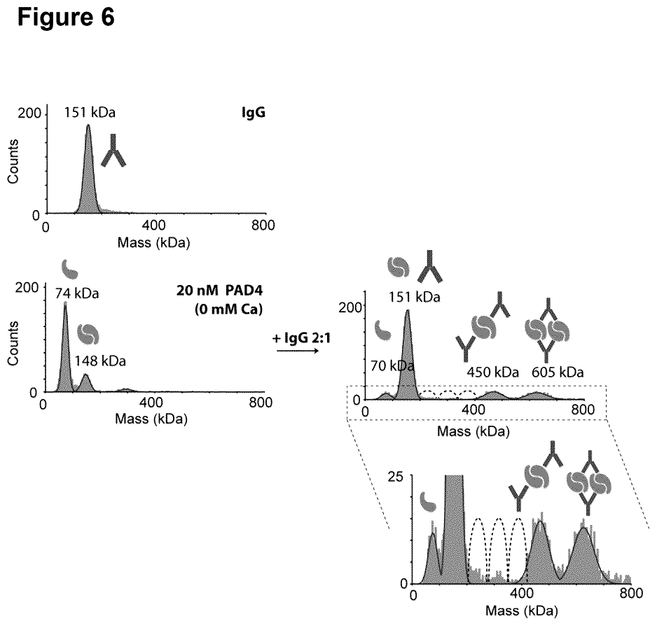

Similar to the Fab, addition of IgG also favours the heterotetramer suggesting that the IgG anti-PAD4 also stabilises the dimeric form of PAD4 (FIG. 6). Here, the larger size of the IgG (no peak overlap) enabled detection of the monomeric PAD4 species. Similar to the Fab anti-PAD4, there was a complete absence of i) PAD4 monomers with IgG and ii) PAD4 dimers with only one IgG. There was also an additional peak at ˜600 kDa which was only detectable using the IgG and not the Fab. These data suggested that a single anti-PAD4 IgG is able to span both binding sites of dimeric PAD4 (FIG. 6).

In summary, mass photometry suggested that both the Fab and the IgG anti-PAD4 affinity optimised antibody strongly favours the PAD4 heterotetramer complex. Furthermore, the IgG but not the Fab induces an additional complex which could correspond to the heterohexamer containing 2×PAD4 dimers and 2×IgG. This configuration would enable multivalency, also in solution, thus making the IgG substantially more potent that the Fab.

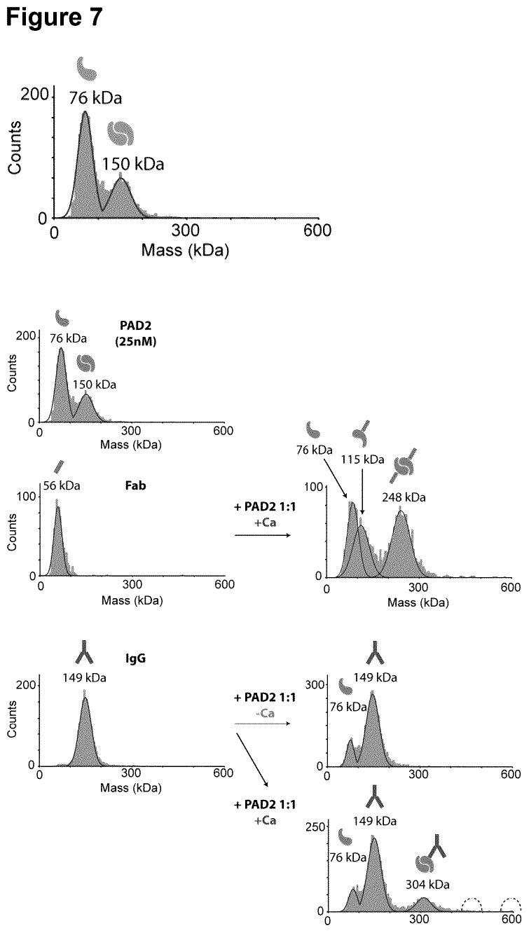

Similar to PAD4, PAD2 also exhibited a monomer-dimer equilibrium (FIG. 7). In contrast to PAD4, addition of anti-PAD2 Fab affinity optimised antibody generate both heterodimers and heterotetramers of PAD2 suggesting a different binding epitope location compared to the anti-PAD4 antibodies. The IgG PAD2 only showed one complex corresponding to a single IgG, suggesting that the binding epitope is placed such that IgG binding create a steric clash with the second binding site (arrow). Furthermore, the complete absence of a peak at 600 kDa suggested that PAD2 IgG is not able to form the multivalent complex as seen for PAD4. This is in line with citrullination assay data which show less “add-on” potency for PAD2 vs PAD4 IgG.

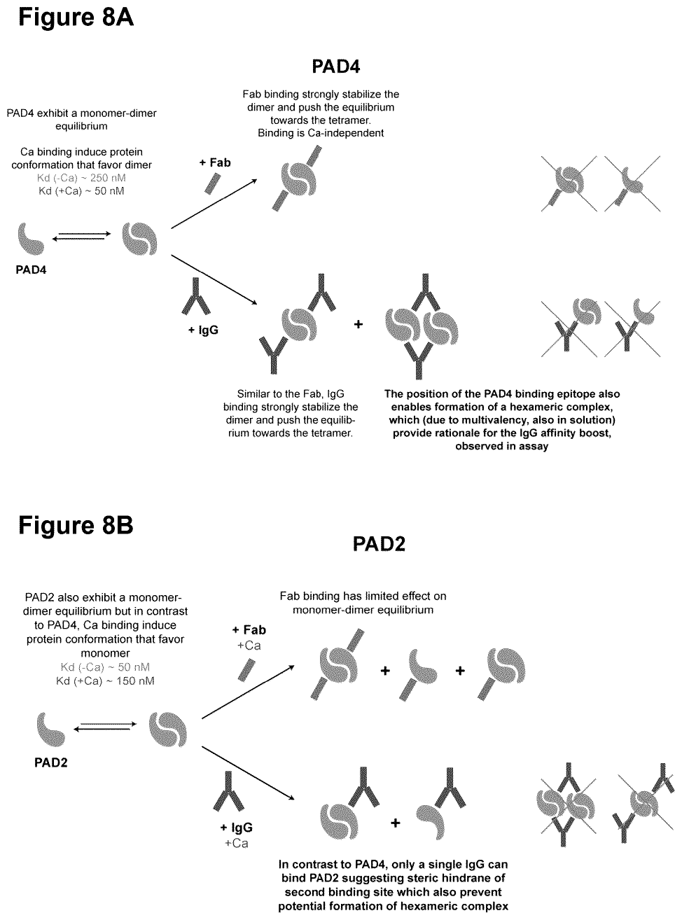

In summary, Fab and IgG anti-PAD4 binding of PAD4 stabilizes PAD4 dimers. The epitope permitted IgG anti-PAD4 to form hexameric complexes with the PAD4 dimers (FIG. 8A). By contrast, whilst PAD2 exhibited a monomer-dimer equilibrium, anti-PAD2 binding had limited effect on dimer stabilisation (FIG. 8B). Furthermore, in contrast to PAD4, only a single IgG was able to bind PAD2, suggesting a steric hindrance of the second binding site which also prevent potential formation of hexameric complexes.

### 12 EXAMPLE 5: GENERATION OF ANTI-PAD2/ANTI-PAD4 BISPECIFICS

Monovalent and bivalent bispecifics were generated as DuetMab or Bis3 formats respectively (FIG. 10A and FIG. 10B).

### 12.1 DuetMab Formats

DuetMabs with 2 different orientations were produced from the affinity optimised anti-PAD2 and anti-PAD4 antibodies (Clone 22 and Clone 42) (FIG. 10A, Table 66). The sequences of example DuetMab bispecifics (Clones 01-06) are shown in Table 58. Clone 06 was additional modified to remove the RF site from the end of the hole Fc (Table 58, SEQ ID NO: 59 and SEQ ID NO: 60), so as to abolish protein A binding. The Fc regions were modified to contain knob-in-hole mutations.

TABLE 66
DuetMab orientations

| | λ (knob) | κ (hole) | Clones |
|---|---|---|---|
| DuetMab 1^{st} orientation | Anit-PAD2 | Anti-PAD4 | Clone 01, |
| | | | Clone 03, |
| | | | Clone 05 |
| DuetMab 2^{nd} orientation | Anti-PAD4 | Anti-PAD2 | Clone 02, |
| | | | Clone 04, |
| | | | Clone 06 |

### 12.2 Bis3 Formats

Bis3 format bispecifics with 2 different orientations were produced from the affinity optimised anti-PAD2 and anti-PAD4 antibodies (Clone 22 and Clone 42) (FIG. 10B, Table 67). The sequences of example Bis3 bispecifics are shown in Table 57 (Clone 07-12).

TABLE 67
Bis3 orientations

| | Fab | scFv | Clones |
|---|---|---|---|
| Bis3 1^{st} orientation | Anti-PAD2 | Anti-PAD4 | Clone 08, |
| | | | Clone 10, |
| | | | Clone 12 |
| Bis 3 2^{nd} orientation | Anti-PAD4 | Anti-PAD2 | Clone 07, |
| | | | Clone 09, |
| | | | Clone 11 |

### 13 EXAMPLE 6: AFFINITY

### 13.1 Affinity of Prior Art Anti-PAD2 and Anti-PAD4 Antibodies

Aosasa et al. describes anti-PAD2 antibodies having an affinities (K_{D}) for PAD2 in the range of 6.33 to 74 nM [13]. The anti-PAD2 antibodies described in the prior art therefore had low affinities for the target.

### 13.2 Anti-PAD2 and Anti-PAD4 Affinities

The affinities of the affinity optimised anti-PAD2 antibodies from Example 3 to human, cynomolgus and mouse PAD2 are shown in Table 68.

TABLE 68
anti-PAD2 Fab affinities

| | PAD2 | kₐ (M⁻¹ s⁻¹) | | k_{d} (s⁻¹) | | K_{D} (nM) | | |
|---|---|---|---|---|---|---|---|---|
| Fab name | species | Average | SD | Average | SD | Average | SD | n= |
| PAD40141 | Human | 4.45E+05 | 1.65E+05 | 1.11E−01 | 3.14E−02 | 261 | 50 | 4 |
| hIgG1 ngl-2 | Cyno | 9.34E+04 | 5.34E+04 | 1.51E−01 | 7.10E−02 | 1706 | 526 | 3 |
| (Parent) | Mouse | n.d. | | | | | | |
| PAD40119 | Human | 8.86E+05 | 1.74E+05 | 1.05E−01 | 1.34E−02 | 120 | 9 | 3 |
| hIgG1 ngl-2 | Cyno | 6.59E+05 | 1.21E+05 | 1.04E−01 | 1.52E−02 | 160 | 18 | 3 |
| | Mouse | | | | | | | |
| PAD40175 | Human | 9.30E+04 | 8.54E+03 | 6.29E−04 | 1.25E−04 | 6.73 | 0.8 | 3 |
| hIgG1 ngl-2 | Cyno | 4.14E+04 | 2.17E+04 | 9.52E−04 | 7.67E−05 | 26.2 | 9.2 | 3 |
| | Mouse | 1.23E+05 | 5.34E+03 | 2.88E−04 | 1.79E−05 | 2.35 | 0.1 | 6 |
| 141LO0002 | Human | 8.80E+05 | 1.20E+05 | 3.28E−03 | 2.01E−04 | 3.78 | 0.5 | 8 |
| hIgG1 ngl-2 | Cyno | 4.56E+05 | 3.29E+04 | 3.76E−03 | 1.82E−04 | 8.26 | 0.5 | 8 |
| | Mouse | n.d. | | | | | | |
| 141LO0002 | Human | 1.07E+06 | 1.13E+05 | 3.50E−03 | 2.07E−04 | 3.31 | 0.5 | 3 |
| hIgG1 pgl-3 | Cyno | 5.00E+05 | 3.19E+04 | 4.27E−03 | 5.17E−05 | 8.57 | 0.4 | 3 |
| (Germlined) | Mouse | 7.77E+05 | 1.66E+05 | 2.60E−02 | 4.03E−03 | 33.8 | 2.8 | 4 |
| 141LO0002 | Human | 1.13E+06 | 8.17E+04 | 3.14E−03 | 1.72E−04 | 2.78 | 0.4 | 2 |
| hIgG1 pgl-4 | Cyno | 5.13E+05 | 4.66E+04 | 3.93E−03 | 5.67E−06 | 7.70 | 0.7 | 2 |
| (Germlined) | Mouse | 6.92E+05 | 1.61E+05 | 2.56E−02 | 3.27E−03 | 38.5 | 8.5 | 6 |
| 141LO0030 | Human | 6.47E+05 | 2.94E+04 | 6.13E−04 | 1.36E−05 | 0.949 | 0.1 | 3 |
| hIgG1 ngl-2 | Cyno | 3.56E+05 | 4.82E+03 | 5.43E−04 | 1.53E−05 | 1.52 | 0.04 | 3 |
| | Mouse | n.d. | | | | | | |
| 141LO0030 | Human | 4.74E+05 | 3.49E+04 | 1.01E−03 | 5.45E−05 | 2.14 | 0.3 | 5 |
| hIgG1 pgl-4 | Cyno | 2.77E+05 | 4.07E+03 | 8.08E−04 | 4.91E−05 | 2.92 | 0.2 | 5 |
| (Germlined) | Mouse | 3.78E+05 | 6.52E+04 | 4.84E−03 | 4.20E−04 | 13.2 | 2.9 | 8 |
| 141LO0035 | Human | 1.04E+06 | 1.97E+04 | 1.92E−04 | 3.11E−05 | 0.185 | 0.03 | 5 |
| hIgG1 ngl-2 | Cyno | 5.93E+05 | 5.49E+04 | 1.41E−04 | 1.96E−05 | 0.241 | 0.05 | 5 |
| | Mouse | 1.14E+06 | 8.75E+04 | 6.46E−04 | 2.70E−05 | 0.571 | 0.05 | 6 |
| 141LO0035 | Human | 1.01E+06 | 2.63E+04 | 2.21E−04 | 1.59E−05 | 0.218 | 0.02 | 6 |
| hIgG1 pgl-4 | Cyno | 6.26E+05 | 3.06E+04 | 1.58E−04 | 2.50E−05 | 0.253 | 0.05 | 6 |
| (Germlined) | Mouse | 9.71E+05 | 9.50E+04 | 7.66E−04 | 6.25E−05 | 0.794 | 0.08 | 9 |
| Clone 22 | | | | | | | | |
| 141LO0039 | Human | 8.40E+05 | 5.11E+04 | 1.57E−03 | 4.68E−05 | 1.87 | 0.1 | 2 |
| hIgG1 ngl-2 | Cyno | 4.67E+05 | 2.26E+03 | 2.03E−03 | 6.50E−05 | 4.35 | 0.2 | 2 |
| | Mouse | n.d. | | | | | | |
| 141LO0039 | Human | 6.29E+05 | 3.05E+04 | 2.45E−03 | 8.60E−05 | 3.90 | 0.3 | 3 |
| hIgG1 pgl-4 | Cyno | 3.90E+05 | 4.49E+04 | 2.97E−03 | 2.65E−04 | 7.65 | 0.3 | 3 |
| (Germlined) | Mouse | 7.22E+05 | 1.20E+05 | 1.25E−02 | 6.39E−04 | 17.8 | 3.5 | 5 |
| 141LO0055 | Human | 1.85E+06 | 5.97E+04 | 6.75E−04 | 1.16E−05 | 0.364 | 0.02 | 2 |
| hIgG1 ngl-2 | Cyno | 1.03E+06 | 2.06E+05 | 1.41E−03 | 3.77E−05 | 1.41 | 0.3 | 2 |

| | |
|---|---|
| Mouse | n.d. |

Biotinylated PAD2 was captured onto a CM5/C1-Streptavidin surface and anti-PAD2 Fabs flowed over. SD = Standard Deviation. n.d. = not determined.

Summary data for the anti-PAD2 antibodies are shown in Table 69 and FIG. 13. In contrast to the prior art anti-PAD2 antibodies [13], ten of the anti-PAD4 clones had affinities for human PAD2 that were below 6 nM (FIG. 13, Table 69). Four of the clones had affinities for human PAD2 that were below 1 nM (FIG. 13, Table 69). Two of the clones had affinities for human, cynomolgus and mouse PAD2 that were below 0.3 nM (FIG. 13, Table 69).

TABLE 69
anti-PAD2 Fab affinities - Summary

| | | | | |
|---|---|---|---|---|
| | | | | Fold |
| | | K_{D} (nM) | | difference |
| Fab Name | Human PAD2 | Cyno PAD2 | Mouse PAD2 | cyno/human |

Human PAD2 K_{D} <1 nM

| | | | | |
|---|---|---|---|---|
| 141LO0035 hIgG1 ngl-2 | 0.185 | 0.241 | 0.571 | 1.3 |
| 141LO0035 hIgG1 pgl-4 | 0.218 | 0.253 | 0.794 | 1.2 |
| (Clone 22) | | | | |
| 141LO0055 hIgG1 ngl-2 | 0.364 | 1.41 | n.d. | 3.9 |
| 141LO0030 hIgG1 ngl-2 | 0.949 | 1.52 | n.d. | 1.6 |

Human PAD2 K_{D} <6 nM

| | | | | |
|---|---|---|---|---|
| 141LO0039 hIgG1 ngl-2 | 1.87 | 4.35 | n.d. | 2.3 |
| 141LO0030 hIgG1 pgl-4 | 2.14 | 2.92 | 13.2 | 1.4 |
| 141LO0002 hIgG1 pgl-4 | 2.78 | 7.70 | 38.5 | 2.8 |
| 141LO0002 hIgG1 pgl-3 | 3.31 | 8.57 | 33.8 | 2.6 |
| 141LO0002 hIgG1 ngl-2 | 3.78 | 8.26 | n.d. | 2.2 |
| 141LO0039 hIgG1 pgl-4 | 3.90 | 7.65 | 17.8 | 2.0 |

Human PAD2 K_{D} >6 nM

| | | | | |
|---|---|---|---|---|
| PAD40175 hIgG1 ngl-2 | 6.73 | 26.2 | 2.35 | 3.9 |
| PAD40119 hIgG1 ngl-2 | 120 | 160 | n.d. | 1.3 |
| PAD40141 hIgG1 ngl-2 | 261 | 1706 | n.d | 6.5 |

Biotinylated PAD2 was captured onto a CM5/C1-Streptavidin surface and anti-PAD2 Fabs flowed over. Data shown are averages of n = 2-9 experiments. n.d. = not determined.

The affinities of the anti-PAD4 antibodies from Example 2 to human and cynomolgus PAD4 are shown in Table 70.

TABLE 70
anti-PAD4 Fab affinities

| | PAD4 | kₐ (M⁻¹ s⁻¹) | | k_{d} (s⁻¹) | | K_{D} (nM) | | |
|---|---|---|---|---|---|---|---|---|
| Fab name | species | Average | SD | Average | SD | Average | SD | n= |
| PAD40048 | Human | 2.04E+06 | 2.84E+05 | 3.01E−02 | 2.50E−03 | 14.9 | 1.9 | 5 |
| hIgG1 ngl-2 | Cyno | 8.77E+05 | 1.97E+05 | 2.84E−01 | 3.01E−02 | 332 | 49 | 5 |
| (Parent) | | | | | | | | |
| PAD40048 | Human | 5.13E+05 | 1.57E+04 | 4.44E−02 | 3.14E+00 | 86.5 | 3.1 | 2 |
| hIgG1 pgl-24 | Cyno | 1.02E+05 | | 1.50E−01 | | 1460 | | 1 |
| (Parent | | | | | | | | |
| germlined) | | | | | | | | |
| 48LO0010 | Human | 1.31E+07 | 4.54E+06 | 1.88E−03 | 2.13E−02 | 0.147 | 0.02 | 2 |
| hIgG1 ngl-2 | Cyno | 1.28E+07 | 6.03E+06 | 9.71E−02 | 4.29E−02 | 7.61 | 0.2 | 1 |
| 48LO0032 | Human | 8.89E+06 | | 5.59E−04 | | 0.063 | | 1 |
| hIgG1 ngl-2 | Cyno | 6.81E+06 | | 4.68E−03 | | 0.686 | | 1 |
| 48LO0033 | Human | 1.20E+07 | | 2.22E−03 | | 0.185 | | 1 |
| hIgG1 ngl-3 | Cyno | 1.38E+07 | | 1.24E−02 | | 0.900 | | 1 |
| 48LO0036 | Human | 9.17E+06 | 1.34E+06 | 4.44E−04 | 4.40E−03 | 0.049 | 0.004 | 2 |
| hIgG1 ngl-3 | Cyno | 7.67E+06 | 4.49E+05 | 1.89E−02 | 7.62E−04 | 2.46 | 0.04 | 2 |
| 48LO0040 | Human | 6.69E+06 | 2.22E+05 | 8.34E−04 | 6.31E−03 | 0.125 | 0.006 | 2 |
| hIgG1 ngl-3 | Cyno | 7.04E+06 | 9.56E+05 | 8.96E−03 | 1.13E−03 | 1.27 | 0.01 | 2 |
| 48LO0048 | Human | 3.44E+06 | | 7.90E−05 | | 0.023 | | 1 |
| hIgG1 ngl-3 | Cyno | 3.97E+06 | 8.42E+05 | 1.92E−03 | 5.29E−04 | 0.479 | 0.03 | 2 |
| 48LO0049 | Human | 4.98E+06 | 8.50E+05 | 1.29E−04 | 8.81E−03 | 0.025 | 0.009 | 4 |
| hIgG1 ngl-3 | Cyno | 4.93E+06 | 6.09E+05 | 1.73E−03 | 3.73E−04 | 0.353 | 0.07 | 4 |
| 48LO0051 | Human | 6.82E+06 | 1.78E+06 | 4.34E−04 | 1.22E−02 | 0.062 | 0.012 | 2 |
| hIgG1 ngl-2 | Cyno | 7.38E+06 | 1.82E+06 | 1.05E−02 | 2.70E−03 | 1.42 | 0.01 | 2 |
| 48LO0060 | Human | 7.52E+06 | | 1.02E−03 | | 0.136 | | 1 |
| hIgG1 ngl-3 | Cyno | 3.83E+06 | | 1.02E−02 | | 2.66 | | 1 |
| 48LO0062 | Human | 1.59E+07 | | 6.95E−04 | | 0.044 | | 1 |
| hIgG1 ngl-3 | Cyno | 1.72E+07 | | 1.03E−02 | | 0.597 | | 1 |
| 48LO0063 | Human | 1.18E+07 | 1.61E+06 | 2.45E−04 | 3.59E−03 | 0.021 | 0.004 | 5 |
| hIgG1 ngl-3 | Cyno | 8.93E+06 | 1.72E+06 | 1.41E−03 | 1.35E−04 | 0.163 | 0.04 | 5 |
| 48LO0049 | Human | 2.37E+06 | 2.63E+05 | 1.51E−04 | 5.16E−05 | 0.057 | 0.014 | 3 |
| hIgG1 pgl-9 | Cyno | 3.05E+06 | 3.31E+05 | 2.09E−03 | 1.95E−04 | 0.685 | 0.02 | 3 |
| 48LO0049 | Human | 3.53E+06 | 3.28E+03 | 2.81E−04 | 1.14E−05 | 0.080 | 0.003 | 2 |
| hIgG1 pgl-10 | Cyno | 4.83E+06 | 2.36E+05 | 7.78E−03 | 6.70E−04 | 1.61 | 0.06 | 2 |
| 48LO0049 | Human | 4.07E+06 | 2.20E+05 | 1.88E−04 | 1.32E−05 | 0.046 | 0.006 | 3 |
| hIgG1 pgl-12 | Cyno | 3.71E+06 | 2.45E+05 | 1.07E−03 | 5.92E−05 | 0.289 | 0.02 | 3 |
| 48LO0049 | Human | 4.53E+06 | 2.94E+05 | 1.05E−04 | 4.81E−06 | 0.023 | 0.003 | 3 |
| hIgG1 fgl-23 | Cyno | 4.50E+06 | 2.48E+05 | 1.10E−03 | 3.76E−05 | 0.246 | 0.02 | 3 |
| 48LO0049 | Human | 3.55E+06 | 1.18E+05 | 1.45E−03 | 7.72E−05 | 0.407 | 0.03 | 3 |
| hIgG1 fgl-25 | Cyno | 5.40E+06 | 9.11E+05 | 2.56E−02 | 1.42E−03 | 4.80 | 0.6 | 3 |
| 48LO0049 | Human | 9.21E+05 | 6.17E+04 | 4.34E−04 | 4.70E−06 | 0.473 | 0.03 | 3 |
| hIgG1 pgl-31 | Cyno | 1.72E+06 | 4.98E+04 | 6.33E−03 | 1.56E−04 | 3.69 | 0.1 | 3 |
| 48LO0049 | Human | 1.88E+06 | 5.52E+04 | 1.24E−04 | 2.87E−05 | 0.066 | 0.02 | 6 |
| hIgG1 fgl-33 | Cyno | 2.08E+06 | 2.50E+05 | 8.37E−04 | 1.58E−04 | 0.400 | 0.04 | 4 |
| 48LO0063 | Human | 5.72E+06 | 1.97E+05 | 3.03E−04 | 1.98E−05 | 0.053 | 0.004 | 4 |
| hIgG1 fgl-4 | Cyno | 4.70E+06 | 4.02E+04 | 1.55E−03 | 3.81E−05 | 0.330 | 0.01 | 3 |
| 48LO0063 | Human | 6.10E+06 | 2.70E+05 | 2.83E−04 | 1.92E−05 | 0.047 | 0.004 | 5 |
| hIgG1 fgl-6 | Cyno | 5.07E+06 | 3.14E+05 | 7.37E−04 | 7.53E−05 | 0.145 | 0.01 | 5 |
| 48LO0063 | Human | 1.04E+07 | 2.20E+05 | 4.59E−03 | 2.59E−05 | 0.440 | 0.01 | 3 |
| hIgG1 fgl-7 | Cyno | 1.67E+07 | 1.05E+06 | 2.87E−02 | 1.64E−03 | 1.72 | 0.04 | 3 |
| 48LO0063 | Human | 1.82E+06 | 2.06E+04 | 2.13E−02 | 6.86E−04 | 11.7 | 0.2 | 2 |
| hIgG1 fgl-8 | Cyno | 1.85E+06 | 1.20E+06 | 4.37E−02 | 2.44E−02 | 24.5 | 2.7 | 2 |
| 48LO0063 | Human | 6.34E+06 | 1.58E+05 | 6.05E−04 | 9.60E−06 | 0.095 | 0.001 | 3 |
| hIgG1 fgl-9 | Cyno | 5.19E+06 | 2.89E+05 | 3.39E−02 | 2.00E−03 | 6.52 | 0.1 | 3 |
| 48LO0063 | Human | 5.61E+06 | 3.29E+05 | 6.56E−04 | 3.13E−05 | 0.117 | 0.002 | 3 |
| hIgG1 fgl-11 | Cyno | 4.68E+06 | 3.82E+05 | 1.75E−02 | 3.10E−04 | 3.75 | 0.3 | 3 |
| 48LO0063 | Human | 1.04E+07 | | 9.50E−04 | | 0.091 | | 1 |
| hIgG1 pgl-38 | Cyno | 3.61E+07 | | 4.77E−02 | | 1.32 | | 1 |
| 48LO0063 | Human | 5.00E+06 | | 8.18E−02 | | 16.3 | | 1 |
| hIgG1 pgl-39 | Cyno | | | | | n.b. | | 1 |
| 48LO0063 | Human | 1.23E+07 | | 1.91E−04 | | 0.016 | | 1 |
| hIgG1 pgl-40 | Cyno | 9.81E+06 | | 2.91E−03 | | 0.297 | | 1 |
| 48LO0063 | Human | 1.19E+07 | | 5.39E−04 | | 0.045 | | 1 |
| hIgG1 pgl-41 | Cyno | 4.86E+07 | | 1.01E−01 | | 2.08 | | 1 |
| 48LO0063 | Human | 1.24E+07 | | 5.43E−03 | | 0.440 | | 1 |
| hIgG1 pgl-42 | Cyno | | | | | n.b. | | 1 |
| 48LO0063 | Human | 5.54E+06 | | 8.19E−02 | | 14.8 | | 1 |
| hIgG1 pgl-43 | Cyno | | | | | n.b. | | 1 |
| 48LO0063 | Human | 8.91E+06 | 1.07E+05 | 1.42E−03 | 2.10E−05 | 0.160 | 0.002 | 3 |
| hIgG1 pgl-47 | Cyno | 3.38E+07 | 7.00E+06 | 6.38E−02 | 9.46E−03 | 1.91 | 0.1 | 3 |
| 48LO0063 | Human | 1.03E+07 | | 5.32E−04 | | 0.052 | | 1 |
| hIgG1 pgl-49 | Cyno | 1.31E+07 | | 7.50E−03 | | 0.570 | | 1 |
| 48LO0063 | Human | 1.02E+07 | | 3.27E−03 | | 0.319 | | 1 |
| hIgG1 pgl-51 | Cyno | 3.80E+07 | | 8.73E−02 | | 2.30 | | 1 |
| 48LO0063 | Human | 1.15E+07 | | 3.33E−04 | | 0.029 | | 1 |
| hIgG1 pgl-52 | Cyno | 1.58E+07 | | 6.70E−03 | | 0.424 | | 1 |
| 48LO0063 | Human | 9.92E+06 | | 8.05E−04 | | 0.081 | | 1 |
| hIgG1 pgl-53 | Cyno | 1.48E+07 | | 1.12E−02 | | 0.756 | | 1 |
| 48LO0063 | Human | 6.10E+06 | 1.69E+05 | 2.78E−04 | 6.93E−05 | 0.045 | 0.010 | 4 |
| hIgG1 fgl-58 | Cyno | 4.06E+06 | 6.03E+05 | 2.16E−03 | 4.30E−04 | 0.532 | 0.05 | 4 |
| 48LO0063 | Human | 5.24E+06 | 2.76E+05 | 4.59E−04 | 3.51E−05 | 0.088 | 0.004 | 3 |
| hIgG1 fgl-59 | Cyno | 4.19E+06 | 4.57E+05 | 2.32E−03 | 3.77E−04 | 0.551 | 0.03 | 3 |
| Clone 42 | | | | | | | | |
| 48LO0063 | Human | 4.89E+06 | 1.14E+05 | 1.38E−03 | 3.57E−05 | 0.282 | 0.001 | 2 |
| hIgG1 fgl-60 | Cyno | 4.12E+06 | 1.86E+05 | 3.36E−03 | 8.27E−06 | 0.818 | 0.04 | 2 |
| 48LO0063 | Human | 4.96E+06 | 2.08E+04 | 7.25E−04 | 4.41E−06 | 0.146 | 0.0003 | 2 |
| hIgG1 fgl-61 | Cyno | 3.88E+06 | 6.07E+05 | 2.33E−03 | 1.52E−04 | 0.605 | 0.06 | 2 |

Biotinylated PAD4 was captured onto a CM5/C1-Streptavidin surface and anti-PAD4 Fabs flowed over. SD = Standard Deviation. n.b. = no binding.

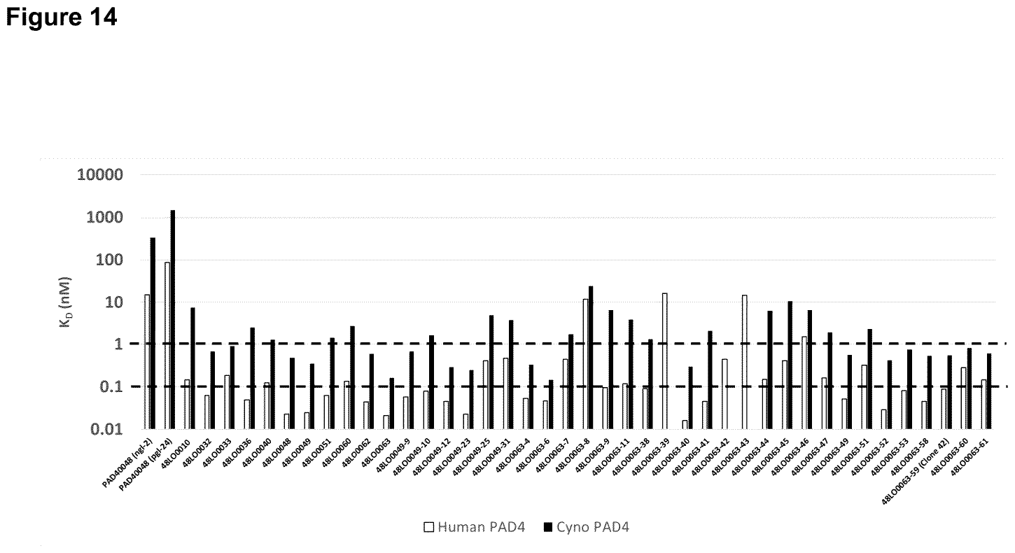

Summary data for the anti-PAD4 antibodies are shown in Table 71 and FIG. 14. The clones with an affinity (K_{D}) for human PAD4 of less than 0.1 and/or 1 nM are highlighted in FIG. 14 (dotted lines).

TABLE 71
anti-PAD4 affinities - Summary

| | | | Fold |
|---|---|---|---|
| | K_{D} (nM) | | difference |
| Fab Name | Human PAD4 | Cyno PAD4 | cyno/human |

Human PAD4 K_{D} <0.1 nM

| | | | |
|---|---|---|---|
| 48LO0063 hIgG1 pgl-40 | 0.016 | 0.297 | 19 |
| 48LO0063 hIgG1 ngl-3 | 0.021 | 0.163 | 7.9 |
| 48LO0048 hIgG1 ngl-3 | 0.023 | 0.479 | 20.8 |
| 48LO0049 hIgG1 fgl-23 | 0.023 | 0.246 | 10.5 |
| 48LO0049 hIgG1 ngl-3 | 0.025 | 0.353 | 14.2 |
| 48LO0063 hIgG1 pgl-52 | 0.029 | 0.424 | 14.7 |
| 48LO0062 hIgG1 ngl-3 | 0.044 | 0.597 | 13.6 |
| 48LO0063 hIgG1 pgl-41 | 0.045 | 2.08 | 46.1 |
| 48LO0063 hIgG1 fgl-58 | 0.045 | 0.532 | 11.7 |
| 48LO0049 hIgG1 pgl-12 | 0.046 | 0.289 | 6.2 |
| 48LO0063 hIgG1 fgl-6 | 0.047 | 0.145 | 3.1 |
| 48LO0036 hIgG1 ngl-3 | 0.049 | 2.46 | 51 |
| 48LO0063 hIgG1 pgl-49 | 0.052 | 0.570 | 11 |
| 48LO0063 hIgG1 fgl-4 | 0.053 | 0.330 | 6.2 |
| 48LO0049 hIgG1 pgl-9 | 0.057 | 0.685 | 12 |
| 48LO0051 hIgG1 ngl-2 | 0.062 | 1.42 | 23 |
| 48LO0032 hIgG1 ngl-2 | 0.063 | 0.686 | 11 |
| 48LO0049 hIgG1 fgl-33 | 0.066 | 0.400 | 6.0 |
| 48LO0049 hIgG1 pgl-10 | 0.080 | 1.61 | 20 |
| 48LO0063 hIgG1 pgl-53 | 0.081 | 0.756 | 9.3 |
| 48LO0063 hIgG1 fgl-59 | 0.088 | 0.551 | 6.3 |
| (Clone 42) | | | |
| 48LO0063 hIgG1 pgl-38 | 0.091 | 1.32 | 15 |
| 48LO0063 hIgG1 fgl-9 | 0.095 | 6.52 | 68 |

Human PAD4 K_{D} <1 nM

| | | | |
|---|---|---|---|
| 48LO0063 hIgG1 fgl-11 | 0.117 | 3.75 | 32 |
| 48LO0040 hIgG1 ngl-3 | 0.125 | 1.27 | 10 |
| 48LO0060 hIgG1 ngl-3 | 0.136 | 2.66 | 20 |
| 48LO0063 hIgG1 fgl-61 | 0.146 | 0.605 | 4.1 |
| 48LO0010 hIgG1 ngl-2 | 0.147 | 7.61 | 52 |
| 48LO0063 hIgG1 pgl-47 | 0.160 | 1.91 | 12 |
| 48LO0033 hIgG1 ngl-3 | 0.185 | 0.900 | 4.9 |
| 48LO0063 hIgG1 fgl-60 | 0.282 | 0.818 | 2.9 |
| 48LO0063 hIgG1 pgl-51 | 0.319 | 2.30 | 7.2 |
| 48LO0049 hIgG1 fgl-25 | 0.407 | 4.80 | 12 |
| 48LO0063 hIgG1 fgl-7 | 0.440 | 1.72 | 3.9 |
| 48LO0063 hIgG1 pgl-42 | 0.440 | n.b. | — |
| 48LO0049 hIgG1 pgl-31 | 0.473 | 3.69 | 7.8 |

Human PAD4 K_{D} >1 nM

| | | | |
|---|---|---|---|
| 48LO0063 hIgG1 fgl-8 | 11.7 | 24.5 | 2.1 |
| 48LO0063 hIgG1 pgl-43 | 14.8 | n.b. | — |
| PAD40048 hIgG1 ngl-2 | 14.9 | 332 | 22 |
| 48LO0063 hIgG1 pgl-39 | 16.3 | n.b. | — |
| PAD40048 hIgG1 pgl-24 | 86.5 | 1460 | 17 |

Biotinylated PAD4 was captured onto a CM5/C1-Streptavidin surface and anti-PAD4 Fabs flowed over. Data shown are averages of n = 1-6 experiments. n.b. = no binding.

### 13.3 Bispecific Affinities

The affinities of the different bispecific formats for PAD2 were measured and are shown in Table 72, and summarised in Table 73. In this assay, the affinity of Clone 22 as a Fab (141LO0035 hIgG1 pgl-4) measured 10× higher than in the previous assay (Table 69). The recombinant PAD2 used here was different compared to when the affinities of the anti-PAD2 antibodies from Example 3 were measured. Here, the tags on the recombinant PAD2 (and PAD4) were on the N-terminus of the proteins, compared to the C-terminus before. This could potentially lead to better folding of the proteins or the epitope being presented better, accounting for the higher affinity measured in this experiment.

The affinities of the different bispecific formats for PAD4 were measured and are shown in Table 74 and summarised in Table 75.

The affinities of the DuetMabs for PAD2 and PAD4 were comparable to the affinities of the respective Fab and IgG, in both orientations. In other words, there was surprisingly no loss of PAD2 or PAD4 affinity when optimised anti-PAD2 and anti-PAD4 antibodies were combined into a DuetMab bispecific (Table 72 to Table 75). The affinity (K_{D}) of the DuetMabs for human PAD2 were in the range of about 10-18 pM (Table 73). The affinity (K_{D}) of the DuetMabs for human PAD4 were in the range of about 40-58 pM (Table 75). The Fc modifications had no effect on the affinity of the DuetMabs. In summary, all of the DuetMab bispecifics retained high affinity for both PAD2 and PAD4 (human, cynomolgus and mouse (PAD2 only)), regardless of orientation (i.e. regardless of which of the anti-PAD2 or anti-PAD4 was the “hole” or “knob”).

All of the Bis3 bispecifics retained high affinity for both PAD2 and PAD4 (human, cynomolgus and mouse (PAD2 only)), regardless of orientation (i.e. regardless of which of the anti-PAD2 or anti-PAD4 was the scFv or Fab) (Table 72 to Table 75).

The Bis3 format bispecifics had a drop in human PAD4 affinity compared to the IgG (Table 75). The Bis3 format with the PAD4 arm in Fab format (e.g. Clone 07) was better at maintaining affinity for both human and cynomolgus PAD4. The affinity (K_{D}) of the Bis3 bispecifics for human PAD4 were in the range of about 8-50 pM (Table 75). Clone 12 had an affinity (K_{D}) for human PAD4 of about 30 pM. The Bis3 format bispecifics had comparable affinity for human PAD2 as the IgG comprising the same PAD4 binding domain (Table 73). The affinity (K_{D}) of the DuetMabs for human PAD2 were in the range of about 6-16 pM (Table 73). The Bis3 format with PAD4 arm in Fab format was better at maintaining affinity for cynomolgus PAD4 compared to the DuetMabs and other Bis3 formats (Table 76). The Fc modifications did not significantly affect affinity, for either the Bis3 or DuetMab format (Table 72 to Table 75).

TABLE 72
Bispecific affinity for PAD2

| | PAD2 | kₐ (M⁻¹ s⁻¹) | | k_{d} (s⁻¹) | | K_{D} (pM) | | |
|---|---|---|---|---|---|---|---|---|
| Name | species | Average | SD | Average | SD | Average | SD | n= |

1) Biotinylated PAD2 captured onto a C1-Streptavidin surface and anti-PAD antibodies flowed over

| | | | | | | | | |
|---|---|---|---|---|---|---|---|---|
| Clone 22 (Fab) | Human | 3.67E+06 | 2.93E+05 | 4.77E−05 | 1.58E−05 | 13.1 | 4.9 | 6 |
| | Cyno | 2.39E+06 | 2.05E+05 | 7.03E−05 | 1.51E−05 | 29.2 | 5.0 | 3 |
| | Mouse | 3.59E+06 | 5.18E+05 | 4.62E−04 | 4.21E−05 | 131 | 26 | 5 |
| Clone 1 | Human | 3.54E+06 | 2.89E+05 | 4.30E−05 | 6.64E−06 | 12.3 | 2.5 | 8 |
| | Cyno | 2.33E+06 | 2.37E+05 | 5.45E−05 | 1.24E−05 | 23.9 | 7.7 | 4 |
| | Mouse | 3.20E+06 | 1.92E+05 | 3.37E−04 | 9.08E−06 | 106 | 8 | 3 |
| Clone 2 | Human | 3.30E+06 | 2.25E+05 | 5.08E−05 | 7.12E−06 | 15.5 | 2.8 | 8 |
| | Cyno | 2.15E+06 | 1.69E+05 | 5.20E−05 | 8.75E−06 | 24.5 | 5.7 | 4 |
| | Mouse | 3.21E+06 | 3.22E+05 | 3.10E−04 | 1.78E−05 | 97.5 | 14 | 3 |
| Clone 5 | Human | 3.36E+06 | 4.18E+05 | 4.62E−05 | 1.18E−05 | 14.2 | 4.9 | 3 |
| | Cyno | 2.53E+06 | 1.94E+05 | 3.00E−05 | 2.38E−06 | 12.0 | 1.7 | 3 |
| | Mouse | 3.13E+06 | 2.61E+05 | 3.50E−04 | 1.40E−05 | 113 | 13 | 4 |
| Clone 6 | Human | 3.23E+06 | 2.49E+05 | 5.56E−05 | 9.42E−06 | 17.4 | 4.1 | 3 |
| | Cyno | 1.91E+06 | 1.55E+05 | 3.85E−05 | 7.31E−06 | 20.4 | 5.5 | 3 |
| | Mouse | 2.94E+06 | 2.71E+05 | 3.27E−04 | 6.33E−06 | 112 | 12 | 4 |

2) Antibody captured on a surface and PAD2 flowed over

| | | | | | | | | |
|---|---|---|---|---|---|---|---|---|
| Clone 22 (IgG) | Human | 4.36E+06 | 9.58E+04 | 1.71E−05 | 5.58E−06 | 3.90 | 1.2 | 5 |
| | Cyno | 3.06E+06 | 3.63E+05 | 3.33E−05 | 2.28E−06 | 11.0 | 1.4 | 3 |
| | Mouse | 4.93E+06 | 2.23E+05 | 2.24E−05 | 4.83E−06 | 4.59 | 1.1 | 7 |
| Clone 1* | Human | 3.48E+06 | 1.38E+05 | 2.97E−05 | 6.61E−06 | 8.53 | 1.7 | 4 |
| | Cyno | 2.11E+06 | 1.22E+05 | 5.52E−05 | 2.08E−06 | 26.2 | 2.5 | 4 |
| | Mouse | 4.46E+06 | 1.93E+05 | 8.37E−05 | 6.07E−06 | 18.8 | 2.1 | 4 |
| Clone 2* | Human | 3.25E+06 | 1.29E+05 | 3.29E−05 | 9.71E−06 | 10.1 | 2.9 | 4 |
| | Cyno | 2.35E+06 | 4.48E+05 | 7.10E−05 | 8.49E−06 | 30.5 | 3.3 | 4 |
| | Mouse | 4.09E+06 | 3.91E+05 | 7.46E−05 | 5.94E−06 | 18.5 | 3.1 | 4 |
| Clone 6^{#} | Human | 3.51E+06 | 3.17E+05 | 8.11E−05 | 4.33E−06 | 23.3 | 3.3 | 2 |
| | Cyno | 1.67E+06 | 9.04E+04 | 4.87E−05 | 1.28E−05 | 28.9 | 6.1 | 2 |
| | Mouse | 4.14E+06 | 2.69E+05 | 1.37E−04 | 7.51E−06 | 33.1 | 0.3 | 2 |
| Clone 7* | Human | 2.00E+06 | 5.33E+04 | 3.21E−05 | 3.09E−06 | 16.1 | 1.6 | 6 |
| | Cyno | 2.61E+06 | 7.42E+05 | 5.46E−05 | 4.93E−06 | 21.9 | 4.7 | 4 |
| | Mouse | 2.76E+06 | 1.51E+05 | 4.45E−05 | 3.62E−06 | 16.2 | 2.0 | 4 |
| Clone 8* | Human | 4.48E+06 | 6.44E+05 | 2.97E−05 | 8.74E−06 | 6.65 | 1.9 | 5 |
| | Cyno | 2.74E+06 | 4.62E+05 | 4.52E−05 | 3.72E−06 | 16.7 | 2.1 | 4 |
| | Mouse | 4.65E+06 | 3.48E+05 | 3.31E−05 | 3.80E−06 | 7.19 | 1.4 | 3 |
| Clone 8^{#} | Human | 5.88E+06 | 1.36E+06 | 4.74E−05 | 3.05E−05 | 7.51 | 3.7 | 3 |
| | Cyno | 9.48E+06 | 6.91E+06 | 1.00E−04 | 4.32E−05 | 12.1 | 2.7 | 2 |
| | Mouse | n.d. | | | | | | |
| Clone 12^{#} | Human | 5.60E+06 | 9.45E+05 | 4.05E−05 | 2.70E−05 | 6.76 | 3.9 | 7 |
| | Cyno | 4.19E+06 | 2.27E+06 | 5.74E−05 | 3.11E−05 | 13.8 | 2.7 | 4 |
| | Mouse | 4.85E+06 | 2.12E+05 | 6.89E−05 | 3.35E−06 | 14.2 | 1.3 | 2 |

*= Antibody captured onto a C1-Protein G′ surface.
^{#}= Biotinylated Antibody captured onto a C1-Streptavidin surface.
SD = Standard Deviation. n.d. = not determined.

TABLE 73
Bispecific affinity for PAD2 - Summary

| | | | | | | | K_{D} (pM) | | |
|---|---|---|---|---|---|---|---|---|---|
| | | | Hole | Knob | | | Human | Cyno | Mouse |
| Name | Format | Fc | (κ) | (λ) | scFv | Fab | PAD2 | PAD2 | PAD2 |

1) Biotinylated PAD2 captured onto a C1-Streptavidin surface and anti-PAD antibodies flowed over

| | | | | | | | | | |
|---|---|---|---|---|---|---|---|---|---|
| Clone 22 | Fab | — | — | — | — | PAD2 | 13.1 | 29.2 | 131 |
| Clone 1 | DuetMab | TM | PAD4 | PAD2 | — | — | 12.3 | 23.9 | 106 |
| Clone 2 | DuetMab | TM | PAD2 | PAD4 | — | — | 15.5 | 24.5 | 97.5 |
| Clone 5 | DuetMab | FQQ YTE | PAD4 | PAD2 | — | — | 14.2 | 12.0 | 113 |
| Clone 6 | DuetMab | FQQ YTE | PAD2 | PAD4 | — | — | 17.4 | 20.4 | 112 |

2) Antibody captured on a surface and PAD2 flowed over

| | | | | | | | | | |
|---|---|---|---|---|---|---|---|---|---|
| Clone 22* | IgG | TM | — | — | — | PAD2 | 3.90 | 11.0 | 4.59 |
| Clone 1* | DuetMab | TM | PAD4 | PAD2 | — | — | 8.53 | 26.2 | 18.8 |
| Clone 2* | DuetMab | TM | PAD2 | PAD4 | — | — | 10.1 | 30.5 | 18.5 |
| Clone 6^{#} | DuetMab | FQQ YTE | PAD2 | PAD4 | — | — | 23.3 | 28.9 | 33.1 |
| Clone 7* | Bis3 | TM | — | — | PAD2 | PAD4 | 16.1 | 21.9 | 16.2 |
| Clone 8* | Bis3 | TV | — | — | PAD4 | PAD2 | 6.65 | 16.7 | 7.19 |
| Clone 8^{#} | Bis3 | TM | — | — | PAD4 | PAD2 | 7.51 | 12.1 | n.d. |
| Clone 12^{#} | Bis3 | FQQ YTE | — | — | PAD4 | PAD2 | 6.76 | 13.8 | 14.2 |

*= Antibody captured onto a C1-Protein G′ surface.
^{#}= Biotinylated Antibody captured onto a C1-Streptavidin surface. Data shown are averages of n = 2-8 experiments. n.d. = not determined.

TABLE 74
Bispecific affinity for PAD4

| | PAD4 | kₐ (M⁻¹ s⁻¹) | | k_{d} (s⁻¹) | | K_{D} (pM) | | |
|---|---|---|---|---|---|---|---|---|
| Name | species | Average | SD | Average | SD | Average | SD | n= |

1) Biotinylated PAD4 captured onto a C1-Streptavidin surface and anti-PAD antibodies flowed over

| | | | | | | | | |
|---|---|---|---|---|---|---|---|---|
| Clone 42 (Fab) | Human | 1.05E+07 | 5.52E+05 | 4.75E−04 | 4.11E−05 | 45.6 | 5.4 | 6 |
| | Cyno | 9.36E+06 | 1.81E+06 | 5.74E−04 | 3.06E−05 | 62.7 | 10.2 | 4 |
| Clone 1 | Human | 7.73E+06 | 8.94E+05 | 3.70E−04 | 2.35E−05 | 48.2 | 4.7 | 5 |
| | Cyno | 7.67E+06 | 4.81E+05 | 4.67E−04 | 4.99E−05 | 61.3 | 10.0 | 3 |
| Clone 2 | Human | 7.90E+06 | 8.75E+05 | 3.68E−04 | 3.02E−05 | 46.8 | 3.5 | 5 |
| | Cyno | 8.04E+06 | 9.95E+05 | 4.62E−04 | 3.48E−05 | 58.4 | 10.9 | 3 |
| Clone 5 | Human | 6.39E+06 | 1.29E+05 | 3.65E−04 | 2.02E−05 | 57.1 | 2.1 | 3 |
| | Cyno | 5.44E+06 | 2.22E+05 | 1.15E−03 | 6.36E−05 | 212 | 8 | 3 |
| Clone 6 | Human | 6.62E+06 | 2.79E+05 | 3.56E−04 | 1.74E−05 | 53.8 | 2.6 | 3 |
| | Cyno | 5.22E+06 | 1.13E+05 | 1.10E−03 | 3.39E−05 | 212 | 11 | 3 |

2) Antibody captured on a surface and PAD4 flowed over

| Clone 42 (IgG)* | Human | 4.94E+06 | 1.46E+05 | 3.49E−05 | 4.90E−06 | 7.08 | 1.1 | 5 |
|---|---|---|---|---|---|---|---|---|
| | Cyno | 1.41E+06 | 1.61E+05 | 3.48E−05 | 4.89E−06 | 25.2 | 6.7 | 3 |
| Clone 1* | Human | 3.78E+06 | 1.48E+05 | 1.42E−04 | 1.32E−05 | 37.5 | 2.6 | 4 |
| | Cyno | 8.87E+05 | 8.60E+04 | 2.56E−04 | 1.41E−05 | 291 | 36 | 4 |
| Clone 2* | Human | 4.19E+06 | 1.78E+05 | 1.18E−04 | 1.40E−05 | 28.2 | 3.6 | 4 |
| | Cyno | 1.12E+06 | 9.47E+04 | 2.36E−04 | 5.99E−06 | 213 | 23 | 4 |
| Clone 7* | Human | 4.30E+06 | 1.97E+05 | 3.83E−05 | 7.45E−06 | 8.92 | 1.8 | 6 |
| | Cyno | 9.99E+05 | 1.69E+05 | 3.87E−05 | 4.51E−06 | 39.3 | 6.6 | 3 |
| Clone 8* | Human | 1.89E+06 | 2.20E+05 | 4.95E−05 | 9.69E−06 | 26.3 | 5.3 | 5 |
| | Cyno | 5.40E+05 | 7.35E+04 | 1.13E−04 | 1.55E−05 | 212 | 40 | 4 |
| Clone 8^{#} | Human | 2.61E+06 | 3.03E+05 | 1.29E−04 | 9.07E−06 | 50.0 | 8.9 | 3 |
| | Cyno | n.d. | | | | | | |
| Clone 12^{#} | Human | 3.55E+06 | 6.99E+05 | 1.20E−04 | 2.86E−05 | 34.0 | 6.6 | 7 |
| | Cyno | 1.29E+06 | 7.84E+05 | 3.42E−04 | 9.29E−05 | 310 | 96 | 5 |

*= Antibody captured onto a C1-Protein G′ surface.
^{#}= Biotinylated Antibody captured onto a C1-Streptavidin. SD = Standard Deviation. n.d. = not determined.

TABLE 75
Bispecific affinity for PAD4 - Summary

| | | | | | | | K_{D} (pM) | |
|---|---|---|---|---|---|---|---|---|
| | | | Hole | Knob | | | Human | Cyno |
| Name | Format | Fc | (κ) | (λ) | scFv | Fab | PAD4 | PAD4 |

1) Biotinylated PAD4 captured onto a C1-Streptavidin surface and anti-PAD antibodies flowed over

| | | | | | | | | |
|---|---|---|---|---|---|---|---|---|
| Clone 42 | Fab | — | — | — | — | PAD4 | 45.6 | 248 |
| Clone 1 | DuetMab | TM | PAD4 | PAD2 | — | — | 48.2 | 242 |
| Clone 2 | DuetMab | TM | PAD2 | PAD4 | — | — | 46.8 | 197 |
| Clone 5 | DuetMab | FQQ YTE | PAD4 | PAD2 | — | — | 57.1 | 212 |
| Clone 6 | DuetMab | FQQ YTE | PAD2 | PAD4 | — | — | 53.8 | 212 |

2) Antibody captured on a surface and PAD4 flowed over

| | | | | | | | | |
|---|---|---|---|---|---|---|---|---|
| Clone 42* | IgG | TM | — | — | — | PAD4 | 7.08 | 25.2 |
| Clone 1* | DuetMab | TM | PAD4 | PAD2 | — | — | 37.5 | 291 |
| Clone 2* | DuetMab | TM | PAD2 | PAD4 | — | — | 28.2 | 213 |
| Clone 7* | Bis3 | TM | — | — | PAD2 | PAD4 | 8.92 | 39.3 |
| Clone 8* | Bis3 | TM | — | — | PAD4 | PAD2 | 26.3 | 212 |
| Clone 8^{#} | Bis3 | TM | — | — | PAD4 | PAD2 | 50.0 | n.d. |
| Clone 12^{#} | Bis3 | FQQ YTE | — | — | PAD4 | PAD2 | 34.0 | 310 |

*= Antibody captured onto a C1-Protein G′ surface.
^{#}= Biotinylated Antibody captured onto a C1-Streptavidin surface. Data shown are averages of n = 3-7 experiments. n.d. = not determined.

TABLE 76
Fold difference in binding affinities of bispecifics

| | | | | | | | Fold difference | | |
|---|---|---|---|---|---|---|---|---|---|
| | | | | | | | cyno/ | | mouse/ |
| | | | Hole | Knob | | | human | | human |
| Name | Format | Fc | (κ) | (λ) | scFv | Fab | PAD2 | PAD4 | PAD2 |

1) Biotinylated PAD captured onto a C1-Streptavidin surface and anti-PAD antibodies flowed over

| | | | | | | | | | |
|---|---|---|---|---|---|---|---|---|---|
| Clone 22 | Fab | — | — | — | — | PAD2 | 2.2 | n/a | 10.0 |
| Clone 42 | Fab | — | — | — | — | PAD4 | n/a | 5.4 | n/a |
| Clone 1 | DuetMab | TM | PAD4 | PAD2 | — | — | 2.0 | 5.0 | 8.6 |
| Clone 2 | DuetMab | TM | PAD2 | PAD4 | — | — | 1.6 | 4.2 | 6.3 |
| Clone 5 | DuetMab | FQQ YTE | PAD4 | PAD2 | — | — | 0.8 | 3.7 | 7.9 |
| Clone 6 | DuetMab | FQQ YTE | PAD2 | PAD4 | — | — | 1.2 | 3.9 | 6.4 |

2) Antibody captured on a surface and PAD flowed over

| | | | | | | | | | |
|---|---|---|---|---|---|---|---|---|---|
| Clone 22* | IgG | TM | — | — | — | PAD2 | 2.8 | n/a | 1.2 |
| Clone 42* | IgG | TM | — | — | — | PAD4 | n/a | 3.6 | n/a |
| Clone 1* | DuetMab | TM | PAD4 | PAD2 | — | — | 3.1 | 7.8 | 2.2 |
| Clone 2* | DuetMab | TM | PAD2 | PAD4 | — | — | 3.0 | 7.6 | 1.8 |
| Clone 7* | Bis3 | TM | — | — | PAD2 | PAD4 | 1.4 | 4.4 | 1.0 |
| Clone 8* | Bis3 | TM | — | — | PAD4 | PAD2 | 2.5 | 8.1 | 1.1 |
| Clone 12^{#} | Bis3 | FQQ YTE | — | — | PAD4 | PAD2 | 2.0 | 9.1 | 2.1 |

*= Antibody captured onto a C1-Protein G′ surface.
^{#}= Biotinylated Antibody captured onto a C1-Streptavidin surface. Data shown are averages of n = 3-7 experiments. n.d. = not determined.

### 13.4 Affinities for Different PAD2 and PAD4 Haplotypes

The affinity of the two most prevalent cynomolgus haplotypes for the Bis3 format (e.g. Clone 08) were equivalent, and approximately 2-fold lower for the least prevalent cynomolgus haplotype (Table 77 and Table 78). The affinity of all cynomolgus PAD4 haplotypes for Clone 08 and Clone 06 were within 2-fold of each other. The affinity of the two most prevalent haplotypes for both the Bis3 and DuetMab format (representative clones 02 and 08) was equivalent. The affinity of the bispecifics (both Bis3 and DuetMab) for the 3 human PAD4 haplotypes was equivalent (Table 79).

TABLE 77
Affinity of cynomolgus PAD2 haplotypes

| | Cyno PAD2 Haplotype | kₐ (M⁻¹ s⁻¹) | | k_{d} (s⁻¹) | | K_{D} (pM) | | |
|---|---|---|---|---|---|---|---|---|
| Name | (Prevalence) | Average | SD | Average | SD | Average | SD | n= |
| Clone 2 | V53M, N55D, N82S (46.9%) | 2.35E+06 | 4.48E+05 | 7.10E−05 | 8.49E−06 | 30.5 | 3.3 | 4 |
| (DuetMab) | N55D, N82S (37.5%) | 1.63E+06 | 9.50E+04 | 4.46E−05 | 8.80E−06 | 27.5 | 6.5 | 3 |
| | N55D, T63A, N82S (9.4%) | 8.96E+05 | 3.17E+05 | 4.66E−05 | 1.02E−05 | 54.7 | 12 | 3 |
| Clone 8 | V53M, N55D, N82S (46.9%) | 2.74E+06 | 4.62E+05 | 4.52E−05 | 3.72E−06 | 16.7 | 2.1 | 4 |
| (Bis3) | N55D, N82S (37.5%) | 4.23E+06 | 1.02E+06 | 2.65E−05 | 4.81E−06 | 6.40 | 1.1 | 4 |
| | N55D, T63A, N82S (9.4%) | 1.58E+06 | 3.82E+05 | 6.76E−05 | 1.02E−05 | 43.4 | 3.6 | 3 |

Antibody was captured onto a C1-Protein G′ surface and PAD2 flowed over. SD = Standard Deviation.

TABLE 78
Affinity of cynomolgus PAD4 haplotypes

| | Cyno PAD4 Haplotype | kₐ (M⁻¹ s⁻¹) | | k_{d} (s⁻¹) | | K_{D} (pM) | | |
|---|---|---|---|---|---|---|---|---|
| Name | (Prevalence) | Average | SD | Average | SD | Average | SD | n= |
| Clone 2 | S118C, R617C (40.6%) | 1.12E+06 | 9.47E+04 | 2.36E−04 | 5.99E−06 | 213 | 23 | 4 |
| (DuetMab) | S118C (28.1%) | 7.16E+05 | 9.65E+04 | 2.61E−04 | 3.10E−05 | 366 | 11 | 6 |
| | S118C, R196K, R617C (12.5%) | 5.22E+05 | 3.37E+04 | 1.87E−04 | 1.51E−05 | 358 | 22 | 7 |
| | REF (6.25%) | 5.70E+05 | 8.06E+04 | 2.14E−04 | 1.92E−05 | 379 | 28 | 7 |
| Clone 8 | S118C, R617C (40.6%) | 5.40E+05 | 7.35E+04 | 1.13E−04 | 1.55E−05 | 212 | 40 | 4 |
| (Bis3) | S118C (28.1%) | 6.29E+05 | 9.89E+04 | 1.55E−04 | 2.61E−05 | 257 | 84 | 5 |
| | S118C, R196K, R617C (12.5%) | 5.89E+05 | 2.46E+05 | 1.83E−04 | 4.88E−05 | 324 | 41 | 5 |
| | REF (6.25%) | 4.02E+05 | 3.51E+04 | 1.63E−04 | 1.23E−05 | 406 | 19 | 4 |

Antibody was captured onto a C1-Protein G′ surface and PAD4 flowed over. SD = Standard Deviation.

TABLE 79
Affinity of Human PAD4 haplotypes

| | Human PAD4 Haplotype | kₐ (M⁻¹ s⁻¹) | | k_{d} (s⁻¹) | | K_{D} (pM) | | |
|---|---|---|---|---|---|---|---|---|
| Name | (Prevalence) | Average | SD | Average | SD | Average | SD | n= |
| Clone 2 | G55S, V82A, G112A (40%) | 4.61E+06 | 5.62E+05 | 1.29E−04 | 1.77E−05 | 28.6 | 6.0 | 8 |
| (DuetMab) | REF (39%) | 4.19E+06 | 1.78E+05 | 1.18E−04 | 1.40E−05 | 28.2 | 3.6 | 4 |
| | G55G, V82A, G112A, | 4.51E+06 | 3.31E+05 | 1.16E−04 | 1.94E−05 | 25.7 | 2.9 | 4 |
| | D260N (0.4%) | | | | | | | |
| Clone 8 | G55S, V82A, G112A (40%) | 1.65E+06 | 7.94E+04 | 9.66E−05 | 7.60E−06 | 58.6 | 5.1 | 8 |
| (Bis3) | REF (39%) | 1.39E+06 | 5.33E+04 | 8.08E−05 | 8.54E−06 | 58.1 | 4.4 | 4 |
| | G55G, V82A, G112A, | 1.55E+06 | 1.62E+05 | 8.62E−05 | 8.43E−06 | 55.7 | 5.8 | 4 |
| | D260N (0.4%) | | | | | | | |

Antibody was captured onto a C1-Protein G′ surface and PAD4 flowed over. SD = Standard Deviation.

### 13.5 Affinities of Mouse Surrogate Anti-PAD4

The affinities of the mouse surrogate anti-PAD4 are shown in Table 80.

TABLE 80
Affinity of mouse surrogate anti-PAD4 antibodies

| | PAD4 | kₐ (M⁻¹ s⁻¹) | | k_{d} (s⁻¹) | | K_{D} (pM) | | |
|---|---|---|---|---|---|---|---|---|
| Name | species | Average | SD | Average | SD | Average | SD | n= |
| PAD40071 | Mouse | 7.30E+05 | 3.09E+03 | 1.63E−01 | 3.09E−03 | 223 | 5.2 | 2 |
| AB1630204 | Mouse | 2.39E+05 | 1.04E+04 | 3.14E−04 | 1.13E−05 | 1.31 | 0.09 | 3 |
| AB1630205 | Mouse | 1.14E+06 | 1.03E+05 | 1.06E−04 | 1.83E−05 | 0.094 | 0.02 | 8 |

Biotinylated antibody was captured onto a C1-Streptavidin surface and PAD4 flowed over. SD = Standard Deviation.

### 14 EXAMPLE 7: POTENCY

### 14.1 Potency of Prior Art Anti-PAD4 Antibodies

The anti-PAD4 antibodies described in WO2012026309A9 [9] (L78-4, L119-5, L198-3 and L207-11) only showed 10-40% inhibition of PAD activity at an antibody concentrations of 1000 nM.

WO 2016/155745 A1 suggests mouse monoclonal antibodies that are cross-reactive for PAD2, PAD4 and PAD3. The cross-reactive antibodies were shown to be only capable of inhibiting PAD2 activity by a maximum of approximately 25% compared to a control antibody, as measured by using a fibrogen citrullination assay.

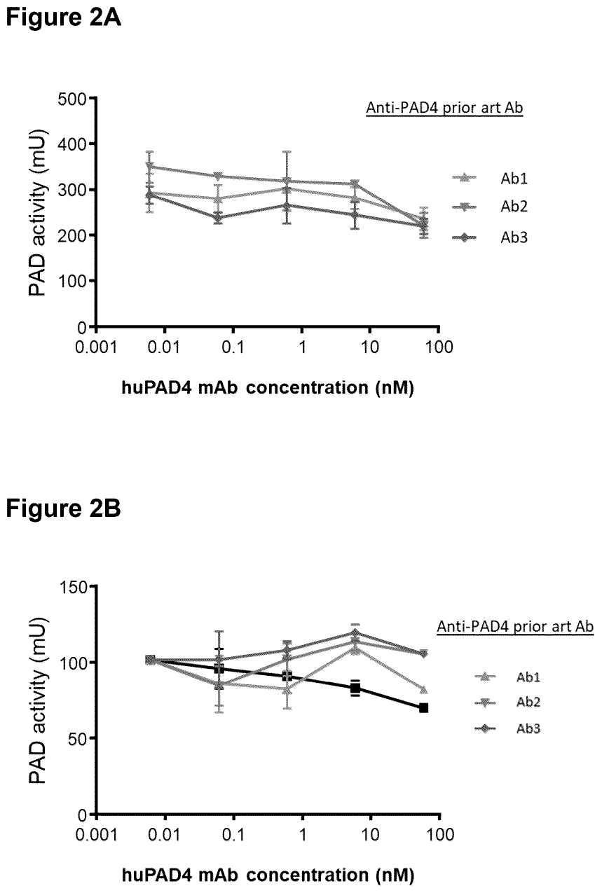

Previously described humanised anti-PAD4 antibodies showed little to no inhibition of PAD4 activity in synovial fluid (FIG. 2), as assessed by H3 histone citrullination assay (FIG. 2A) or BAEE PAD activity assay (Cayman Chemical) (FIG. 2B).

### 14.2 Potency of Anti-PAD4 and Anti-PAD2 Antibodies

Using a PAD4 histone H3 citrullination potency assay, it initially proved challenging to discriminate between the potency of the generated anti-PAD4 antibodies and the parent antibody described in Example 3. It was noticed that using the assay, the IgGs stacked with IC₅₀ values that were close to the concentration of PAD4 in the system (i.e. 50 ng/ml, 0.65 nM).

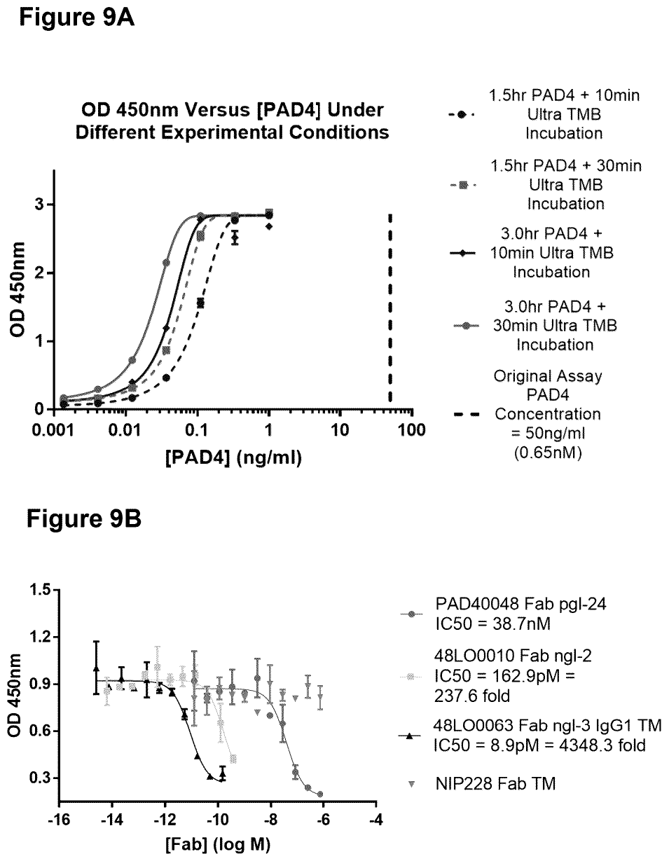

It was found that greatly reducing the concentration of PAD4 used in the assay (from 50 ng/ml to 15 pg/ml, ×3,333 fold reduction), improved the assay so that differences in potency could be more easily assessed. Reducing the concentration of the PAD4 used in the assay required optimisation of other parameters in the assay, in particular extending the PAD4 enzyme reaction incubation time and using ultra-TMB HRP detection (FIG. 9A). Under these revised conditions, the affinity optimised IgGs showed improved potency relative to the parent. However, assay stacking was then observed in the low 100's fM range. The inventors hypothesised that this was due to bivalent binding of the antibodies to PAD4 homodimer. This hypothesis was confirmed by testing the potency of the antibodies as Fab fragments (FIG. 9A) (see also Section 9, EXAMPLE 4).

Using the improved potency assay and testing the antibodies as Fab fragment, the improved potency of the affinity optimised anti-PAD4 antibodies could be observed (Table 81).

TABLE 81
Potency of anti-PAD4 antibodies

| | | | Fold potency increase |
|---|---|---|---|
| Clone | IC₅₀ | | from parent |
| PAD40048 | 38.7 | nM | — |
| 48LO0010 Fab ngl-2 | 162.9 | pM | 237.6 |
| 48LO0063 Fab ngl-3 IgG1 TM | 8.9 | pM | 4348.3 |

Anti-PAD4 Fab in Histone H3 Assay, 3 hr 45 min PAD4 incubation, concentration of PAD4: 0.15 ng/ml

### 14.3 PAD2 & PAD4 Activity in RA Synovial Fluid

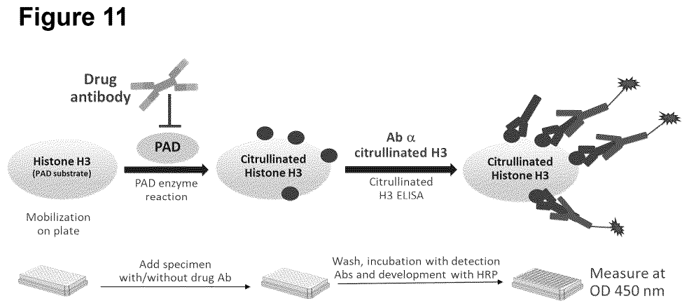

Potency of anti-PAD2 and anti-PAD4 antibodies can be assessed using a Histon-H3 activity assay for PAD activity in, for example, synovial fluid or whole blood from RA patients (FIG. 11). PAD2 and PAD4 both contribute to PAD activity (as determined by Histon-H3 activity assay) in synovial fluid from RA patients (Table 82).

TABLE 82
PAD2 and PAD4 levels in RA synovial fluid

| RA Synovial Fluid | PAD4 (ng/ml) | PAD2 (ng/ml) |
|---|---|---|
| Sample 1 | 2159 | 1943 |
| Sample 2 | 298 | 21 |
| Sample 3 | 719 | 1231 |
| Sample 4 | 3 | 46 |
| Sample 5 | 208 | 41 |

PAD4 and PAD2 levels were determined using Cayman ELISA kit.

### 14.4 PAD2 & PAD4 Activity in Whole Blood of RA Patients

PAD2 and PAD4 can also be detected in the whole blood of RA patients at varying concentrations (Table 83).

TABLE 83
PAD levels in whole blood from RA patients

| RA sample | CCP status | RF status | PAD4 (ng/ml) | PAD2 (ng/ml) |
|---|---|---|---|---|
| Sample 1 | − | − | 4.2 | 0.7 |
| Sample 2 | − | − | 13.7 | 1.3 |
| Sample 3 | + | + | 78 | 60 |

### 14.5 Potency Against Recombinant PAD

The potency of the DuetMab and Bis3 bispecifics were directly compared using the optimised Histone-H3 citrullination ELISA (see Section 12.2) and recombinant PAD2 and PAD4 (Table 84). In DuetMab format, the orientation did not affect potency (e.g. Clone 01 versus Clone 02).

The IC₅₀ of all the bispecifics formats and the IgG Clone 22 to inhibit histone citrullination of recombinant PAD2 was lower than that reported for prior art anti-PAD2 antibodies [13]. Particularly, Aosasa et al. reported the potency of anti-PAD2 antibodies (S4, S10, S24, S108, S170 and S309) at inhibiting recombinant PAD2 histone citrullination as being in the range of 7.0-75 nM [27]. By contrast the potency (IC₅₀) of the DuetMabs, Bis3 and Clone 22 against recombinant PAD2 in an equivalent assay were all less than 1 nM (Table 84).

TABLE 84
Potency of bispecifics (recombinant PAD2/PAD4)

| | | | | | | Potency | |
|---|---|---|---|---|---|---|---|
| | | Hole | Knob | | | (IC₅₀) (pM) | |
| Name | Format | (κ) | (λ) | scFv | Fab | PAD2 | PAD4 |
| Clone 01 | DuetMab | PAD4 | PAD2 | — | — | 533 | 91 |
| Clone 02 | DuetMab | PAD2 | PAD4 | — | — | 630 | 89 |
| Clone 07 | Bis3 | — | — | PAD2 | PAD4 | 238 | 10 |
| Clone 08 | Bis3 | — | — | PAD4 | PAD2 | 94 | 25 |
| Clone 22 | IgG | — | — | — | — | 138 | — |
| Clone 42 | IgG | — | — | — | — | — | 8 |

Histone-H3 assay. Human PAD4 (RD223) used at 0.15 ng/mL; Human PAD2 (RD220) used at 0.02 ng/mL; 30 minutes pre-incubation of PAD and Bispecific; 3 hour incubation

In DuetMab format, there was a 3-4-fold PAD2 potency loss versus the optimised anti-PAD2 lead in IgG format (Clone 22) and a 7-11-fold PAD4 potency loss versus the optimised anti-PAD4 lead in IgG format (Clone 42) (Table 84). There was either no meaningful potency loss or a potency increase for PAD2 and PAD4 for the Bis3 format (Table 84). The PAD2-Fab Bis3 (e.g. Clone 08) performed better than the PAD4 Fab Bis3 (e.g. Clone 07) (Table 84).

### 14.6 Potency Against Pad Activity in Synovial Fluid of Ra Patients

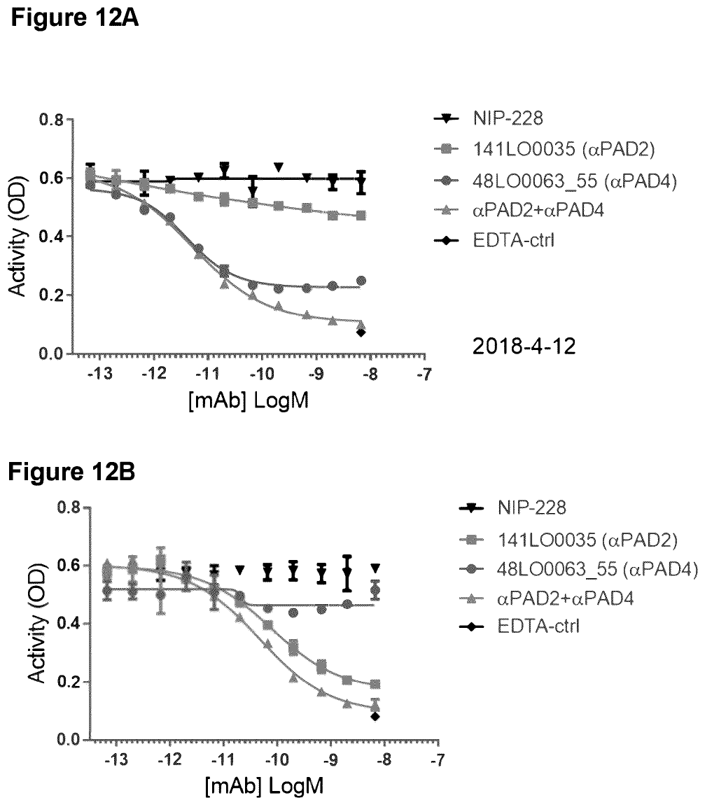

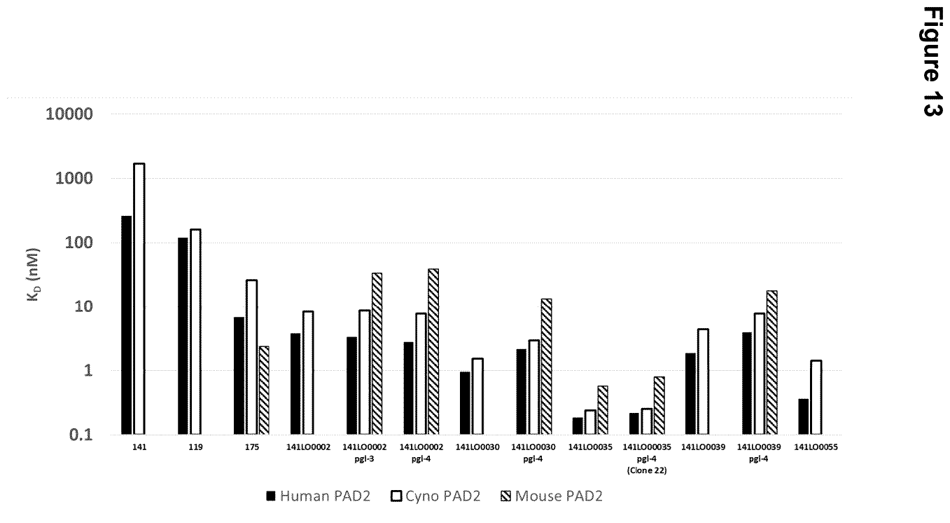

The anti-PAD2 and PAD4 Abs are effector null (TM) antibodies were shown to be effective at blocking extracellular PAD activity. Surprisingly, blocking PAD2 and PAD4 in the synovial fluid of RA patients showed that PAD2 and PAD4 activity were non-redundant. Blocking PAD2 and PAD4 with an anti-PAD2 and anti-PAD4 was shown to inhibit all PAD activity in RA synovial fluid (FIG. 12).

Potency analysis of DuetMabs in both orientations showed that the bispecifics inhibited PAD (PAD2 and PAD4 combined) activity in synovial fluid (Table 85).

TABLE 85
DuetMab potency (Synovial fluid)

| ID | Description | Potency pM (CI95) | |
|---|---|---|---|
| 48LO0063/141LO0035 | anti-PAD2/anti-PAD4 | 6.2 | (4.1-8.7) |
| PAD240002 | PAD2 (hole) - | 130 | (79-200) |
| | PAD4 (knob) | | |
| PAD240001 | PAD4 (hole) - | 88 | (49-155) |
| | PAD2 (knob) | | |

Histon-H3 activity assay, synovial fluid, 1:2000 dilution; CI95: 95% Confidence Interval

Potency analysis of Bis-3 Ab formats in two orientations showed that the bispecifics inhibited combined PAD2 and PAD4 activity in synovial fluid (Table 86). The ability of the Bis3 bispecifics to inhibit combined PAD activity in synovial fluid from RA patients was high, and dose dependent.

Surprisingly, the “scFv (PAD4)-Fab(PAD2)” Bis3 format (Clones 08, 10 and 12) performed better than a combination of the optimised anti-PAD2 (Clone 22) and anti-PAD4 (Clone 42) antibodies (Table 87).

TABLE 86
Bis3 potency (synovial fluid)

| | | | | Potency (pM) | |
|---|---|---|---|---|---|
| Name | Format | scFv | Fab | (CI95) | |
| PAD240007 | Bis3 | PAD2 | PAD4 | 21 | (19-24) |
| PAD240008 | Bis3 | PAD4 | PAD2 | 5 | (1.9-8.5) |
| PAD240009 | Bis3 | PAD2 | PAD4 | 30 | (24-37) |
| PAD240010 | Bis3 | PAD4 | PAD2 | 8 | (6-11) |
| PAD240011 | Bis3 | PAD2 | PAD4 | 26 | (21-31) |
| PAD240012 | Bis3 | PAD4 | PAD2 | 10 | (8.4-11.7) |

| | | |
|---|---|---|
| 141LO0035 & | IgG | 16 |
| 48LO0063 | | |

Histon-H3 activity assay, synovial fluid, 1:2000 dilution, CI95: 95% Confidence Interval

TABLE 87
Comparative Bis3 potency (synovial fluid)

| Format | Fab | Potency (pM) |
|---|---|---|
| Bis3 | PAD4 | 21-30 |
| Bis3 | PAD2 | 5-8 |

### 14.7 Potency Against PAD Activity in Whole Blood

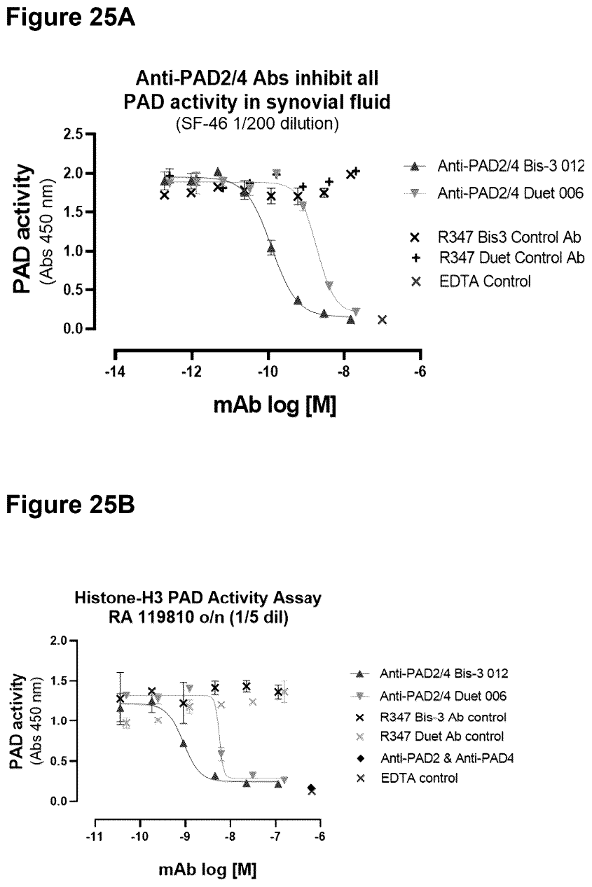

Potency of the bispecifics was also assessed in plasma samples from whole blood. The Bis3 format was more potent in this assay than the DuetMab format (Clone 12, PAD240012) (FIG. 25B). Both the Duet and Bis3 format bispecifics were able to fully inhibit PAD activity in both synovial fluid and whole blood (FIG. 25).

TABLE 88
Potency of bispecifics (whole blood)

| | Bis-3 (Clone 12) | Duet (Clone 06) | Fold |
|---|---|---|---|
| RA sample | IC₅₀ pM | IC₅₀ pM | difference |
| RA 20913 | 837 | 3788 | x 4.5 |
| RA 32310 | 475 | 5675 | X 11.9 |
| RA 119810 | 900 | 5610 | X 6.2 |
| Mean ± SE | 737± | 5024± | 7.5 ± 2.2 |

Histone-H3 PAD activity assay, Antibodies were incubated overnight in RA whole blood

### 14.8 Potency In Vivo

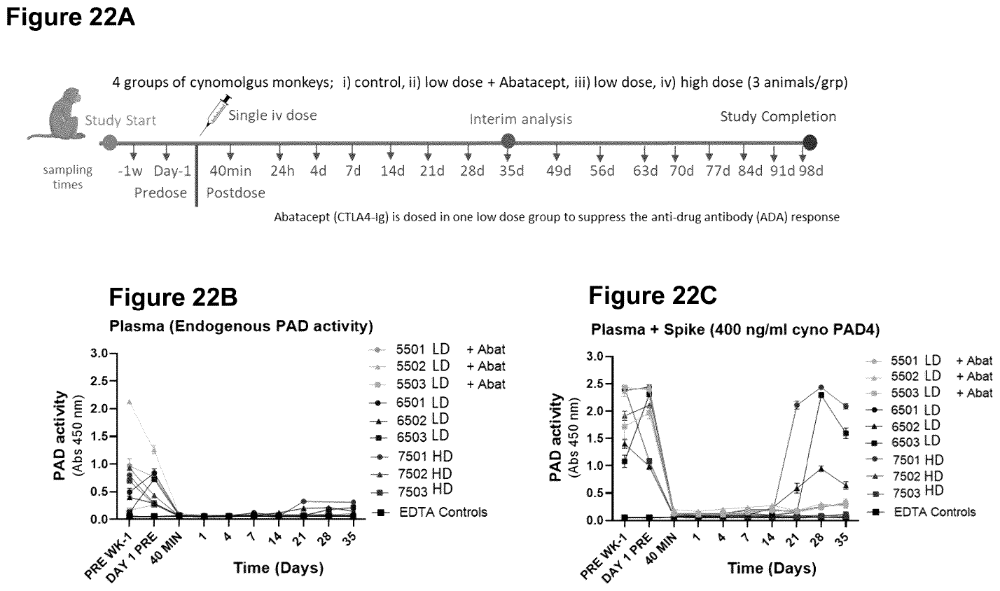

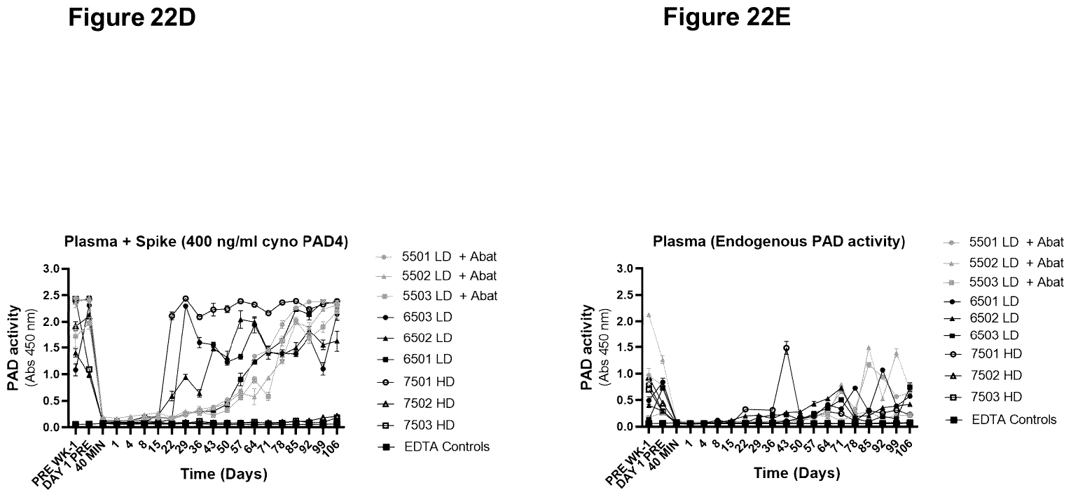

The in vivo potency of Bis3 PAD2/PAD4 bispecifics at low and high doses was assessed (FIG. 22A). The Bis3 bispecific was able to suppress endogenous PAD activity in vivo, with rapid target engagement, and almost complete target engagement through day 57 with a low dose of the Bis3 bispecific with the PAD activity returning to the pre-dose levels by approximately Day 85. No detectable return of PAD activity was observed at study completion (Day 106) at high dose (FIG. 22C, FIG. 22E).

The Bis3 bispecific was able to suppress PAD activity in spiked plasma with rapid and almost complete target engagement through day 29 with a low dose of the Bis3 bispecific, with the PAD activity returning to pre-dose levels by approximately Day 85. No detectable return of PAD activity was observed at study completion (Day 106) at high dose (FIG. 22B, FIG. 22D).

### 14.9 Potency Against Different Haplotypes

The Bis3 format retained similar potency (within 2-fold) against haplotype of human and cynomolgus PAD4, as well as cynomolgus PAD2 (Table 89).

TABLE 89
Bis3 potency (IC₅₀) against PAD2 and PAD4 haplotypes

| PAD Haplotype | | | | | |
|---|---|---|---|---|---|
| Species | | | | | |
| (Overall Population | Assay Conc | Haplotype | | | |
| coverage %) | (ng/mL) | (Prevalence) | Bis3 | PAD2 IgG | PAD4 IgG |
| Human PAD4 | 0.3 | REF (39%) | 36 | — | 6.8 |
| (79.4%) | 0.3 | 55G > S, 82V > A, 112G > A, 260D > N | 22 | — | 9 |
| | | (0.4%) | | | |
| | 0.3 | 55G > S, 82V > A, 112G > A (40%) | 58 | — | 19 |
| Human PAD2 | 0.05 | REF (96.8%) | 127 | 184 | — |
| (96.8%) | | | | | |
| Cyno PAD4 | 0.8 | 118S > C, 617R > C (40.6% Chinese) | 32 | — | 10 |
| (87.5%) | 0.8 | 118S > C (28.1% Chinese) | 50 | — | 15 |
| | 0.8 | REF (6.25% Chinese) | 35 | — | 13 |
| | 0.8 | 118S > C, 196R > K, 617R > C | 43 | — | 15 |
| | | (12.5% Chinese) | | | |
| Cyno PAD2 | 0.15 | 53V > M, 55N > D, 82N > S (46.9% | 295 | 357 | — |
| (93.8%) | | Chinese) | | | |
| | 0.15 | 55N > D, 82N > S (37.5% Chinese) | 329 | 303 | — |
| | 0.15 | 55N > D, 63T > A, 82N > S (9.4% | 222 | 363 | — |
| | | Chinese) | | | |
| Mouse PAD2 | 0.1 | REF | 74 | 136 | — |
| (100%) | | | | | |

Bis3 = scFv (PAD4)-Fab(PAD2), Clone 12

### 14.10 Potency of Anti-PAD2 and Anti-PAD4 Antibodies

The following antibodies described in the art were cloned, expressed, and purified: anti-PAD2 antibody mAb2 [10]; anti-PD4 antibodies G8H4 and H7H4 [12]; and 4R147 [38].

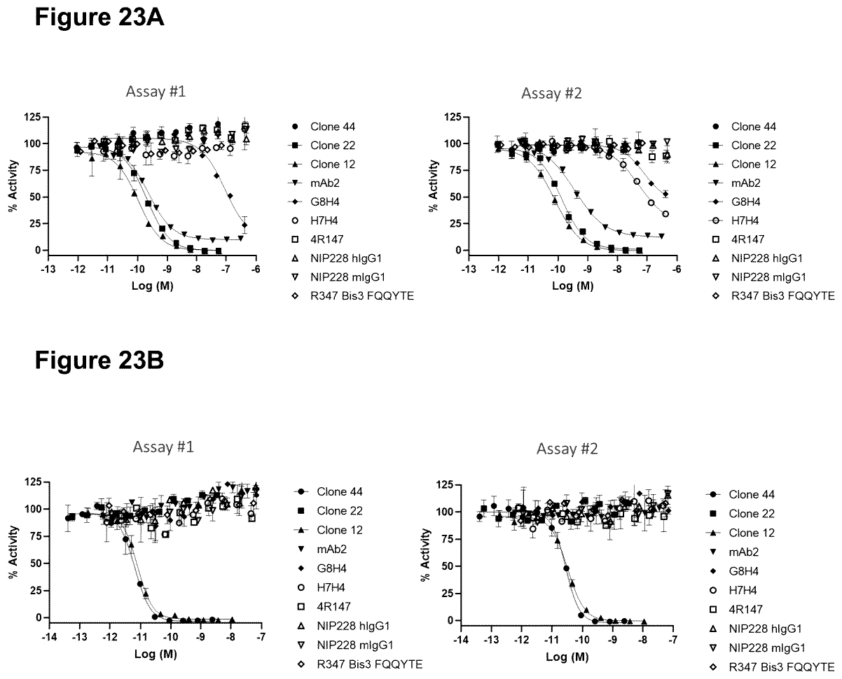

The potency of clones 12, 22 and 42 was directly compared to mAb2, G8H4, H7H4 and 4R147 using the optimised Histone-H3 citrullination ELISA (see Section 12.2) and recombinant PAD2 and PAD4 (FIG. 23A and FIG. 23B, respectively).

Clones 12 and 22 completely inhibited hPAD2 enzymatic activity, whereas anti-PAD2 antibody mAb2 only partially inhibited said activity. Clones 12 and 44 completely inhibited hPAD4 enzymatic activity, whereas the anti-PAD4 antibodies G8H4, H7H4 and 4R147 did not show any inhibitory activity against hPAD4 enzymatic activity up to 50 nM.

Similarly, the potency of clone 12 was directly compared to mAb2, G8H4, H7H4 and 4R147 using the optimised Histone-H3 citrullination ELISA in synovial fluid (FIG. 26). Clone 12 completely inhibited PAD enzymatic activity, whereas none of the other antibodies showed any inhibitory activity.

### 15 EXAMPLE 8: SPECIFICITY

WO 2016/155745 A1 [11] suggests monoclonal antibodies that are cross-reactive for PAD2, PAD4 and PAD3.

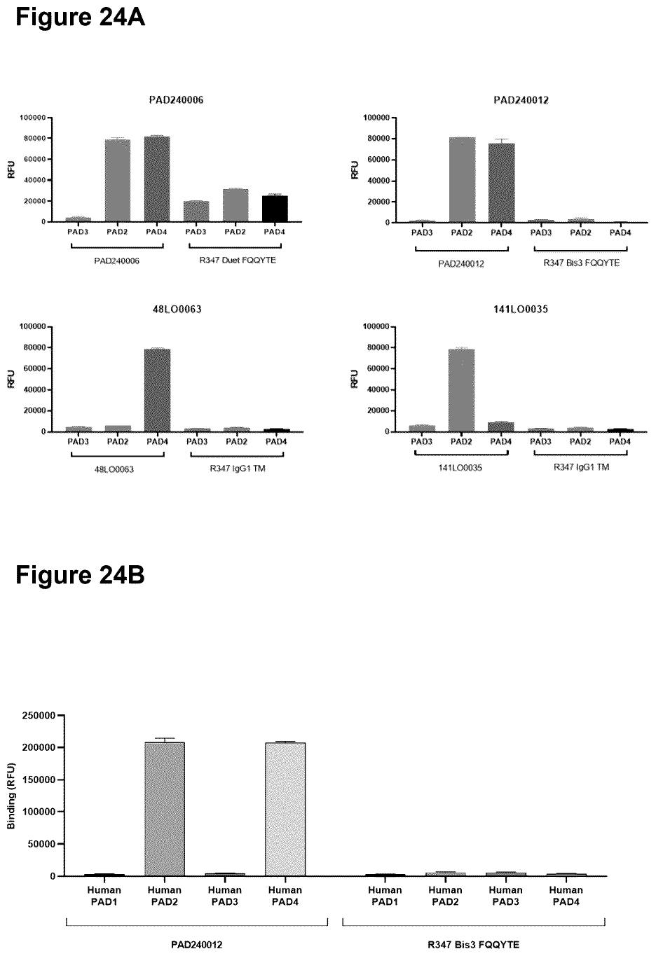

The affinity optimised anti-PAD4 antibodies and bispecific formats were specific for PAD2 and/or PAD4, and did not bind PAD3 (FIG. 24A) or PAD1 (FIG. 24B), as assessed by a specificity ELISA assay (Section 6.2).

### 16 EXAMPLE 9: BINDING BEHAVIOUR OF BISPECIFICS

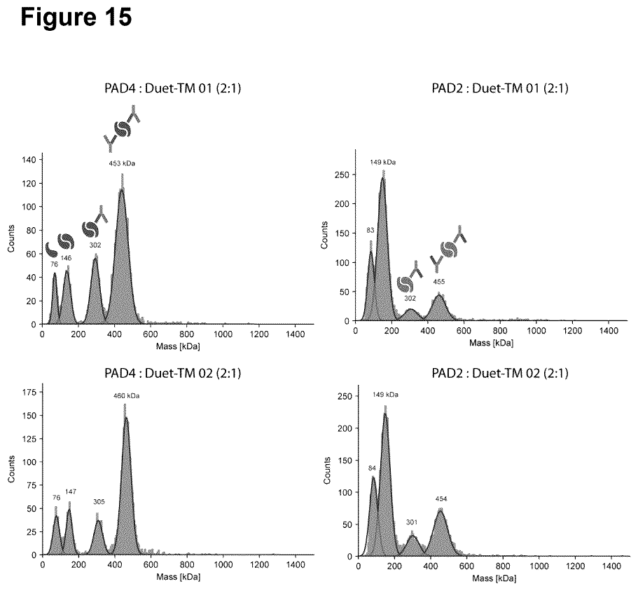

The binding properties of both DuetMab orientations (PAD2λ/PAD4κ and PAD4λ/PAD2κ) were assessed. Both of the DuetMab orientations had similar binding behaviours. Similar to PAD4 IgG and Fab (FIG. 5, FIG. 8), the Duets also stabilised the PAD4 dimer (FIG. 15). In contrast, but similar to PAD2 IgG and Fab (FIG. 7, FIG. 8), binding of the DuetMabs to PAD2, suggesting a different epitope location for PAD2 (FIG. 15). Constant region modifications (YTE, TM, FQQ) had no effect on binding properties of the DuetMabs.

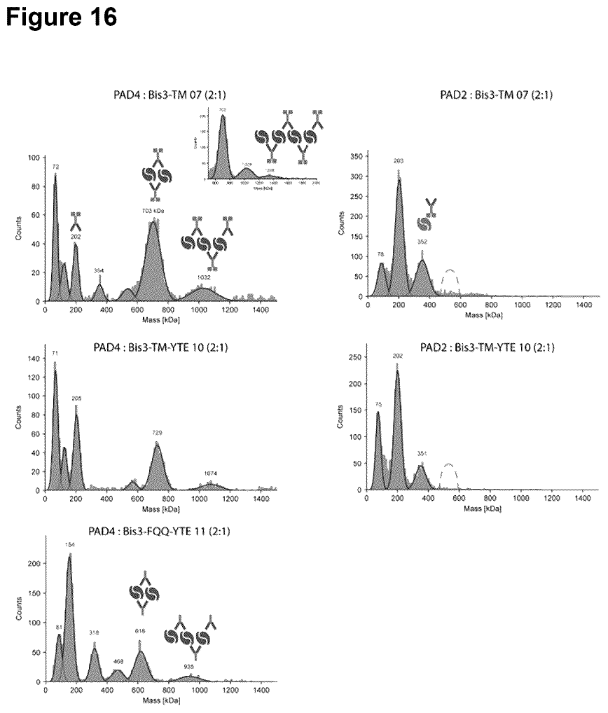

All the Bis3 orientations had similar binding behaviours to PAD2 and PAD4 (FIG. 16) as assessed by iSCAT (Section 6.3). Similar to PAD4 IgG, the Bis3 format favoured the highly stable hexameric complex, i.e. 2× Bis3+2×PAD4 dimers. The Fc mutations had no effect on binding behaviour (FIG. 16). In contrast, but similar to PAD2 IgG, the Bis3 does not have on effect on the PAD2 dimer state. In addition, steric hindrance seems to block the formation of the tetramer (FIG. 16, dotted lines), as previously observed for PAD2 IgG but in contrast to the DuetMabs (FIG. 15).

iSCAT data showed that fibrillation was possible with the Bis3 formats but was absent for DuetMabs (FIG. 15 and FIG. 16). These data show that the Bis3 format with PAD2 arm in Fab format had less propensity to form high complexes (FIG. 16, “PAD2 Bis-TM-YTE 10”), and therefore the Bis3 format with PAD2 arm in Fab has a lower aggregation risk. As expected, the Fc modifications had no effect on binding activity (FIG. 15; FIG. 16, e.g. compare PAD2 Bis-TM-YTE 07 and PAD2 Bis-TM-YTE 11).

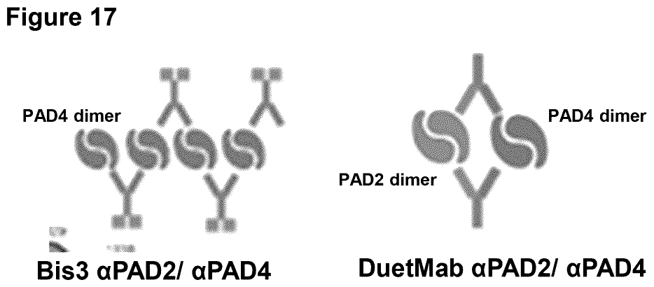

In the presence of PAD4 alone, Bis3 constructs but not Duets enabled formation of the stable hexamer, but also higher order complexes (FIG. 15 and FIG. 16). In contrast, in the presence of PAD2 alone, no hexamer or higher order complexes are observed. Therefore, the dual PAD4 interaction is the main driver of oligomers including the highly stable hexamer and potentially higher order complexes, suggesting that “fibrillation” is possible. In the presence of both PAD4 and PAD2, the DuetMabs were also able to form the stable hexamer but no higher order complexes were detected (FIG. 15 and FIG. 16). The dual interaction seen with the Bis3 format is not possible with the DuetMab format (FIG. 17) and hence, no higher order complexes is observed (Table 90).

TABLE 90
Higher complex formation potential

| | | Fc | Higher complex |
|---|---|---|---|
| Ref. | Format | modification | formation potential |
| Clone 01 | Duet | TM | No |
| Clone 02 | Duet | TM | No |
| Clone 03 | Duet | TM-YTE | No |
| Clone 04 | Duet | TM-YTE | No |
| Clone 05 | Duet | FQQ-YTE | No |
| Clone 06 | Duet | FQQ-YTE | No |
| Clone 07 | Bis3 | TM | Yes |
| Clone 08 | Bis3 | TM | Yes |
| Cone 09 | Bis3 | TM-YTE | Yes |
| Clone 10 | Bis3 | TM-YTE | Yes |
| Clone 11 | Bis3 | FQQ-YTE | Yes |
| Clone 12 | Bis3 | FQQ-YTE | Yes |

### 17 EXAMPLE 10: STABILITY

### 17.1 Thermostability

Aggregation is closely related to the thermal stability of antibody molecules. The lower the thermal stability, the less stable the product, and the higher degree of aggregation, whereas the higher thermal stability of the product can reduce the degree of aggregation. Promethus Nano differential scanning fluorimetry (Nano-DSF) was used to analyse thermal stability, using standard protocols.

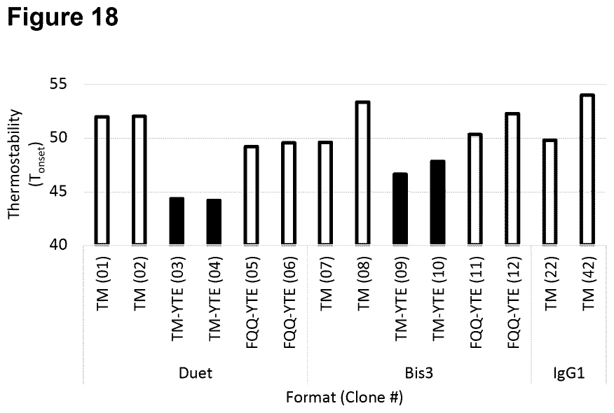

The YTE Fc mutation (M252Y/S254T/T256E) extends the half-life of antibody molecules. The triple mutation (TM, L234F/L235E/P331S) attenuates effector function of the constant region. TM and the YTE mutation were introduced to the Bis3 and DuetMab bispecifics, the sequences of which are provided in Table 57 and Table 58. Tₒₙₛₑₜ stability data demonstrated that the TM-YTE Fc mutation was instable compared to bispecifics with the TM modification alone, for both the Bis3 and DuetMab formats (Table 91, Clones 09, 10, 03 and 04). An alternative effector null modification (FQQ, L234F/L235Q/K322Q) did not demonstrate the same instability, regardless of bispecific format (Clones 05, 06, 11 and 12) (FIG. 18).

The highest onset temperature (Tₒₙₛₑₜ) was seen for the TM and FQQ-YTE DuetMab and Bis3 formats (Table 91). Clones 01, 02, 05, 06, 07, 08, 11 and 12 all had a Tₒₙₛₑₜ of greater than about 48° C. (FIG. 18, dotted-line).

TABLE 91
Thermal stability of bispecifics

| | | Fc | NanoDSF |
|---|---|---|---|
| Clone ID | Format | Modification | (Tₒₙₛₑₜ) (° C.) |
| PAD240001 | Duet | TM | 51.97 |
| PAD240002 | Duet | TM | 52.01 |
| PAD240003 | Duet | TM-YTE | 44.32 |
| PAD240004 | Duet | TM-YTE | 44.13 |
| PAD240005 | Duet | FQQ-YTE | 49.16 |
| PAD240006 | Duet | FQQ-YTE | 49.49 |
| PAD240007 | Bis3 | TM | 49.55 |
| PAD240008 | Bis3 | TM | 53.35 |
| PAD240009 | Bis3 | TM-YTE | 46.59 |
| PAD240010 | Bis3 | TM-YTE | 47.79 |
| PAD240011 | Bis3 | FQQ-YTE | 50.29 |
| PAD240012 | Bis3 | FQQ-YTE | 52.27 |
| 141LO0035 pgl-3 | IgG1 | TM | 49.74 |
| 48LO0063 fgl-55 | IgG1 | TM | 53.98 |

### 17.2 Aggregation

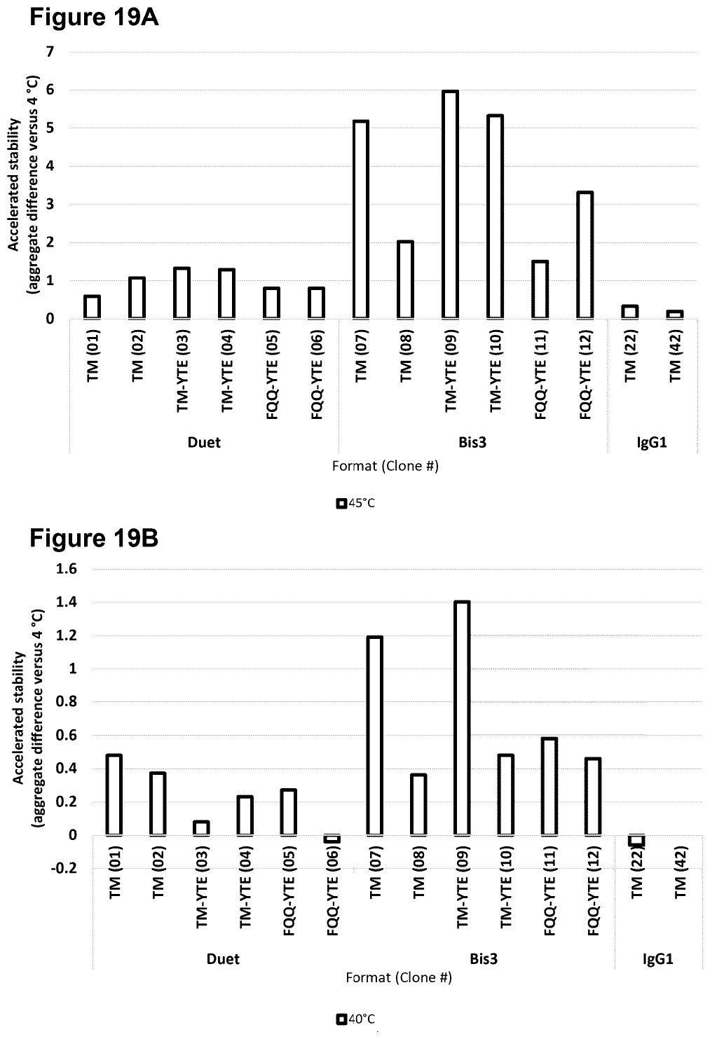

An accelerated stability study using HP-SEC compared the stability at 40° C. and 45° C. incubation to 4ºC (Table 92, FIG. 19). Surprisingly, the DuetMab formats showed higher stability at both 45° C. than all Bis3 formats. In the context of a TM Fc modification, the Bis3 Clone 09 format showed the lower aggregation at 45° C. In the context of a TM-YTE modification, the Bis3 showed the highest aggregation, regardless of Bis3 orientation at 45° C. In the context of a FQQ-YTE Fc modification, the Clone 11 Bis3 format showed the least aggregation at 45° C.

Surprisingly, at 40° C. the DuetMab format showed the least aggregation in the context of a TM-YTE or FQQ-YTE Fc modification, regardless of DuetMab orientation. In the context of an FQQ-YTE Fc modification, the PAD2(hole)-PAD4(knob) orientation showed comparable aggregation with the respective IgGs (TM).

For the Bis3 format, there was acceptable aggregation in the context of an FQQ-YTE modification, regardless of Bis3 orientation. The PAD2-Fab/PAD4-scFv (Clones 08, 10 and 12) Bis3 format showed the least aggregation regardless of Fc modification.

TABLE 92
Accelerated stability

| | | | Accelerated stability | |
|---|---|---|---|---|
| | | | (aggregate difference) | |
| Ref. | Format | Fc modification | 45° C. | 40° C. |
| Clone 01 | Duet | TM | 0.58 | 0.48 |
| Clone 02 | Duet | TM | 1.07 | 0.37 |
| Clone 03 | Duet | TM-YTE | 1.32 | 0.08 |
| Clone 04 | Duet | TM-YTE | 1.29 | 0.23 |
| Clone 05 | Duet | FQQ-YTE | 0.79 | 0.27 |
| Clone 06 | Duet | FQQ-YTE | 0.79 | −0.04 |
| Clone 07 | Bis3 | TM | 5.17 | 1.19 |
| Clone 08 | Bis3 | TM | 2.01 | 0.36 |
| Cone 09 | Bis3 | TM-YTE | 5.95 | 1.4 |
| Clone 10 | Bis3 | TM-YTE | 5.31 | 0.48 |
| Clone 11 | Bis3 | FQQ-YTE | 1.5 | 0.58 |
| Clone 12 | Bis3 | FQQ-YTE | 3.3 | 0.46 |
| 1411LO0035 pgl-3 | IgG1 | TM | 0.32 | −0.06 |
| 48LO0063 fgl-55 | IgG1 | TM | 0.18 | 0 |
| ELK90084 | Bis3 | P | 1.2 | 6.04 |
| Anti-CCR9 | Duet | None | 0 | 0 |
| NIP228005 | IgG1 | TM | 0 | 0 |

2 week study, concentration of antibody ~1 mg/ml

### 17.3 Stability Conclusions

The stability data surprisingly showed that the bispecific formats with a TM-YTE Fc modification demonstrated a drop in thermostability that was not observed in bispecifics with TM or FQQ-TM modifications (FIG. 18). The stability data further showed that the DuetMab format was surprisingly less prone to aggregation than the Bis3 format, regardless of DuetMab or Bis3 orientation of Fc modifications. Furthermore, the PAD2-Fab/PAD4-scFv (Clones 08, 10 and 12) Bis3 format showed the least aggregation regardless of Fc modification.

### 18 EXAMPLE 11: SAFETY

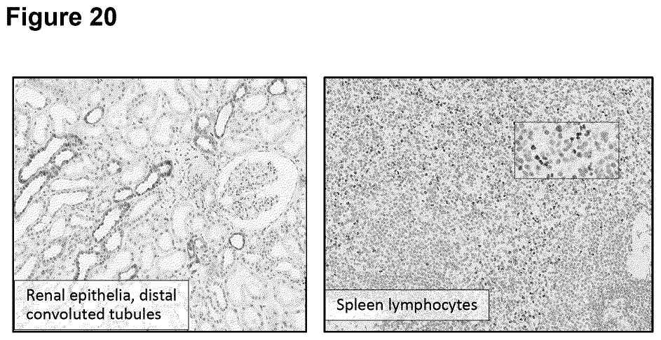

The Bis3 format was well tolerated. In a cynomolgus study, PAD2/4 were shown to be expressed intracellularly and on the nuclear membrane of tissue immune cells. There were no findings of concern (FIG. 20). PAD2 was detected in the CNS, but there were no effects in enhanced behaviour observations in the cynomolgus study. There were further no effects (e.g. adverse cytokine release) observed in healthy and RA donor blood (FIG. 20).

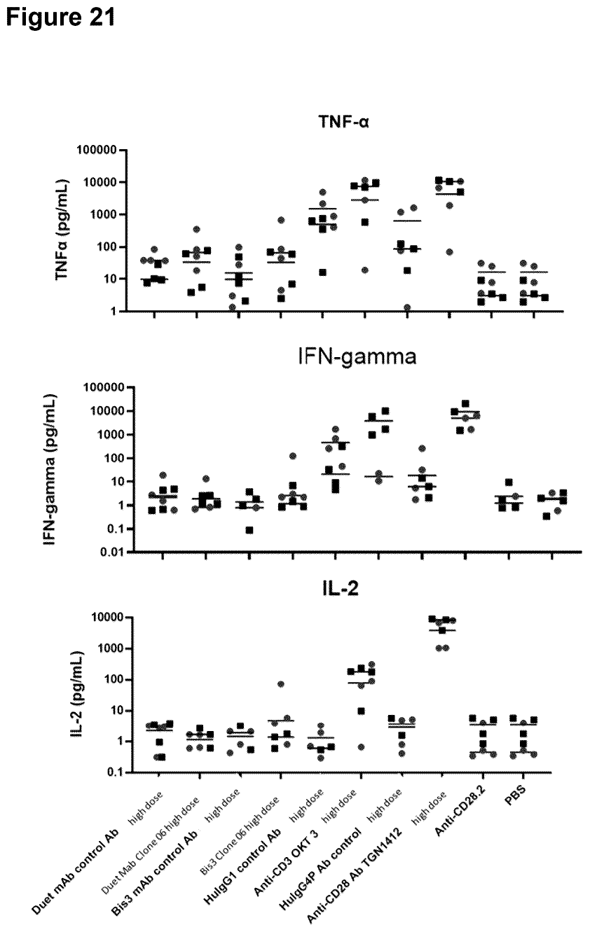

There were further no safety concerns (e.g. adverse cytokine release) observed in healthy and RA donor blood following exposure to the Bis3 or DuetMab formats (Clone 12 and Clone 06) (FIG. 21). None of the test samples had measured cytokine levels above the upper limit of quantitation of 20000.0 pg/ml.

### 19 EXAMPLE 12: EFFECTOR NULL MUTATION

The constant region modifications had no effect on the potency or PAD2/PAD4 affinities of the bispecific molecules. The Triple Mutation (TM) (L234F/L235E/P331S) abolishes Fc effector function (Table 93). The YTE mutation (M252Y/S254T/T256E) increases the half-life of antibodies. The YTE was also shown to partly rescue the Fc affinity for huFcRn in the context of a TM mutation. FQQ (L234F /L235Q/K322Q) is an alternative effective null mutation to TM.

TABLE 93
Affinity for huFcRn

| | | | | | | | HPLC | |
|---|---|---|---|---|---|---|---|---|
| | | | | | | | retention | Relative |
| Name | Format | Hole | Knob | scFv | Fab | Fc | (min) | retention |
| Clone 09 | Bis3 | — | — | PAD2 | PAD4 | TM-YTE | 23.293 | 0.828 |
| Clone 10 | Bis3 | — | — | PAD4 | PAD2 | TM-YTE | 22.834 | 0.753 |
| Clone 12 | Bis3 | — | — | PAD4 | PAD2 | FQQ-YTE | 22.795 | 0.746 |
| Clone 05 | Duet | — | — | PAD2 | PAD4 | FQQ-YTE | 22.161 | 0.642 |
| Clone 06 | Duet | PAD2 | PAD4 | — | — | FQQ-YTE | 21.943 | 0.606 |
| Clone 03 | Duet | PAD4 | PAD2 | — | — | TM-YTE | 21.768 | 0.577 |
| Clone 04 | Duet | PAD2 | PAD4 | — | — | TM-YTE | 21.702 | 0.566 |
| Clone 07 | Bis3 | — | — | PAD2 | PAD4 | TM | 17.767 | −0.081 |
| Clone 01 | Duet | PAD4 | PAD2 | — | — | TM | 17.107 | −0.19 |
| Clone 08 | Bis3 | — | — | PAD4 | PAD2 | TM | 17.003 | −0.207 |
| Clone 02 | Duet | PAD2 | PAD4 | — | — | TM | 16.933 | −0.219 |

Affinity chromatography (Section 6.7).

### REFERENCES

All publications mentioned in the specification are herein incorporated by reference.
[1] M. E. Weinblatt et al., New England Journal of Medicine 340, 253 (1999).
[2] S. Mpofu, F. Fatima, and R. J. Moots, Rheumatology 44, 271 (2005).
[3] E. H. Choy, A. F. Kavanaugh, and S. A. Jones, Nat Rev Rheumatol 9, 154 (2013).
[4] J. L. Nam, Current Opinion in Rheumatology 28, 267 (2016).
[5] M. H. Pillinger and S. B. Abramson, Rheumatic Disease Clinics of North America 21, 691 (1995).
[6] M. Nagashima et al., Rheumatology International 18, 113 (1998). [6]
[7] N. Thieblemont et al., Semin Immunol 28, 159 (2016).
[8] K. L. Bicker and P. R. Thompson, Biopolymers 99, 155 (2013).
[9] M. Sato et al., WO2012026309A9 (10 May 2012).
[10] C. H. Nielsen and D. Damgaard, WO2014086365A1 (12 Jun. 2014).
[11] C. H. Nielsen and D. Damgaard, WO2016155745A1 (6 Oct. 2016).
[12] Sato et al., WO2016143753A1 (15 Sep. 2016).
[13] M. Aosasa et al., J Immunol Res 2021, U.S. Pat. No. 6,659,960 (2021).
[14] E. A. Kabat, Sequences of Proteins of Immunological Interest (U.S. Department of Health and Human Services, Public Health Service, National Institutes of Health, 1991).
[15] P. Tsui et al., WO2015175874 (A2) (19 Nov. 2015).
[16] W. Dall'acqua, L. S. Johnson, and E. S. Ward, WO2002060919A2 (8 Aug. 2002).
[17] P. Tsui, M. BORROK, and W. Dall'acqua, WO2013165690A1 (7 Nov. 2013).
[18] P. T. Jones et al., Nature 321, 522 (1986).
[19] M. Verhoeyen, C. Milstein, and G. Winter, Science 239, 1534 (1988).
[20] L. Riechmann et al., Nature 332, 323 (1988).
[21] F. RÜCKER, G. Himmler, and G. Wozniak-Knopp, WO2008003103A2 (10 Jan. 2008).
[22] C. Wu, WO2015103072A1 (9 Jul. 2015).
[23] U. Brinkmann and R. E. Kontermann, MAbs 9, 182 (2017).
[24] Y. Mazor et al., MAbs 7, 377 (2015).
[25] V. Faid et al., Eur J Pharm Sci 159, 105730 (2021).
[26] E. A. Kabat and T. T. Wu, Ann N Y Acad Sci 190, 382 (1971).
[27] C. Chothia and A. M. Lesk, J Mol Biol 196, 901 (1987).
[28] S. J. Coales et al., Rapid Commun Mass Spectrom 23, 639 (2009).
[29] V. Oganesyan et al., Acta Cryst D 64, 700 (2008).
[30] M. J. Borrok et al., Journal of Pharmaceutical Sciences 106, 1008 (2017).
[31] P. J. Carter, L. G. Presta, and J. B. Ridgway, U.S. Pat. No. 8,216,805B2 (10 Jul. 2012).
[32] P. J. Carter, L. G. Presta, and J. B. Ridgway, U.S. Pat. No. 5,731,168A (24 Mar. 1998).
[33] G. Chattopadhyay and R. Varadarajan, Protein Sci 28, 1127 (2019).
[34] S. J et al., Biotechnology and bioengineering 112, (2015).
[35] Y. Takizawa et al., Ann Rheum Dis 65, 1013 (2006).
[36] A. Kinloch et al., Arthritis Res Ther 7, R1421 (2005).
[37] V. Tj et al., Nature biotechnology 14, (1996).
[38] M. Miyamoto et al., WO2022176970 (25 Aug. 2022)

1. An antibody comprising a PAD2 binding domain that specifically binds to PAD2 and/or a PAD4 binding domain that specifically binds to PAD4.

2. The antibody of claim 1, wherein the antibody:
(a) inhibits PAD-mediated citrullination of proteins;
(b) inhibits PAD activity in the synovial fluid; and/or
(c) inhibits PAD2 and/or PAD4 in immune cells.

3. (canceled)

4. (canceled)

5. (canceled)

6. (canceled)

7. (canceled)

8. (canceled)

9. (canceled)

10. (canceled)

11. The antibody of claim 1, wherein the PAD2 binding domain does not specifically bind PAD3 and/or does not specifically bind PAD1.

12. The antibody of claim 1, wherein the PAD4 binding domain does not specifically bind PAD3 and/or does not specifically bind PAD1.

13. (canceled)

14. (canceled)

15. (canceled)

16. (canceled)

17. (canceled)

18. (canceled)

19. The antibody of claim 1, wherein:
(a) the PAD2 domain specifically binds mouse PAD2;
(b) the PAD4 domain specifically binds mouse PAD4;
(c) the PAD2 domain specifically binds cynomolgus PAD2; and/or
(d) the PAD4 domain specifically binds cynomolgus PAD4.

20. (canceled)

21. (canceled)

22. (canceled)

23. The antibody of claim 1, wherein the antibody is a bispecific comprising the PAD2 binding domain and the PAD4 binding domain.

24. (canceled)

25. (canceled)

26. (canceled)

27. (canceled)

28. (canceled)

29. The antibody of claim 23, wherein the antibody is a bivalent bispecific.

30. (canceled)

31. (canceled)

32. (canceled)

33. (canceled)

34. (canceled)

35. (canceled)

36. The antibody of claim 29, wherein the antibody is bivalent bispecific comprising:
a) an IgG comprising first and second Fab domains and an Fc domain, wherein the first and second Fab domains each comprise a PAD2 binding domain which specifically binds PAD2, and
b) first and second scFvs, wherein the first and second scFvs are each respectively linked to the carboxy terminal of one of the heavy chains of the Fc domain of the IgG, and wherein the first and second scFvs each comprise a PAD4 binding domain which specifically binds PAD4.

37. (canceled)

38. (canceled)

39. (canceled)

40. (canceled)

41. The antibody of claim 29, wherein the antibody is bivalent bispecific comprising:
a) an IgG comprising first and second Fab domains and an Fc domain, wherein the first and second Fab domains each comprise a PAD4 binding domain that specifically binds PAD4, and
b) first and second scFvs, wherein the first and second scFvs are each respectively linked to the carboxy terminal of one of the heavy chains of the Fc domain of the IgG, and wherein the first and second scFvs each comprise a PAD2 binding domain that specifically binds PAD2.

42. (canceled)

43. (canceled)

44. (canceled)

45. (canceled)

46. (canceled)

47. (canceled)

48. (canceled)

49. (canceled)

50. The antibody of claim 23, wherein the antibody is a monovalent bispecific.

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. The antibody of claim 1, wherein the PAD2 binding domain comprises a variable heavy (VH) domain sequence comprising complementarity determining regions (CDRs) HCDR1, HCDR2 and HCDR3, and a variable light (VL) domain sequence comprising CDRs LCDR1, LCDR2 and LCDR3, and wherein:
a) the HCDR1 amino acid sequence is SEQ ID NO: 3;
b) the HCDR2 amino acid sequence is SEQ ID NO: 4;
c) the HCDR3 amino acid sequence is SEQ ID NO: 5;
d) the LCDR1 amino acid sequence is SEQ ID NO: 10;
e) the LCDR2 amino acid sequence is SEQ ID NO: 11; and/or
f) the LCDR3 amino acid sequence is SEQ ID NO: 12.

67. (canceled)

68. (canceled)

69. (canceled)

70. (canceled)

71. (canceled)

72. (canceled)

73. The antibody of claim 1, wherein the PAD4 binding domain comprises a variable heavy (VH) domain sequence comprising complementarity determining regions (CDRs) HCDR1, HCDR2 and HCDR3, and a variable light (VL) domain sequence comprising CDRs LCDR1, LCDR2 and LCDR3, and wherein:
a) the HCDR1 amino acid sequence is SEQ ID NO: 17,
b) the HCDR2 amino acid sequence is SEQ ID NO: 18,
c) the HCDR3 amino acid sequence is SEQ ID NO: 19,
d) the LCDR1 amino acid sequence is SEQ ID NO: 24,
e) the LCDR2 amino acid sequence is SEQ ID NO: 25, and/or
f) the LCDR3 amino acid sequence is SEQ ID NO: 26.

74. (canceled)

75. (canceled)

76. (canceled)

77. (canceled)

78. (canceled)

79. (canceled)

80. The antibody of claim 1, wherein the antibody comprises an Fc domain.

81. (canceled)

82. (canceled)

83. (canceled)

84. (canceled)

85. (canceled)

86. The antibody of claim 80, wherein the Fc domain has null effector function and comprises at least one half life extension conferring mutation.

87. (canceled)

88. (canceled)

89. (canceled)

90. (canceled)

91. (canceled)

92. (canceled)

93. (canceled)

94. (canceled)

95. (canceled)

96. (canceled)

97. A polypeptide comprising the antibody of claim 1.

98. A nucleic acid encoding one or more chains of an antibody as defined in claim 1.

99. (canceled)

100. A vector comprising the nucleic acid of claim 98.

101. A host cell comprising the vector of claim 100.

102. A pharmaceutical composition comprising an antibody according to claim 1 and a pharmaceutically acceptable carrier.

103. A kit comprising an antibody according to claim 1.

104. A method of treating a disease in a subject comprising administering the antibody of claim 1 to the subject.

105. (canceled)

106. (canceled)

107. (canceled)

108. The method of claim 104, wherein the disease is an autoimmune disorder.

109. (canceled)

110. (canceled)

111. (canceled)

112. (canceled)
